(19) United States
(12) Patent Application Publication
(10) Pub. No.: US 2026/0234136 A1
(43) Pub. Date: Aug. 13, 2026

(54) ISOXAZOLIDINES AS RIPK1 INHIBITORS AND USE THEREOF

(71) Applicant: GENZYME CORPORATION, Cambridge, MA (US)

(72) Inventors: Elisabeth DEFOSSA, Frankfurt am Main (DE); Nils RACKELMANN, Frankfurt am Main (DE); Uwe HEINELT, Frankfurt am Main (DE); Hans MATTER, Frankfurt am Main (DE); Maria MENDEZ-PEREZ, Frankfurt am Main (DE); Kurt RITTER, Frankfurt am Main (DE); Hauke SZILLAT, Frankfurt am Main (DE); Gernot ZECH, Frankfurt am Main (DE)

(21) Appl. No.: 19/383,239

(22) Filed: Nov. 7, 2025

Related U.S. Application Data
(63) Continuation of application No. PCT/US2024/028142, filed on May 7, 2024.

(30) Foreign Application Priority Data
May 10, 2023 (EP) 23172644.9

(51) Int. Cl. C07D 413/14; A61K 31/506; A61K 31/53; C07D 417/14

(52) U.S. Cl. CPC C07D 413/14; A61K 31/506; A61K 31/53; C07D 417/14

(57) ABSTRACT

The disclosure relates to a compound of formula (I)

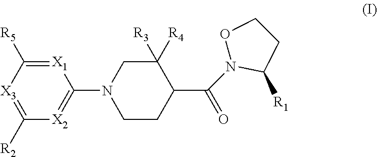

wherein X₁, X₂, and X₃ are independently selected from CR₆ or N; R₁ represents a 5 or 6-membered heteroaryl group, wherein said heteroaryl is optionally substituted; R₂ represents a 4-, 5- or 6-membered heteroaryl in which 1 to 3 ring atoms are independently selected from nitrogen, oxygen or sulfur, a 5 or 6-membered heterocycle in which 1 to 3 ring atoms are independently selected from nitrogen and oxygen or a spiro(C₇-C₁₀)heterobicyclic; R₃ represents H or a (C₁-C₄)alkyl group; R₄ represents H or a (C₁-C₄)alkyl group; or alternatively R₃ and R₄ together form with the carbon atom to which they are attached a cyclopropyl or a cyclobutyl ring; R₅ and R₆ independently represents H, a (C₁-C₆)alkyl group or a halogen; and/or a pharmaceutically acceptable salt, solvate, or stereoisomer thereof.

It further relates to the pharmaceutical compositions containing said new compounds.

### RELATED APPLICATIONS

This application is a continuation of International Patent Application No. PCT/US2024/028142, filed May 7, 2024, which claims priority to European Patent Application No. 23172644.9, filed May 10, 2023, the entire disclosures of which are hereby incorporated herein by reference.

### TECHNICAL FIELD

The disclosure relates to new isoxazolidine derivatives useful as a medicament. Said new compounds are in particular useful as kinase inhibitors, and even particularly as RIPK1 inhibitors. They are efficient for treating and/or preventing acute and chronic neurodegenerative diseases like Parkinson's disease, Alzheimer's disease, amyotrophic lateral sclerosis (ALS) or multiple sclerosis (MS).

The disclosure further relates to the pharmaceutical compositions containing said new compounds.

### TECHNICAL BACKGROUND

Although inflammation can be a protective mechanism in response to harmful stimuli such as invasion of pathogens and tissue damages, chronic inflammation is an important underlying factor in many human diseases such as neurodegeneration, rheumatoid arthritis, autoimmune and inflammatory diseases, and cancer. Similarly, the activation of cell death pathways, such as necrosis and apoptosis which are useful in eliminating infected or damaged cells, is also an important underlying mechanism for human diseases, including acute and chronic neurodegenerative diseases. Receptor-interacting protein kinase 1 (UniProtKB Q13546) is a key regulator of inflammation, apoptosis and necroptosis. Receptor-interacting protein kinase 1 has an important role in modulating inflammatory responses mediated by nuclear-factor kappa-light chain enhancer of activated B cells (NF-κB). More recent research has shown that its kinase activity controls necroptosis, a form of necrotic cell death, which was traditionally thought to be passive and unregulated, and is characterized by a unique morphology. Further, receptor-interacting protein kinase 1 is part of a pro-apoptotic complex indicating its activity in regulating apoptosis.

The receptor-interacting protein kinase 1 is subject to complex and intricate regulatory mechanisms, including ubiquitylation, deubiquitylation, and phosphorylation. These regulatory events collectively determine whether a cell will survive and activate an inflammatory response or die through apoptosis or necroptosis. Dysregulation of receptor-interacting protein kinase 1 signalling can lead to excessive inflammation or cell death, and conversely, research has shown that inhibition of receptor-interacting protein kinase 1 can be effective therapies for diseases involving inflammation or cell death.

RIPK1 inhibition has been identified as a promising principle to address different diseases like rheumatoid arthritis (RA), psoriasis, multiple sclerosis, Alzheimer's disease, inflammatory bowel disease such as Crohn's disease, amyotrophic lateral sclerosis (ALS) or ulcerative colitis (UC). To treat some of these diseases like multiple sclerosis (MS) and Alzheimer's disease, access to the central nervous system (CNS) is required, while for other diseases like rheumatoid arthritis, psoriasis, inflammatory bowel disease (IBD) such as Crohn's disease or UC access to the CNS is not essentially required.

Different RIPK1 inhibitors were already described, for example in patent applications WO 2014/125444, WO 2016/185423 or WO 2016/027253 (GSK).

The RIPK1 inhibitor GSK2982772 (oxazepinone derivative disclosed in WO 2014/125444), was evaluated for RA, psoriasis and UC in phase II clinical trials.

Dihydropyrazole compounds with phenyl substituent on dihydropyrazole and a pyrimidine-piperidine element are disclosed as RIPK1 inhibitors by GSK in WO 2018/092089. Other Dihydropyrazole compounds as RIPK1 inhibitors are disclosed in WO2020224656.

Isoxazolidine compounds with phenyl substituent on isoxazolidine and a pyrimidine-piperidine element are disclosed as RIPK1 inhibitors by GSK in WO 2019/130230. Similar isoxazolidine compounds are disclosed in KR 2020-087922 (Voronoi) and in WO 2020/043173.

Isoxazolidine compounds with a reduced ability to cross the blood-brain-barrier as RIPK1 inhibitors are disclosed in WO 2021/245070.

Compounds with a cycloalkyl element as RIPK1 inhibitor are disclosed in WO 2022194259.

### SUMMARY

According to one of its objects, the present disclosure relates to a compound of formula (I):

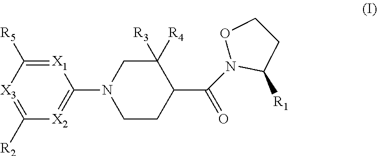

wherein
X₁, X₂, and X₃ are independently selected from CR₆ or N;
R₁ represents a 5 or 6-membered heteroaryl group, wherein said heteroaryl is optionally substituted by 1 or 2 groups independently selected from: a halogen a (C₁-C₆)alkyl group and a —CN group;
R₂ represents
   a 4-, 5- or 6-membered heteroaryl in which 1 to 3 ring atoms are independently selected from nitrogen, oxygen or sulfur, and wherein said heteroaryl is optionally substituted by 1 or 2 R₇;
   a 5 or 6-membered heterocycle in which 1 to 3 ring atoms are independently selected from nitrogen and oxygen, and wherein the heterocycle is optionally substituted by 1, 2 or 3 R₃; or
   a spiro(C₇-C₁₀)heterobicyclic in which 1 or 2 ring atoms are selected from nitrogen or oxygen, and wherein the heterocycle is optionally substituted by 1 or 2 R₉;
R₃ represents H or a (C₁-C₄)alkyl group;
R₄ represents H or a (C₁-C₄)alkyl group;
or alternatively R₃ and R₄ together form with the carbon atom to which they are attached a cyclopropyl or a cyclobutyl ring;
R₅ and R₆ independently represents H, a (C₁-C₆)alkyl group or a halogen;
each R₇ independently represents a (C₁-C₆)alkyl group, a halogen, —NH₂ or —OH;
each R₉ independently represents a (C₁-C₆)alkyl group, a halogen or oxo; and
each R₈ independently represents OH, oxo, a halogen, a (C₁-C₆)alkyl, O—(C₁-C₆)alkyl, —NH₂, —NH(C₁-C₆)alkyl, —N[(C₁-C₆)alkyl]₂ or a (C₁-C₆)fluoroalkyl;
or a pharmaceutically acceptable salt, solvate, or stereoisomer thereof.

In a related aspect, provided herein pharmaceutical composition comprising a compound of formula (I) or a pharmaceutically acceptable salt, solvate, or stereoisomer thereof, and a pharmaceutically acceptable excipient.

In another aspect provided herein is a process for manufacturing a compound of formula (I) and intermediates thereof.

In another aspect, is provided herein a compound of formula (I) or a pharmaceutically acceptable salt, solvate, or stereoisomer thereof, for use as medicament.

In another aspect, is provided herein a compound of formula (I) or a pharmaceutically acceptable salt, solvate, or stereoisomer thereof, for use in the treatment and/or prevention of a disease, disorder or condition that is at least partly mediated by receptor-interacting protein kinase 1.

In another aspect, is provided herein a compound of formula (I), or a pharmaceutically acceptable salt, solvate, or stereoisomer thereof, for use in the treatment and/or prevention of a disease selected from Parkinson's disease, Alzheimer's disease, amyotrophic lateral sclerosis (ALS) or multiple sclerosis (MS).

In another aspect, provided herein is a method of inhibiting receptor-interacting protein kinase 1. Further provided are methods for treating a disease, disorder or condition that is at least partly mediated by receptor-interacting protein kinase 1, comprising administering a therapeutically effective amount of a compound of formula (I) or a pharmaceutical composition as described herein to a subject in need thereof. The disclosure also provides uses of the compounds of formula (I) or compositions thereof in the manufacture of a medicament for the treatment of a disease, disorder or condition that is at least partly mediated by receptor-interacting protein kinase 1.

It has been furthermore observed that the compounds of formula (I) of the present disclosure exhibit developability properties of great interest. In particular solubility of the compounds of formula (I) according to the present disclosure has been assessed and considered convenient according to the pharmacopeial standards.

### DETAILED DESCRIPTION

### Definitions

In this specification the term “alkyl” refers to straight or branched, saturated, aliphatic hydrocarbon groups having the number of atoms indicated. More particularly, a (Cr-Cy) alkyl group, where x and y are integers, x<y, is a linear or branched saturated aliphatic group comprising from x to y carbon atoms. For example (C₁-C₄)alkyl includes, but is not limited to, methyl, ethyl, n-propyl, isopropyl, n-butyl, isobutyl, sec-butyl and tert-butyl groups, and the like.

“Cycloalkyl” refers to saturated or partially unsaturated, optionally substituted, cyclic hydrocarbon groups having the number of atoms indicated. More particularly, a (C₃—Cz)-cycloalkyl group, where z is an integer greater than or equal to 4, comprises from 3 to z carbon atoms. For example, (C₃-C₃)-cycloalkyl groups contain from 3 to 8 carbon atoms and are for example cyclopropyl, cyclobutyl, cyclopentyl, cyclohexyl, cycloheptyl or cyclooctyl.

The term “halogen” refers to a chlorine, fluorine, bromine, or iodine atom, and in particular denotes a chlorine or fluorine atom.

“fluoroalkyl” refers to an alkyl group as previously defined where the alkyl group is substituted with at least one fluorine atom. In other terms, at least one hydrogen atom of the alkyl group is replaced by a fluorine atom. By way of example, mention may be made of CH2F, CHF2, CH2CHF2, —CH2CH2F and the like. When all the hydrogen atoms belonging to the alkyl group are replaced by fluorine atoms, the fluoroalkyl group can be named perfluoroalkyl group. By way of example, mention may be made of trifluoromethyl group or trifluoroethyl group and the like.

The term “heterocycloalkyl” refers to a 4 to 7-membered cycloalkyl group, in particular a 4 to 6 membered cycloalkyl group, saturated or partially unsaturated, comprising 1 to 2 heteroatoms independently selected from oxygen, nitrogen and sulfur, in particular being oxygen or nitrogen. By way of examples, mention may be made of, but not limited to: morpholinyl, piperazinyl, piperidinyl, pyrrolidinyl, aziridinyl, oxanyl, oxetanyl, tetrahydropyranyl, morpholinyl, tetrahydrofuranyl, oxepanyl, diazepanyl, dioxanyl, dihydropyranyl, tetrahydropyranyl, and tetrahydrothiopyranyl. The heterocycloalkyl is advantageously tetrahydrofuranyl or tetrahydropyranyl.

The term “heteroaryl” group refers to a cyclic 5 to 6-membered aromatic group containing between 2 and 5 carbon atoms and containing between 1 and 3 heteroatoms, such as nitrogen, oxygen or sulfur. Such nitrogen atom may be substituted with an oxygen atom in order to form a —N—O bond. Such —N—O bond can be in a form of a N-oxide (N+-O—). Said heteroaryl group is monocyclic in the framework of the present disclosure. By way of examples of heteroaryl groups, mention may be made of, but not limited to: thiophenyl, furanyl, thiadiazolyl, thiazolyl, imidazolyl, pyridazinyl, triazinyl, pyrazinyl, oxadiazolyl, pyrazolyl, isothiazolyl, oxazolyl, isoxazolyl, pyridinyl, pyrimidinyl, pyridonyl groups and the like. The heteroaryl group is advantageously pyridinyl, pyrazinyl, pyrimidinyl and thiazolyl.

The term “heterocycloalkyl” group refers to a 4 to 6-membered cycloalkyl group, saturated or partially unsaturated, comprising 1 to 2 heteroatoms independently selected from oxygen, nitrogen and sulfur, in particular being oxygen or nitrogen. By way of examples, mention may be made of, but not limited to: morpholinyl, piperazinyl, piperidinyl, pyrrolidinyl, oxazolidinyl, isoxazolidinyl, aziridinyl, oxanyl, oxetanyl, tetrahydropyranyl, morpholinyl, tetrahydrofuranyl, oxepanyl, diazepanyl, dioxanyl, dihydropyranyl, tetrahydropyranyl, and tetrahydrothiopyranyl. The heterocycloalkyl is advantageously pyrrolidinyl, oxazolidinyl, groups and the like.

The term “spiro(C₇-C₁₀)heterobicyclic” ring refers to two rings connected through a defining single common atom comprising 7 to 10 carbon atoms, wherein 1 to 3 carbon atoms of the rings are replaced by heteroatom(s) such as oxygen, nitrogen or sulphur, and more particularly such as a nitrogen atom and oxygen atom. By way of examples, mention may be made of, but not limited to: 7-azaspiro[3.5]nonanyl, 7-oxa-2-azaspiro[3.5]nonan-2-yl and 2-oxa-7-azaspiro[3.5]nonan-7-yl, 2-oxa-6-azaspiro[3.3]heptan-yl; 1-oxa-6-azaspiro[3.3]heptan-yl; 6,6-difluoro-2-azaspiro[3.3]heptanyl; 1-oxa-6-azaspiro[3.3]heptan-yl; 7,7-difluoro-2-azaspiro[3.3]heptanyl; 6,6-difluoro-2-azaspiro[3.3]heptan-3-onyl; 7,7-difluoro-2-azaspiro[3.3]heptan-3-on-yl; 2-oxa-6-azaspiro[3.3]heptan-7-on-yl; 5-oxa-2-azaspiro[3.4]octany-yl 5-oxa-2-azaspiro[3.4]octan-3-on-yl; 6-oxa-2-azaspiro[3.4]octanyl, 5-azaspiro[2.5]octan-4-on-yl and a 5-azaspiro[2.4]heptan-4-on-yl group. The spiro(C₇-C₁₀)heterobicyclic ring is advantageously 7-oxa-2-azaspiro[3.5]nonan-2-yl or 2-oxa-7-azaspiro[3.5]nonan-7-yl.

This specification may also make use of several composite terms to describe groups comprising more than one functionality. Such terms will be understood by a person skilled in the art. For example, heterocyclylC₁-C₄alkyl comprises C₁-C₄alkyl substituted by heterocyclyl.

The term “optionally substituted” refers to either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

It is understood that where there are multiple substituents, the substituents chosen may be the same or different.

Where numerical ranges are given, it is understood that the ranges are inclusive of the endpoints.

The phrase “compound of the disclosure” means those compounds which are disclosed herein, both generically and specifically.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response or other problem or complication, commensurate with a reasonable benefit/risk ratio.

The term “pharmaceutically acceptable salts” in this respect refers to the relatively non-toxic, inorganic and organic acid addition salts of compounds of the present disclosure. These salts can be prepared in situ in the administration vehicle or the dosage form manufacturing process or by separately reacting a purified compound of the disclosure in its free base form with a suitable organic or inorganic acid and isolating the salt thus formed during subsequent purification.

As used herein, the term “pharmaceutically acceptable excipient” refers to a substance that aids the administration of an active agent to a subject. By “pharmaceutically acceptable”, it is meant that the excipient is compatible with the other ingredients of the formulation and is not deleterious to the recipient thereof. Pharmaceutical excipients useful in the present disclosure include, but are not limited to, binders, fillers, disintegrants, lubricants, glidants, coatings, sweeteners, flavors and colors.

The phrase “pharmaceutically acceptable carrier” as used herein means a pharmaceutically acceptable material, composition or vehicle, such as a liquid or solid filler, diluent, excipient or solvent encapsulating material, involved in carrying or transporting the subject compound from one organ or portion of the body, to another organ or portion of the body.

“It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder, or condition developing in a human that may be afflicted with or predisposed to the state, disorder, or condition but does not yet experience or display clinical or subclinical symptoms of the state, disorder, or condition, (2) inhibiting the state, disorder, or condition, i.e., arresting, reducing, or delaying the development of the disease or a relapse thereof (in case of maintenance treatment) or at least one clinical or subclinical symptom thereof, or (3) relieving or attenuating the disease, i.e., causing regression of the state, disorder, or condition or at least one of its clinical or subclinical symptoms.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to a human, that has been or will be the object of treatment, observation, or experiment. The methods described herein may be useful in human therapy.

A “therapeutically effective amount” means the amount of a compound that, when administered to a mammal for treating a disease, is sufficient to effect such treatment for the disease. The “therapeutically effective amount” will vary depending on the compound, the disease and its severity, and the age, weight, etc., of the mammal to be treated, which can readily be determined by one of ordinary skill in the art.

As used herein chemical nomenclature as not defined otherwise have the meanings as being used in the technical field.

### Compounds

Disclosed herein is a compound of formula (I):

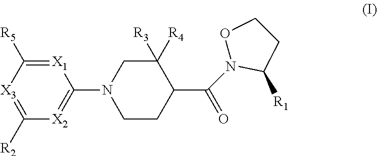

wherein
X₁, X₂, and X₃ are independently selected from CR₆ or N;
R₁ represents a 5 or 6-membered heteroaryl group, wherein said heteroaryl is optionally substituted by 1 or 2 groups independently selected from: a halogen a (C₁-C₆)alkyl group and a —CN group;
R₂ represents
   a 5- or 6-membered heteroaryl in which 1 to 3 ring atoms are independently selected from nitrogen, oxygen or sulfur, and wherein said heteroaryl is optionally substituted by 1 or 2 R₇;
   a 5 or 6-membered heterocycle in which 1 to 3 ring atoms are independently selected from nitrogen and oxygen, and wherein the heterocycle is optionally substituted by 1 or 2 R₈; or
   a spiro(C₇-C₁₀)heterobicyclic in which 1 or 2 ring atoms are selected from nitrogen or oxygen, and wherein the heterocycle is optionally substituted by 1 or 2 R₉;
R₃ represents H or a (C₁-C₄)alkyl group;
R₄ represents H or a (C₁-C₄)alkyl group;
or alternatively R₃ and R₄ together form with the carbon atom to which they are attached a cyclopropyl or a cyclobutyl ring;
R₅ and R₆ independently represents H, a (C₁-C₆)alkyl group or a halogen;
each R₇ and R₉ independently represents a (C₁-C₆)alkyl group or a halogen;
each R₈ independently represents OH, oxo, a halogen, a (C₁-C₆)alkyl, O—(C₁-C₆)alkyl, —NH₂, —NH(C₁-C₆)alkyl, —N[(C₁-C₆)alkyl]₂ or a (C₁-C₆)fluoroalkyl;
or a pharmaceutically acceptable salt, solvate, or stereoisomer thereof.

Disclosed herein is a compound of formula (I):

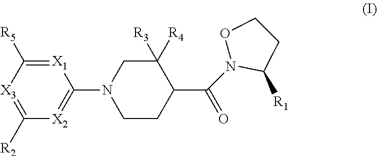

wherein
X₁, X₂, and X₃ are independently selected from CR₆ or N;
R₁ represents a 5 or 6-membered heteroaryl group, wherein said heteroaryl is optionally substituted by 1 or 2 groups independently selected from: a halogen a (C₁-C₆)alkyl group and a —CN group;
R₂ represents
   a 4-, 5- or 6-membered heteroaryl in which 1 to 3 ring atoms are independently selected from nitrogen, oxygen or sulfur, and wherein said heteroaryl is optionally substituted by 1 or 2 R₇;
   a 5 or 6-membered heterocycle in which 1 to 3 ring atoms are independently selected from nitrogen and oxygen, and wherein the heterocycle is optionally substituted by 1, 2 or 3 R₈; or
   a spiro(C₇-C₁₀)heterobicyclic in which 1 or 2 ring atoms are selected from nitrogen or oxygen, and wherein the heterocycle is optionally substituted by 1 or 2 R₉;
R₃ represents H or a (C₁-C₄)alkyl group;
R₄ represents H or a (C₁-C₄)alkyl group;
or alternatively R₃ and R₄ together form with the carbon atom to which they are attached a cyclopropyl or a cyclobutyl ring;
R₅ and R₆ independently represents H, a (C₁-C₆)alkyl group or a halogen;
each R₇ independently represents a (C₁-C₆)alkyl group, a halogen, —NH₂ or —OH;
each R₉ independently represents a (C₁-C₆)alkyl group, a halogen or an oxo; and
each R₃ independently represents OH, an oxo, a halogen, a (C₁-C₆)alkyl, O—(C₁-C₆)alkyl, —NH₂, —NH(C₁-C₆)alkyl, —N[(C₁-C₆)alkyl]₂ or a (C₁-C₆)fluoroalkyl;
or a pharmaceutically acceptable salt, solvate, or stereoisomer thereof.

One embodiment disclosed herein is a compound of formula (I):
wherein
X₁, X₂, and X₃ are independently selected from CR₆ or N;
R₁ represents a 5 or 6-membered heteroaryl group, wherein said heteroaryl is optionally substituted by 1 or 2 groups independently selected from: a halogen a (C₁-C₆)alkyl group and a —CN group;
R₂ represents
   a 5- or 6-membered heteroaryl in which 1 to 3 ring atoms are independently selected from nitrogen, oxygen or sulfur, and wherein said heteroaryl is optionally substituted by 1 or 2 R₇;
   a 5 or 6-membered heterocycle in which 1 to 3 ring atoms are independently selected from nitrogen and oxygen, and wherein the heterocycle is optionally substituted by 1, 2 or 3 R₃; or
   a spiro(C₇-C₁₀)heterobicyclic in which 1 or 2 ring atoms are selected from nitrogen or oxygen, and wherein the heterocycle is optionally substituted by 1 or 2 R₉;
R₃ represents H or a (C₁-C₄)alkyl group;
R₄ represents H or a (C₁-C₄)alkyl group;
or alternatively R₃ and R₄ together form with the carbon atom to which they are attached a cyclopropyl or a cyclobutyl ring;
R₅ and R₆ independently represents H, a (C₁-C₆)alkyl group or a halogen;
each R₇ and R₉ independently represents a (C₁-C₆)alkyl group or a halogen;
each R₈ independently represents OH, oxo, a halogen, a (C₁-C₆)alkyl, O—(C₁-C₆)alkyl, —NH₂, —NH(C₁-C₆)alkyl, —N[(C₁-C₆)alkyl]₂ or a (C₁-C₆)fluoroalkyl;
or a pharmaceutically acceptable salt, solvate, or stereoisomer thereof.

According to one embodiment of a compound of formula (I),
X₁, X₂, and X₃ are N; or
X₁ and X₂ are N and X₃ is CR₆; or
X₂ and X₃ are N and X₁ is CR₆; or
X₁ and X₃ are N and X₂ is CR₆.

According to one embodiment of a compound of formula (I),
X₁, X₂, and X₃ are N.

According to another embodiment of a compound of formula (I),
X₁ and X₂ are N and X₃ is CR₆; R₆ is H or fluorine.

According to another embodiment of a compound of formula (I),
X₂ and X₃ are N and X₁ is CR₆: R₆ is H or fluorine.

According to another embodiment of a compound of formula (I),
X₁ and X₃ are N and X₂ is CR₆; R₆ is H or fluorine.

According to one embodiment, R₁ in formula (I) is a pyridinyl, a pyrazinyl, a pyrimidinyl, an oxazolyl or a thiazolyl group, optionally substituted by 1 or 2 groups independently selected from: a halogen, a (C₁-C₂)alkyl group and a —CN group; or a pharmaceutically acceptable salt, solvate or stereoisomer thereof.

According to another embodiment, R₂ in formula (I) is an imidazolyl, a pyrazolyl, a triazolyl, an oxazolyl, a thiazolyl, a pyridinyl or a pyrimidinyl group, optionally substituted by 1 or 2 R₇, said R₇ being independently selected from: a CH₃ group and a halogen, in particular being a CH₃ group; or a pharmaceutically acceptable salt, solvate, or stereoisomer thereof.

According to one embodiment, R₂ in formula (I) is a piperidinyl, a morpholinyl, a pyrrolidinyl, a oxazolidinyl group, an azetidinyl or an oxetanyl, optionally substituted by 1, 2 or 3 R₃, said R₃ being independently selected from: OH, oxo, a halogen, a (C₁-C₂)alkyl group, a O—(C₁-C₂)alkyl group, and CF₃, in particular optionally substituted by a oxo, or a pharmaceutically acceptable salt, solvate, or stereoisomer thereof.

According to one embodiment, R₂ in formula (I) is a piperidinyl, a morpholinyl, a pyrrolidinyl, a oxazolidinyl group, an azetidinyl or an oxetanyl, optionally substituted by 1 or 2 R₃, said R₃ being independently selected from: OH, oxo, a halogen, a (C₁-C₂)alkyl group and CF₃, in particular optionally substituted by a oxo, or a pharmaceutically acceptable salt, solvate, or stereoisomer thereof.

According to one embodiment, R₂ in formula (I) R₂ is a 5-azaspiro[2.5]octan-4-on-yl group, a 5-azaspiro[2.4]heptan-4-on-yl group, 7-azaspiro[3.5]nonanyl, 7-oxa-2-azaspiro[3.5]nonan-2-yl and 2-oxa-7-azaspiro[3.5]nonan-7-yl, 2-oxa-6-azaspiro[3.3]heptan-yl; 1-oxa-6-azaspiro[3.3]heptan-yl; 6,6-difluoro-2-azaspiro[3.3]heptanyl; 1-oxa-6-azaspiro[3.3]heptan-yl; 7,7-difluoro-2-azaspiro[3.3]heptanyl; 6,6-difluoro-2-azaspiro[3.3]heptan-3-onyl; 7,7-difluoro-2-azaspiro[3.3]heptan-3-on-yl; 2-oxa-6-azaspiro[3.3]heptan-7-on-yl; 5-oxa-2-azaspiro[3.4]octany-yl and 5-oxa-2-azaspiro[3.4]octan-3-on-yl; or a 6-oxa-2-azaspiro[3.4]octanyl, and is optionally substituted by 1 or 2 R₉; or a pharmaceutically acceptable salt, solvate, or stereoisomer thereof.

According to one embodiment, R₂ in formula (I) R₂ is a 7-oxa-2-azaspiro[3.5]nonan-2-yl group, a 5-azaspiro[2.5]octan-4-on-yl group, a 5-azaspiro[2.4]heptan-4-on-yl group or a 2-oxa-7-azaspiro[3.5]nonan-7-yl group, in particular a 5-azaspiro[2.5]octan-4-on-yl group, a 5-azaspiro[2.4]heptan-4-on-yl group, and is optionally substituted by 1 or 2 R₉ selected from methyl or fluorine.

According to another embodiment, R₃ is H or a (C₁-C₂)alkyl group; R₄ represents H, or a (C₁-C₂)alkyl group; or alternatively R₃ and R₄ together form with the carbon atom to which they are attached a cyclopropyl ring.

According to another embodiment of a compound of formula (I), R₃ is H and R₄ is H.

According to another embodiment of a compound of formula (I), R₃ is a methyl group; R₄ is a methyl group.

According to another embodiment of a compound of formula (I), R₃ and R₄ together form with the carbon atom to which they are attached a cyclopropyl ring.

According to another embodiment of a compound of formula (I), R₅ is H, methyl or fluorine.

According to another embodiment of a compound of formula (I), R₅ is H.

According to another embodiment of a compound of formula (I), R₅ fluorine.

According to another embodiment of a compound of formula (I), each R₇ and R₉ independently represents a methyl group, fluorine or chlorine.

According to another embodiment of a compound of formula (I), each R₃ independently represents OH, oxo, a halogen, a methyl group, O—CH₃, —NH₂, —NH—CH₃, —N[—CH₃]₂ or a CF₃.

Herein is further provided a compound of formula (I):

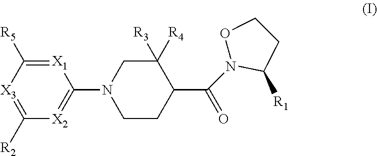

wherein
X₁ represents N;
X₂ and X₃ are as described above;
R₁ represents a pyridinyl, a pyrazinyl, an oxazolyl, or a thiazolyl group, optionally substituted by 1 or 2 groups independently selected from: a fluorine atom and a methyl;
R₂ represents
   a 5-membered heteroaryl in which 2 or 3 ring atoms are independently selected from nitrogen, oxygen or sulfur, and wherein said heteroaryl is optionally substituted by 1 R₇;
   a 5-membered heterocycle in which 1 or 2 ring atoms are independently selected from nitrogen and oxygen, and wherein the heterocycle is optionally substituted by 1 or 2 R₃; or
   a spiro(C₇-C₈)heterobicyclic in which 1 atom is a nitrogen, and wherein the heterocycle is optionally substituted by 1 R₉;
R₃ and R₄ represents H;
or alternatively R₃ and R₄ together form with the carbon atom to which they are attached a cyclopropyl;
R₅ represents H;
R₇ represent a methyl;
R₉ represents an oxo; and
each R₈ independently represents an oxo or a methyl;
or a pharmaceutically acceptable salt, solvate, or stereoisomer thereof.

Herein is further provided a compound of formula (I):

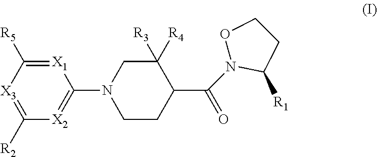

wherein:
X₁ represents N;
X₂ and X₃ are as described above;
R₁ represents a pyridinyl, a pyrazinyl, or a thiazolyl group, optionally substituted by 1 or 2 groups independently selected from: a fluorine atom and a methyl;
R₂ represents
   a 5-membered heteroaryl in which 2 or 3 ring atoms are independently selected from nitrogen, oxygen or sulfur, and wherein said heteroaryl is optionally substituted by 1 R₇; or
a 5-membered heterocycle in which 1 ring atom is nitrogen, and wherein the heterocycle is optionally substituted by 1 R₈;
R₃ and R₄ represents H;
   or alternatively R₃ and R₄ together form with the carbon atom to which they are attached a cyclopropyl;
R₅ represents H;
R₇ represent a methyl;
R₉ represents an oxo; and
R₈ represents an oxo;
or a pharmaceutically acceptable salt, solvate, or stereoisomer thereof.

The nomenclature of the following compounds (1) to (116) was generated according to the principles of the International Union of Pure and Applied Chemistry. “cis” and “trans” prefixes were also used to assign the relative stereochemistry of two adjacent chiral centers.

According to a preferred embodiment the compound of formula (I) is chosen from:
(1) (S)-(3-(5-fluoropyridin-3-yl)isoxazolidin-2-yl)(1-(6-(pyridin-3-yl)pyrimidin-4-yl)piperidin-4-yl)methanone,
(2) (S)-3-(2-(4-(3-(pyrazin-2-yl)isoxazolidine-2-carbonyl)piperidin-1-yl)pyrimidin-4-yl)oxazolidin-2-one,
(3) (S)-1-(4-(4-(3-(pyrazin-2-yl)isoxazolidine-2-carbonyl)piperidin-1-yl)pyrimidin-2-yl)pyrrolidin-2-one,
(4) (S)-1-(2-(4-(3-(pyrazin-2-yl)isoxazolidine-2-carbonyl)piperidin-1-yl)pyrimidin-4-yl)pyrrolidin-2-one,
(5) 3-[5-fluoro-2-[4-[(3S)-3-pyrazin-2-ylisoxazolidine-2-carbonyl]-1-piperidyl]pyrimidin-4-yl]oxazolidin-2-one,
(6) 1-[5-fluoro-2-[4-[(3S)-3-pyrazin-2-ylisoxazolidine-2-carbonyl]-1-piperidyl]pyrimidin-4-yl]pyrrolidin-2-one,
(7) 1-[6-(2-methylimidazol-1-yl)pyrimidin-4-yl]-4-piperidyl]-[(3S)-3-(2-methylthiazol-4-yl)isoxazolidin-2-yl]methanone,
(8) 1-[4-(2-methylpyrazol-3-yl)-1,3,5-triazin-2-yl]-4-piperidyl]-[(3S)-3-(6-methyl-3-pyridyl)isoxazolidin-2-yl]methanone,
(9) [1-[4-(4-methylpyrazol-1-yl)pyrimidin-2-yl]-4-piperidyl]-[(3S)-3-(6-methyl-3-pyridyl)isoxazolidin-2-yl]methanone,
(10) [1-(5-fluoro-4-pyrazol-1-yl-pyrimidin-2-yl)-4-piperidyl]-[(3S)-3-(6-methyl-3-pyridyl)isoxazolidin-2-yl]methanone,
(11) [1-(4-oxazol-2-ylpyrimidin-2-yl)-4-piperidyl]-[(3S)-3-pyrazin-2-ylisoxazolidin-2-yl]methanone,
(12) [1-(5-fluoro-4-oxazol-2-yl-pyrimidin-2-yl)-4-piperidyl]-[(3S)-3-pyrazin-2-ylisoxazolidin-2-yl]methanone,
(13) [1-(5-fluoro-2-oxazol-2-yl-pyrimidin-4-yl)-4-piperidyl]-[(3S)-3-pyrazin-2-ylisoxazolidin-2-yl]methanone,
(14) [(3S)-3-pyrazin-2-ylisoxazolidin-2-yl]-[1-(4-thiazol-2-yl-1,3,5-triazin-2-yl)-4-piperidyl]methanone,
(15) [1-[4-(2-methylpyrazol-3-yl)pyrimidin-2-yl]-4-piperidyl]-[(3S)-3-(6-methyl-3-pyridyl)isoxazolidin-2-yl]methanone,
(16) [1-[2-(4-methylpyrazol-1-yl)pyrimidin-4-yl]-4-piperidyl]-[(3S)-3-(6-methyl-3-pyridyl)isoxazolidin-2-yl]methanone,
(17) [1-[6-(4-methylpyrazol-1-yl)pyrimidin-4-yl]-4-piperidyl]-[(3S)-3-(6-methyl-3-pyridyl)isoxazolidin-2-yl]methanone,
(18) [1-[6-(2-methylpyrazol-3-yl)pyrimidin-4-yl]-4-piperidyl]-[(3S)-3-(6-methyl-3-pyridyl)isoxazolidin-2-yl]methanone,
(19) [1-[5-fluoro-4-(2-methylpyrazol-3-yl)pyrimidin-2-yl]-4-piperidyl]-[(3S)-3-(6-methyl-3-pyridyl)isoxazolidin-2-yl]methanone,
(20) [(3S)-3-(5-fluoro-6-methyl-3-pyridyl)isoxazolidin-2-yl]-[1-[4-(2-methylimidazol-1-yl)pyrimidin-2-yl]-4-piperidyl]methanone,
(21) [(3S)-3-(5-fluoro-6-methyl-3-pyridyl)isoxazolidin-2-yl]-[1-[4-(2-methylimidazol-1-yl)pyrimidin-2-yl]-4-piperidyl]methanone,
(22) [1-[4-(2,4-dimethylimidazol-1-yl)-5-fluoro-pyrimidin-2-yl]-4-piperidyl]-[(3S)-3-pyrazin-2-ylisoxazolidin-2-yl]methanone,
(23) [1-[4-(2-methylimidazol-1-yl)-1,3,5-triazin-2-yl]-4-piperidyl]-[(3S)-3-(2-methylthiazol-4-yl)isoxazolidin-2-yl]methanone,
(24) [1-[2-(2-methylpyrazol-3-yl)pyrimidin-4-yl]-4-piperidyl]-[(3S)-3-pyrazin-2-ylisoxazolidin-2-yl]methanone,
(25) [1-(5-fluoro-2-pyrimidin-5-yl-pyrimidin-4-yl)-4-piperidyl]-[(3S)-3-pyrazin-2-ylisoxazolidin-2-yl]methanone,
(26) (S)-(1-(4-(2-methyl-1H-imidazol-1-yl)-1,3,5-triazin-2-yl)piperidin-4-yl)(3-(pyrazin-2-yl)isoxazolidin-2-yl)methanone,
(27) (S)-(1-(5-fluoro-4-(thiazol-5-yl)pyrimidin-2-yl)piperidin-4-yl)(3-(pyrazin-2-yl)isoxazolidin-2-yl)methanone,
(28) (S)-(1-(6-(1-methyl-1H-pyrazol-5-yl)pyrimidin-4-yl)piperidin-4-yl)(3-(pyrazin-2-yl)isoxazolidin-2-yl)methanone,
(29) (S)-(1-(2-(2-methyl-1H-imidazol-1-yl)pyrimidin-4-yl)piperidin-4-yl)(3-(pyrazin-2-yl)isoxazolidin-2-yl)methanone,
(30) (S)-(1-(4-(2-methyl-1H-imidazol-1-yl)pyrimidin-2-yl)piperidin-4-yl)(3-(pyrazin-2-yl)isoxazolidin-2-yl)methanone,
(31) (S)-(1-(6-(2-methyl-1H-imidazol-1-yl)pyrimidin-4-yl)piperidin-4-yl)(3-(pyrazin-2-yl)isoxazolidin-2-yl)methanone,
(32) (S)-(1-(5-fluoro-4-(1H-pyrazol-1-yl)pyrimidin-2-yl)piperidin-4-yl)(3-(pyrazin-2-yl)isoxazolidin-2-yl)methanone,
(33) [5-[6-(2-methylimidazol-1-yl)pyrimidin-4-yl]-5-azaspiro[2.5]octan-8-yl]-[(3S)-3-(6-methylpyridin-3-yl)-1,2-oxazolidin-2-yl]methanone,
(34) [5-[6-(2-methylpyrazol-3-yl)pyrimidin-4-yl]-5-azaspiro[2.5]octan-8-yl]-[(3S)-3-(6-methylpyridin-3-yl)-1,2-oxazolidin-2-yl]methanone,
(35) [1-[4-(2-methylimidazol-1-yl)-1,3,5-triazin-2-yl]piperidin-4-yl]-[(3S)-3-(6-methylpyridin-3-yl)-1,2-oxazolidin-2-yl]methanone,
(36) 3-[2-[4-[(3S)-3-(5-methyl-3-pyridyl)isoxazolidine-2-carbonyl]-1-piperidyl]pyrimidin-4-yl]oxazolidin-2-one,
(37) [(3S)-3-(6-methylpyridin-3-yl)-1,2-oxazolidin-2-yl]-[1-[4-(1,3-oxazol-2-yl)pyrimidin-2-yl]piperidin-4-yl]methanone,
(38) [(3S)-3-(6-methylpyridin-3-yl)-1,2-oxazolidin-2-yl]-[1-[4-(1,3-thiazol-2-yl)-1,3,5-triazin-2-yl]piperidin-4-yl]methanone,
(39) [(8R)-5-[6-(2-methylpyrazol-3-yl)pyrimidin-4-yl]-5-azaspiro[2.5]octan-8-yl]-[(3S)-3-pyrazin-2-yl-1,2-oxazolidin-2-yl]methanone,
(40) [(3S)-3-(2-methyl-1,3-thiazol-4-yl)-1,2-oxazolidin-2-yl]-[1-[6-(2-methyl-1,2,4-triazol-3-yl)pyrimidin-4-yl]piperidin-4-yl]methanone,
(41) [(3S)-3-(2-methyl-1,3-thiazol-4-yl)-1,2-oxazolidin-2-yl]-[(8R)-5-[6-(2-methyl-1,2,4-triazol-3-yl)pyrimidin-4-yl]-5-azaspiro[2.5]octan-8-yl]methanone,
(42) [(3S)-3-(2-methyl-1,3-thiazol-4-yl)-1,2-oxazolidin-2-yl]-[(8R)-5-[6-(3-methyltriazol-4-yl)pyrimidin-4-yl]-5-azaspiro[2.5]octan-8-yl]methanone,
(43) [(3S)-3-(5-fluoro-6-methylpyridin-3-yl)-1,2-oxazolidin-2-yl]-[(8R)-5-[6-(2-methyl-1,2,4-triazol-3-yl)pyrimidin-4-yl]-5-azaspiro[2.5]octan-8-yl]methanone,
(44) 1-[2-[(8R)-8-[(3S)-3-(6-methylpyridin-3-yl)-1,2-oxazolidine-2-carbonyl]-5-azaspiro[2.5]octan-5-yl]pyrimidin-4-yl]pyrrolidin-2-one,
(45) [(3S)-3-(5-fluoro-6-methylpyridin-3-yl)-1,2-oxazolidin-2-yl]-[1-[4-(2-methylpyrazol-(3-yl)-1,3,5-triazin-2-yl]piperidin-4-yl]methanone,
(46) 1-[2-[4-[(3S)-3-(5-fluoropyridin-3-yl)-1,2-oxazolidine-2-carbonyl]piperidin-1-yl]pyrimidin-4-yl]pyrrolidin-2-one,
(47) 1-[2-[4-[(3S)-3-pyrazin-2-yl-1,2-oxazolidine-2-carbonyl]piperidin-1-yl]pyrimidin-4-yl]piperidin-2-one,
(48) [(3S)-3-(5-fluoro-6-methylpyridin-3-yl)-1,2-oxazolidin-2-yl]-[(8 R)-5-[4-(2-methylpyrazol-3-yl)-1,3,5-triazin-2-yl]-5-azaspiro[2.5]octan-8-yl]methanone,
(49) [(3S)-3-(5-fluoro-6-methylpyridin-3-yl)-1,2-oxazolidin-2-yl]-[1-[6-(2-methyl-1,2,4-triazol-3-yl)pyrimidin-4-yl]piperidin-4-yl]methanone,
(50) 1-[2-[4-[(3S)-3-(5-methylpyridin-3-yl)-1,2-oxazolidine-2-carbonyl]piperidin-1-yl]pyrimidin-4-yl]pyrrolidin-2-one,
(51) 5-[2-[4-[(3S)-3-(5-methylpyridin-3-yl)-1,2-oxazolidine-2-carbonyl]piperidin-1-yl]pyrimidin-4-yl]-5-azaspiro[2.4]heptan-4-one,
(52) 5-[2-[4-[(3S)-3-(2-methyl-1,3-thiazol-4-yl)-1,2-oxazolidine-2-carbonyl]piperidin-1-yl]pyrimidin-4-yl]-5-azaspiro[2.4]heptan-4-one,
(53) 5-[2-[4-[(3S)-3-(5-fluoropyridin-3-yl)-1,2-oxazolidine-2-carbonyl]piperidin-1-yl]pyrimidin-4-yl]-5-azaspiro[2.4]heptan-4-one,
(54) 5-[2-[4-[(3S)-3-(2-methyl-1,3-oxazol-4-yl)-1,2-oxazolidine-2-carbonyl]piperidin-1-yl]pyrimidin-4-yl]-5-azaspiro[2.4]heptan-4-one,
(55) 1-[2-[4-[(3S)-3-(5-methylpyrazin-2-yl)-1,2-oxazolidine-2-carbonyl]piperidin-1-yl]pyrimidin-4-yl]pyrrolidin-2-one,
(56) 1-[2-[4-[(3S)-3-(5-fluoro-6-methylpyridin-3-yl)-1,2-oxazolidine-2-carbonyl]piperidin-1-yl]pyrimidin-4-yl]pyrrolidin-2-one,
(57) 5-[2-[4-[(3S)-3-pyrazin-2-yl-1,2-oxazolidine-2-carbonyl]piperidin-1-yl]pyrimidin-4-yl]-5-azaspiro[2.5]octan-4-one,
(58) 3,3-difluoro-1-[2-[4-[(3S)-3-pyrazin-2-yl-1,2-oxazolidine-2-carbonyl]piperidin-1-yl]pyrimidin-4-yl]pyrrolidin-2-one,
(59) 3,3-difluoro-1-[2-[4-[(3S)-3-(2-methyl-1,3-thiazol-4-yl)-1,2-oxazolidine-2-carbonyl]piperidin-1-yl]pyrimidin-4-yl]pyrrolidin-2-one,
(60) 1-[2-[4-[(3S)-3-(5-fluoro-6-methylpyridin-3-yl)-1,2-oxazolidine-2-carbonyl]piperidin-1-yl]pyrimidin-4-yl]-3,3-dimethylpyrrolidin-2-one,
(61) 5-[2-[4-[(3S)-3-(5-fluoro-6-methylpyridin-3-yl)-1,2-oxazolidine-2-carbonyl]piperidin-1-yl]pyrimidin-4-yl]-5-azaspiro[2.4]heptan-4-one,
(62) 1-[2-[4-[(3S)-3-(5-fluoropyridin-3-yl)-1,2-oxazolidine-2-carbonyl]piperidin-1-yl]pyrimidin-4-yl]-3-methylimidazolidin-2-one,
(63) 4-[2-[(8R)-8-[(3S)-3-(6-methylpyridin-3-yl)-1,2-oxazolidine-2-carbonyl]-5-azaspiro[2.5]octan-5-yl]pyrimidin-4-yl]morpholin-3-one,
(64) [1-[5-fluoro-4-(2-methylimidazol-1-yl)pyrimidin-2-yl]piperidin-4-yl]-[(3S)-3-(5-fluoropyridin-3-yl)-1,2-oxazolidin-2-yl]methanone,
(65) 3-[2-[4-[(3S)-3-(5-fluoro-6-methylpyridin-3-yl)-1,2-oxazolidine-2-carbonyl]piperidin-1-yl]pyrimidin-4-yl]-1,3-oxazolidin-2-one,
(66) 3,3-difluoro-1-[2-[4-[(3S)-3-(6-methylpyridin-3-yl)-1,2-oxazolidine-2-carbonyl]piperidin-1-yl]pyrimidin-4-yl]piperidin-2-one,
(67) [1-[5-fluoro-2-(1,3-oxazol-2-yl)pyrimidin-4-yl]piperidin-4-yl]-[(3S)-3-(6-methylpyridin-3-yl)-1,2-oxazolidin-2-yl]methanone,
(68) 5-[5-fluoro-2-[4-[(3S)-3-(5-fluoro-6-methylpyridin-3-yl)-1,2-oxazolidine-2-carbonyl]piperidin-1-yl]pyrimidin-4-yl]-5-azaspiro[2.4]heptan-4-one,
(69) [(8R)-5-[4-(2-methylpyrazol-3-yl)-1,3,5-triazin-2-yl]-5-azaspiro[2.5]octan-8-yl]-[(3S)-3-(6-methylpyridin-3-yl)-1,2-oxazolidin-2-yl]methanone,
(70) 5-[2-[4-[(3S)-3-(6-methylpyridin-3-yl)-1,2-oxazolidine-2-carbonyl]piperidin-1-yl]pyrimidin-4-yl]-5-azaspiro[2.4]heptan-4-one,
(71) 1-[4-[4-[(3S)-3-(5-fluoropyridin-3-yl)-1,2-oxazolidine-2-carbonyl]piperidin-1-yl]pyrimidin-2-yl]pyrrolidin-2-one,
(72) 5-[2-[4-[(3S)-3-(5-fluoropyridin-3-yl)-1,2-oxazolidine-2-carbonyl]piperidin-1-yl]pyrimidin-4-yl]-5-azaspiro[2.5]octan-4-one,
(73) 3,3-difluoro-1-[2-[4-[(3S)-3-(6-methylpyridin-3-yl)-1,2-oxazolidine-2-carbonyl]piperidin-1-yl]pyrimidin-4-yl]pyrrolidin-2-one,
(74) 3,3-difluoro-1-[2-[4-[(3S)-3-(5-methylpyridin-3-yl)-1,2-oxazolidine-2-carbonyl]piperidin-1-yl]pyrimidin-4-yl]pyrrolidin-2-one,
(75) 3,3-difluoro-1-[2-[4-[(3S)-3-(5-fluoropyridin-3-yl)-1,2-oxazolidine-2-carbonyl]piperidin-1-yl]pyrimidin-4-yl]pyrrolidin-2-one,
(76) 3,3-difluoro-1-[2-[4-[(3S)-3-(5-methylpyridin-3-yl)-1,2-oxazolidine-2-carbonyl]piperidin-1-yl]pyrimidin-4-yl]piperidin-2-one,
(77) [1-[5-fluoro-4-(2-methylimidazol-1-yl)pyrimidin-2-yl]piperidin-4-yl]-[(3S)-3-(6-methylpyridin-3-yl)-1,2-oxazolidin-2-yl]methanone,
(78) [5-[6-(2-methylimidazol-1-yl)pyrimidin-4-yl]-5-azaspiro[2.5]octan-8-yl]-[(3S)-3-(6-methylpyridin-3-yl)-1,2-oxazolidin-2-yl]methanone,
(79) [5-[6-(2-methylimidazol-1-yl)pyrimidin-4-yl]-5-azaspiro[2.5]octan-8-yl]-[(3S)-3-(6-methylpyridin-3-yl)-1,2-oxazolidin-2-yl]methanone,
(80) [5-[6-(2-methylpyrazol-3-yl)pyrimidin-4-yl]-5-azaspiro[2.5]octan-8-yl]-[(3S)-3-(6-methylpyridin-3-yl)-1,2-oxazolidin-2-yl]methanone,
(81) 1-[2-[4-[(3S)-3-(6-methylpyridin-3-yl)-1,2-oxazolidine-2-carbonyl]piperidin-1-yl]pyrimidin-4-yl]pyrrolidin-2-one,
(82) 1-[6-[4-[(3S)-3-(6-methylpyridin-3-yl)-1,2-oxazolidine-2-carbonyl]piperidin-1-yl]pyrimidin-4-yl]pyrrolidin-2-one,
(83) [(8R)-5-[4-(4-methylpyrazol-1-yl)pyrimidin-2-yl]-5-azaspiro[2.5]octan-8-yl]-[(3 S)-3-pyrazin-2-yl-1,2-oxazolidin-2-yl]methanone,
(84) 3,3-dimethyl-1-[4-[4-[(3S)-3-(6-methylpyridin-3-yl)-1,2-oxazolidine-2-carbonyl]piperidin-1-yl]pyrimidin-2-yl]pyrrolidin-2-one,
(85) 1-[4-[(8R)-8-[(3S)-3-(6-methylpyridin-3-yl)-1,2-oxazolidine-2-carbonyl]-5-azaspiro[2.5]octan-5-yl]pyrimidin-2-yl]pyrrolidin-2-one,
(86) 1-[6-[4-[(3S)-3-(5-fluoropyridin-3-yl)-1,2-oxazolidine-2-carbonyl]piperidin-1-yl]pyrimidin-4-yl]pyrrolidin-2-one,
(87) 1-[4-[4-[(3S)-3-(6-methylpyridin-3-yl)-1,2-oxazolidine-2-carbonyl]piperidin-1-yl]pyrimidin-2-yl]pyrrolidin-2-one,
(88) 5,5-dimethyl-1-[2-[4-[(3S)-3-(6-methylpyridin-3-yl)-1,2-oxazolidine-2-carbonyl]piperidin-1-yl]pyrimidin-4-yl]pyrrolidin-2-one,
(89) 1-[2-[4-[(3S)-3-(5-fluoro-6-methylpyridin-3-yl)-1,2-oxazolidine-2-carbonyl]piperidin-1-yl]pyrimidin-4-yl]-5,5-dimethylpyrrolidin-2-one,
(90) 5-[4-[4-[(3S)-3-(6-methylpyridin-3-yl)-1,2-oxazolidine-2-carbonyl]piperidin-1-yl]pyrimidin-2-yl]-5-azaspiro[2.5]octan-4-one,
(91) 3-[2-[4-[(3 S)-3-(5-fluoropyridin-3-yl)-1,2-oxazolidine-2-carbonyl]piperidin-1-yl]pyrimidin-4-yl]-1,3-oxazolidin-2-one,
(92) 1-[2-[4-[(3S)-3-(2-methyl-1,3-thiazol-4-yl)-1,2-oxazolidine-2-carbonyl]piperidin-1-yl]pyrimidin-4-yl]pyrrolidin-2-one,
(93) (3R)-3-methoxy-1-[2-[4-[(3S)-3-(6-methylpyridin-3-yl)-1,2-oxazolidine-2-carbonyl]piperidin-1-yl]pyrimidin-4-yl]piperidin-2-one,
(94) (3S)-3-methoxy-1-[2-[4-[(3S)-3-(6-methylpyridin-3-yl)-1,2-oxazolidine-2-carbonyl]piperidin-1-yl]pyrimidin-4-yl]piperidin-2-one,
(95) 1-[2-[4-[(3S)-3-(2-methyl-1,3-oxazol-4-yl)-1,2-oxazolidine-2-carbonyl]piperidin-1-yl]pyrimidin-4-yl]pyrrolidin-2-one,
(96) (3R)-1-[2-[4-[(3S)-3-(5-fluoropyridin-3-yl)-1,2-oxazolidine-2-carbonyl]piperidin-1-yl]pyrimidin-4-yl]-3-methoxypiperidin-2-one,
(97) (3S)-1-[2-[4-[(3S)-3-(5-fluoropyridin-3-yl)-1,2-oxazolidine-2-carbonyl]piperidin-1-yl]pyrimidin-4-yl]-3-methoxypiperidin-2-one,
(98) [(3S)-3-(5-fluoro-6-methylpyridin-3-yl)-1,2-oxazolidin-2-yl]-[1-[4-(oxetan-2-yl)pyrimidin-2-yl]piperidin-4-yl]methanone,
(99) [(3S)-3-(5-fluoro-6-methylpyridin-3-yl)-1,2-oxazolidin-2-yl]-[1-[4-(oxetan-2-yl)pyrimidin-2-yl]piperidin-4-yl]methanone,
(100) 1-[2-[(8R)-8-[(3S)-3-pyrazin-2-yl-1,2-oxazolidine-2-carbonyl]-5-azaspiro[2.5]octan-5-yl]pyrimidin-4-yl]pyrrolidin-2-one,
(101) [(3S)-3-(5-fluoro-6-methylpyridin-3-yl)-1,2-oxazolidin-2-yl]-[1-[4-(oxetan-3-yl)pyrimidin-2-yl]piperidin-4-yl]methanone,
(102) [1-[4-(5-amino-2-methylpyrazol-3-yl)pyrimidin-2-yl]piperidin-4-yl]-[(3S)-3-(5-fluoro-6-methylpyridin-3-yl)-1,2-oxazolidin-2-yl]methanone,
(103) 5-[5-fluoro-2-[4-[(3S)-3-(5-methylpyridin-3-yl)-1,2-oxazolidine-2-carbonyl]piperidin-1-yl]pyrimidin-4-yl]-5-azaspiro[2.4]heptan-4-one,
(104) 5-[5-fluoro-2-[4-[(3S)-3-(5-fluoropyridin-3-yl)-1,2-oxazolidine-2-carbonyl]piperidin-1-yl]pyrimidin-4-yl]-5-azaspiro[2.4]heptan-4-one,
(105) 5-[5-fluoro-2-[4-[(3S)-3-(5-methylpyrazin-2-yl)-1,2-oxazolidine-2-carbonyl]piperidin-1-yl]pyrimidin-4-yl]-5-azaspiro[2.4]heptan-4-one,
(106) [1-[4-[(3S)-3-fluoropyrrolidin-1-yl]pyrimidin-2-yl]piperidin-4-yl]-[(3S3-(5-methylpyrazin-2-yl)-1,2-oxazolidin-2-yl]methanone,
(107) [1-[4-[(3R)-3-methoxypyrrolidin-1-yl]pyrimidin-2-yl]piperidin-4-yl]-[(3S)-3-(5-methylpyrazin-2-yl)-1,2-oxazolidin-2-yl]methanone
(108) [1-[4-[(3S)-3-methoxypyrrolidin-1-yl]pyrimidin-2-yl]piperidin-4-yl]-[(3S)-3-(5-methylpyrazin-2-yl)-1,2-oxazolidin-2-yl]methanone,
(109) 5-[5-fluoro-2-[4-[(3S)-3-(6-methylpyridin-3-yl)-1,2-oxazolidine-2-carbonyl]piperidin-1-yl]pyrimidin-4-yl]-5-azaspiro[2.4]heptan-4-one,
(110) 1-[5-fluoro-2-[4-[(3S)-3-(5-methylpyrazin-2-yl)-1,2-oxazolidine-2-carbonyl]piperidin-1-yl]pyrimidin-4-yl]pyrrolidin-2-one,
(111) [1-[4-(4-amino-5-methylpyrazol-1-yl)pyrimidin-2-yl]piperidin-4-yl]-[(3S)-3-(5-fluoro-6-methylpyridin-3-yl)-1,2-oxazolidin-2-yl]methanone,
(112) [1-[4-(4-amino-5-methylpyrazol-1-yl)pyrimidin-2-yl]piperidin-4-yl]-[(3S)-3-(6-methylpyridin-3-yl)-1,2-oxazolidin-2-yl]methanone,
(113) [1-[4-(3-methoxyazetidin-1-yl)pyrimidin-2-yl]piperidin-4-yl]-[(3S)-3-(5-methylpyrazin-2-yl)-1,2-oxazolidin-2-yl]methanone,
(114) 3-[5-fluoro-2-[4-[(3S)-3-(5-fluoropyridin-3-yl)-1,2-oxazolidine-2-carbonyl]piperidin-1-yl]pyrimidin-4-yl]-1,3-oxazolidin-2-one,
(115) 3-[5-fluoro-2-[4-[(3S)-3-(5-fluoro-6-methylpyridin-3-yl)-1,2-oxazolidine-2-carbonyl]piperidin-1-yl]pyrimidin-4-yl]-1,3-oxazolidin-2-one, and
(116) 3-[5-fluoro-2-[4-[(3S)-3-(5-methylpyridin-3-yl)-1,2-oxazolidine-2-carbonyl]piperidin-1-yl]pyrimidin-4-yl]-1,3-oxazolidin-2-one, or a pharmaceutically acceptable salt, solvate, or stereoisomer thereof.

According to an even more preferred embodiment of the present disclosure, the compound of formula (I) is chosen from the group consisting of compounds (1) to (3), (5) to (33) and (35) or a pharmaceutically acceptable salt, solvate, or stereoisomer thereof.

According to an even more preferred embodiment of the present disclosure, the compound of formula (I) is chosen from the group consisting of compounds (5) to (8), (10) to (12), (14), (15), (19), (20), (22), (23), (25) to (27), (29), (30), (32) and (35) or a pharmaceutically acceptable salt, solvate, or stereoisomer thereof.

According to an even more preferred embodiment of the present disclosure, the compound of formula (I) is chosen from the group consisting of compounds (3), (11), (14), (15), (38), (39), (40), (42), (43), (45), (48), (49), (52), (53), (54), (55), (57), (62), (65), (69) and (91) or a pharmaceutically acceptable salt, solvate, or stereoisomer thereof.

According to an even more preferred embodiment of the present disclosure, the compound of formula (I) is chosen from the group consisting of compounds (3), (11), (14), (15), (38), (39) to (40), (42), (43), (45) and (48) or a pharmaceutically acceptable salt, solvate, or stereoisomer thereof.

Some compounds of the disclosure are described with their structure in Table 1, which is merely illustrative and does not limit the scope of the present disclosure.

NMR and LC/MS data in Table 1 were obtained according to methods which are detailed in the experimental part provided for the detailed examples synthesis.

TABLE 1
Structure, and analytical characterization of compounds (1) to (116).
Observed ion type is [M + H]+ and LC/MS mentions the used method.

| | | | tR; | | |
|---|---|---|---|---|---|
| Cpd | | | (λ = | mass | LC/ |
| No | Structure | NMR Description | 220 nm) | m/z | MS |
| 1 | 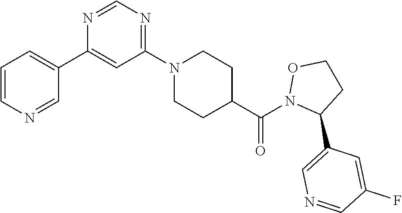 [SMILES: O=C(C1CCN(c2cc(-c3cccnc3)ncn2)CC1)N1OCC[C@H]1c1cncc(F)c1] | ¹H NMR (400 MHz, DMSO-d₆) δ ppm: 9.32 (d, J = 1.9 Hz, 1 H), 8.67 (dd, J = 4.7 Hz, 1.4 Hz, 1 H), 8.59 (s, 1 H), 8.50-8.48 (m, 2 H), 8.41 (s, 1 H), 7.60 (m, 1 H), 7.52 (dd, J = 7.9 Hz, 4.7 Hz, 1 H), 7.44 (s, 1 H), 5.43 (dd, 1 H), 4.56 (m, 1 H), 4.33 (m, 1 H), 3.96 (m, 1 H), ~3.30 (m, 1 H, below water signal), 3.15-3.07 (m, 3 H), 2.93 (m, 1 H), 2.29 (m, 1 H), 1.96 (m, 1 H), 1.78 (m, 1 H), 1.57-1.51 (m, 2 H). | 1.17 | 435.30 | A |
| 2 | 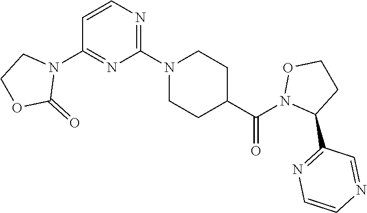 [SMILES: O=C1OCCN1c1ccnc(N2CCC(C(=O)N3OCC[C@H]3c3cnccn3)CC2)n1] | ¹H NMR (400 MHz, DMSO-d₆) δ ppm: 8.62 (dd, J = 2.5 Hz, 1.5 Hz, 1 H), 8.60 (d, J = 1.5 Hz, 1 H), 8.57 (d, J = 2.5 Hz, 1 H), 8.23 (d, J = 5.6 Hz, 1 H), 7.23 (d, J = 5.6 Hz, 1 H), 5.43 (dd, J = 8.8 Hz, 6.2 Hz, 1 H), 4.60 (m, 2 H), 4.44 (t, J = 8.0 Hz, 2 H), 4.34 (m, 1 H), 4.11 (t, J = 8.0 Hz, 2 H), 4.00 (m, 1 H), 3.08-2.95 (m, 3 H), 2.84 (m, 1 H), ~2.52 (m, 1 H, below solvent signal), 1.86 (m, 1 H), 1.74 (m, 1 H), 1.47 (m, 2 H). | 1.18 | 426.20 | A |
| 3 | 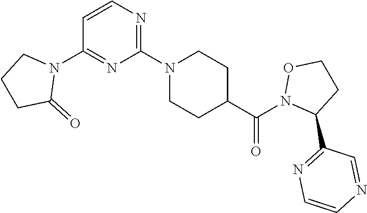 [SMILES: O=C1CCCN1c1ccnc(N2CCC(C(=O)N3OCC[C@H]3c3cnccn3)CC2)n1] | ¹H NMR (400 MHz, DMSO-d₆) δ ppm: 8.62 (bdd, J ~ 2.5 Hz, 1.3 Hz, 1 H), 8.60 (bd, J ~ 1.3 Hz, 1 H), 8.57 (d, J = 2.5 Hz, 1 H), 8.21 (d, J = 5.6 Hz, 1 H), 7.44 (d, J = 5.6 Hz, 1 H), 5.43 (dd, J = 8.8 Hz, 6.2 Hz, 1 H), 4.60 (m, 2 H), 4.34 (m, 1 H), 4.00 (m, 1 H), 3.93 (t, J = 7.1 Hz, 2 H), 3.07-2.93 (m, 3 H), 2.84 (m, 1 H), 2.56 (t, J = 8.1 Hz, 2 H), ~2.50 (m, 1 H, below solvent signal), 2.01 (m, 2 H), 1.86 (m, 1 H), 1.74 (m, 1 H), 1.47 (m, 2 H). | 1.25 | 424.2 | A |
| 4 | 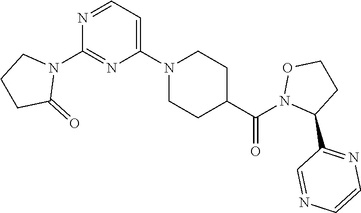 [SMILES: O=C1CCCN1c1nccc(N2CCC(C(=O)N3OCC[C@H]3c3cnccn3)CC2)n1] | ¹H NMR (400 MHz, DMSO-d₆) δ ppm: 8.62 (bdd, J ~ 2.5 Hz, 1.4 Hz, 1 H), 8.60 (bd, J ~ 1.4 Hz, 1 H), 8.57 (d, J = 2.5 Hz, 1 H), 8.08 (d, J = 6.2 Hz, 1 H), 6.57 (d, J = 6.2 Hz, 1 H), 5.43 (dd, J = 8.8 Hz, 6.2 Hz, 1 H), ~4.39 (bm, 2 H), 4.34 (m, 1 H), 4.00 (m, 1 H), 3.88 (t, J = 7.1 Hz, 2 H), 3.10- 2.98 (m, 3 H), 2.85 (m, 1 H), ~2.50 (m, 1 H, below solvent signal), 2.47 (t, J = 8.1 Hz, 2 H), 1.97 (m, 2 H), 1.88 (m, 1 H), 1.76 (m, 1 H), 1.48 (m, 2 H). | 0.94 | 424.20 | A |
| 5 | 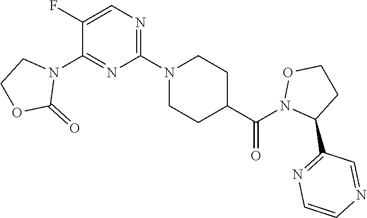 [SMILES: O=C1OCCN1c1nc(N2CCC(C(=O)N3OCC[C@H]3c3cnccn3)CC2)ncc1F] | ¹H NMR (400 MHz, DMSO-d₆) δ ppm: 8.62 (dd, J = 2.5 Hz, 1.5 Hz, 1 H), 8.60 (d, J = 1.5 Hz, 1 H), 8.57 (d, J = 2.5 Hz, 1 H), 8.39 (d, J = 3.3 Hz, 1 H), 5.43 (dd, J = 8.8 Hz, 6.2 Hz, 1 H), ~4.50 (m, 2 H), 4.50 (t, J = 7.2 Hz, 2 H), 4.34 (m, 1 H), 4.12 (t, J = 7.2 Hz, 2 H), 4.00 (m, 1 H), 3.07-2.97 (m, 3 H), 2.84 (m, 1 H), ~2.52 (m, 1 H, below solvent signal), 1.87 (m, 1 H), 1.75 (m, 1 H), 1.49 (m, 2 H). | 1.54 | 444.20 | A |
| 6 | 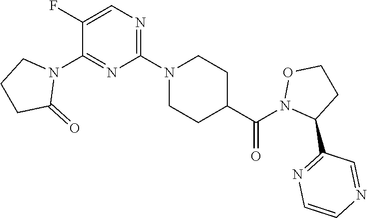 [SMILES: O=C1CCCN1c1nc(N2CCC(C(=O)N3OCC[C@H]3c3cnccn3)CC2)ncc1F] | ¹H NMR (400 MHz, DMSO-d₆) δ ppm: 8.62 (dd, J = 2.5 Hz, 1.5 Hz, 1 H), 8.60 (d, J = 1.5 Hz, 1 H), 8.57 (d, J = 2.5 Hz, 1 H), 8.37 (d, J = 3.1 Hz, 1 H), 5.43 (dd, J = 8.8 Hz, 6.2 Hz, 1 H), 4.50 (m, 2 H), 4.34 (m, 1 H), 4.00 (m, 1 H), 3.87 (t, J = 7.0 Hz, 2 H), 3.05-2.95 (m, 3 H), 2.84 (m, 1 H), ~2.52 (m, 1 H, below solvent signal), 2.48 (t, J = 8.0 Hz, 2 H), 2.10 (m, 2 H), 1.86 (m, 1 H), 1.74 (m, 1 H), 1.48 (m, 2 H). | 1.61 | 441.20 | A |
| 7 | 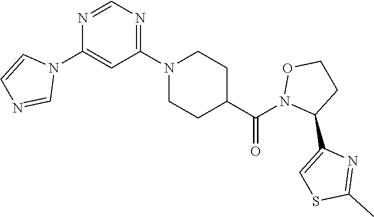 [SMILES: Cc1nc([C@@H]2CCON2C(=O)C2CCN(c3cc(-n4ccnc4)ncn3)CC2)cs1] | ¹H NMR (400 MHz, DMSO-d₆) δ ppm: 8.48 (d, J = 0.5 Hz, 1 H), 7.64 (d, J = 1.5 Hz, 1 H), 7.22 (bs, 1 H), 6.90 (d, J = 1.5 Hz, 1 H), 6.89 (d, J = 0.5 Hz, 1 H), 5.39 (bdd, 1 H), 4.47 (bm, 2 H), 4.28 (m, 1 H), 3.92 (m, 1 H), 3.14- 3.05 (m, 3 H), 2.74 (m, 1 H), 2.63 (s, 3 H), 2.55 (s, 3 H), 2.43 (m, 1 H), 1.90 (m, 1 H), 1.75 (m, 1 H), 1.53 (m, 2 H). | 1.12 | 440.20 | A |
| 8 | 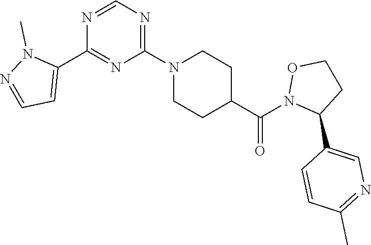 [SMILES: Cc1ccc([C@@H]2CCON2C(=O)C2CCN(c3ncnc(-c4ccnn4C)n3)CC2)cn1] | ¹H NMR (400 MHz, DMSO-d₆) δ ppm: 8.64 (s, 1 H), 8.37 (bd, J ~ 2 Hz, 1 H), 7.55 (dd, J = 8.0 Hz, 2.2 Hz, 1 H), 7.52 (d, J = 1.6 Hz, 1 H), 7.22 (d, J = 8.0 Hz, 1 H), 7.04 (d, J = 1.6 Hz, 1 H), 5.33 (bdd, 1 H), 4.66 (m, 2 H), 4.31 (m, 1 H), 4.22 (s, 3 H), 3.94 (m, 1 H), 3.23-3.08 (m, 3 H), 2.89 (m, 1 H), 2.44 (s, 3 H), 2.23 (m, 1 H), 1.97 (m, 1 H), 1.81 (m, 1 H), 1.53 (m, 2 H). | 0.666 | 435.30 | C |
| 9 | 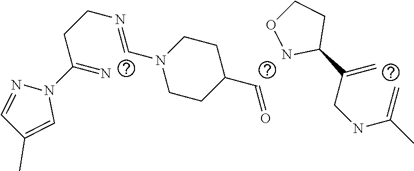 | ¹H NMR (400 MHz, DMSO-d₆) δ ppm: 8.45 (s, 1 H), 8.44 (bd, J ~ 1.5 Hz, 1 H), 8.39 (d, J = 5.4 Hz, 1 H), 7.72 (bd, 1 H), 7.68 (s, 1 H), 7.36 (d, J ~ 8 Hz, 1 H), 6.98 (d, J = 5.4 Hz, 1 H), 5.37 (bdd, 1 H), 4.70 (m, 2 H), 4.32 (m, 1 H), 3.95 (m, 1 H), 3.11-3.01 (m, 3 H), 2.90 (m, 1 H), ~2.50 (s, 3 H, below solvent signal? ), 2.25 (m, 1 H), 2.10 (s, 3 H), 1.92 (m, 1 H), 1.76 (m, 1 H), 1.51 (m, 2 H). | 1.5 | Not found | A |
| 10 | 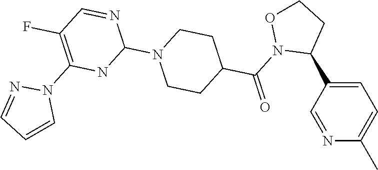 [SMILES: Cc1ccc([C@@H]2CCON2C(=O)C2CCN(C3N=CC(F)=C(n4cccn4)N3)CC2)cn1] | ¹H NMR (400 MHz, DMSO-d₆) δ ppm: 8.63 (d, J = 2.7 Hz, 1 H), 8.57 (d, J = 4.0 Hz, 1 H), 8.49 (bs, 1 H), 7.92 (d, J = 1.2 Hz, 1 H), 7.84 (bd, 1 H), 7.47 (bd, 1 H), 6.63 (bdd, 1 H), 5.39 (bdd, 1 H), 4.60 (m, 2 H), 4.32 (m, 1 H), 3.96 (m, 1 H), 3.12-3.02 (m, 3 H), 2.91 (m, 1 H), 2.53 (s, 3 H), 2.27 (m, 1 H), 1.92 (m, 1 H), 1.77 (m, 1 H), 1.52 (m, 2 H). | 1.4 | 438.20 | A |
| 11 | 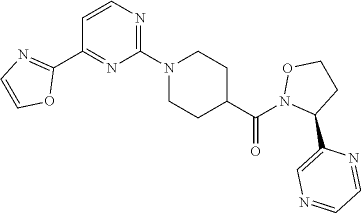 [SMILES: O=C(C1CCN(c2nccc(-c3ncco3)n2)CC1)N1OCC[C@H]1c1cnccn1] | ¹H NMR (400 MHz, DMSO-d₆) δ ppm: 8.63-8.61 (m, 2 H), 8.57 (d, J = 2.4 Hz, 1 H), 8.53 (d, J = 5.0 Hz, 1 H), 8.35 (d, J = 0.5 Hz, 1 H), 7.50 (d, J = 0.5 Hz, 1 H), 7.19 (d, J = 5.0 Hz, 1 H), 5.44 (dd, J = 8.7 Hz, 6.2 Hz, 1 H), 4.71 (m, 2 H), 4.35 (m, 1 H), 4.01 (m, 1 H), 3.12-3.03 (m, 3 H), 2.85 (m, 1 H), 2.52 (m, 1 H, below solvent signal), 1.92 (m, 1 H), 1.80 (m, 1 H), 1.51 (m, 2 H). | 1.59 | 408.20 | A |
| 12 | 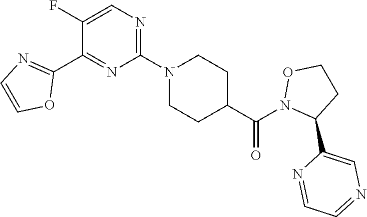 [SMILES: O=C(C1CCN(c2ncc(F)c(-c3ncco3)n2)CC1)N1OCC[C@H]1c1cnccn1] | ¹H NMR (400 MHz, DMSO-d₆) δ ppm: 8.64 (d, J = 2.9 Hz, 1 H), 8.62 (dd, J = 2.4 Hz, 1.5 Hz, 1 H), 8.60 (d, J = 1.5 Hz, 1 H), 8.57 (d, J = 2.4 Hz, 1 H), 8.42 (d, J = 0.5 Hz, 1 H), 7.57 (d, J = 0.5 Hz, 1 H), 5.44 (dd, J = 8.8 Hz, 6.2 Hz, 1 H), 4.61 (m, 2 H), 4.35 (m, 1 H), 4.01 (m, 1 H), 3.11- 3.03 (m, 3 H), 2.85 (m, 1 H), 2.52 (m, 1 H, below solvent signal), 1.91 (m, 1 H), 1.79 (m, 1 H), 1.52 (m, 2 H). | 1.68 | 426.10 | A |
| 13 | 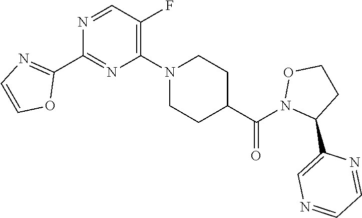 [SMILES: O=C(C1CCN(c2nc(-c3ncco3)ncc2F)CC1)N1OCC[C@H]1c1cnccn1] | ¹H NMR (400 MHz, DMSO-d₆) δ ppm: 8.62-8.60 (m, 2 H), 8.57 (d, J = 2.4 Hz, 1 H), 8.37 (d, J = 6.8 Hz, 1 H), 8.27 (d, J = 0.5 Hz, 1 H), 7.43 (d, J = 0.5 Hz, 1 H), 5.44 (dd, J = 8.8 Hz, 6.2 Hz, 1 H), 4.48 (m, 2 H), 4.35 (m, 1 H), 4.01 (m, 1 H), 3.29-3.09 (m, 3 H), 2.85 (m, 1 H), 2.51 (m, 1 H, below solvent signal), 1.96 (m, 1 H), 1.84 (m, 1 H), 1.63 (m, 2 H). | 1.46 | 426.10 | A |
| 14 | 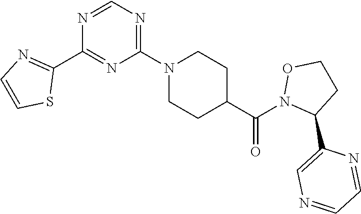 [SMILES: O=C(C1CCN(c2ncnc(-c3nccs3)n2)CC1)N1OCC[C@H]1c1cnccn1] | ¹H NMR (400 MHz, DMSO-d₆) δ ppm: 8.68 (s, 1 H), 8.63-8.61 (m, 2 H), 8.58 (d, J = 2.2 Hz, 1 H), 8.11 (d, J = 3.0 Hz, 1 H), 8.04 (d, J = 3.0 Hz, 1 H), 5.44 (dd, J = 8.8 Hz, 6.2 Hz, 1 H), 4.70 (m, 2 H), 4.35 (m, 1 H), 4.02 (m, 1 H), 3.24-3.09 (m, 3 H), 2.86 (m, 1 H), 2.51 (m, 1 H, below solvent signal), 1.97 (m, 1 H), 1.86 (m, 1 H), 1.55 (m, 2 H). | 1.5 | 425.10 | A |
| 15 | 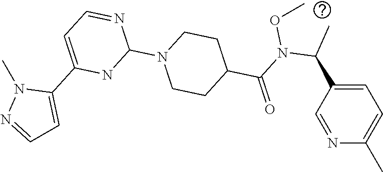 | ¹H NMR (400 MHz, DMSO-d₆) δ ppm: 8.41 (d, J = 5.1 Hz, 1 H), 8.36 (d, J = 2.2 Hz, 1 H), 7.55 (dd, J = 8.0 Hz, 2.2 Hz, 1 H), 7.50 (d, J = 2.0 Hz, 1 H), 7.22 (d, J = 8.0 Hz, 1 H), 6.99 (d, J = 5.1 Hz, 1 H), 6.93 (d, J = 2.0 Hz, 1 H), 5.33 (bdd, 1 H), 4.65 (m, 2 H), 4.30 (m, 1 H), 4.19 (s, 3 H), 3.94 (m, 1 H), 3.13-3.04 (m, 3 H), 2.88 (m, 1 H), 2.44 (s, 3 H), 2.23 (m, 1 H), 1.91 (m, 1 H), 1.76 (m, 1 H), 1.52 (m, 2 H). | 1.35 | 434.20 | A |
| 16 | 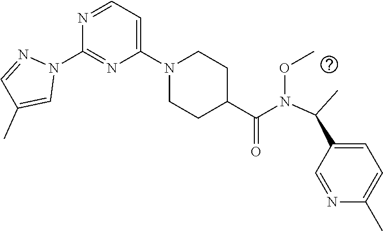 [SMILES: CON(C(=O)C1CCN(c2ccnc(-n3cc(C)cn3)n2)CC1)[C@@H](C)c1ccc(C)nc1] | ¹H NMR (400 MHz, DMSO-d₆) δ ppm: 8.46 (s, 1 H), 8.42 (bd, J ~ 1.5 Hz, 1 H), 8.39 (d, J = 5.3 Hz, 1 H), 7.68 (s, 1 H), 7.67 (bd, H), 7.33 (bd, J ~ 8.0 Hz, 1 H), 6.98 (d, J = 5.3 Hz, 1 H), 5.36 (bdd, 1 H), 4.70 (m, 2 H), 4.32 (m, 1 H), 3.95 (m, 1 H), 3.11-3.01 (m, 3 H), 2.90 (m, 1 H), 2.48 (s, 3 H), 2.25 (m, 1 H), 2.10 (s, 3 H), 1.92 (m, 1 H), 1.76 (m, 1 H), 1.51 (m, 2 H). | 1.53 | 434.20 | A |
| 17 | 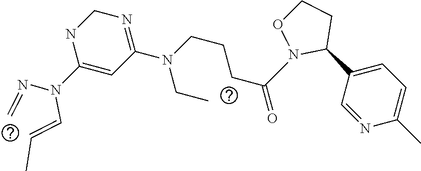 | ¹H NMR (400 MHz, DMSO-d₆) δ ppm: 8.46 (bd, J ~ 1.5 Hz, 1 H), 8.41 (s, 1 H), 8.33 (s, 1 H), 7.77 (bd, H), 7.67 (s, 1 H), 7.41 (bd, J ~ 8.0 Hz, 1 H), 7.06 (s, 1 H), 5.37 (bdd, 1 H), 4.40 (bm, 2 H), 4.31 (m, 1 H), 3.95 (m, 1 H), 3.16- 3.06 (m, 3 H), 2.90 (m, 1 H), ~2.51 (s, 3 H, below solvent signal), 2.25 (m, 1 H), 2.09 (s, 3 H), 1.94 (m, 1 H), 1.78 (m, 1 H), 1.52 (m, 2 H). | 1.46 | 434.20 | A |
| 18 | 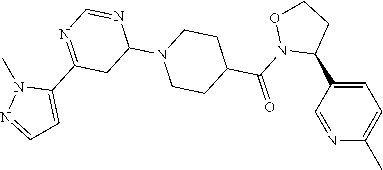 | ¹H NMR (400 MHz, DMSO-d₆) δ ppm: 8.59 (s, 1 H), 8.52 (bs, 1 H), 7.91 (bd, 1 H), 7.53 (bd, J ~ 8.0 Hz, 1 H), 7.50 (d, J = 1.9 Hz, 1 H), 7.14 (s, 1 H), 6.93 (d, J = 1.9 Hz, 1 H), 5.41 (bdd, 1 H), 4.49 (bm, 2 H), 4.33 (m, 1 H), 4.13 (s, 3 H), 3.97 (m, 1 H), 3.15- 3.07 (m, 3 H), 2.92 (m, 1 H), 2.56 (s, 3 H), 2.28 (m, 1 H), 1.94 (m, 1 H), 1.78 (m, 1 H), 1.52 (m, 2 H). | 1.02 | 434.20 | A |
| 19 | 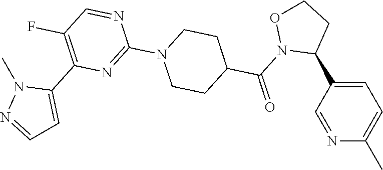 | ¹H NMR (400 MHz, DMSO-d₆) δ ppm: 8.54 (d, J = 2.9 Hz, 1 H), 8.46 (bs, 1 H), 7.77 (bd, 1 H), 7.59 (d, J = 2.0 Hz, 1 H), 7.40 (bd, J ~ 8.0 Hz, 1 H), 6.84 (dd, J = 4.0 Hz, 2.0 Hz, 1 H), 5.37 (bdd, 1 H), 4.56 (bm, 2 H), 4.31 (m, 1 H), 4.17 (s, 3 H), 3.95 (m, 1 H), 3.14- 3.04 (m, 3 H), 2.90 (m, 1 H), ~2.50 (s, 3 H, below solvent signal), 2.25 (m, 1 H), 1.92 (m, 1 H), 1.76 (m, 1 H), 1.53 (m, | 1.56 | 452.20 | A |
| | | 2 H). | | | |
| 20 | 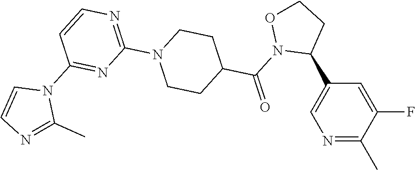 [SMILES: Cc1ncc([C@@H]2CCON2C(=O)C2CCN(c3nccc(-n4ccnc4C)n3)CC2)cc1F] | ¹H NMR (400 MHz, DMSO-d₆) δ ppm: 8.46 (d, J = 5.4 Hz, 1 H), 8.25 (s, 1 H), 7.69 (d, J = 1.4 Hz, 1 H), 7.49 (dd, J = 10.4 Hz, 1.4 Hz, 1 H), 6.93 (s, 1 H), 6.84 (d, J = 5.4 Hz, 1 H), 5.38 (bdd, 1 H), 4.62 (bm, 2 H), 4.31 (m, 1 H), 3.95 (m, 1 H), 3.14-3.05 (m, 3 H), 2.89 (m, 1 H), 2.62 (s, 3 H), 2.42 (d, J = 2.8 Hz, 3 H), 2.27 (m, 1 H), 1.93 (m, 1 H), 1.77 (m, 1 H), 1.52 (m, 2 H). | 1.32 | 452.20 | A |
| 21 | 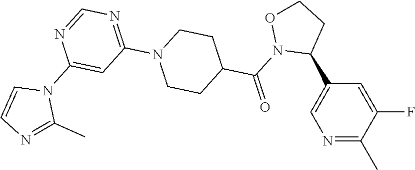 [SMILES: Cc1ncc([C@@H]2CCON2C(=O)C2CCN(c3cc(-n4ccnc4C)ncn3)CC2)cc1F] | ¹H NMR (400 MHz, DMSO-d₆) δ ppm: 8.48 (s, 1 H), 8.25 (bs, 1 H), 7.66 (d, J = 1.4 Hz, 1 H), 7.49 (dd, J = 10.4 Hz, 1.4 Hz, 1 H), 6.93 (d, J = 1.0 Hz, 1 H), 6.90 (s, 1 H), 5.38 (bdd, 1 H), 4.47 (bm, 2 H), 4.31 (m, 1 H), 3.95 (m, 1 H), 3.16-3.07 (m, 3 H), 2.89 (m, 1 H), 2.56 (s, 3 H), 2.42 (d, J = 2.8 Hz, 3 H), 2.27 (m, 1 H), 1.93 (m, 1 H), 1.77 (m, 1 H), 1.52 (m, 2 H). | 1.26 | 452.20 | A |
| 22 | 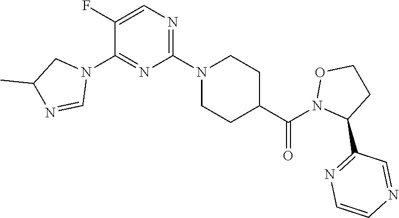 [SMILES: CC1CN(c2nc(N3CCC(C(=O)N4OCC[C@H]4c4cnccn4)CC3)ncc2F)C=N1] | ¹H NMR (400 MHz, DMSO-d₆) δ ppm: 8.69 (d, J = 2.7 Hz, 1 H), 8.62 (dd, J = 2.4 Hz, 1.3 Hz, 1 H), 8.60 (bd, J ~ 1.3 Hz, 1 H), 8.57 (d, J = 2.4 Hz, 1 H), 7.39 (bs, 1 H), 5.43 (dd, J = 8.6 Hz, 6.2 Hz, 1 H), 4.50 (m, 2 H), 4.34 (m, 1 H), 4.00 (m, 1 H), 3.14-3.06 (m, 3 H), 2.85 (m, 1 H), 2.56 (s, 3 H), ~2.50 (m, 1 H, below solvent signal), 2.18 (s, 3 H), 1.90 (m, 1 H), 1.77 (m, 1 H), 1.52 (m, 2 H). | 1.25 | 453.20 | A |
| 23 | 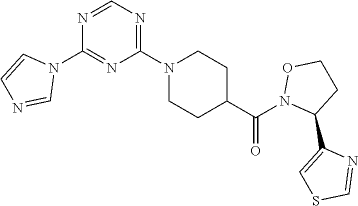 [SMILES: O=C(C1CCN(c2ncnc(-n3ccnc3)n2)CC1)N1OCC[C@H]1c1cscn1] | ¹H NMR (400 MHz, DMSO-d₆) δ ppm: 8.68 (d, J = 1.0 Hz, 1 H), 8.05 (bs, 1 H), 7.27 (bs, 1 H), 7.23 (bs, 1 H), 5.39 (bdd, 1 H), 4.67 (m, 1 H), 4.59 (m, 1 H), 4.28 (m, 1 H), 3.94 (m, 1 H), 3.27-3.10 (m, 3 H), 2.82 (s, 3 H), 2.75 (m, 1 H), 2.63 (s, 3 H), 2.45 (m, 1 H), 1.97 (m, 1 H), 1.81 (m, 1 H), 1.56 (m, 2 H). | 1.3 | 441.20 | A |
| 24 | 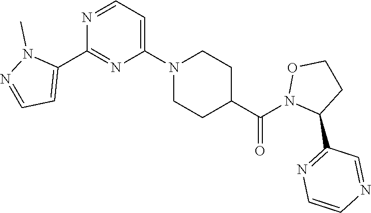 [SMILES: Cn1nccc1-c1nccc(N2CCC(C(=O)N3OCC[C@H]3c3cnccn3)CC2)n1] | ¹H NMR (400 MHz, DMSO-d₆) δ ppm: 8.62-8.60 (m, 2 H), 8.58 (d, J = 2.4 Hz, 1 H), 8.30 (d, J = 6.8 Hz, 1 H), 7.54 (d, J = 1.9 Hz, 1 H), 7.00 (bd, J ~ 1.9 Hz, 1 H), 6.93 (d, J = 6.8 Hz, 1 H), 5.44 (dd, J = 8.8 Hz, 6.2 Hz, 1 H), 4.46 (bm, 2 H), 4.35 (m, 1 H), 4.20 (s, 3 H), 4.02 (m, 1 H), 3.29-3.14 (m, 3 H), 2.86 (m, 1 H), ~2.51 (m, 1 H, below solvent signal), 1.98 (m, 1 H), 1.85 (m, 1 H), 1.58 (m, 2 H). | 1.06 | 421.20 | A |
| 25 | 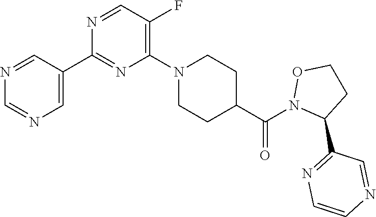 [SMILES: O=C(C1CCN(c2nc(-c3cncnc3)ncc2F)CC1)N1OCC[C@H]1c1cnccn1] | ¹H NMR (400 MHz, DMSO-d₆) δ ppm: 9.51 (s, 2 H), 9.29 (s, 1 H), 8.62-8.60 (m, 2 H), 8.57 (d, J = 2.5 Hz, 1 H), 8.41 (d, J = 6.9 Hz, 1 H), 5.44 (dd, J = 8.8 Hz, 6.2 Hz, 1 H), 4.54 (m, 2 H), 4.35 (m, 1 H), 4.02 (m, 1 H), 3.28-3.23 (m, 2 H), 3.14 (m, 1 H), 2.85 (m, 1 H), ~2.51 (m, 1 H, below solvent signal), 1.98 (m, 1 H), 1.86 (m, 1 H), 1.65 (m, 2 H). | 1.63 | 437.20 | A |
| 26 | 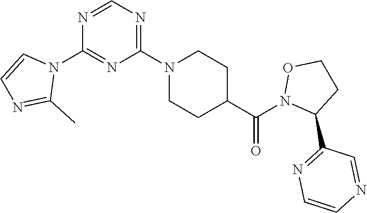 [SMILES: Cc1nccn1-c1ncnc(N2CCC(C(=O)N3OCC[C@H]3c3cnccn3)CC2)n1] | ¹H NMR (400 MHz, DMSO-d₆) δ ppm: 8.62-8.60 (m, 3 H), 8.58 (d, J = 2.4 Hz, 1 H), ~7.85/7.84 (d, J ~ 1 Hz, 1 H), 6.895/6.890 (d, J ~ 1 Hz, 1 H), 5.44 (dd, J = 8.8 Hz, 6.2 Hz, 1 H), 4.66 (m, 1 H), 4.58 (m, 1 H), 4.35 (m, 1 H), 4.02 (m, 1 H), 3.25-3.10 (m, 3 H), 2.85 (m, 1 H), 2.70/2.69 (s, 3 H), ~2.52 (m, 1 H, below solvent signal), 1.96 (m, 1 H), 1.84 (m, 1 H), 1.55 (m, 2 H). | 1.04 | 422.20 | A |
| 27 | 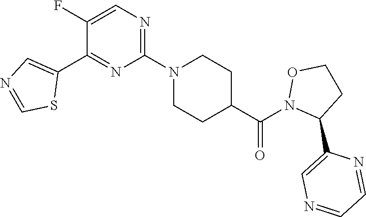 [SMILES: O=C(C1CCN(c2ncc(F)c(-c3cncs3)n2)CC1)N1OCC[C@H]1c1cnccn1] | ¹H NMR (400 MHz, DMSO-d₆) δ ppm: 9.34 (s, 1 H), 8.62 (dd, J = 2.4 Hz, 1.5 Hz, 1 H), 8.60 (d, J = 1.5 Hz, 1 H), 8.58-8.56 (m, 3 H), 5.44 (dd, J = 8.8 Hz, 6.2 Hz, 1 H), 4.57 (m, 2 H), 4.34 (m, 1 H), 4.01 (m, 1 H), 3.10-3.02 (m, 3 H), 2.85 (m, 1 H), ~2.51 (m, 1 H, below solvent signal), 1.91 (m, 1 H), 1.79 (m, 1 H), 1.52 (m, 2 H). | 1.89 | 442.20 | A |
| 28 | 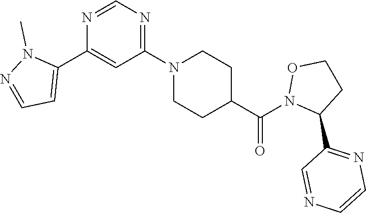 [SMILES: Cn1nccc1-c1cc(N2CCC(C(=O)N3OCC[C@H]3c3cnccn3)CC2)ncn1] | ¹H NMR (400 MHz, DMSO-d₆) δ ppm: 8.62 (dd, J = 2.4 Hz, 1.5 Hz, 1 H), 8.60 (d, J = 1.5 Hz, 1 H), 8.58-8.57 (m, 2 H), 7.49 (d, J = 2.0 Hz, 1 H), 7.13 (d, J ~ 0.9 Hz, 1 H), 6.93 (d, J = 2.0 Hz, 1 H), 5.44 (dd, J = 8.8 Hz, 6.2 Hz, 1 H), 4.48 (bm, 2 H), 4.35 (m, 1 H), 4.13 (s, 3 H), 4.01 (m, 1 H), 3.14-3.05 (m, 3 H), 2.85 (m, 1 H), ~2.52 (m, 1 H, below solvent signal), 1.91 (m, 1 H), 1.79 (m, 1 H), 1.52 (m, 2 H). | 1.19 | 421.20 | A |
| 29 | 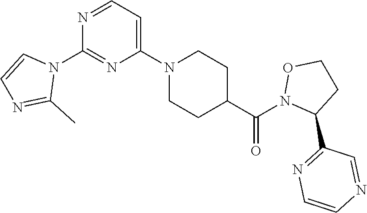 [SMILES: Cc1nccn1-c1nccc(N2CCC(C(=O)N3OCC[C@H]3c3cnccn3)CC2)n1] | ¹H NMR (400 MHz, DMSO-d₆) δ ppm: 8.62 (dd, J = 2.4 Hz, 1.5 Hz, 1 H), 8.61 (d, J = 1.5 Hz, 1 H), 8.58 (d, J = 2.4 Hz, 1 H), 8.21 (d, J = 6.3 Hz, 1 H), 7.77 (d, J = 1.5 Hz, 1 H), 6.83 (d, J = 1.5 Hz, 1 H), 6.78 (d, J = 6.3 Hz, 1 H), 5.44 (dd, J = 8.8 Hz, 6.2 Hz, 1 H), ~4.36 (bm, 2 H), 4.35 (m, 1 H), 4.01 (m, 1 H), 3.19-3.08 (m, 3 H), 2.85 (m, 1 H), 2.66 (s, 3 H), ~2.52 (m, 1 H, below solvent signal), 1.93 (m, 1 H), 1.81 (m, 1 H), 1.54 (m, 2 H). | 1.03 | 421.30 | A |
| 30 | 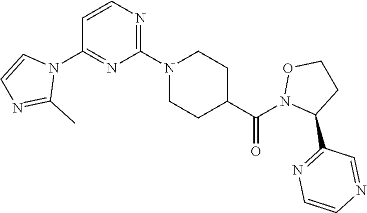 [SMILES: Cc1nccn1-c1ccnc(N2CCC(C(=O)N3OCC[C@H]3c3cnccn3)CC2)n1] | ¹H NMR (400 MHz, DMSO-d₆) δ ppm: 8.62 (dd, J = 2.4 Hz, 1.5 Hz, 1 H), 8.60 (d, J = 1.5 Hz, 1 H), 8.57 (d, J = 2.4 Hz, 1 H), 8.45 (d, J = 5.4 Hz, 1 H), 7.69 (d, J = 1.6 Hz, 1 H), 6.92 (d, J = 1.6 Hz, 1 H), 6.83 (d, J = 5.4 Hz, 1 H), 5.43 (dd, J = 8.8 Hz, 6.2 Hz, 1 H), 4.61 (bm, 2 H), 4.34 (m, 1 H), 4.01 (m, 1 H), 3.13-3.05 (m, 3 H), 2.85 (m, 1 H), 2.62 (s, 3 H), ~2.51 (m, 1 H, below solvent signal), 1.91 (m, 1 H), 1.79 (m, 1 H), 1.51 (m, 2 H). | 1.04 | 421.30 | A |
| 31 | 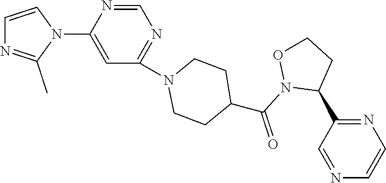 [SMILES: Cc1nccn1-c1cc(N2CCC(C(=O)N3OCC[C@H]3c3cnccn3)CC2)ncn1] | ¹H NMR (400 MHz, DMSO-d₆) δ ppm: 8.62 (dd, J = 2.4 Hz, 1.5 Hz, 1 H), 8.60 (d, J = 1.5 Hz, 1 H), 8.58 (d, J = 2.4 Hz, 1 H), 8.56 (d, J ~ 0.4 Hz, 1 H), 8.06 (d, J = 2.0 Hz, 1 H), 7.65 (bd, J ~ 2.0 Hz, 1 H), 7.12 (s, 1 H), 5.43 (dd, J = 8.8 Hz, 6.2 Hz, 1 H), ~4.47 (bm, 2 H), 4.35 (m, 1 H), 4.01 (m, 1 H), 3.22-3.12 (m, 3 H), 2.85 (m, 1 H), 2.77 (s, 3 H), ~2.51 (m, 1 H, below solvent signal), 1.94 (m, 1 H), 1.81 (m, 1 H), 1.54 (m, 2 H). | 0.99 | 421.30 | A |
| 32 | 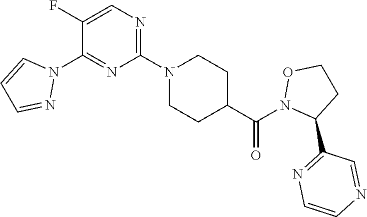 [SMILES: O=C(C1CCN(c2ncc(F)c(-n3cccn3)n2)CC1)N1OCC[C@H]1c1cnccn1] | ¹H NMR (400 MHz, DMSO-d₆) δ ppm: 8.63-8.60 (m, 3 H), 8.58-8.56 (m, 2 H), 7.22 (bd, J ~ 1.7 Hz, 1 H), 6.63 (dd, J = 2.5 Hz, 1.7 Hz, 1 H), 5.44 (dd, J = 8.8 Hz, 6.2 Hz, 1 H), 4.60 (m, 2 H), 4.35 (m, 1 H), 4.01 (m, 1 H), 3.11- 3.03 (m, 3 H), 2.85 (m, 1 H), ~2.51 (m, 1 H, below solvent signal), 1.91 (m, 1 H), 1.78 (m, 1 H), 1.52 (m, 2 H). | 1.76 | 425.20 | A |
| 33 | 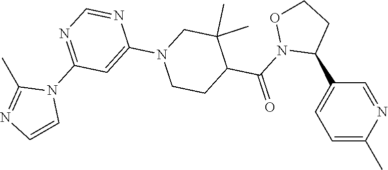 | ¹H NMR (400 MHz, CDCl₃) δ ppm = 8.49 (s, 2H), 7.53 (m, 1H), 7.31 (s, 1H), 7.17 (br d, J ~ 8.2 Hz, 1H), 7.17 (d, J = 1.4 Hz, 1H), 6.34 (d, J = 3.3 Hz, 1H), 5.41 (bdd, 1H), 4.29 (m, 1H), 4.03-3.84 (m, 3H), 3.49 (m, 1H), 2.93-2.85 (m, 2H), 2.74 (s, 3H), 2.64 (m, 1H), 2.57 (s, 3H), 2.36 (m, 1H), 2.17-1.93 (m, 2H), 0.77-0.37 (m, 4H). | 0.594 | 460.30 | C |
| 34 | 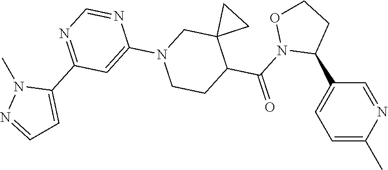 | ¹H NMR (400 MHz, CDCl₃) δ ppm = 8.60 (s, 1H), 8.51 (m, 1H), 7.64 (m, 1H), 7.49 (d, J = 1.9 Hz, 1H), 7.24 (m, 1H), 6.64 (s, 1H), 6.57 (d, J = 1.9 Hz, 1H), 5.43 (br dd, 1H), 4.30 (m, 1 H), 4.21 (s, 3H), 3.99-3.82 (m, 3H), 3.48 (m, 1H), 2.96-2.87 (m, 2H), 2.65 (m, 1H), 2.63 (s, 3H), 2.37 (m, 1H), 2.13- 1.93 (m, 2H), 0.72-0.36 (m, 4H). | 0.572 | 460.30 | C |
| 34 I | 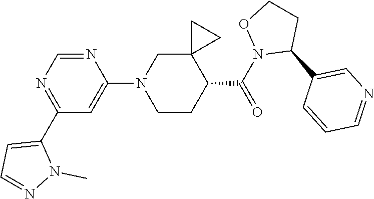 [SMILES: Cn1nccc1-c1cc(N2CC[C@@H](C(=O)N3OCC[C@H]3c3cccnc3)C3(CC3)C2)ncn1] | 1H NMR (400 MHz, Chloroform-d) δ ppm = 8.59 (s, 1H), 8.47 (d, J = 2.0 Hz, 1H), 7.52 (dd, J = 8.3, 2.4 Hz, 1H), 7.49 (d, J = 2.0 Hz, 1H), 7.15 (d, J = 8.3 Hz, 1H), 6.63 (s, 1H), 6.56 (d, J = 2.4 Hz, 1H), 5.45-5.38 (m, 1H), 4.33- 4.24 (m, 1H), 4.20 (s, 3H), 4.00-3.86 (m, 2H), 3.58-3.39 (m, 2H), 2.95- 2.82 (m, 1H), 2.72-2.61 (m, 1H), 2.55 (s, 3H), 2.41-2.28 (m, 1H), 2.21-2.01 (m, 2H), 2.00-1.75 (m, 1H), 0.72- 0.48 (m, 3H), 0.43-0.30 (m, 1H). | 0.404 | 460.3 | Q |
| 35 | 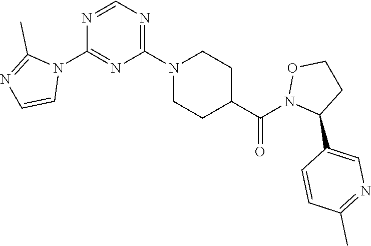 [SMILES: Cc1ccc([C@@H]2CCON2C(=O)C2CCN(c3ncnc(-n4ccnc4C)n3)CC2)cn1] | ¹H NMR (400 MHz, DMSO-d₆) δ ppm: 8.62 (s, 1 H), 8.37 (bd, J ~ 2.2 Hz, 1 H), 7.85 (bs, 1 H), 7.56 (dd, J = 8.0 Hz, 2.2 Hz, 1 H), 7.23 (d, J = 8.0 Hz, 1 H), 6.90 (s, 1 H), 5.34 (bdd, 1 H), 4.70-4.56 (m, 2 H), 4.31 (m, 1 H), 3.95 (m, 1 H), 3.25-3.10 (m, 3 H), 2.89 (m, 1 H), 2.705/2.700 (s, 3 H), 2.44 (s, 3 H), 2.24 (m, 1 H), 1.98 (m, 1 H), 1.82 (m, 1 H), 1.55 (m, 2 H). | 0.691 | 435.30 | D |
| 36 | 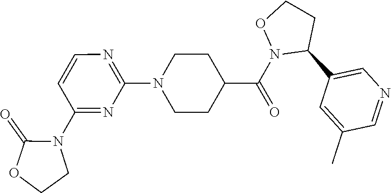 [SMILES: Cc1cncc([C@@H]2CCON2C(=O)C2CCN(c3nccc(N4CCOC4=O)n3)CC2)c1] | 1H NMR (400 MHz, CDCl3) δ = 8.39 (d, J = 2.0 Hz, 1H), 8.35 (s, 1H), 8.21 (d, J = 5.6 Hz, 1H), 7.41 (s, 1H), 7.36 (d, J = 5.6 Hz, 1H), 5.41 (dd, J = 8.8, 6.4 Hz, 1H), 4.77-4.66 (m, 2H), 4.54-4.44 (m, 2H), 4.32 (td, J = 8.0, 3.6 Hz, 1H), 4.26-4.17 (m, 2H), 4.04-3.85 (m, 1H), 3.10-2.95 (m, 3H), 2.93-2.83 (m, 1H), 2.43-2.35 (m, 1H), 2.33 (s, 3H), 2.07-1.94 (m, 1H), 1.79-1.68 (m, 3H). | 0.446 | 439.3 | Q |
| 37 | 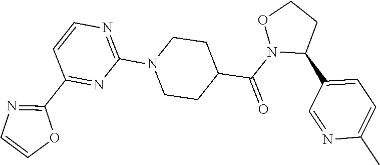 [SMILES: Cc1ccc([C@@H]2CCON2C(=O)C2CCN(c3nccc(-c4ncco4)n3)CC2)cn1] | ¹H NMR (400 MHz, CDCl3) δ ppm = 8.48-8.42 (m, 2H), 7.82 (s, 1H), 7.51 (dd, J = 7.8, 2.4 Hz, 1H), 7.34 (s, 1H), 7.19 (d, J = 4.9 Hz, 1H), 7.13 (d, J = 8.3 Hz, 1H), 5.46-5.37 (m, 1H), 5.00- 4.80 (m, 2H), 4.41-4.25 (m, 1H), 4.05-3.89 (m, 1H), 3.20-3.00 (m, 3H), 2.95-2.80 (m, 1H), 2.54 (s, 3H), 2.45-2.33 (m, 1H), 2.10-1.99 (m, 1H), 1.91-1.71 (m, 3H) | 0.446 | 421.2 | Q |
| 38 | 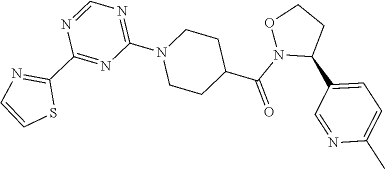 [SMILES: Cc1ccc([C@@H]2CCON2C(=O)C2CCN(c3ncnc(-c4nccs4)n3)CC2)cn1] | 1H NMR (400 MHz, CDCl3) δ ppm = 8.64 (s, 1H), 8.45 (d, J = 2.0 Hz, 1H), 8.06 (d, J = 3.1 Hz, 1H), 7.56 (d, J = 3.1 Hz, 1H), 7.50 (dd, J = 8.1, 2.3 Hz, 1H), 7.12 (d, J = 8.0 Hz, 1H), 5.45- 5.36 (m, 1H), 5.02-4.71 (m, 2H), 4.39-4.27 (m, 1H), 4.01-3.87 (m, 1H), 3.29-3.03 (m, 3H), 2.95-2.83 (m, 1H), 2.53 (s, 3H), 2.44-2.30 (m, 1H), 2.13-2.00 (m, 1H), 1.87-1.71 (m, 3H) | 0.489 | 483.2 | Q |
| 39 | 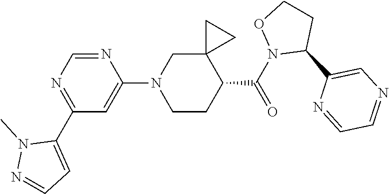 [SMILES: Cn1nccc1-c1cc(N2CC[C@@H](C(=O)N3OCC[C@H]3c3cnccn3)C3(CC3)C2)ncn1] | 1H NMR (400 MHz, DMSO-d6) δ ppm = 8.66-8.61 (m, 2H), 8.58 (d, J = 2.4 Hz, 1H), 8.52 (s, 1H), 7.47 (d, J = 2.0 Hz, 1H), 7.07 (s, 1H), 6.90 (d, J = 2.0 Hz, 1H), 5.44 (dd, J = 5.9, 8.8 Hz, 1H), 4.34-4.27 (m, 1H), 4.13 (s, 3H), 4.01-3.93 (m, 1H), 3.87-3.78 (m, 1H), 3.63- 3.40 (m, 3H), 2.90-2.79 (m, 1H), 2.79-2.73 (m, 1H), 2.46-2.40 (m, 1H), 1.99-1.82 (m, 2H), 0.57-0.43 (m, 3H), 0.43-0.30 (m, 1H) | 0.416 | 447.3 | Q |
| 40 | 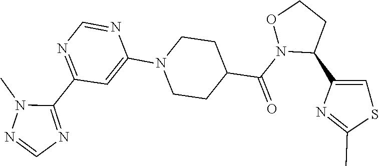 [SMILES: Cc1nc([C@@H]2CCON2C(=O)C2CCN(c3cc(-c4ncnn4C)ncn3)CC2)cs1] | 1H NMR (400 MHz, DMSO-d6) δ ppm = 8.69-8.60 (m, 1H), 8.06 (s, 1H), 7.46-7.37 (m, 1H), 7.22 (br s, 1H), 5.47-5.33 (m, 1H), 4.62-4.38 (m, 2H), 4.33-4.27 (m, 1H), 4.26 (s, 3H), 3.98-3.88 (m, 1H), 3.21-3.02 (m, 3H), 2.81-2.69 (m, 1H), 2.63 (s, 3H), 2.47-2.37 (m, 1H), 1.98-1.87 (m, 1H), 1.83-1.70 (m, 1H), 1.61- 1.46 (m, 2H) | 0.534 | 441.2 | Q |
| 41 | 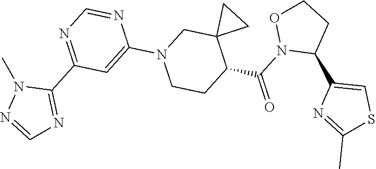 [SMILES: Cc1nc([C@@H]2CCON2C(=O)[C@@H]2CCN(c3cc(-c4ncnn4C)ncn3)CC23CC3)cs1] | 1H NMR (400 MHz, DMSO-d6) δ ppm = 8.59 (d, J = 1.0 Hz, 1H), 7.98 (s, 1H), 7.32 (d, J = 1.0 Hz, 1H), 7.20 (s, 1H), 5.53-5.38 (m, 1H), 4.29- 4.17 (m, 4H), 4.06-3.85 (m, 3H), 3.69-3.59 (m, 1H), 3.53-3.44 (m, 1H), 2.80-2.68 (m, 2H), 2.64 (s, 3H), 2.48-2.40 (m, 1H), 2.07-1.88 (m, 2H), 0.61-0.44 (m, 3H), 0.40-0.33 (m, 1H) | 0.852 | 467.1 | R |
| 42 | 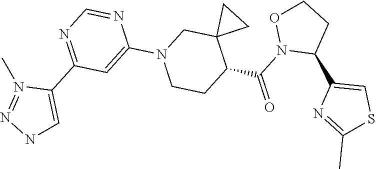 | 1H NMR (400 MHz, DMSO-d6) δ ppm = 8.56 (s, 1H), 8.36 (s, 1H), 7.32-7.18 (m, 2H), 5.40 (dd, J = 8.7, 5.3 Hz, 1H), 4.31 (s, 3H), 4.23 (td, J = 7.6, 4.4 Hz, 1H), 4.18-3.83 (m, 3H), 3.64-3.42 (m, 2H), 2.78- 2.69 (m, 2H), 2.63 (s, 3H), 2.45- 2.32 (m, 1H), 2.01-1.85 (m, 2H), 0.57-0.26 (m, 4H) | 0.853 | 467.1 | R |
| 43 | 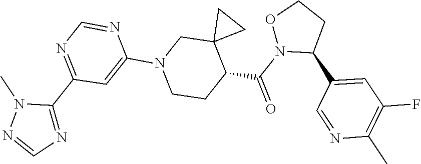 [SMILES: Cc1ncc([C@@H]2CCON2C(=O)[C@@H]2CCN(c3cc(-c4ncnn4C)ncn3)CC23CC3)cc1F] | 1H NMR (400 MHz, DMSO-d6) δ ppm = 8.59 (s, 1H), 8.27 (s, 1H), 7.98 (s, 1H), 7.47 (d, J = 10.5 Hz, 1H), 7.33 (s, 1H), 5.60-5.34 (m, 1H), 4.31-4.26 (m, 1H), 4.25 (s, 3H), 4.10-3.98 (m, 1H), 3.98-3.90 (m, 1H), 3.90-3.83 (m, 1H), 3.62-3.46 (m, 2H), 2.94-2.83 (m, 1H), 2.82- 2.75 (m, 1H), 2.43 (d, J = 2.5 Hz, 3H), 2.35-2.22 (m, 1H), 2.07-1.90 (m, 2H), 0.62-0.33 (m, 4H) | 0.563 | 479.3 | Q |
| 44 | 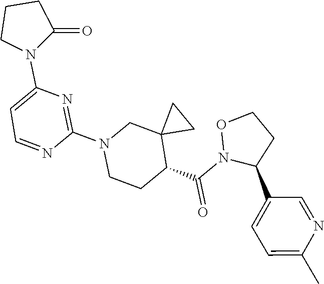 [SMILES: Cc1ccc([C@@H]2CCON2C(=O)[C@@H]2CCN(c3nccc(N4CCCC4=O)n3)CC23CC3)cn1] | 1H NMR (400 MHz, DMSO-d6) δ ppm = 8.37 (d, J = 2.0 Hz, 1H), 8.17 (d, J = 5.9 Hz, 1H), 7.56 (dd, J = 7.8, 2.4 Hz, 1H), 7.40 (d, J = 5.4 Hz, 1H), 7.23 (d, J = 7.8 Hz, 1H), 5.40-5.29 (m, 1H), 4.31-4.19 (m, 1H), 4.18- 4.06 (m, 1H), 3.96-3.78 (m, 4H), 3.64-3.54 (m, 1H), 3.54-3.44 (m, 1H), 2.93-2.81 (m, 1H), 2.79-2.69 (m, 1H), 2.59-2.52 (m, 2H), 2.44 (s, 3H), 2.27-2.13 (m, 1H), 2.06-1.94 (m, 2H), 1.94-1.81 (m, 2H), 0.56- 0.37 (m, 3H), 0.36-0.23 (m, 1H) | 0.422 | 463.3 | Q |
| 45 | 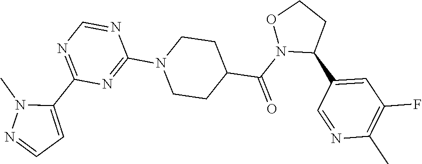 [SMILES: Cc1ncc([C@@H]2CCON2C(=O)C2CCN(c3ncnc(-c4ccnn4C)n3)CC2)cc1F] | 1H NMR (400 MHz, DMSO-d6) δ ppm = 8.65 (s, 1H), 8.25 (s, 1H), 7.63-7.43 (m, 2H), 7.04 (d, J = 1.8 Hz, 1H), 5.46-5.30 (m, 1H), 4.77-4.60 (m, 2H), 4.38-4.27 (m, 1H), 4.24 (s, 3H), 4.01-3.89 (m, 1H), 3.25-3.06 (m, 3H), 2.98-2.85 (m, 1H), 2.42 (d, J = 2.8 Hz, 3H), 2.33-2.21 (m, 1H), 2.04-1.94 (m, 1H), 1.90-1.72 (m, 1H), | 0.506 | 453.3 | Q |
| | | 1.66-1.39 (m, 2H) | | | |
| 46 | 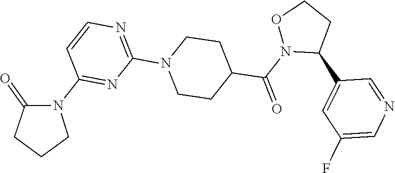 [SMILES: O=C1CCCN1c1ccnc(N2CCC(C(=O)N3OCC[C@H]3c3cncc(F)c3)CC2)n1] | 1H NMR (400 MHz, CDCl3) δ ppm = 8.43-8.40 (m, 1H), 8.39 (d, J = 2.9 Hz, 1H), 8.22 (d, J = 5.4 Hz, 1H), 7.57 (d, J = 5.4 Hz, 1H), 7.35 (td, J = 9.0, 2.3 Hz, 1H), 5.48 (dd, J = 6.1, 8.6 Hz, 1H), 4.81-4.68 (m, 2H), 4.33 (dt, J = 7.9, 3.2 Hz, 1H), 4.04 (t, J = 7.1 Hz, 2H), 3.99-3.88 (m, 1H), 3.11-2.85 (m, 4H), 2.64 (t, J = 8.1 Hz, 2H), 2.47-2.31 (m, 1H), 2.17-2.06 | 0.911 | 441.2 | S |
| | | (m, 2H), 2.04-1.96 (m, 1H), | | | |
| | | 1.81-1.69 (m, 3H) | | | |
| 47 | 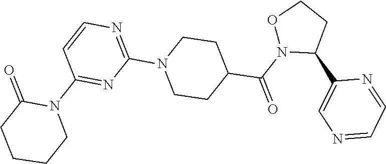 [SMILES: O=C1CCCCN1c1ccnc(N2CCC(C(=O)N3OCC[C@H]3c3cnccn3)CC2)n1] | 1H NMR (400 MHz, CDCl3) δ ppm = 8.67 (d, J = 1.3 Hz, 1H), 8.56-8.52 (m, 1H), 8.50 (d, J = 2.5 Hz, 1H), 8.20 (d, J = 5.6 Hz, 1H), 7.32 (d, J = 5.6 Hz, 1H), 5.62-5.51 (m, 1H), 4.81- 4.64 (m, 2H), 4.45-4.29 (m, 1H), 4.06-3.92 (m, 3H), 3.14-2.95 (m, 3H), 2.90-2.80 (m, 1H), 2.80-2.68 (m, 1H), 2.60 (t, J = 6.6 Hz, 2H), 2.02-1.67 (m, 8H) | 0.484 | 438.3 | Q |
| 48 | 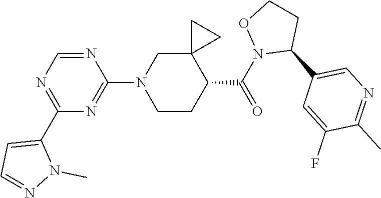 [SMILES: Cc1ncc([C@@H]2CCON2C(=O)[C@@H]2CCN(c3ncnc(-c4ccnn4C)n3)CC23CC3)cc1F] | 1H NMR (400 MHz, DMSO-d6) δ ppm = 8.67-8.52 (m, 1H), 8.31-8.23 (m, 1H), 7.59-7.47 (m, 2H), 7.03 (dd, J = 10.4, 1.9 Hz, 1H), 5.45-5.31 (m, 1H), 4.36-4.15 (m, 5H), 3.98-3.85 (m, 2H), 3.71 (br d, J = 13.3 Hz, 1H), 3.59-3.47 (m, 1H), 2.96-2.82 (m, 1H), 2.81-2.69 (m, 1H), 2.43 (d, J = 2.8 Hz, 3H), 2.31-2.18 (m, 1H), 2.08-1.89 (m, 2H), 0.60-0.42 (m, 3H), 0.42-0.30 (m, 1H) | 0.485 | 479.2 | Q |
| 49 | 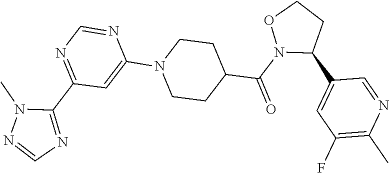 | 1H NMR (400 MHz, DMSO-d6) δ ppm = 8.64 (s, 1H), 8.25 (s, 1H), 8.06 (s, 1H), 7.55-7.44 (m, 1H), 7.39 (s, 1H), 5.43-5.29 (m, 1H), 4.54-4.36 (m, 2H), 4.33-4.28 (m, 1H), 4.25 (s, 3H), 4.01-3.89 (m, 1H), 3.18-3.05 (m, 3H), 2.95-2.83 (m, 1H), 2.45- 2.38 (m, 3H), 2.32-2.22 (m, 1H), 2.00-1.90 (m, 1H), 1.83-1.72 (m, 1H), 1.60-1.44 (m, 2H) | 0.425 | 453.2 | Q |
| 50 | 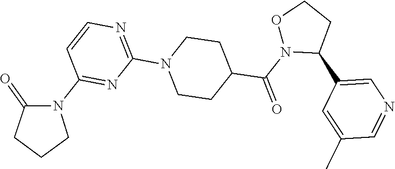 [SMILES: Cc1cncc([C@@H]2CCON2C(=O)C2CCN(c3nccc(N4CCCC4=O)n3)CC2)c1] | 1H NMR (400 MHz, DMSO-d6) δ ppm = 8.35-8.27 (m, 2H), 8.22 (d, J = 5.6 Hz, 1H), 7.49 (s, 1H), 7.45 (d, J = 5.6 Hz, 1H), 5.40-5.23 (m, 1H), 4.67-4.53 (m, 2H), 4.37-4.24 (m, 1H), 3.99-3.87 (m, 3H), 3.12- 2.83 (m, 4H), 2.60-2.53 (m, 2H), 2.30 (s, 3H), 2.27-2.17 (m, 1H), 2.06-1.96 (m, 2H), 1.92-1.84 (m, 1H), 1.78-1.63 (m, 1H), 1.56- | 0.369 | 437.2 | Q |
| | | 1.39 (m, 2H) | | | |
| 51 | 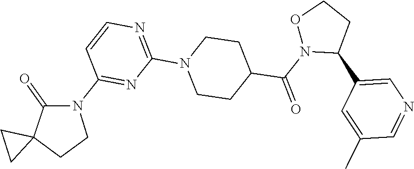 [SMILES: Cc1cncc([C@@H]2CCON2C(=O)C2CCN(c3nccc(N4CCC5(CC5)C4=O)n3)CC2)c1] | 1H NMR (400 MHz, MeOD-d4) δ ppm = 8.35-8.29 (m, 2H), 8.17 (d, J = 5.9 Hz, 1H), 7.66-7.61 (m, 1H), 7.51 (d, J = 5.4 Hz, 1H), 5.48-5.34 (m, 1H), 4.78-4.67 (m, 2H), 4.39 (td, J = 7.8, 2.9 Hz, 1H), 4.16-4.08 (m, 2H), 4.08- 3.99 (m, 1H), 3.24-3.13 (m, 1H), 3.24-3.12 (m, 1H), 3.10-2.92 (m, 3H), 2.44-2.32 (m, 4H), 2.26-2.18 (m, 2H), 2.06-1.92 (m, 1H), 1.88- | 0.962 | 463.2 | S |
| | | 1.74 (m, 1H), 1.72-1.57 (m, 2H), | | | |
| | | 1.24-1.14 (m, 2H), 1.01-0.91 (m, | | | |
| | | 2H) | | | |
| 52 | 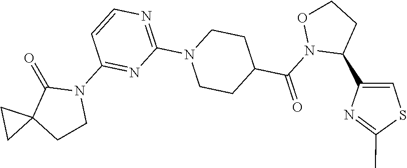 [SMILES: Cc1nc([C@@H]2CCON2C(=O)C2CCN(c3nccc(N4CCC5(CC5)C4=O)n3)CC2)cs1] | 1H NMR (400 MHz, CDCl3) δ ppm = 8.27-8.15 (m, 1H), 7.62-7.50 (m, 1H), 7.01 (s, 1H), 5.57-5.49 (m, 1H), 4.82-4.69 (m, 2H), 4.38-4.25 (m, 1H), 4.14-4.05 (m, 2H), 4.01-3.91 (m, 1H), 3.12-2.92 (m, 3H), 2.82- 2.62 (m, 5H), 2.24-2.15 (m, 2H), 2.04-1.92 (m, 1H), 1.86-1.70 (m, 3H), 1.31-1.23 (m, 2H), 0.93-0.83 (m, 2H) | 0.443 | 469.2 | Q |
| 53 | 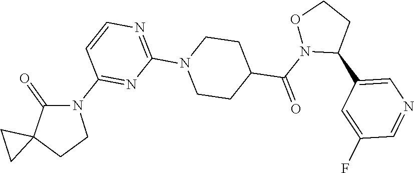 [SMILES: O=C(C1CCN(c2nccc(N3CCC4(CC4)C3=O)n2)CC1)N1OCC[C@H]1c1cncc(F)c1] | 1H NMR (400 MHz, MeOD-d4) δ ppm = 8.45-8.34 (m, 2H), 8.15 (d, J = 5.9 Hz, 1H), 7.64-7.55 (m, 1H), 7.49 (d, J = 5.9 Hz, 1H), 5.52-5.41 (m, 1H), 4.77-4.66 (m, 2H), 4.45- 4.32 (m, 1H), 4.16-3.98 (m, 3H), 3.24-3.12 (m, 1H), 3.12-2.94 (m, 3H), 2.49-2.31 (m, 1H), 2.30-2.16 (m, 2H), 2.05-1.90 (m, 1H), 1.88- 1.78 (m, 1H), 1.71-1.53 (m, 2H), 1.22-1.12 (m, 2H), 1.02-0.90 (m, | 0.442 | 467.2 | Q |
| | | 2H) | | | |
| 54 | 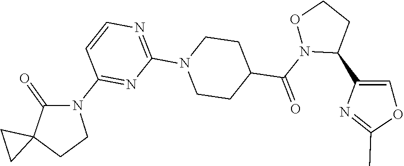 [SMILES: Cc1nc([C@@H]2CCON2C(=O)C2CCN(c3nccc(N4CCC5(CC5)C4=O)n3)CC2)co1] | 1H NMR (400 MHz, CDCl3) δ ppm = 8.20 (d, J = 5.9 Hz, 1H), 7.54 (d, J = 5.9 Hz, 1H), 7.48 (s, 1H), 5.38 (t, J = 7.1 Hz, 1H), 4.84-4.65 (m, 2H), 4.39-4.27 (m, 1H), 4.16-4.05 (m, 2H), 3.94 (q, J = 7.5 Hz, 1H), 3.11- 2.89 (m, 3H), 2.76-2.58 (m, 2H), 2.44 (s, 3H), 2.23-2.12 (m, 2H), 2.03-1.89 (m, 1H), 1.85-1.67 (m, 3H), 1.34-1.20 (m, 2H), 0.96-0.82 (m, 2H) | 0.431 | 453.2 | Q |
| 55 | 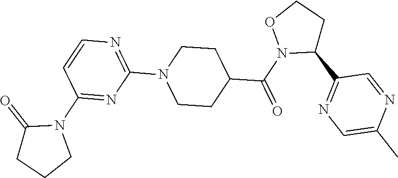 [SMILES: Cc1cnc([C@@H]2CCON2C(=O)C2CCN(c3nccc(N4CCCC4=O)n3)CC2)cn1] | 1H NMR (400 MHz, CDCl3) δ ppm = 8.53 (d, J = 1.3 Hz, 1 H), 8.40 (bd, J ~ 1.0 Hz, 1 H), 8.21 (d, J = 5.7 Hz, 1 H), 7.56 (d, J = 5.7 Hz, 1 H), 5.51 (dd, J = 8.8 Hz, 5.7 Hz, 1 H), 4.72 (m, 2 H), 4.36 (m, 1 H), 4.04 (t, J = 7.2 Hz, 2 H), 4.00 (m, 1 H), 3.10-2.96 (m, 3 H), 2.81 (m, 1 H), 2.71 (m, 1 H), 2.64 (t, J = 8.0 Hz, 2 H), 2.56 (s, 3 H), 2.11 (m, 2 H), 1.97 (m, 1 H), 1.83- 1.68 (m, 3 H). | 0.403 | 438.3 | Q |
| 56 | 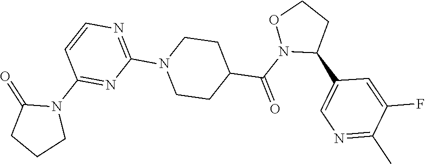 [SMILES: Cc1ncc([C@@H]2CCON2C(=O)C2CCN(c3nccc(N4CCCC4=O)n3)CC2)cc1F] | 1H NMR (400 MHz, DMSO-d6) δ ppm = 8.29-8.23 (m, 1H), 8.21 (d, J = 5.6 Hz, 1H), 7.54-7.46 (m, 1H), 7.44 (d, J = 5.6 Hz, 1H), 5.47-5.33 (m, 1H), 4.71-4.53 (m, 2H), 4.35- 4.23 (m, 1H), 4.00-3.88 (m, 3H), 3.10-2.82 (m, 4H), 2.56 (s, 2H), 2.42 (d, J = 2.6 Hz, 3H), 2.32-2.20 (m, 1H), 2.11-1.94 (m, 2H), 1.94-1.82 (m, 1H), 1.78-1.64 (m, 1H), 1.54- 1.38 (m, 2H) | 0.413 | 455.2 | Q |
| 57 | 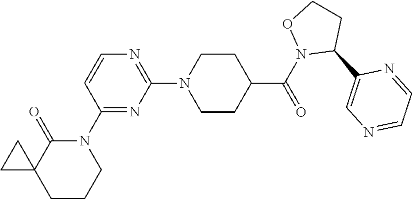 [SMILES: O=C(C1CCN(c2nccc(N3CCCC4(CC4)C3=O)n2)CC1)N1OCC[C@H]1c1cnccn1] | 1H NMR (400 MHz, MeOD-d4) δ ppm = 8.62 (s, 1H), 8.61-8.56 (m, 1H), 8.51 (d, J = 2.4 Hz, 1H), 8.14 (d, J = 5.9 Hz, 1H), 7.15 (d, J = 5.9 Hz, 1H), 5.49 (dd, J = 8.8, 5.9 Hz, 1H), 4.76-4.63 (m, 2H), 4.46-4.34 (m, 1H), 4.16-4.01 (m, 3H), 3.23-3.11 (m, 1H), 3.08-2.96 (m, 2H), 2.96-2.86 (m, 1H), 2.68- 2.54 (m, 1H), 2.10-2.00 (m, 2H), 1.99-1.89 (m, 1H), 1.87-1.76 (m, 3H), 1.71-1.53 (m, 2H), 1.38-1.27 (m, 2H), 0.76 (q, J = 3.4 Hz, 2H) | 0.522 | 464.3 | Q |
| 58 | 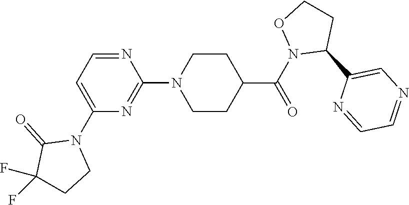 [SMILES: O=C(C1CCN(c2nccc(N3CCC(F)(F)C3=O)n2)CC1)N1OCC[C@H]1c1cnccn1] | 1H NMR (400 MHz, CDCl3) δ ppm = 8.71-8.63 (m, 1H), 8.59-8.53 (m, 1H), 8.53-8.48 (m, 1H), 8.35-8.27 (m, 1H), 7.61-7.51 (m, 1H), 5.59- 5.52 (m, 1H), 4.84-4.64 (m, 2H), 4.46-4.30 (m, 1H), 4.14-4.05 (m, 2H), 4.05-3.96 (m, 1H), 3.16-2.97 (m, 3H), 2.92-2.80 (m, 1H), 2.80- 2.68 (m, 1H), 2.68-2.51 (m, 2H), 2.07-1.95 (m, 1H), 1.90-1.67 (m, 3H) | 0.482 | 460.3 | Q |
| 59 | 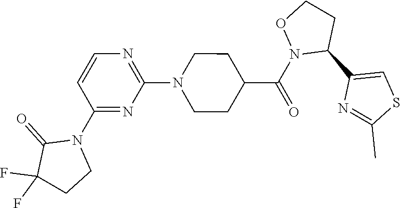 [SMILES: Cc1nc([C@@H]2CCON2C(=O)C2CCN(c3nccc(N4CCC(F)(F)C4=O)n3)CC2)cs1] | 1H NMR (400 MHz, DMSO-d6) δ ppm = 8.36 (d, J = 5.4 Hz, 1H), 7.40 (d, J = 5.9 Hz, 1H), 7.22 (s, 1H), 5.45-5.31 (m, 1H), 4.69-4.53 (m, 2H), 4.33-4.20 (m, 1H), 4.01 (t, J = 6.6 Hz, 2H), 3.96-3.83 (m, 1H), 3.01 (br d, J = 12.2 Hz, 3H), 2.79- 2.64 (m, 3H), 2.63 (s, 3H), 2.47- 2.36 (m, 1H), 1.94-1.81 (m, 1H), 1.79-1.61 (m, 1H), 1.59-1.41 (m, 2H) | 0.485 | 479.3 | Q |
| 60 | 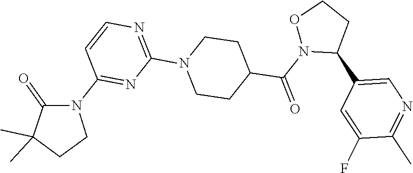 | 1H NMR (400 MHz, DMSO-d6) δ ppm = 8.27-8.24 (m, 1H), 8.22 (d, J = 5.9 Hz, 1H), 7.49 (dd, J = 10.5, 1.7 Hz, 1H), 7.46 (d, J = 5.4 Hz, 1H), 5.43-5.30 (m, 1H), 4.69-4.53 (m, 2H), 4.39-4.27 (m, 1H), 4.01-3.79 (m, 3H), 3.11-2.83 (m, 4H), 2.42 (d, J = 2.4 Hz, 3H), 2.34-2.18 (m, 1H), 1.95-1.82 (m, 3H), 1.77-1.66 (m, 1H), 1.57-1.36 (m, 2H), 1.14 (s, 6H) | 0.534 | 483.3 | Q |
| 61 | 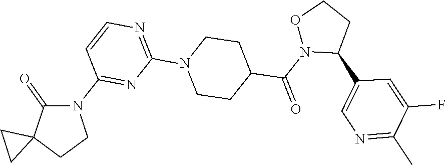 [SMILES: Cc1ncc([C@@H]2CCON2C(=O)C2CCN(c3nccc(N4CCC5(CC5)C4=O)n3)CC2)cc1F] | 1H NMR (400 MHz, CDCl3) δ ppm = 8.28 (s, 1H), 8.20 (d, J = 5.4 Hz, 1H), 7.55 (d, J = 5.4 Hz, 1H), 7.30-7.23 (m, 1H, under solvent signal), 5.43 (dd, J = 8.6, 6.1 Hz, 1H), 4.82-4.69 (m, 2H), 4.37-4.28 (m, 1H), 4.16- 4.05 (m, 2H), 3.99-3.87 (m, 1H), 3.11-2.95 (m, 3H), 2.94-2.84 (m, 1H), 2.50 (d, J = 2.9 Hz, 3H), 2.43- 2.31 (m, 1H), 2.22-2.13 (m, 2H), 2.03-1.93 (m, 1H), 1.82-1.67 (m, | 0.441 | 481.2 | Q |
| | | 3H), 1.31-1.22 (m, 2H), 0.92-0.86 | | | |
| | | (m, 2H) | | | |
| 62 | 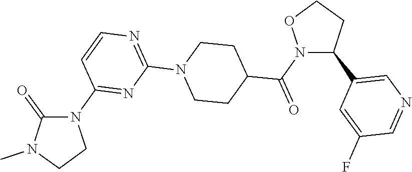 [SMILES: CN1CCN(c2ccnc(N3CCC(C(=O)N4OCC[C@H]4c4cncc(F)c4)CC3)n2)C1=O] | 1H NMR (400 MHz, CDCl3) δ ppm = 8.47-8.36 (m, 2H), 8.11 (d, J = 5.9 Hz, 1H), 7.53-7.44 (m, 1H), 7.39-7.32 (m, 1H), 5.54-5.41 (m, 1H), 4.83-4.66 (m, 2H), 4.37-4.26 (m, 1H), 4.06-3.88 (m, 3H), 3.54- 3.42 (m, 2H), 3.11-2.87 (m, 7H), 2.47-2.32 (m, 1H), 2.08-1.94 (m, 1H), 1.84-1.69 (m, 3H) | 0.403 | 456.2 | Q |
| 63 | 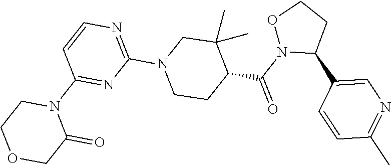 [SMILES: Cc1ccc([C@@H]2CCON2C(=O)[C@@H]2CCN(c3nccc(N4CCOCC4=O)n3)CC2(C)C)cn1] | 1H NMR (400 MHz, CDCl3) δ ppm = 8.58-8.40 (m, 1H), 8.25-8.11 (m, 1H), 7.61-7.52 (m, 1H), 7.46 (d, J = 5.9 Hz, 1H), 7.21-7.13 (m, 1H), 5.47-5.38 (m, 1H), 4.32 (s, 2H), 4.31-4.35 (m, 2H), 4.07-3.99 (m, 4H), 3.98-3.83 (m, 2H), 3.65 (d, J = 13.2 Hz, 1H), 3.56-3.43 (m, 1H), 2.94-2.79 (m, 1H), 2.70- 2.61 (m, 1H), 2.57 (s, 3H), 2.44- 2.25 (m, 1H), 2.17-1.94 (m, 2H), | 0.922 | 479.2 | S |
| | | 0.67-0.55 (m, 2H), 0.54-0.45 (m, | | | |
| | | 1H), 0.34-0.25 (m, 1H) | | | |
| 64 | 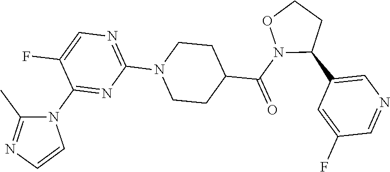 [SMILES: Cc1nccn1-c1nc(N2CCC(C(=O)N3OCC[C@H]3c3cncc(F)c3)CC2)ncc1F] | 1H NMR (400 MHz, DMSO-d6) δ ppm = 8.63 (d, J = 2.9 Hz, 1H), 8.49 (d, J = 2.9 Hz, 1H), 8.40 (s, 1H), 7.68-7.53 (m, 1H), 7.49-7.37 (m, 1H), 6.96 (d, J = 1.5 Hz, 1H), 5.47-5.37 (m, 1H), 4.59-4.45 (m, 2H), 4.38-4.27 (m, 1H), 4.02-3.90 (m, 1H), 3.18-3.00 (m, 3H), 2.98- 2.84 (m, 1H), 2.49 (s, 3H), 2.36-2.21 (m, 1H), 1.94 (br d, J = 11.2 Hz, 1H), 1.76 (br d, J = 12.2 Hz, 1H), 1.63- | 0.490 | 456.3 | Q |
| | | 1.45 (m, 2H) | | | |
| 65 | 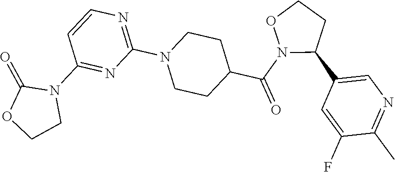 [SMILES: Cc1ncc([C@@H]2CCON2C(=O)C2CCN(c3nccc(N4CCOC4=O)n3)CC2)cc1F] | 1H NMR (400 MHz, DMSO-d6) δ ppm = 8.28-8.21 (m, 2H), 7.49 (dd, J = 10.5, 1.2 Hz, 1H), 7.22 (d, J = 5.4 Hz, 1H), 5.43-5.33 (m, 1H), 4.70- 4.55 (m, 2H), 4.43 (t, J = 8.1 Hz, 2H), 4.34-4.24 (m, 1H), 4.11 (t, J = 7.8 Hz, 2H), 3.99-3.88 (m, 1H), 3.13-2.82 (m, 4H), 2.41 (d, J = 2.9 Hz, 3H), 2.35-2.20 (m, 1H), 1.88 (br d, J = 11.2 Hz, 1H), 1.72 (br d, J = 11.7 Hz, 1H), 1.56-1.38 | 0.483 | 457.3 | Q |
| | | (m, 2H) | | | |
| 66 | 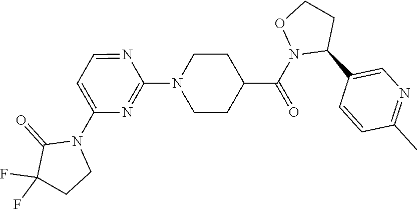 [SMILES: Cc1ccc([C@@H]2CCON2C(=O)C2CCN(c3nccc(N4CCC(F)(F)C4=O)n3)CC2)cn1] | 1H NMR (400 MHz, CDCl3) δ ppm = 8.45 (d, J = 2.0 Hz, 1H), 8.26 (d, J = 5.9 Hz, 1H), 7.50 (dd, J = 8.1, 2.2 Hz, 1H), 7.29 (d, J = 5.4 Hz, 1H), 7.12 (d, J = 7.8 Hz, 1H), 5.40 (dd, J = 8.8, 5.9 Hz, 1H), 4.74-4.58 (m, 2H), 4.36- 4.26 (m, 1H), 4.04 (t, J = 6.1 Hz, 2H), 3.99-3.89 (m, 1H), 3.12-2.93 (m, 3H), 2.93-2.79 (m, 1H), 2.53 (s, 3H), 2.47-2.29 (m, 3H), 2.17-2.06 (m, 2H), 2.05-1.90 (m, 1H), 1.83-1.62 (m, 3H) | 0.460 | 487.3 | Q |
| 67 | 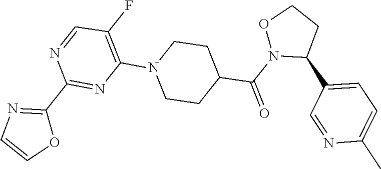 [SMILES: Cc1ccc([C@@H]2CCON2C(=O)C2CCN(c3nc(-c4ncco4)ncc3F)CC2)cn1] | 1H NMR (400 MHz, MeOD-d4) δ ppm = 8.37 (d, J = 2.0 Hz, 1H), 8.20 (d, J = 6.9 Hz, 1H), 8.08 (s, 1H), 7.68 (dd, J = 8.1, 2.3 Hz, 1H), 7.39 (s, 1H), 7.29 (d, J = 8.1 Hz, 1H), 5.46-5.32 (m, 1H), 4.78-4.67 (m, 2H), 4.41-4.29 (m, 1H), 4.11-3.97 (m, 1H), 3.29- 3.17 (m, 2H), 3.03-2.89 (m, 1H), 2.51 (s, 3H), 2.41-2.24 (m, 1H), 2.11-2.00 (m, 1H), 1.96-1.85 (m, 1H), 1.85-1.69 (m, 2H) | 0.408 | 439.1 | Q |
| 68 | 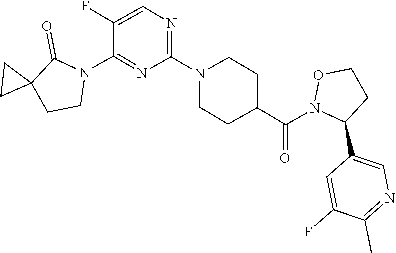 [SMILES: Cc1ncc([C@@H]2CCON2C(=O)C2CCN(c3ncc(F)c(N4CCC5(CC5)C4=O)n3)CC2)cc1F] | 1H NMR (400 MHz, CDCl3) δ = 8.28 (s, 1H), 8.17 (d, J = 3.2 Hz, 1H), 7.27-7.24 (m, 1H), 5.43 (dd, J = 8.8, 6.4 Hz, 1H), 4.64 (t, J = 12.4 Hz, 2H), 4.32 (td, J = 8.0, 3.6 Hz, 1H), 4.02 (t, J = 7.6 Hz, 2H), 3.97-3.90 (m, 1H), 3.07- 2.94 (m, 3H), 2.94-2.84 (m, 1H), 2.51 (d, J = 2.8 Hz, 3H), 2.42-2.31 (m, 1H), 2.25 (t, J = 7.6 Hz, 2H), 2.02-1.94 (m, 1H), 1.82-1.70 (m, 3H), 1.28-1.25 (m, 2H), 0.92-0.86 (m, 2H). | 0.580 | 499.3 | Q |
| 69 | 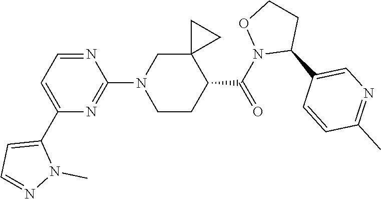 [SMILES: Cc1ccc([C@@H]2CCON2C(=O)[C@@H]2CCN(c3nccc(-c4ccnn4C)n3)CC23CC3)cn1] | 1H NMR (400 MHz, DMSO-d6) δ ppm = 8.59 (s, 1H), 8.40 (d, J = 2.1 Hz, 1H), 7.57 (dd, J = 8.0, 2.4 Hz, 1H), 7.49 (d, J = 2.0 Hz, 1H), 7.22 (d, J = 8.0 Hz, 1H), 7.02 (d, J = 1.5 Hz, 1H), 5.38 (dd, J = 8.7, 5.7 Hz, 1H), 4.34-4.18 (m, 5H), 4.02-3.86 (m, 2H), 3.80-3.57 (m, 2H), 2.94-2.83 (m, 1H), 2.80 (t, J = 5.2 Hz, 1H), 2.47 (s, 3H), 2.35-2.21 (m, 1H), 2.11- 1.90 (m, 2H), 0.63-0.47 (m, 3H), 0.44-0.33 (m, 1H) | 0.454 | 461.3 | Q |
| 70 | 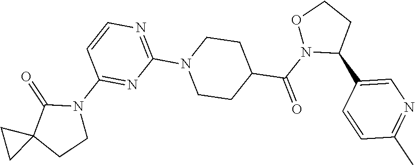 [SMILES: Cc1ccc([C@@H]2CCON2C(=O)C2CCN(c3nccc(N4CCC5(CC5)C4=O)n3)CC2)cn1] | 1H NMR (400 MHz, CDCl3) δ ppm = 8.46 (d, J = 2.2 Hz, 1H), 8.20 (d, J = 5.6 Hz, 1H), 7.55-7.49 (m, 1H), 7.13 (d, J = 7.9 Hz, 1H), 5.42 (dd, J = 8.7, 6.1 Hz, 1H), 4.80-4.67 (m, 2H), 4.39-4.27 (m, 1H), 4.15-4.06 (m, 2H), 4.04-3.89 (m, 1H), 3.09-2.93 (m, 3H), 2.92-2.79 (m, 1H), 2.54 (s, 3H), 2.44-2.30 (m, 1H), 2.22-2.11 (m, 2H), 2.02-1.92 (m, 1H), 1.83-1.66 (m, 3H), 1.31-1.22 (m, 2H), 0.92- | 0.404 | 463.2 | Q |
| | | 0.85 (m, 2H) | | | |
| 71 | 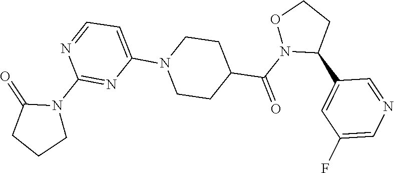 [SMILES: O=C1CCCN1c1nccc(N2CCC(C(=O)N3OCC[C@H]3c3cncc(F)c3)CC2)n1] | 1H NMR (400 MHz, CDCl3) δ ppm = 8.50-8.34 (m, 2H), 8.20 (d, J = 5.9 Hz, 1H), 7.35 (dt, J = 9.0, 2.3 Hz, 1H), 6.28 (d, J = 5.9 Hz, 1H), 5.48 (dd, J = 8.8, 5.9 Hz, 1H), 4.51- 4.30 (m, 3H), 4.04 (t, J = 7.1 Hz, 2H), 4.00-3.91 (m, 1H), 3.16- 3.00 (m, 3H), 2.99-2.88 (m, 1H), 2.65 (t, J = 8.1 Hz, 2H), 2.47-2.35 (m, 1H), 2.17-2.01 (m, 3H), 1.91-1.70 (m, 3H) | 0.837 | 441.2 | S |
| 72 | 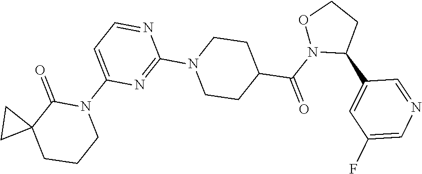 [SMILES: O=C(C1CCN(c2nccc(N3CCCC4(CC4)C3=O)n2)CC1)N1OCC[C@H]1c1cncc(F)c1] | 1H NMR (400 MHz, DMSO-d6) δ ppm = 8.49 (d, J = 2.7 Hz, 1H), 8.43-8.39 (m, 1H), 8.18 (d, J = 5.7 Hz, 1H), 7.60 (dt, J = 9.8, 2.1 Hz, 1H), 7.19 (d, J = 5.7 Hz, 1H), 5.46-5.38 (m, 1H), 4.68- 4.53 (m, 2H), 4.36-4.27 (m, 1H), 4.04-3.89 (m, 3H), 3.11-2.83 (m, 4H), 2.34-2.23 (m, 1H), 2.03-1.85 (m, 3H), 1.79-1.70 (m, 3H), 1.55-1.40 (m, 2H), | 0.441 | 481.2 | Q |
| | | 1.19 (q, J = 3.3 Hz, 2H), 0.73 | | | |
| | | (q, J = 3.4 Hz, 2H) | | | |
| 73 | 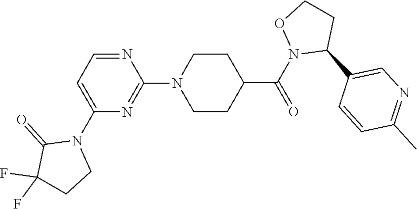 [SMILES: Cc1ccc([C@@H]2CCON2C(=O)C2CCN(c3nccc(N4CCC(F)(F)C4=O)n3)CC2)cn1] | 1H NMR (400 MHz, CDCl3) δ ppm = 8.47 (s, 1H), 8.30 (d, J = 5.4 Hz, 1H), 7.57-7.50 (m, 2H), 7.14 (d, J = 7.8 Hz, 1H), 5.41 (dd, J = 8.6, 6.1 Hz, 1H), 4.76-4.63 (m, 2H), 4.35-4.28 (m, 1H), 4.08 (t, J = 6.8 Hz, 2H), 3.99-3.90 (m, 1H), 3.09-2.95 (m, 3H), 2.93-2.82 (m, 1H), 2.66-2.51 (m, 5H), 2.43-2.31 (m, 1H), 2.03- 1.94 (m, 1H), 1.82-1.67 (m, 3H) | 0.453 | 473.3 | Q |
| 74 | 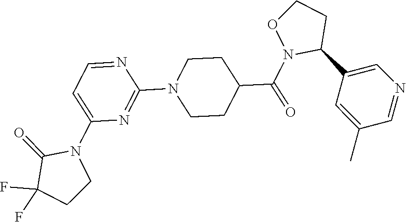 [SMILES: Cc1cncc([C@@H]2CCON2C(=O)C2CCN(c3nccc(N4CCC(F)(F)C4=O)n3)CC2)c1] | 1H NMR (400 MHz, DMSO-d6) δ ppm = 8.37 (d, J = 5.5 Hz, 1H), 8.34- 8.26 (m, 2H), 7.50-7.46 (m, 1H), 7.41 (d, J = 5.5 Hz, 1H), 5.40-5.30 (m, 1H), 4.71-4.55 (m, 2H), 4.37- 4.28 (m, 1H), 4.02 (br t, J = 6.7 Hz, 2H), 3.98-3.87 (m, 1H), 3.14-2.96 (m, 3H), 2.96-2.85 (m, 1H), 2.79- 2.61 (m, 2H), 2.30 (s, 3H), 2.27-2.18 (m, 1H), 1.97-1.86 (m, 1H), 1.78- 1.70 (m, 1H), 1.57-1.42 (m, 2H) | 0.451 | 473.3 | Q |
| 75 | 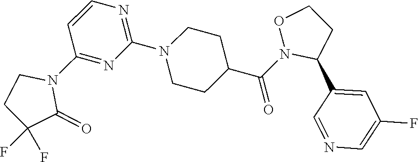 [SMILES: O=C(C1CCN(c2nccc(N3CCC(F)(F)C3=O)n2)CC1)N1OCC[C@H]1c1cncc(F)c1] | 1H NMR (400 MHz, DMSO-d6) δ ppm = 8.49 (d, J = 2.8 Hz, 1H), 8.42- 8.39 (m, 1H), 8.36 (d, J = 5.5 Hz, 1H), 7.63-7.56 (m, 1H), 7.40 (d, J = 5.5 Hz, 1H), 5.47-5.37 (m, 1H), 4.68-4.56 (m, 2H), 4.37-4.26 (m, 1H), 4.01 (s, 3H), 3.14-2.97 (m, 3H), 2.96-2.88 (m, 1H), 2.69 (s, 2H), 2.34-2.21 (m, 1H), 1.96-1.85 (m, 1H), 1.80-1.70 (m, 1H), 1.56- | 0.507 | 477.3 | Q |
| | | 1.41 (m, 2H) | | | |
| 76 | 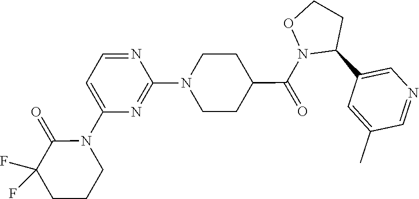 [SMILES: Cc1cncc([C@@H]2CCON2C(=O)C2CCN(c3nccc(N4CCCC(F)(F)C4=O)n3)CC2)c1] | 1H NMR (400 MHz, CDCl3) δ ppm = 8.38 (br d, J = 15.8 Hz, 2H), 8.28 (d, J = 5.6 Hz, 1H), 7.44 (s, 1H), 7.31 (d, J = 5.6 Hz, 1H), 5.42 (dd, J = 8.7, 6.2 Hz, 1H), 4.79-4.61 (m, 2H), 4.38-4.27 (m, 1H), 4.06 (t, J = 6.1 Hz, 2H), 4.00-3.90 (m, 1H), 3.11-2.96 (m, 3H), 2.94-2.83 (m, 1H), 2.47-2.31 (m, 6H), 2.17- 2.08 (m, 2H), 2.05-1.96 (m, 1H), 1.85-1.68 (m, 3H) | 0.503 | 487.3 | Q |
| 77 | 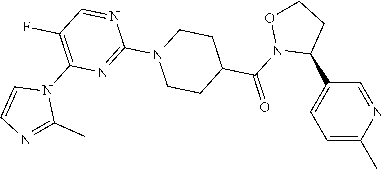 [SMILES: Cc1ccc([C@@H]2CCON2C(=O)C2CCN(c3ncc(F)c(-n4ccnc4C)n3)CC2)cn1] | 1H NMR (400 MHz, DMSO-d6) δ ppm = 8.63 (d, J = 2.9 Hz, 1H), 8.36 (d, J = 2.0 Hz, 1H), 7.54 (dd, J = 8.0, 2.3 Hz, 1H), 7.45 (dd, J = 3.4, 1.5 Hz, 1H), 7.22 (d, J = 7.9 Hz, 1H), 6.96 (d, J = 1.5 Hz, 1H), 5.39-5.25 (m, 1H), 4.58-4.45 (m, 2H), 4.34-4.24 (m, 1H), 3.97-3.87 (m, 1H), 3.15-2.99 (m, 3H), 2.94-2.82 (m, 1H), 2.48 (s, 3H), 2.43 (s, 3H), 2.27-2.17 (m, 1H), 1.95-1.85 (m, 1H), 1.79-1.68 (m, | 0.442 | 452.3 | Q |
| | | 1H), 1.59-1.44 (m, 2H) | | | |
| 78 | 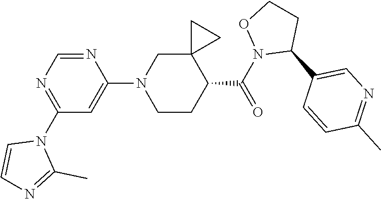 [SMILES: Cc1ccc([C@@H]2CCON2C(=O)[C@@H]2CCN(c3cc(-n4ccnc4C)ncn3)CC23CC3)cn1] | 1H NMR (400 MHz, MeOD-d4) δ ppm = 8.49-8.28 (m, 2H), 7.69 (dd, J = 8.1, 2.3 Hz, 1H), 7.46 (d, J = 1.6 Hz, 1H), 7.31 (d, J = 8.1 Hz, 1H), 6.95 (d, J = 1.6 Hz, 1H), 6.74 (s, 1H), 5.40 (dd, J = 8.6, 6.4 Hz, 1H), 4.32 (dt, J = 7.8, 3.4 Hz, 1H), 4.18-4.04 (m, 1H), 4.05-3.89 (m, 2H), 3.67-3.48 (m, 2H), 3.04-2.89 (m, 1H), 2.87-2.73 (m, 1H), 2.57 (s, 3H), 2.52 (s, 3H), 2.43-2.26 (m, 1H), 2.19-1.99 (m, 2H), 0.69-0.49 (m, 3H), 0.47-0.32 | 0.399 | 460.3 | Q |
| | | (m, 1H) | | | |
| 79 | 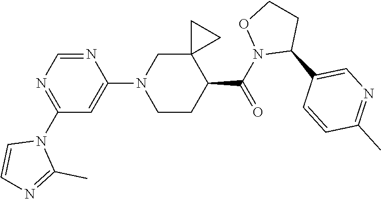 [SMILES: Cc1ccc([C@@H]2CCON2C(=O)[C@H]2CCN(c3cc(-n4ccnc4C)ncn3)CC23CC3)cn1] | 1H NMR (400 MHz, CDCl3) δ ppm = 8.55-8.42 (m, 2H), 7.50 (dd, J = 8.0, 2.4 Hz, 1H), 7.15 (d, J = 8.1 Hz, 1H), 7.00 (d, J = 1.5 Hz, 1H), 6.31 (s, 1H), 5.41 (dd, J = 8.7, 6.1 Hz, 1H), 4.28 (td, J = 7.8, 3.5, 1H), 4.05-3.74 (m, 4H), 3.54-3.34 (m, 1H), 3.00-2.80 (m, 2H), 2.63 (s, 3H), 2.55 (s, 3H), 2.40-2.31 (m, 1H), 2.09-1.88 (m, 2H), 0.80-0.68 (m, 1H), 0.65-0.48 (m, 3H) | 0.403 | 460.3 | Q |
| 80 | 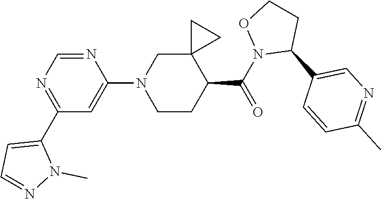 [SMILES: Cc1ccc([C@@H]2CCON2C(=O)[C@H]2CCN(c3cc(-c4ccnn4C)ncn3)CC23CC3)cn1] | 1H NMR (400 MHz, CDCl3) δ ppm = 8.59 (d, J = 0.9 Hz, 1H), 8.47 (d, J = 2.3 Hz, 1H), 7.56-7.46 (m, 2H), 7.16 (d, J = 8.1 Hz, 1H), 6.64 (s, 1H), 6.56 (d, J = 2.1 Hz, 1H), 5.42 (dd, J = 8.7, 6.1 Hz, 1H), 4.33-4.25 (m, 1H), 4.21 (s, 3H), 4.04-3.87 (m, 2H), 3.87- 3.75 (m, 2H), 3.53-3.37 (m, 1H), 2.97-2.81 (m, 2H), 2.56 (s, 3H), 2.44-2.30 (m, 1H), 2.07-1.89 (m, 2H), 0.75-0.49 (m, 4H) | 0.404 | 460.3 | Q |
| 81 | 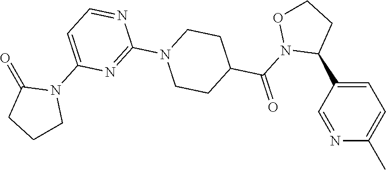 [SMILES: Cc1ccc([C@@H]2CCON2C(=O)C2CCN(c3nccc(N4CCCC4=O)n3)CC2)cn1] | 1H NMR (400 MHz, MeOD-d4) δ ppm = 8.36 (d, J = 1.8 Hz, 1H), 8.15 (d, J = 5.7 Hz, 1H), 7.67 (dd, J = 8.1, 2.3 Hz, 1H), 7.51 (d, J = 5.7 Hz, 1H), 7.29 (d, J = 8.1 Hz, 1H), 5.42-5.35 (m, 1H), 4.75-4.63 (m, 2H), 4.36 (td, J = 7.8, 3.2 Hz, 1H), 4.14-3.94 (m, 3H), 3.20-3.09 (m, 1H), 3.05-2.91 (m, 3H), 2.68-2.55 (m, 2H), 2.51 (s, 3H), 2.40-2.28 (m, 1H), 2.19-2.05 (m, 2H), 2.00-1.89 (m, 1H), 1.84-1.72 | 0.376 | 437.2 | Q |
| | | (m, 1H), 1.70-1.52 (m, 2H) | | | |
| 82 | 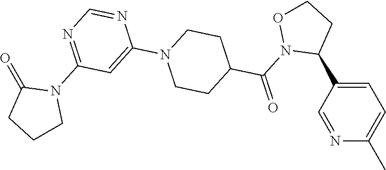 [SMILES: Cc1ccc([C@@H]2CCON2C(=O)C2CCN(c3cc(N4CCCC4=O)ncn3)CC2)cn1] | 1H NMR (400 MHz, MeOD-d4) δ ppm = 8.37 (d, J = 2.0 Hz, 1H), 8.30 (s, 1H), 7.71-7.61 (m, 2H), 7.30 (d, J = 8.3 Hz, 1H), 5.45-5.31 (m, 1H), 4.48-4.29 (m, 3H), 4.10-3.95 (m, 3H), 3.24-2.89 (m, 4H), 2.63 (t, J = 8.1 Hz, 2H), 2.51 (s, 3H), 2.38- 2.29 (m, 1H), 2.18-2.05 (m, 2H), 2.03-1.94 (m, 1H), 1.89-1.77 (m, 1H), 1.71-1.56 (m, 2H) | 0.367 | 437.2 | Q |
| 83 | 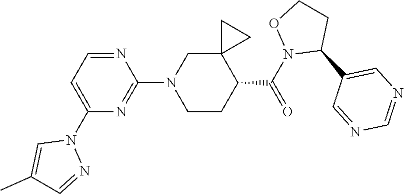 [SMILES: Cc1cnn(-c2ccnc(N3CC[C@@H](C(=O)N4OCC[C@H]4c4cncnc4)C4(CC4)C3)n2)c1] | 1H NMR (400 MHz, CDCl3) δ ppm = 8.69 (d, J = 1.2 Hz, 1H), 8.59-8.50 (m, 2H), 8.29 (d, J = 5.5 Hz, 1H), 8.22 (s, 1H), 7.55 (s, 1H), 7.00 (d, J = 5.5 Hz, 1H), 5.62-5.50 (m, 1H), 4.41-4.29 (m, 2H), 4.05- 3.92 (m, 2H), 3.79-3.69 (m, 1H), 3.68-3.56 (m, 1H), 2.90-2.76 (m, 1H), 2.76-2.63 (m, 2H), 2.20- 2.01 (m, 5H), 0.72-0.51 (m, 3H), 0.36-0.29 (m, 1H) | 0.483 | 447.3 | Q |
| 84 | 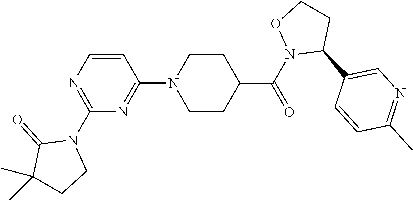 | 1H NMR (400 MHz, DMSO-d6) δ ppm = 8.36 (d, J = 2.1 Hz, 1H), 8.08 (d, J = 6.1 Hz, 1H), 7.55 (dd, J = 8.0, 2.4 Hz, 1H), 7.22 (d, J = 8.0 Hz, 1H), 6.55 (d, J = 6.3 Hz, 1H), 5.42-5.25 (m, 1H), 4.51-4.25 (m, 3H), 3.99- 3.88 (m, 1H), 3.82 (t, J = 6.9 Hz, 2H), 3.11-2.94 (m, 3H), 2.94-2.81 (m, 1H), 2.43 (s, 3H), 2.32-2.16 (m, 1H), 1.82-1.82 (m, 1H), 1.94-1.81 (m, 2H), 1.80-1.68 (m, 1H), 1.54-1.41 (m, 2H), 1.10 (s, 6H) | 0.403 | 465.3 | Q |
| 85 | 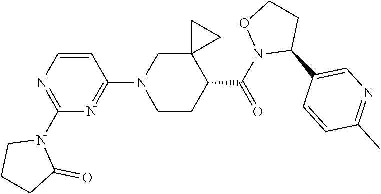 [SMILES: Cc1ccc([C@@H]2CCON2C(=O)[C@@H]2CCN(c3ccnc(N4CCCC4=O)n3)CC23CC3)cn1] | 1H NMR (400 MHz, CDCl3) δ = 8.46 (d, J = 2.0 Hz, 1H), 8.14 (d, J = 6.0 Hz, 1H), 7.51 (dd, J = 8.0, 2.0 Hz, 1H), 7.14 (d, J = 8.0 Hz, 1H), 6.18 (d, J = 6.0 Hz, 1H), 5.45-5.36 (m, 1H), 4.31-4.22 (m, 1H), 4.13-3.77 (m, 5H), 3.64-3.21 (m, 2H), 2.91-2.81 (m, 1H), 2.66-2.60 (m, 3H), 2.55 (s, 3H), 2.40-2.30 (m, 1H), 2.13-2.02 (m, 4H), 0.67-0.56 (m, 2H), 0.54- 0.48 (m, 1H), 0.36-0.28 (m, 1H). | 0.387 | 463.2 | Q |
| 86 | 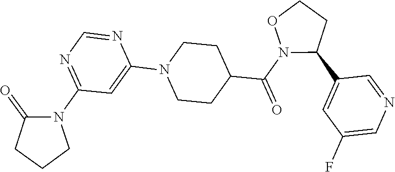 [SMILES: O=C1CCCN1c1cc(N2CCC(C(=O)N3OCC[C@H]3c3cncc(F)c3)CC2)ncn1] | 1H NMR (400 MHz, CDCl3) δ ppm = 8.48-8.30 (m, 3H), 7.71 (s, 1H), 7.34 (td, J = 9.3, 2.0 Hz, 1H), 5.47 (dd, J = 8.8, 6.4 Hz, 1H), 4.52-4.39 (m, 2H), 4.33 (td, J = 7.8, 3.4 Hz, 1H), 4.07 (t, J = 7.1 Hz, 2H), 4.01-3.89 (m, 1H), 3.14-2.98 (m, 3H), 2.97-2.87 (m, 1H), 2.64 (t, J = 8.1 Hz, 2H), 2.45- 2.31 (m, 1H), 2.11 (quin, J = 7.6 Hz, 2H), 2.06-1.96 (m, 1H), 1.87-1.68 (m, 3H), 1.59 (s, 6H) | 0.516 | 441.3 | Q |
| 87 | 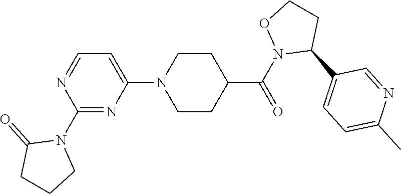 [SMILES: Cc1ccc([C@@H]2CCON2C(=O)C2CCN(c3ccnc(N4CCCC4=O)n3)CC2)cn1] | 1H NMR (400 MHz, DMSO-d6) δ ppm = 8.36 (d, J = 2.0 Hz, 1H), 8.08 (d, J = 6.4 Hz, 1H), 7.55 (dd, J = 7.8, 2.4 Hz, 1H), 7.22 (d, J = 7.8 Hz, 1H), 6.55 (d, J = 6.4 Hz, 1H), 5.41-5.24 (m, 1H), 4.50-4.23 (m, 3H), 4.02- 3.81 (m, 3H), 3.10-2.94 (m, 3H), 2.92-2.81 (m, 1H), 2.49-2.41 (m, 5H), 2.31-2.16 (m, 1H), 2.03-1.91 (m, 2H), 1.91-1.84 (m, 1H), 1.77- 1.62 (m, 1H), 1.54-1.40 (m, 2H) | 0.430 | 437.3 | Q |
| 88 | 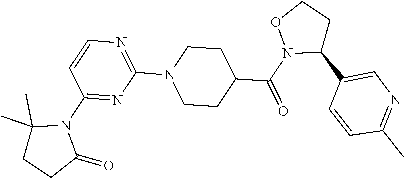 | 1H NMR (400 MHz, DMSO-d6) δ ppm = 8.37 (d, J = 2.0 Hz, 1H), 8.24 (d, J = 5.4 Hz, 1H), 7.55 (dd, J = 8.1, 2.2 Hz, 1H), 7.23 (d, J = 8.3 Hz, 1H), 7.13 (d, J = 5.9 Hz, 1H), 5.38-5.27 (m, 1H), 4.61-4.48 (m, 2H), 4.35- 4.23 (m, 1H), 4.02-3.88 (m, 1H), 3.14-2.96 (m, 3H), 2.88 (td, J = 6.0, 2.7 Hz, 1H), 2.56-2.52 (m, 2H), 2.44 (s, 3H), 2.29-2.17 (m, 1H), 1.95- 1.83 (m, 3H), 1.79-1.65 (m, 1H), 1.58 (s, 6H), 1.54-1.39 (m, 2H) | 0.387 | 465.3 | Q |
| 89 | 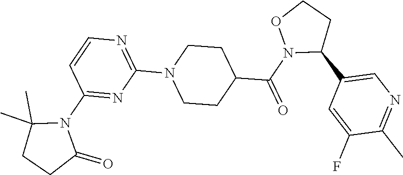 | 1H NMR (400 MHz, DMSO-d6) δ ppm = 8.26-8.20 (m, 2H), 7.49 (d, J = 10.5 Hz, 1H), 7.12 (d, J = 5.6 Hz, 1H), 5.41-5.33 (m, 1H), 4.60-4.49 (m, 2H), 4.34-4.26 (m, 1H), 4.01- 3.87 (m, 1H), 3.12-2.96 (m, 3H), 2.94-2.83 (m, 1H), 2.56-2.51 (m, 2H), 2.42 (d, J = 2.8 Hz, 3H), 2.34- 2.20 (m, 1H), 1.93-1.83 (m, 3H), 1.78-1.68 (m, 1H), 1.57 (s, 6H), 1.53-1.40 (m, 2H) | 0.443 | 483.3 | Q |
| 90 | 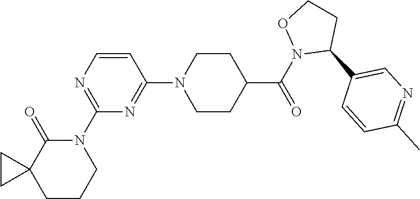 [SMILES: Cc1ccc([C@@H]2CCON2C(=O)C2CCN(c3ccnc(N4CCCC5(CC5)C4=O)n3)CC2)cn1] | 1H NMR (400 MHz, DMSO-d6) δ ppm = 8.36 (d, J = 2.0 Hz, 1H), 8.13 (d, J = 6.1 Hz, 1H), 7.55 (dd, J = 8.0, 2.4 Hz, 1H), 7.22 (d, J = 8.0 Hz, 1H), 6.68 (d, J = 6.3 Hz, 1H), 5.40-5.24 (m, 1H), 4.44-4.22 (m, 3H), 3.99- 3.87 (m, 1H), 3.77 (t, J = 6.0 Hz, 2H), 3.14-2.93 (m, 3H), 2.93-2.83 (m, 1H), 2.43 (s, 3H), 2.30-2.14 (m, 1H), 2.10-1.84 (m, 3H), 1.80-1.67 (m, 3H), 1.57-1.38 (m, 2H), 1.08 (q, J = 3.2 Hz, 2H), 0.63 (q, J = 3.5 Hz, 2H) | 0.851 | 477.2 | R |
| 91 | 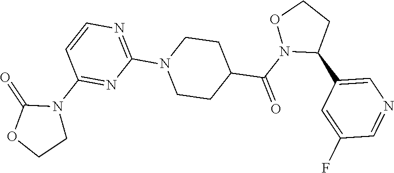 [SMILES: O=C1OCCN1c1ccnc(N2CCC(C(=O)N3OCC[C@H]3c3cncc(F)c3)CC2)n1] | 1H NMR (400 MHz, DMSO-d6) δ ppm = 8.49 (d, J = 2.9 Hz, 1H), 8.40 (s, 1H), 8.23 (d, J = 5.9 Hz, 1H), 7.68-7.49 (m, 1H), 7.23 (d, J = 5.9 Hz, 1H), 5.48-5.33 (m, 1H), 4.68-4.55 (m, 2H), 4.44 (t, J = 8.1 Hz, 2H), 4.37-4.28 (m, 1H), 4.17-4.05 (m, 2H), 4.00-3.87 (m, 1H), 3.13-2.84 (m, 4H), 2.38-2.19 (m, 1H), 1.96-1.88 (m, 1H), 1.78-1.67 (m, 1H), | 0.489 | 443.2 | Q |
| | | 1.56-1.40 (m, 2H) | | | |
| 92 | 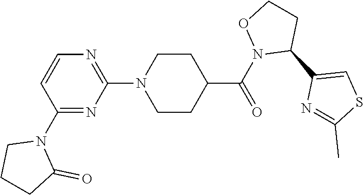 [SMILES: Cc1nc([C@@H]2CCON2C(=O)C2CCN(c3nccc(N4CCCC4=O)n3)CC2)cs1] | 1H NMR (400 MHz, CDCl3) δ ppm = 8.21 (d, J = 5.8 Hz, 1H), 7.61-7.53 (m, 1H), 7.01 (s, 1H), 5.53 (dd, J = 8.6, 5.3 Hz, 1H), 4.80-4.66 (m, 2H), 4.32 (td, J = 7.5, 4.7 Hz, 1H), 4.04 (t, J = 7.2 Hz, 2H), 3.96 (q, J = 7.8 Hz, 1H), 3.12-2.92 (m, 3H), 2.81-2.60 (m, 7H), 2.16-2.06 (m, 2H), 2.03- 1.94 (m, 1H), 1.86-1.69 (m, 3H) | 0.489 | 443.2 | Q |
| 93 | 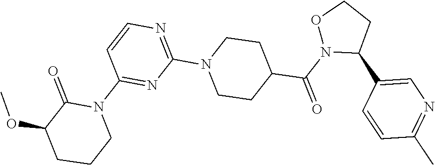 | 1H NMR (400 MHz, MeOD-d4) δ ppm = 8.36 (d, J = 2.0 Hz, 1H), 8.18 (d, J = 5.7 Hz, 1H), 7.67 (dd, J = 8.1, 2.3 Hz, 1H), 7.29 (d, J = 8.2 Hz, 1H), 7.25 (d, J = 5.7 Hz, 1H), 5.43-5.35 (m, 1H), 4.74-4.64 (m, 2H), 4.40-4.32 (m, 1H), 4.28-4.18 (m, 1H), 4.07- 3.94 (m, 2H), 3.85-3.74 (m, 1H), 3.53 (s, 3H), 3.20-3.10 (m, 1H), 3.08-2.92 (m, 3H), 2.51 (s, 3H), | 0.457 | 481.3 | Q |
| | | 2.40-2.28 (m, 1H), 2.28-2.17 (m, | | | |
| | | 1H), 2.05-1.73 (m, 5H), 1.70- | | | |
| | | 1.54 (m, 2H) | | | |
| 94 | 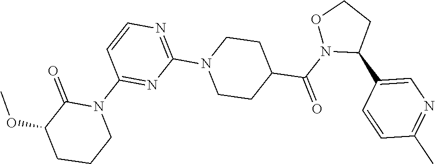 | 1H NMR (400 MHz, MeOD-d4) δ ppm = 8.38 (d, J = 1.6 Hz, 1H), 8.20 (d, J = 5.7 Hz, 1H), 7.69 (dd, J = 7.9, 2.3 Hz, 1H), 7.31 (d, J = 7.9 Hz, 1H), 7.27 (d, J = 5.7 Hz, 1H), 5.45-5.36 (m, 1H), 4.75-4.67 (m, 2H), 4.38 (t, J = 7.8, 3.1 Hz, 1H), 4.25 (dt, J = 13.1, 5.1 Hz, 1H), 4.08-3.94 (m, 2H), 3.89-3.77 (m, 1H), 3.55 (s, 3H), 3.22-3.13 (m, 1H), 3.09-2.94 (m, | 0.466 | 481.3 | Q |
| | | 3H), 2.53 (s, 3H), 2.42-2.31 (m, | | | |
| | | 1H), 2.30-2.20 (m, 1H), 2.07- | | | |
| | | 1.76 (m, 5H), 1.72-1.57 (m, 2H) | | | |
| 95 | 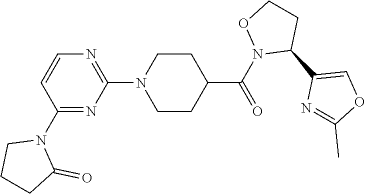 [SMILES: Cc1nc([C@@H]2CCON2C(=O)C2CCN(c3nccc(N4CCCC4=O)n3)CC2)co1] | 1H NMR (400 MHz, MeOD-d4) δ ppm = 8.16 (d, J = 5.8 Hz, 1H), 7.67 (s, 1H), 7.51 (d, J = 5.8 Hz, 1H), 5.36-5.27 (m, 1H), 4.86 (s, 3H), 4.74-4.65 (m, 2H), 4.33 (td, J = 7.6, 4.4 Hz, 1H), 4.08-3.96 (m, 3H), 3.15-3.04 (m, 1H), 3.04-2.92 (m, 2H), 2.80-2.69 (m, 1H), 2.63 (t, J = 8.1 Hz, 2H), 2.56-2.45 (m, 1H), 2.42 (s, 3H), 2.17-2.05 (m, 2H), 1.95-1.87 (m, 1H), 1.82-1.74 (m, 1H), | 0.479 | 427.3 | Q |
| | | 1.68-1.54 (m, 2H) | | | |
| 96 | 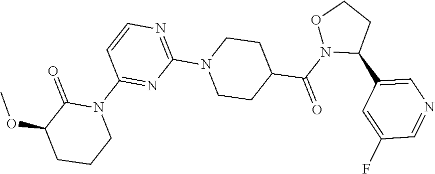 [SMILES: CO[C@@H]1CCCN(c2ccnc(N3CCC(C(=O)N4OCC[C@H]4c4cncc(F)c4)CC3)n2)C1=O] | 1H NMR (400 MHz, MeOD-d4) δ ppm = 8.45-8.34 (m, 2H), 8.20 (d, J = 5.8 Hz, 1H), 7.65-7.56 (m, 1H), 7.27 (d, J = 5.6 Hz, 1H), 5.52-5.44 (m, 1H), 4.77-4.67 (m, 2H), 4.40 (td, J = 7.8, 2.9 Hz, 1H), 4.31-4.20 (m, 1H), 4.11- 3.95 (m, 2H), 3.82 (s, 1H), 3.55 (s, 3H), 3.24-3.13 (m, 1H), 3.12-2.96 (m, 3H), 2.46-2.33 (m, 1H), 2.31- 2.19 (m, 1H), 2.09-1.80 (m, 5H), | 0.505 | 485.3 | Q |
| | | 1.73-1.58 (m, 2H) | | | |
| 97 | 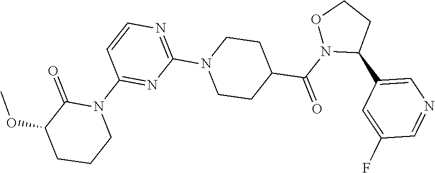 [SMILES: CO[C@H]1CCCN(c2ccnc(N3CCC(C(=O)N4OCC[C@H]4c4cncc(F)c4)CC3)n2)C1=O] | 1H NMR (400 MHz, MeOD-d4) δ ppm = 8.42-8.35 (m, 2H), 8.18 (d, J = 5.7 Hz, 1H), 7.59 (dt, J = 9.3, 2.1 Hz, 1H), 7.25 (d, J = 5.7 Hz, 1H), 5.55-5.41 (m, 1H), 4.74-4.65 (m, 2H), 4.42- 4.33 (m, 1H), 4.28-4.19 (m, 1H), 4.08-3.95 (m, 2H), 3.85- 3.75 (m, 1H), 3.53 (s, 3H), 3.22-3.12 (m, 1H), 3.09-2.94 (m, 3H), 2.43-2.31 (m, 1H), | 0.915 | 485.3 | S |
| | | 2.29-2.17 (m, 1H), 2.04-1.75 | | | |
| | | (m, 5H), 1.70-1.56 (m, 2H) | | | |
| 98 | 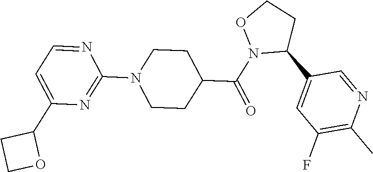 [SMILES: Cc1ncc([C@@H]2CCON2C(=O)C2CCN(c3nccc(C4CCO4)n3)CC2)cc1F] | 1H NMR (400 MHz, MeOD-d4) δ ppm = 8.37 (d, J = 5.0 Hz, 1H), 8.25 (s, 1H), 7.51 (dd, J = 10.1, 1.5 Hz, 1H), 6.83 (d, J = 5.0 Hz, 1H), 5.61- 5.50 (m, 1H), 5.47-5.40 (m, 1H), 4.87-4.74 (m, 3H), 4.73-4.66 (m, 1H), 4.42-4.34 (m, 1H), 4.11-3.98 (m, 1H), 3.22-2.94 (m, 5H), 2.73- 2.60 (m, 1H), 2.50 (d, J = 2.8 Hz, 3H), 2.44-2.32 (m, 1H), 2.02- 1.92 (m, 1H), 1.88-1.75 (m, 1H), | 0.523 | 428.3 | Q |
| | | 1.73-1.54 (m, 2H) | | | |
| 99 | 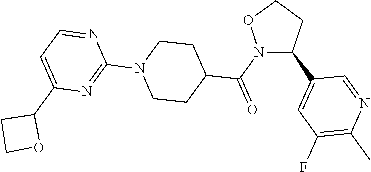 [SMILES: Cc1ncc([C@@H]2CCON2C(=O)C2CCN(c3nccc(C4CCO4)n3)CC2)cc1F] | 1H NMR (400 MHz, MeOD-d4) δ ppm = 8.35 (d, J = 5.0 Hz, 1H), 8.23 (s, 1H), 7.49 (dd, J = 10.1, 1.3 Hz, 1H), 6.81 (d, J = 5.0 Hz, 1H), 5.54 (dd, J = 8.3, 6.8 Hz, 1H), 5.46-5.34 (m, 1H), 4.85-4.72 (m, 3H), 4.68 (dt, J = 9.0, 6.0 Hz, 1H), 4.37 (dt, J = 7.8, 3.7 Hz, 1H), 4.08-3.96 (m, 1H), 3.21-2.90 (m, 5H), 2.73-2.59 (m, 1H), 2.48 (d, J = 2.8 Hz, 3H), 2.43-2.30 (m, 1H), | 0.526 | 428.3 | Q |
| | | 2.01-1.91 (m, 1H), 1.85-1.73 | | | |
| | | (m, 1H), 1.70-1.53 (m, 2H) | | | |
| 100 | 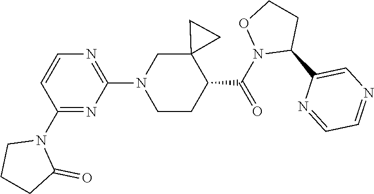 [SMILES: O=C1CCCN1c1ccnc(N2CC[C@@H](C(=O)N3OCC[C@H]3c3cnccn3)C3(CC3)C2)n1] | 1H NMR (400 MHz, CDCl3) δ ppm = 8.68 (s, 1H), 8.58-8.53 (m, 1H), 8.52 (d, J = 2.4 Hz, 1H), 8.18 (d, J = 5.9 Hz, 1H), 7.57-7.48 (m, 1H), 5.56 (dd, J = 9.0, 5.6 Hz, 1H), 4.33 (td, J = 7.7, 4.2 Hz, 1H), 4.29-4.17 (m, 1H), 4.06-3.89 (m, 4H), 3.69-3.51 (m, 2H), 2.91-2.77 (m, 1H), 2.75-2.56 (m, 4H), 2.10 (s, 4H), 0.67-0.48 (m, 3H), 0.34-0.27 (m, 1H) | 0.487 | 450.3 | Q |
| 101 | 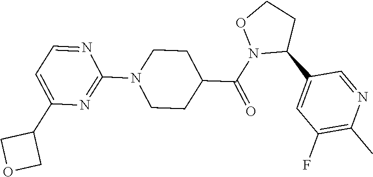 [SMILES: Cc1ncc([C@@H]2CCON2C(=O)C2CCN(c3nccc(C4COC4)n3)CC2)cc1F] | 1H NMR (400 MHz, CDCl3) δ = 8.28 (s, 1H), 8.25 (d, J = 4.8 Hz, 1H), 7.27- 7.25 (m, 1H), 6.40 (d, J = 4.8 Hz, 1H), 5.43 (dd, J = 8.8, 6.4 Hz, 1H), 4.98-4.83 (m, 6H), 4.32 (dt, J = 8.0, 3.2 Hz, 1H), 4.20-4.10 (m, 1H), 3.98- 3.90 (m, 1H), 3.12-2.96 (m, 3H), 2.94-2.83 (m, 1H), 2.51 (d, J = 3.2 Hz, 3H), 2.43-2.31 (m, 1H), 2.00 (dd, J = 13.6, 2.8 Hz, 1H), 1.84-1.71 (m, 3H). | 0.501 | 429.3 | Q |
| 102 | 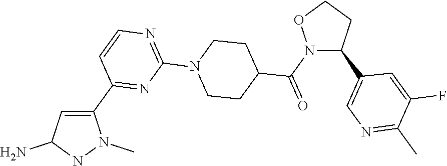 [SMILES: Cc1ncc([C@@H]2CCON2C(=O)C2CCN(c3nccc(C4=CC(N)NN4C)n3)CC2)cc1F] | 1H NMR (400 MHz, CD3OD) δ = 8.32 (d, J = 5.2 Hz, 1H), 8.24 (s, 1H), 7.49 (dd, J = 10.0, 1.2 Hz, 1H), 6.80 (d, J = 5.2 Hz, 1H), 6.11 (s, 1H), 5.41 (t, J = 7.6 Hz, 1H), 4.80-4.71 (m, 2H), 4.37 (td, J = 7.6, 3.2 Hz, 1H), 4.06- 3.99 (m, 4H), 3.25-3.15 (m, 1H), 3.14-3.03 (m, 2H), 3.02-2.92 (m, 1H), 2.48 (d, J = 2.8 Hz, 3H), 2.41- 2.31 (m, 1H), 2.03-1.95 (m, 1H), 1.90-1.75 (m, 1H), 1.73-1.58 (m, | 0.421 | 467.2 | Q |
| | | 2H). | | | |
| 103 | 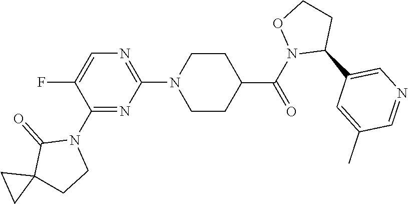 [SMILES: Cc1cncc([C@@H]2CCON2C(=O)C2CCN(c3ncc(F)c(N4CCC5(CC5)C4=O)n3)CC2)c1] | 1H NMR (400 MHz, CDCl3) δ = 8.40 (d, J = 1.6 Hz, 1H), 8.36 (d, J = 1.6 Hz, 1H), 8.17 (d, J = 2.8 Hz, 1H), 7.42 (s, 1H), 5.42 (dd, J = 8.8, 6.4 Hz, 1H), 4.69-4.58 (m, 2H), 4.32 (td, J = 7.6, 3.2 Hz, 1H), 4.05-3.99 (m, 2H), 3.98-3.90 (m, 1H), 3.08-2.94 (m, 3H), 2.93-2.83 (m, 1H), 2.43-2.35 (m, 1H), 2.34 (s, 3H), 2.25 (t, J = 7.2 Hz, 2H), 2.04-1.95 (m, 1H), 1.81-1.71 (m, 3H), 1.29-1.25 (m, 2H), 0.93- 0.85 (m, 2H). | 0.520 | 481.3 | Q |
| 104 | 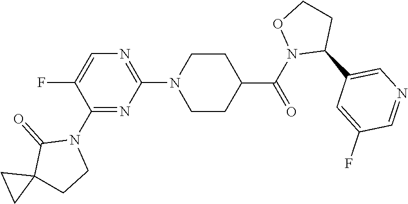 [SMILES: O=C(C1CCN(c2ncc(F)c(N3CCC4(CC4)C3=O)n2)CC1)N1OCC[C@H]1c1cncc(F)c1] | 1H NMR (400 MHz, CDCl3) δ = 8.42 (s, 1H), 8.39 (d, J = 2.8 Hz, 1H), 8.17 (d, J = 2.8 Hz, 1H), 7.35 (dt, J = 9.2, 2.8 Hz, 1H), 5.48 (dd, J = 8.8, 6.4 Hz, 1H), 4.71-4.58 (m, 2H), 4.33 (td, J = 7.6, 3.6 Hz, 1H), 4.02 (t, J = 7.2 Hz, 2H), 3.98-3.89 (m, 1H), 3.08-2.87 (m, 4H), 2.44-2.31 (m, 1H), 2.25 (t, J = 7.2 Hz, 2H), 2.05-1.93 (m, 1H), 1.83-1.68 (m, 3H), 1.30-1.22 (m, 2H), 0.92-0.84 (m, 2H). | 0.580 | 485.3 | Q |
| 105 | 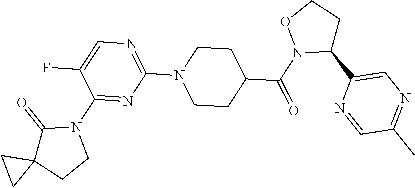 [SMILES: Cc1cnc([C@@H]2CCON2C(=O)C2CCN(c3ncc(F)c(N4CCC5(CC5)C4=O)n3)CC2)cn1] | 1H NMR (400 MHz, CDCl3) δ = 8.40 (d, J = 1.6 Hz, 1H), 8.36 (d, J = 1.6 Hz, 1H), 8.17 (d, J = 2.8 Hz, 1H), 7.42 (s, 1H), 5.42 (dd, J = 8.8, 6.4 Hz, 1H), 4.69-4.58 (m, 2H), 4.32 (td, J = 7.6, 3.2 Hz, 1H), 4.05-3.99 (m, 2H), 3.98- 3.90 (m, 1H), 3.08-2.94 (m, 3H), 2.93-2.83 (m, 1H), 2.43-2.35 (m, 1H), 2.34 (s, 3H), 2.25 (t, J = 7.2 Hz, 2H), 2.04-1.95 (m, 1H), 1.81-1.71 (m, 3H), 1.29-1.25 (m, 2H), 0.93- 0.85 (m, 2H). | 0.568 | 482.3 | Q |
| 106 | 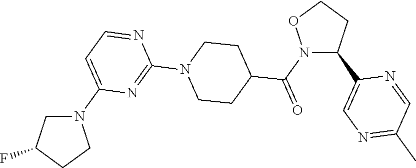 [SMILES: Cc1cnc([C@@H]2CCON2C(=O)C2CCN(c3nccc(N4CC[C@H](F)C4)n3)CC2)cn1] | 1H NMR (400 MHz, MeOD) δ = 8.48 (d, J = 8.13 Hz 1H), 7.79 (d, J = 6.1 Hz Hz, 1H), 5.89 (m, 1H), 5.46 (m, 1H), 4.65 (dd, J = 13.1, 3.3, Hz, 2H), 4.40 (m, 2H), 4.08 (dd, J = 18.19, 1.44, Hz, 2H), 3.8-3.4 (bm, 4H), 3.13 (m, 1H), 2.91 (m, 3H), 2.56 (s, 1H), 2.54 (s, 3H), 2.28-2.11 (m, 2H), 1.91 (m, 1H), 1.77 (m, 1H), 1.60 (m, 2H). | 0.404 | 442.3 | Q |
| 107 | 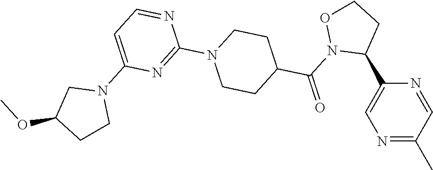 | 1H NMR (400 MHz, CD3OD) δ = 8.49 (s, 1H), 8.47 (s, 1H), 7.76 (d, J = 6.0 Hz, 1H), 5.79 (d, J = 6.0 Hz, 1H), 5.44 (dd, J = 8.4, 6.0 Hz, 1H), 4.69-4.60 (m, 2H), 4.38 (td, J = 7.6, 4.0 Hz, 1H), 4.13-4.04 (m, 2H), 3.90-3.37 (m, 4H), 3.35 (s, 3H), 3.16-3.03 (m, 1H), 2.99-2.83 (m, 3H), 2.67-2.56 (m, 1H), 2.54 (s, 3H), 2.21-1.98 (m, 2H), 1.94-1.85 (m, 1H), 1.93-1.85 (m, 1H), 1.68-1.51 (m, 2H). | 0.406 | 454.3 | Q |
| 108 | 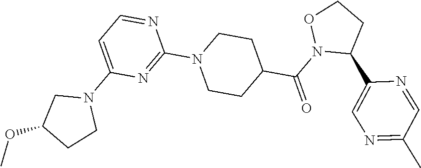 | 1H NMR (400 MHz, CD3OD) δ = 8.49 (s, 1H), 8.47 (s, 1H), 7.76 (d, J = 6.0 Hz, 1H), 5.79 (d, J = 6.0 Hz, 1H), 5.44 (dd, J = 8.4, 6.0 Hz, 1H), 4.70-4.61 (m, 2H), 4.39 (td, J = 7.6, 4.0 Hz, 1H), 4.13-4.04 (m, 2H), 3.80-3.37 (m, 4H), 3.35 (s, 3H), 3.16-3.05 (m, 1H), 2.98-2.83 (m, 3H), 2.64-2.56 (m, 1H), 2.54 (s, 3H), 2.19-1.99 (m, 2H), 1.93-1.85 (m, 1H), 1.84-1.70 (m, 1H), 1.68-1.51 (m, 2H). | 0.406 | 454.3 | Q |
| 109 | 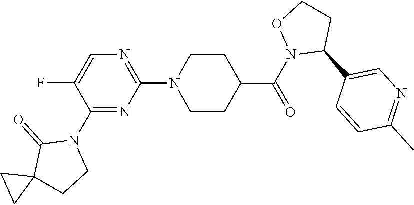 [SMILES: Cc1ccc([C@@H]2CCON2C(=O)C2CCN(c3ncc(F)c(N4CCC5(CC5)C4=O)n3)CC2)cn1] | 1H NMR (400 MHz, CDCl3) δ = 8.46 (d, J = 2.0 Hz, 1H), 8.16 (d, J = 2.8 Hz, 1H), 7.52 (dd, J = 7.6, 2.8 Hz, 1H), 7.13 (d, J = 7.6 Hz, 1H), 5.45- 5.37 (m, 1H), 4.63 (t, J = 13.6 Hz, 2H), 4.37-4.26 (m, 1H), 4.01 (t, J = 6.8 Hz, 2H), 3.98-3.91 (m, 1H), 3.06- 2.93 (m, 3H), 2.92-2.82 (m, 1H), 2.55 (s, 3H), 2.42-2.32 (m, 1H), 2.28-2.22 (m, 2H), 2.02-1.93 (m, 1H), 1.79-1.70 (m, 3H), 1.28-1.25 (m, 2H), 0.91-0.87 (m, 2H). | 0.510 | 481.3 | Q |
| 110 | 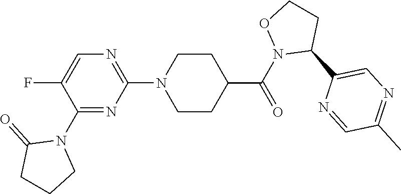 [SMILES: Cc1cnc([C@@H]2CCON2C(=O)C2CCN(c3ncc(F)c(N4CCCC4=O)n3)CC2)cn1] | 1H NMR (400 MHz, CDCl3) δ = 8.53 (s, 1H), 8.41 (s, 1H), 8.18 (d, J = 2.8 Hz, 1H), 5.51 (dd, J = 8.8, 6.0 Hz, 1H), 4.68-4.54 (m, 2H), 4.36 (td, J = 7.6, 4.0 Hz, 1H), 4.04-3.92 (m, 3H), 3.08-2.93 (m, 3H), 2.87-2.77 (m, 1H), 2.75-2.65 (m, 1H), 2.60 (t, J = 8.0 Hz, 2H), 2.56 (s, 3H), 2.27-2.16 (m, 2H), 2.01-1.91 (m, 1H), 1.81- 1.64 (m, 3H). | 0.542 | 456.3 | Q |
| 111 | 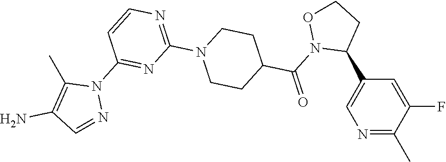 [SMILES: Cc1ncc([C@@H]2CCON2C(=O)C2CCN(c3nccc(-n4ncc(N)c4C)n3)CC2)cc1F] | 1H NMR (400 MHz, CD3OD) δ = 8.27 (d, J = 5.6 Hz, 1H), 8.24 (s, 1H), 7.53- 7.47 (m, 1H), 7.41 (s, 1H), 7.00 (d, J = 5.6 Hz, 1H), 5.42 (t, J = 7.6 Hz, 1H), 4.76-4.68 (m, 2H), 4.37 (td, J = 7.6, 3.2 Hz, 1H), 4.08-3.98 (m, 1H), 3.23-3.03 (m, 3H), 3.02-2.92 (m, 1H), 2.61 (s, 3H), 2.48 (d, J = 2.8 Hz, 3H), 2.42-2.30 (m, 1H), 2.05-1.93 (m, 1H), 1.88-1.79 (m, 1H), 1.73- 1.58 (m, 2H). | 0.421 | 467.2 | Q |
| 112 | 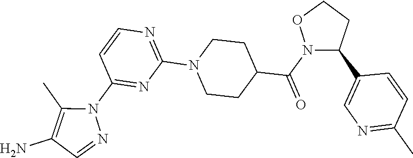 [SMILES: Cc1ccc([C@@H]2CCON2C(=O)C2CCN(c3nccc(-n4ncc(N)c4C)n3)CC2)cn1] | 1H NMR (400 MHz, CD3OD) δ = 8.36 (d, J = 2.0 Hz, 1H), 8.26 (d, J = 5.6 Hz, 1H), 7.67 (dd, J = 8.0, 2.0 Hz, 1H), 7.41 (s, 1H), 7.29 (d, J = 8.0 Hz, 1H), 6.99 (d, J = 5.6 Hz, 1H), 5.39 (t, J = 7.2 Hz, 1H), 4.75-4.67 (m, 2H), 4.36 (td, J = 7.6, 3.2 Hz, 1H), 4.03 (ddd, J = 9.2, 7.6, 6.4 Hz, 1H), 3.24- 3.02 (m, 3H), 2.96 (dddd, J = 12.0, 9.2, 6.4, 3.2 Hz, 1H), 2.60 (s, 3H), | 0.364 | 449.3 | Q |
| | | 2.51 (s, 3H), 2.40-2.29 (m, 1H), 2.03- | | | |
| | | 1.93 (m, 1H), 1.90-1.75 (m, 1H), | | | |
| | | 1.72-1.59 (m, 2H). | | | |
| 113 | 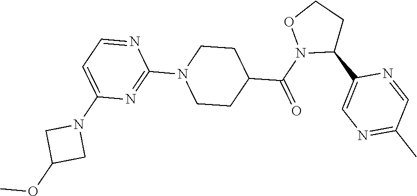 | 1H NMR (400 MHz, CDCl3) δ = 8.53 (d, J = 1.2 Hz, 1H), 8.40 (s, 1H), 7.90 (d, J = 6.0 Hz, 1H), 5.56 (d, J = 6.0 Hz, 1H), 5.51 (dd, J = 8.8, 6.0 Hz, 1H), 4.75 (t, J = 13.2 Hz, 2H), 4.38- 4.28 (m, 2H), 4.22-4.13 (m, 2H), 4.02-3.94 (m, 1H), 3.89 (dd, J = 9.2, 3.6 Hz, 2H), 3.33 (s, 3H), 3.07-2.98 (m, 1H), 2.97-2.86 (m, 2H), 2.85- 2.77 (m, 1H), 2.75-2.65 (m, 1H), 2.56 (s, 3H), 1.99-1.87 (m, 1H), 1.82-1.67 (m, 3H). | 0.487 | 440.3 | Q |
| 114 | 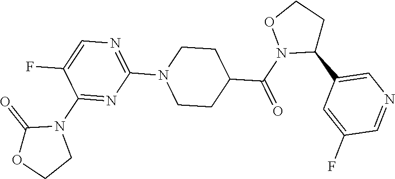 [SMILES: O=C1OCCN1c1nc(N2CCC(C(=O)N3OCC[C@H]3c3cncc(F)c3)CC2)ncc1F] | ¹H NMR (400 MHz, CDCl3) δ = 8.41 (t, J = 1.6 Hz, 1H), 8.39 (d, J = 2.8 Hz, 1H), 8.19 (d, J = 2.4 Hz, 1H), 7.34 (dt, J = 8.8, 2.4 Hz, 1H), 5.47 (dd, J = 8.8, 6.4 Hz, 1H), 4.68-4.57 (m, 2H), 4.56- 4.49 (m, 2H), 4.33 (td, J = 7.6, 3.6 Hz, 1H), 4.17 (t, J = 7.6 Hz, 2H), 3.99- 3.90 (m, 1H), 3.09-2.97 (m, 3H), 2.96-2.88 (m, 1H), 2.44-2.30 (m, 1H), 2.04-1.95 (m, 1H), 1.81-1.67 (m, 3H). | 0.547 | 461.3 | Q |
| 115 | 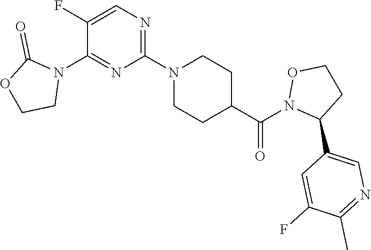 [SMILES: Cc1ncc([C@@H]2CCON2C(=O)C2CCN(c3ncc(F)c(N4CCOC4=O)n3)CC2)cc1F] | 1H NMR (400 MHz, CDCl3) δ = 8.27 (s, 1H), 8.18 (d, J = 2.8 Hz, 1H), 7.27- 7.23 (m, 1H), 5.42 (dd, J = 8.8, 6.4 Hz, 1H), 4.67-4.56 (m, 2H), 4.53 (t, J = 7.6 Hz, 2H), 4.32 (td, J = 7.6, 3.2 Hz, 1H), 4.17 (t, J = 7.6 Hz, 2H), 3.98-3.90 (m, 1H), 3.08-2.95 (m, 3H), 2.94-2.84 (m, 1H), 2.50 (d, J = 2.8 Hz, 3H), 2.42- 2.31 (m, 1H), 2.02-1.94 (m, 1H), 1.81-1.67 (m, 3H). | 0.547 | 475.3 | Q |
| 116 | 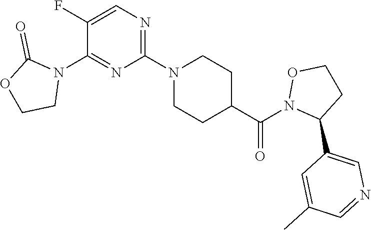 [SMILES: Cc1cncc([C@@H]2CCON2C(=O)C2CCN(c3ncc(F)c(N4CCOC4=O)n3)CC2)c1] | 1H NMR (400 MHz, CDCl3) δ = 8.39 (d, J = 1.6 Hz, 1H), 8.35 (s, 1H), 8.18 (d, J = 3.2 Hz, 1H), 7.41 (s, 1H), 5.41 (dd, J = 8.8, 6.4 Hz, 1H), 4.67-4.57 (m, 2H), 4.53 (t, J = 7.6 Hz, 2H), 4.32 (td, J = 7.6, 3.6 Hz, 1H), 4.17 (t, J = 7.6 Hz, 2H), 3.98-3.89 (m, 1H), 3.08- 2.95 (m, 3H), 2.93-2.83 (m, 1H), 2.43-2.35 (m, 1H), 2.33 (s, 3H), 2.03- 1.94 (m, 1H), 1.81-1.68 (m, 3H). | 0.494 | 457.3 | Q |

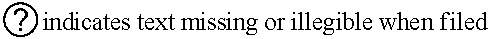

### Process of Making

The compounds of formula (I) may be prepared using the methods disclosed herein and routine modifications thereof, which will be apparent given the disclosure herein and methods well known in the art.

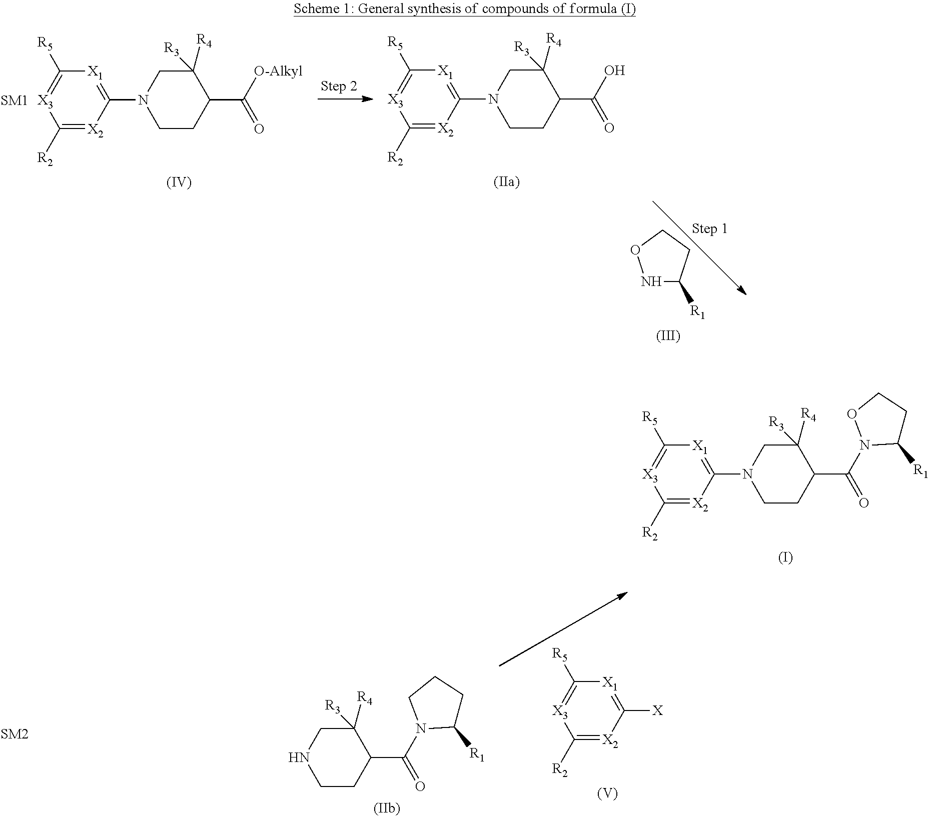

According to a first Synthetic Method (SM1), compounds of formula (I) can be obtained from a compound of formula (IV).

Step 2 consists in hydrolysis of the alkyl ester of formula (IV) to form the compound of formula (IIa) and can be achieved under well-known conditions using e.g. lithium hydroxide or sodium hydroxide in water or a solvent mixture such as THF/water or THF/water/MeOH.

Step 1 is an Amide coupling between an appropriate substituted isoxazolidine compound of formula (III), under the form of a free base or as a salt thereof, for example a halide salt thereof, and the compound of formula (IIa). It can be achieved by standard acid activation methods using for example ethyl cyanohydroxyiminoacetate (Oxyma), acid chloride, HOBt, HATU, HBTU, PyBOP or 1-propanephosphonic anhydride (T₃P) under basic conditions, e.g. in presence of diisopropylethylamine (DIEA) or triethylamine (TEA) or the like, in aprotic solvents like DMF, DMSO, acetonitrile, or the like to form compounds of formula (I). The reaction may be stirred for 2 to 12 hours, until full conversion to product was observed by LC/MS.

Substituted isoxazolidines of formula (III) can be synthesized by known procedures from the literature, e.g. WO2017096301, WO2019130230 and WO2021245070.

According to a fourth Synthetic Method (SM2), represented in scheme 1, compounds of formula (I) can be obtained by reacting an isoxazolidine derivative of formula (IIb) with a compound of formula (V). The step may consist in a nucleophilic substitution, in particular an aromatic nucleophilic substitution under well-known conditions, X being a leaving group, in particular an halogen, for example a chlorine atom, a fluorine atom, a bromine atom or a iodine atom, in the presence of a base, for example diisopropylethylamine, K₂CO₃, Et₃N or tBuOK, in an aprotic solvent like THF, acetonitrile, dioxane, NMP, DMSO or DMF or the like, under a temperature ranging from 60° C. to 120° C., for example under microwave conditions, to form compounds of formula (I).

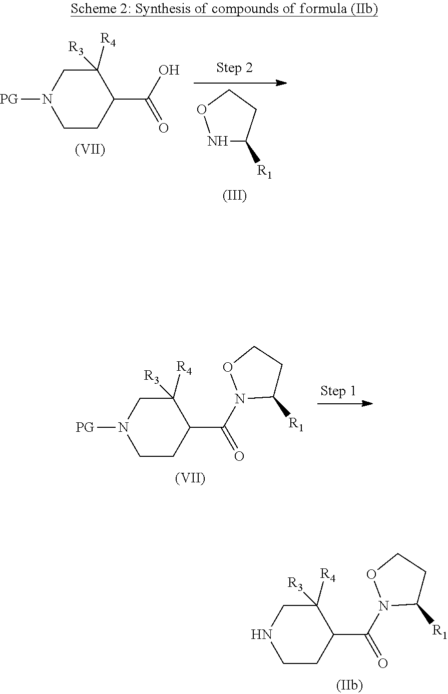

Compounds of the general formula (IIb) can be synthesized starting from compound (VI) in a deprotection step, step 1, PG of formula (VI) being a protecting group, for example a tert-butoxycarbonyle. Said step 1 may be performed under the two following acid conditions:
compound (VI) may be dissolved in dioxane and HCl may be added with stirring to yield the corresponding HCl salt of compound of formula (IIb), or
compound (VI) may be dissolved in DCM and TFA may be added with stirring to yield the corresponding TFA salt of compound of formula (IIb).

Alternatively, step 1 may be performed with a compound of formula (VI), wherein PG is a benzyloxycarbonyl group under metal catalyzed hydrogenation conditions, for example with Pd/C.

Compounds of general formula (VI) may be obtained in step 2 by reacting a compound of formula (VII) with an isoxazolidine of formula (III) under the same amide coupling conditions as described for step 1 of SM1 in scheme 1.

Compounds of formula (VII) and (Ill) can be synthesized by known procedures from the literature.

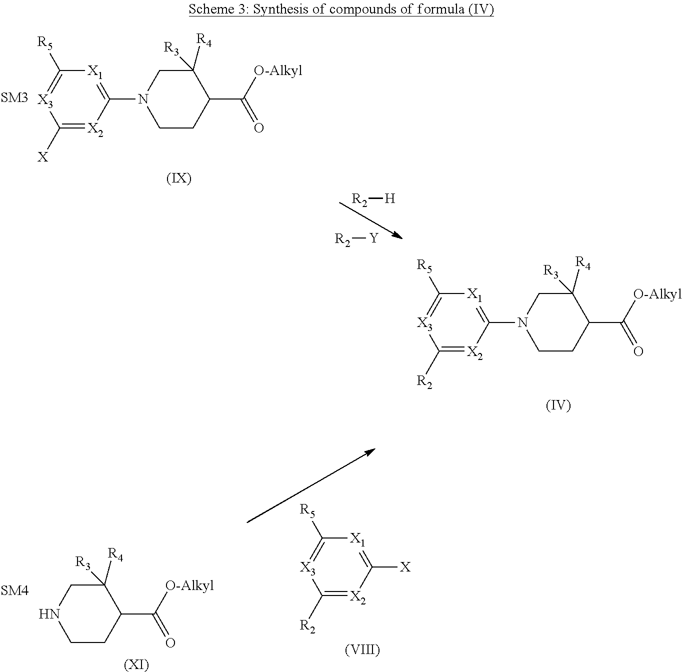

According to a third Synthetic Method (SM3), compound of the general formula (IV) may be obtained by reacting compound of the general formula (IX) with the compound R₂—H or the compound R₂—Y, R₂ being as defined in formula (I). Depending on the nature of the bond between R₂ and the other part of the molecule for compound (IV), the reaction may be performed:

If the bond is C—C bond,
under Suzuki coupling conditions in presence of a catalyst, typically palladium catalyst, in presence of a base for example K₂CO₃, for example in dioxane and/or water, by reacting compound of formula (IX) with R₂—Y, wherein Y is boronic acid or boronic ester; or
under coupling conditions in presence of a catalyst, typically palladium catalyst, in presence of a base for example K₂CO₃, for example in dioxane, by reacting a R₂—Y, wherein Y is a stannane derivative, a boronic acid or ester or other organometallic reagent; or

If the bond is C—N bond, under the same conditions as described in Synthetic Method (SM2), X of the compound (IX) being a leaving group, in particular a halogen, for example a chlorine atom.

According to a fourth Synthetic Method (SM4), compound of the general formula (IV) may be obtained by reacting compound of the general formula (XI) with the compound the general formula (VIII), X being a leaving group, in particular a halogen, for example a chlorine atom, a fluorine atom, a bromine atom or a iodine atom, under the same conditions as described in Synthetic Method (SM2).

Compounds of formula (IX) and (XI) can be synthesized by known procedures from the literature. The synthesis of Intermediates I-AA1 to I-AA6, as representative of such compounds of formula is in particular illustrated herein after in the examples.

Functional groups like acids, esters, amides, nitriles, halogens in compounds can be transformed (functional group interconversion) into other functional groups with standard methods like esterification, saponification, halogenation, Suzuki reaction to yield further compounds of formula (I).

The synthesis of typical compounds described herein may be accomplished as described in the following examples. If available, reagents may be purchased commercially, e.g., from Sigma Aldrich or other chemical suppliers. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedure.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006), Greene's protective groups in organic synthesis, Hoboken, N.J., Wiley-Interscience, and references cited therein.

Furthermore, the compounds of this disclosure may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) Organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989). The terms “solvent,” “inert organic solvent” or “inert solvent” refer to a solvent inert under the conditions of the reaction being described in conjunction therewith (including, for example, benzene, toluene, acetonitrile, tetrahydrofuran (“THF”), dimethylformamide (“DMF”), chloroform, methylene chloride (or dichloromethane, “DCM”), diethyl ether, methanol, pyridine and the like). Unless specified to the contrary, the solvents used in the reactions of the present disclosure are inert organic solvents, and the reactions are carried out under an inert gas, preferably argon.

### Pharmaceutical Compositions

The compounds provided herein is usually administered in the form of pharmaceutical compositions. Thus, provided herein are also pharmaceutical compositions that contain one or more of the compounds described herein or a pharmaceutically acceptable salt, tautomer, stereoisomer, mixture of stereoisomers, prodrug, or deuterated analog thereof and one or more pharmaceutically acceptable vehicles selected from carriers, adjuvants and excipients.

According to another aspect, pharmaceutical compositions are disclosed that include a compound described herein as an active ingredient. The compounds of the disclosure will normally, but not necessarily, be formulated into pharmaceutical compositions prior to administration to a patient. These pharmaceutical compositions comprise an effective dose of at least one compound of the disclosure as defined herein, or a pharmaceutically acceptable salt thereof, and one or more pharmaceutically acceptable excipients.

The said excipients are selected, in accordance with the pharmaceutical form and method of administration desired, from the customary excipients, which are known to a person skilled in the art.

In these pharmaceutical compositions for oral, sublingual, subcutaneous, intramuscular, intravenous, topical, local, intra-tracheal, intranasal, transdermal or rectal administration, the active ingredient is a compound of formula (I), or its salt or solvate where appropriate may be administered in a unit administration form, in a mixture with conventional pharmaceutical excipients, to a subject like human beings for the prophylaxis or treatment of a disease or disorder or condition that is at least partly mediated by receptor-interacting protein kinase 1 and in particular acute and chronic neurodegenerative diseases like Parkinson's disease, Alzheimer's disease, amyotrophic lateral sclerosis (ALS) or multiple sclerosis (MS).

### Unit Administration

The unit administration forms appropriate include oral forms such as tablets, soft or hard gel capsules, powders, granules and oral solutions or suspensions, sublingual, buccal, intratracheal, intra-ocular and intranasal administration forms, intra-muscular or intravenous administration, rectal administration forms and implants.

When prepared in unit administration form, the pharmaceutical compositions of the disclosure typically contain from 1 mg to 1000 mg of the active ingredient. The amount of active ingredient that is combined with one or more excipients to produce a single unit administration form will necessarily vary depending upon the host treated and the particular route of administration. For example, a formulation intended for oral administration to humans will generally contain, for example, from 0.5 mg to 0.5 g of active agent compounded with an appropriate and convenient amount of excipients which may vary from about 5 to about 98 percent by weight of the total composition.

As an example, a unit administration form of a herein described compound in tablet form may comprise the following components:

| | | |
|---|---|---|
| Compound | 50.0 | mg |
| Mannitol | 223.75 | mg |
| Sodium croscarmellose | 6.0 | mg |
| Corn starch | 15.0 | mg |
| Hydroxypropylmethylcellulose | 2.25 | mg |
| Magnesium stearate | 3.0 | mg |

In using a compound of the disclosure for therapeutic or prophylactic purposes it will generally be administered so that a daily dose in the range, for example, from 0.1 mg/kg to 75 mg/kg body weight is received, given if required in divided doses.

In general, lower doses will be administered when a parenteral route is employed. Thus, for example, for intravenous or intraperitoneal administration, a dose in the range, for example, from 0.1 mg/kg to 30 mg/kg body weight will generally be used. Oral administration may also be suitable, particularly in tablet form. Typically, unit dosage forms will contain about from 0.5 mg to 0.5 g of a compound of this disclosure.

There may be particular cases in which higher or lower dosages are appropriate; such dosages do not depart from the scope of the disclosure. According to usual practice, the dosage that is appropriate for each patient is determined by the doctor according to the mode of administration and the weight and response of the said patient.

### Methods of Treatment

In other embodiments, provided herein is a method of treating a receptor-interacting protein kinase 1-mediated disease or disorder. The method includes administering a therapeutically effective amount of a compound or pharmaceutical composition as described herein to a subject in need thereof. In some embodiments, the receptor-interacting protein kinase 1-mediated disease or disorder is Parkinson's disease, Alzheimer's disease, amyotrophic lateral sclerosis (ALS) or multiple sclerosis (MS).

The receptor-interacting protein kinase 1 inhibitors of the present disclosure are therefore useful for treating diseases and conditions mediated by receptor-interacting protein kinase 1, including but not limited to neurodegenerative diseases, central nervous system (CNS) diseases.

### Neurodegenerative and CNS Diseases

The receptor-interacting protein kinase 1 inhibitors described herein may also be used to treat neurodegenerative diseases. Neurodegenerative diseases can affect many of the body's activities, such as balance, movement, talking, breathing, and heart function. Neurodegenerative diseases can be genetic or caused by medical conditions such as alcoholism, tumors, strokes, toxins, chemicals, and viruses. Non-limiting examples of neurodegenerative diseases include Alzheimer's disease, amyotrophic lateral sclerosis (ALS) and Parkinson's disease

In certain embodiments, the compounds and compositions of the present disclosure are useful for treating Alzheimer's disease. In certain embodiments, the compounds and compositions of the present disclosure are useful for treating Parkinson's disease. In certain embodiments, the compounds and compositions of the present disclosure are useful for treating amyotrophic lateral sclerosis (ALS).

More generally, the receptor-interacting protein kinase 1 inhibitors described herein can be used to preserve neuron viability and promote axon growth and nerve functions within the central nervous system (CNS). Accordingly, the compounds may be used to reduce or even reverse the loss of cognitive, motor, and sensory functions associated with a CNS disease or disorder, by preserving neuron viability and/or promoting axon regeneration and/or nerve functions.

If desired, the effective daily dose of the active compound may be administered as two, three, four, five, six or more sub-doses administered separately at appropriate intervals throughout the day, optionally, in unit dosage forms. In certain embodiments, the present disclosure relates to compounds for inhibiting cell death, wherein the compounds are represented by formula (I). In certain embodiments, the compounds of the present disclosure are inhibitors of cell death. In any event, the compounds of the present disclosure in another embodiment exert their effect on inhibiting cell death at a concentration less than about 50 micromolar, more in another embodiment at a concentration less than about 10 micromolar and most in another embodiment at a concentration less than 1 micromolar. The compounds of the disclosure can be tested in standard animal models of stroke and standard protocols such as described by Hara, H., et al. Proc. Natl. Acad. Sci. USA, 1997.94(5): 2007-12.

When the compounds of the present disclosure are administered as pharmaceuticals, to humans and animals, they can be given per se or as a pharmaceutical composition containing, for example, 0.1% to 99.5% (more in another embodiment, 0.5% to 90%) of active ingredient in combination with a pharmaceutically acceptable carrier.

The compounds of the present disclosure or the compositions thereof may be administered once, twice, three or four times daily. Also, administration or treatment with the compounds may be continued for a number of days; for example, commonly treatment would continue for at least 7 days, 14 days or 28 days, for one cycle of treatment. Treatment cycles are well known and are frequently alternated with resting periods of about 1 to 28 days, commonly about 7 days or about 14 days, between cycles. The treatment cycles, in certain embodiments, may also be continuous.

When administered orally, the total daily dosage for a human subject may be between 1 mg and 1,000 mg, between about 1,000-2,000 mg/day, between about 10-500 mg/day, between about 50-300 mg/day, between about 75-200 mg/day or between about 100-150 mg/day.

The daily dosage may also be described as a total amount of a compound described herein administered per dose or per day. Daily dosage of a compound may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day or between about 15 to 150 mg/day. In certain embodiments, the method comprises administering to the subject an initial daily dose of about 1 to 800 mg of a compound described herein and increasing the dose by increments until clinical efficacy is achieved. Increments of about 5, 10, 25, 50 or 100 mg can be used to increase the dose. The dosage can be increased daily, every other day, twice per week or once per week.

In certain embodiments, a compound or pharmaceutical preparation is administered orally. In certain embodiments, the compound or pharmaceutical preparation is administered intravenously. Alternative routes of administration include sublingual, intramuscular and transdermal administrations.

The preparations of the present disclosure may be given orally, parenterally, topically or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. In certain embodiments, the administration is oral.

### EXAMPLES

The examples which follow describe the preparation of certain compounds. These examples are not limitative and merely illustrative.

Numerous modifications and alternative compositions, methods, and systems may be devised by those skilled in the art without departing from the spirit and scope of the present disclosure.

### Abbreviations

ACN acetonitrile
Ar Argon
aq. Aqueous
n-BuOH n-Butanol
DCM dichloromethan
DEA diethylamine
DIBAL-H diisobutylaluminium hydride
DIPEA N,N-diisopropylethylamine
DIAD diisopropyl azodicarboxylate
DMF N,N-dimethylformamide
DMSO dimethyl sulfoxide
EA or EtOAc ethyl acetate
EE Ethyl Ether
eq. equivalent(s)
ESI electro spray ionization
EtOH ethanol
FA formic acid
H or hr or hrs hours
HATU 1-[bis(dimethylamino)methylene]-1H-1,2,3-RTiazolo[4,5-b]pyridinium 3-oxide hexafluorophosphate
nHept n-Heptane
HBTU (2-(1H-benzotriazol-1-yl)-1,1,3,3-teRTamethyluronium-hexafluorophosphat)
HOBt 1-hydroxybenzotriazole
(RP) HPLC (reversed-phase) high pressure liquid chromatography
IBX 2-iodoxybezoic acid
IPA isopropyl amine
RP LC reversed-phase liquid chromatography
LC/MS liquid chromatography/mass spectrometry
M molar
MW Microwave
m/z mass-to-charge ratio
MeOH methanol
min minute(s)
N normal
NMR nuclear magnetic resonance
PE petroleum ether
prep. preparative
PyBOP benzotriazol-1-yl-oxytripyrrolidinophosphonium hexafluorophosphate
rt room temperature
sat. saturated
SFC supercritical fluid chromatography
TEA Triethylamine
TFA Trifluoroacetic acid
TFAA Trifluoroacetic anhydride
TH F Tetrahydrofurane
TLC thin layer chromatography
RT retention time
UV Ultraviolet

### Silica Gel Chromatography

Silica gel chromatography was performed using CombiFlash® Rf (Teledyne ISCO), Biotage Isolera One automated flash purification system or two Buchi systems (C-660, C-605, C-620, C-635 combination and C-660, C-605, C-615, C-630 combination) with pre-packed cartridges.

### Preparative Reversed-Phase HPLC

For preparative reversed phase HPLC an Agilent 1200 preparative HPLC machine, Gilson equipment (GX-271 liquid handler, 331/332-pump, UV/VIS-155), a Waters Autopurification LC Prep System or a Biotage equipment using C18 columns and a water (0.1% FA)/ACN gradient was used.

### NMR

400 MHz: NMR spectra were recorded on a Bruker AVANCE II 400 spectrometer operating at a proton frequency of 400.23 MHz. The instrument was equipped with a 5 mm BBI room temperature probe head. Alternatively, a Bruker AVANCE Ill HD 400 MHz, or a Bruker AVANCE NEO 400 MHz was used.

600 MHz: NMR spectra were recorded on a Bruker AVANCE Ill 600 spectrometer operating at a proton frequency of 600.05 MHz. The instrument was equipped with a 5 mm BBI room temperature probe head.

### Analytical LC/MS Equipment for Method a

Retention time and mass detection were done on a Waters Acquity UHPLC system coupled with a Waters SQD mass detector. The injection volume was 1.0 μl. Molecular weights are given in gram per mol [g/mol], detected masses in mass per charge [m/z].

### Analytical LC/MS Equipment for Methods B, D, E, and Chiral Method H

For retention time and mass detection a LC/MS-system from Agilent (LC 1200 Series/MS 6120 quadrupole LC/MS, LC 1260 infinity/MS 6120 quadrupole LC/MS or LC 1260 Infinity II/MSD Infinity Lab) was used. Molecular weights are given in gram per mol [g/mol], detected masses in mass per charge [m/z].

### Analytical LC/MS Equipment for Methods C, Q, R, S

Retention time and mass detection were done on a SHIMADZU LC-30AD system coupled with a PDA or DAD mass detector

For retention time and mass detection a LC/MS-system from Agilent (LC 1200 Series/MS 6120 quadrupole LC/MS, LC 1260 infinity/MS 6120 quadrupole LC/MS or LC 1260 Infinity II/MSD Infinity Lab) was used. Molecular weights are given in gram per mol [g/mol], detected masses in mass per charge [m/z].

### Chiral Analytical Equipment for Method F, G, I, J, K, L, M, T, U, Y, AA, AB, AD, AE, AF, AG, AI, AJ, AK, AM, AN Und AP

For retention time a SFC: SHIMADZU LC-30AD sf system was used.

### Analytical LC/MS Equipment for Method H, N, O, P, W, X, Z, AC Und AH

For retention time a LC system from Agilent (Agilent-1260 series system) was used

### LC/MS—Method a

Gradient: 98% H₂O (0.05% FA/2% ACN (0.035% FA) for 0.2 min.; then from 98% H₂O (0.05% FA) to 98% ACN (0.035% FA) in 3.6 min, then 98% ACN (0.035% FA) for 0.5 min, flow rate: 1.0 ml/min, column: 2.1×50 mm Waters ACQUITY UPLC BEH C18, 1.7 μm, 55° C.
UV data: retention time ad λ=220 nm given in min
MS data: ES+ ionisation, m/z given as [M+H]+ unless otherwise noted

### LC/MS—Method B

Gradient: From 93% H₂O (0.05% TFA)/7% acetonitrile to 95% acetonitrile in 1.0 min, then 95% acetonitrile for 0.45 min, flow rate: 1.1 ml/min, column: 2.0×10 mm LunaC18, 3 μm, 30° C., injection volume 0.2 μl UV data: retention time ad λ 220 nm given in min MS data: ES+ ionisation, m/z given as [M+H]⁺ unless otherwise noted

### LC/MS—Method C

Gradient: From 95% H₂O (0.0375% TFA)/5% ACN (0.01875% TFA) to 5% H₂O (0.0375% TFA)/95% ACN (0.01875% TFA) in 0.8 min; flow rate: 1.5 ml/min, column: Kinetex EVO C18 2.1×30 mm, 5 μm, 50° C. UV data: retention time ad λ 220 nm given in min MS data: ES+ ionisation, m/z given as [M+H]⁺ unless otherwise noted

### LC/MS—Method D

Gradient: From 100% H₂O (0.0375% TFA)/0% ACN (0.01875% TFA) to 40% H₂O (0.0375% TFA)/60% ACN (0.01875% TFA) in 0.8 min; then 40% H₂O (0.0375% TFA)/60% ACN (0.01875% TFA) for 0.4 min; flow rate: 1.5 ml/min, column: Kinetex EVO C18 2.1×30 mm, 5 μm, 50° C. UV data: retention time ad λ 220 nm given in min MS data: ES+ ionisation, m/z given as [M+H]⁺ unless otherwise noted

### LC/MS—Method E

Gradient: from 99% H₂O (0.05% TFA)/1% ACN to 7% ACN in 0.3 min; then from 7% ACN to 95% ACN 1.3 min; flow rate: 1.1 ml/min, column: 2.0×10 mm LunaC18, 3 μm, 30° C., injection volume 0.2 μl UV data: retention time ad λ 220 nm given in min MS data: ES+ ionisation, m/z given as [M+H]+ unless otherwise noted

### LC/MS Method Q

Gradient: From 95% H₂O (0.0375% TFA)/5% ACN (0.01875% TFA) to 5% H₂O (0.0375% TFA)/95% ACN (0.01875% TFA) in 1.05 min; flow rate: 2 ml/min, column: HALO C¹⁸ 3.0×30 mm, 5.0 um 50° C. UV data: retention time ad λ 220 nm given in min. Detector: PDA
MS data: ES+ ionisation, m/z given as [M+H]+ unless otherwise noted

### LC/MS Method R

Gradient: From 95% H2O (0.025% NH3·H2O)/5% ACN to 5% H2O (0.025% NH3·H2O)/5% ACN in 1.05 min; Maintain 5% A:95% B for 0.4 min, then back to 95% A:5% B in 0.3 min flow rate: 2 ml/min, column: Kinetex EVO C18 2.1×30 mm, 5 μm, 50° C. UV data: retention time ad λ 220 nm given in min. Detector: PDA
MS data: ES+ ionisation, m/z given as [M+H]+ unless otherwise noted

### LC/MS Method S

Gradient: From 95% H2O (0.025% NH3·H2O)/5% ACN to 5% H2O (0.025% NH3·H2O)/5% ACN in 1.05 min; Maintain 5% A:95% B for 0.4 min, then back to 95% A:5% B in 0.3 min flow rate: 2 ml/min, column: Kinetex EVO C18 2.1×30 mm, 5 μm, 40° C. UV data: retention time ad λ 220 nm given in min. Detector: PDA
MS data: ES+ ionisation, m/z given as [M+H]+ unless otherwise noted

### Chiral Analytical Methods:

### Method F

Mobile phase: Phase A for CO2, and Phase B for MeOH (0.05% DEA); Gradient: MeOH (0.05% DEA) in CO2 from 5% to 40%; flow rate: 3 mL/min, column: Chiralcel OJ-3 50×4.6 mm I.D., 3 μm, 35° C.; Back pressure: 100 Bar; Detector: PDA
UV data: retention time ad λ 220 nm given in min

### Method G

Mobile phase: Phase A for CO2, and Phase B for EtOH (0.05% DEA); Gradient: EtOH (0.05% DEA) in CO2 from 5% to 40%; flow rate: 3 mL/min, column: Chiralpak IF-3 50×4.6 mm, 3 μm, 35° C.; Back pressure: 100 Bar; Detector: DAD
UV data: retention time ad λ 220 nm given in min

### Method H

Mobile phase: Phase A for CO2, and Phase B for MeOH (0.05% DEA); Gradient: MeOH (0.05% DEA) in CO2 from 5% to 40%; flow rate: 3 mL/min, column: Chiralcel OJ-3 50×4.6 mm I.D., 3 μm, 35° C.; Back pressure: 100 Bar; Detector: DAD
UV data: retention time ad λ 220 nm given in min

### Method I

Mobile phase: Phase A for CO2, and Phase B for MeOH (0.05% DEA); Gradient: MeOH (0.05% DEA) in CO2 from 5% to 40%; flow rate: 3 mL/min, column: Chiralpak AD-3 50×4.6 mm I.D., 3 μm, 35° C.; Back pressure: 100 Bar; Detector: PDA
UV data: retention time ad λ 220 nm given in min

### Method J

Mobile phase: Phase A for CO2, and Phase B for iPrOH (0.05% DEA); Gradient: iPrOH (0.05% DEA) in CO2 from 5% to 40%; flow rate: 3 mL/min, column: Chiralpak IC-3 50×4.6 mm I.D., 3 μm, 35° C.; Back pressure: 100 Bar; Detector: PDA
UV data: retention time ad λ 220 nm given in min

### Method K

Mobile phase: Phase A for CO2, and Phase B for MeOH (0.05% DEA); Gradient: MeOH (0.05% DEA) in CO2 from 5% to 40%; flow rate: 3 mL/min, column: Chiralpak IC-3 50×4.6 mm I.D., 3 μm, 35° C.; Back pressure: 100 Bar; Detector: PDA
UV data: retention time ad λ 220 nm given in min

### Method L

Mobile phase: Phase A for CO2, and Phase B for MeOH (0.05% DEA); Gradient: MeOH (0.05% DEA) in CO2 from 5% to 40%; flow rate: 3 mL/min, column: Chiralpak IG-3 50×4.6 mm I.D., 3 μm, 35° C.; Back pressure: 100 Bar; Detector: PDA
UV data: retention time ad λ 220 nm given in min

### Method M

Mobile phase: Phase A for CO2, and Phase B for EtOH (0.05% DEA); Gradient: EtOH (0.05% DEA) in CO2 from 5% to 40%; flow rate: 3 mL/min, column: Chiralpak AD-3 50×4.6 mm I.D, 3 μm, 35° C.; Back pressure: 100 Bar; Detector: PDA
UV data: retention time ad λ 220 nm given in min

### Method N

Mobile phase: Phase A for CO2, and Phase B for MeOH (0.05% DEA); Gradient: EtOH (0.05% DEA) in CO2 from 5% to 40%; flow rate: 3 mL/min, column: Chiralpak AD-3 50×4.6 mm I.D, 3 μm, 35° C.; Back pressure: 100 Bar; Detector: PDA
UV data: retention time ad λ 220 nm given in min

### Method O

Mobile phase: Phase A for CO2, and Phase B for MeOH (0.05% DEA); Gradient: EtOH (0.05% DEA) in CO2 from 5% to 40%; flow rate: 3 mL/min, column: Chiralpak AD-3 50×4.6 mm I.D, 3 μm, 35° C.; Back pressure: 100 Bar; Detector: PDA
UV data: retention time ad λ 220 nm given in min

### Method P

Mobile phase: Phase A for CO2, and Phase B for MeOH (0.05% DEA); Gradient: EtOH (0.05% DEA) in CO2 from 5% to 40%; flow rate: 3 mL/min, column: Chiralcel OD-3 50×4.6 mm, 3 μm, 35° C.; Back pressure: 100 Bar; Detector: DAD
UV data: retention time ad λ 220 nm given in min

### Method T

Mobile phase: Phase A for CO2, and Phase B for EOH (0.05% DEA); Gradient: EtOH (0.05% DEA) in CO2 from 5% to 40%; flow rate: 3 mL/min, column: Cellulose-2 50×4.6 mm I.D., 3 um35° C.; Back pressure: 100 Bar; Detector: PDA
UV data: retention time ad λ 220 nm given in min

### Method U

Mobile phase: Phase A for CO2, and Phase B for iPrOH+ACN (0.05% DEA); Gradient: iPrOH+CAN (0.05% DEA) in CO2 from 5% to 40%; flow rate: 3 mL/min, column: Chiralpak IK-3 50×4.6 mm I.D., 3 μm, 35° C.; Back pressure: 100 Bar; Detector: PDA
UV data: retention time ad λ 220 nm given in min

### Method W

Mobile phase: Phase A for CO2, and Phase B for MeOH (0.05% DEA); Gradient: MeOH (0.05% DEA) in CO2 from 5% to 40%; flow rate: 3 mL/min, column: Chiralcel OJ-3 50×4.6 mm I.D., 3 μm, 35° C.; Back pressure: 100 Bar; Detector: PDA
UV data: retention time ad λ 220 nm given in min

### Method X

Mobile phase: Phase A for CO2, and Phase B for EtOH (0.05% DEA); Gradient: EtOH (0.05% DEA) in CO2 from 5% to 40%; flow rate: 3 mL/min, column: Chiralcel OJ-3 50×4.6 mm I.D., 3 μm, 35° C.; Back pressure: 100 Bar; Detector: PDA
UV data: retention time ad λ 220 nm given in min

### Method Y

Mobile phase: Phase A for CO2, and Phase B for iPrOH (0.05% DEA); Gradient: iPrOH (0.05% DEA) in CO2 from 5% to 40%; flow rate: 3 mL/min, column: Chiralcel OJ-3 50×4.6 mm I.D., 3 μm, 35° C.; Back pressure: 100 Bar; Detector: PDA
UV data: retention time ad λ 220 nm given in min

### Method Z

Mobile phase: Phase A for CO2, and Phase B for EtOH (0.05% DEA); Gradient: EtOH (0.05% DEA) in CO2 from 5% to 40%; flow rate: 3 mL/min, column: Chiralcel OD-3 50×4.6 mm, 3 μm, 35° C.; Back pressure: 100 Bar; Detector: PDA
UV data: retention time ad λ 220 nm given in min

### Method AA

Mobile phase: Phase A for CO2, and Phase B for iPrOH+ACN (0.05% DEA); Gradient: iPrOH+ACN (0.05% DEA); (0.05% DEA) in CO2 from 5% to 40%; flow rate: 3 mL/min, column: Chiralcel OD-3 50×4.6 mm, 3 μm, 35° C.; Back pressure: 100 Bar; Detector: PDA
UV data: retention time ad λ 220 nm given in min

### Method AB

Mobile phase: Phase A for CO2, and Phase B for iPrOH+ACN (0.05% DEA); Gradient: iPrOH+ACN (0.05% DEA); (0.05% DEA) in CO2 from 5% to 40%; flow rate: 3 mL/min, column: Chiralpak AY-3 50×4.6 mm I.D, 3 μm, 35° C.; Back pressure: 100 Bar; Detector: PDA
UV data: retention time ad λ 220 nm given in min

### Method AC

Mobile phase: Phase A for CO2, and Phase B for iPrOH (0.05% DEA); Gradient: iPrOH (0.05% DEA) in CO2 from 5% to 40%; flow rate: 3 mL/min, column: Chiralpak AD-3 50×4.6 mm I.D., 3 μm, 35° C.; Back pressure: 100 Bar; Detector: PDA
UV data: retention time ad λ 220 nm given in min

### Method AD

Mobile phase: Phase A for CO2, and Phase B for iPrOH (0.05% DEA); Gradient: iPrOH (0.05% DEA) in CO2 from 5% to 40%; flow rate: 3 mL/min, column: Chiralpak AS-3 50×4.6 mm I.D., 3 μm, 35° C.; Back pressure: 100 Bar; Detector: PDA
UV data: retention time ad λ 220 nm given in min

### Method AE

Mobile phase: Phase A for CO2, and Phase B for EtOH (0.05% DEA); Gradient: EtOH (0.05% DEA) in CO2 from 5% to 40%; flow rate: 3 mL/min, column: Chiralcel OJ-3 50×4.6 mm, 3 μm, 35° C.; Back pressure: 100 Bar; Detector: PDA
UV data: retention time ad λ 220 nm given in min

### Method AF

Mobile phase: Phase A for CO2, and Phase B for iPrOH+ACN (0.05% DEA); Gradient: iPrOH+ACN (0.05% DEA); (0.05% DEA) in CO2 from 5% to 40%; flow rate: 3 mL/min, column: Kromasil (S,S)Whelk-O1 50×4.6 mm I.D., 3 μm, 35° C.; Back pressure: 100 Bar; Detector: PDA
UV data: retention time ad λ 220 nm given in min

### Method AG

Mobile phase: Phase A for CO2, and Phase B for EtOH (0.05% DEA); Gradient: EtOH (0.05% DEA) in CO2 from 5% to 40%; flow rate: 3 mL/min, column: Chiralpak AS-3 50×4.6 mm I.D., 3 μm, 35° C.; Back pressure: 100 Bar; Detector: PDA
UV data: retention time ad λ 220 nm given in min

### Method AH

Mobile phase: Phase A for CO2, and Phase B for EtOH (0.05% DEA); Gradient: EtOH (0.05% DEA) in CO2 from 5% to 40%; flow rate: 3 mL/min, column: Chiralpak AD-3 50×4.6 mm I.D., 3 μm, 35° C.; Back pressure: 100 Bar; Detector: PDA
UV data: retention time ad λ 220 nm given in min

### Method AI

Mobile phase: Phase A for CO2, and Phase B for EtOH (0.05% DEA); Gradient: EtOH (0.05% DEA) in CO2 from 5% to 40%; flow rate: 3 mL/min, column: Chiralpak IC-3 50×4.6 mm I.D., 3 μm, 35° C.; Back pressure: 100 Bar; Detector: PDA
UV data: retention time ad λ 220 nm given in min

### Method AJ

Mobile phase: Phase A for CO2, and Phase B for iPrOH+ACN (0.05% DEA); Gradient: iPrOH+ACN (0.05% DEA) in CO2 from 5% to 50%; flow rate: 3 mL/min, column: Chiralpak AS-3 50×4.6 mm I.D., 3 μm, 35° C.; Back pressure: 100 Bar; Detector: PDA
UV data: retention time ad λ 220 nm given in min

### Method AK

Mobile phase: Phase A for CO2, and Phase B for EtOH+ACN (0.05% DEA); Gradient: EtOH+ACN (0.05% DEA) in CO2 from 5% to 40%; flow rate: 3 mL/min, column: Chiralpak IG-3 50×4.6 mm I.D., 3 μm, 35° C.; Back pressure: 100 Bar; Detector: PDA
UV data: retention time ad λ 220 nm given in min

### Method AL

Mobile phase: Phase A for CO2, and Phase B for iPrOH+ACN (0.05% DEA); Gradient: iPrOH+ACN (0.05% DEA) in CO2 from 5% to 40%; flow rate: 3 mL/min, column: Kromasil (S,S)Whelk-O1 50×4.6 mm I.D., 3 um, 35° C.; Back pressure: 100 Bar; Detector: PDA
UV data: retention time ad λ 220 nm given in min

### Method AM

Mobile phase: Phase A for CO2, and Phase B for EtOH+ACN (0.05% DEA); Gradient: EtOH+ACN (0.05% DEA) in CO2 from 5% to 50%; flow rate: 3 mL/min, column: Chiralpak AD-3 50×4.6 mm I.D., 3 μm, 35° C.; Back pressure: 100 Bar; Detector: PDA
UV data: retention time ad λ 220 nm given in min

### Method AN

Mobile phase: Phase A for CO2, and Phase B for iPrOH+ACN (0.05% DEA); Gradient: iPrOH+ACN (0.05% DEA) in CO2 from 5% to 40%; flow rate: 3 mL/min, column: Chiralpak AD-3 50×4.6 mm I.D., 3 μm, 35° C.; Back pressure: 100 Bar; Detector: PDA
UV data: retention time ad λ 220 nm given in min

### Method AP

Mobile phase: Phase A for CO2, and Phase B for EtOH (0.05% DEA); Gradient: EtOH (0.05% DEA) in CO2 from 5% to 40%; flow rate: 3 mL/min, column: Chiralcel OD-3 50×4.6 mm I.D., 3 μm, 35° C.; Back pressure: 100 Bar; Detector: DAD
UV data: retention time ad λ 220 nm given in min

### Salts

In compounds described as HCl—, TFA- or as another salt the exact amount of the respective salt is usually not determined. Therefore, the amount of the salt can range from as low as 0.01 eq. up to 5.0 eq. depending on the chemical structure (e.g. number of basic centres).

### Chiral Purity

Compounds are drawn and named as a single enantiomer, if the enantiomeric ratio exceeded 90:10. For enantiomeric ratios below 90:10 the racemic form is used.

### Synthetic Methods

### General Procedures

### General Procedure 1: Amide Couplings with HATU

The carboxylic acid (1 eq) and and HATU (1.5 eq) were dissolved in dry DMF (2 ml/mmol carboxylic acid). Then the corresponding base (6-10 eq), was added with stirring. After 15 min the corresponding amine (as free base or as HX salt, 1 eq) dissolved in dry DMF was added with stirring. The reaction was stirred for 2-12 hours, until full conversion to product was observed by LC/MS. After that, diluted sodium bicarbonate solution was added and the aqueous phase was extracted with EA (3×). The combined organic phases were washed with brine, dried over sodium sulphate, filtered, and concentrated in vacuo. The residue was purified by silica gel chromatography and/or by RP prep-HPLC to give the corresponding products. The common bases used for amide coupling can be DIPEA or TEA.

### General Procedure 2: Deprotection of Boc Groups to Give HCl Salts

Tert-butyl (3S)-3-pyrazin-2-ylisoxazolidine-2-carboxylate (205 mg) was dissolved in dioxane (6 ml) and HCl (9.42 ml, 4 M in dioxane) was added at rt with stirring. After standing overnight the solvent was removed in vacuo, the residue dissolved in ACN/water and lyophilised overnight to yield the corresponding HCl salt.

### General Procedure 3: Deprotection of Boc Groups to Give TFA Salts

The corresponding building block (155 mg) was dissolved in DCM (8 ml) and TFA (442 μl) was added. After stirring for 3 h at rt, the solvent was removed, and the residue lyophilized from water/ACN to yield the corresponding crude material as a TFA salt.

### General Procedure 4: Hydrolysis of Me or Ethyl Esters to Carboxylic Acids

To a solution of the corresponding ethyl or methyl ester (14.80 mmol) in THF (50 ml) LiOH·H₂O (1 M, 29.60 ml, 2 eq.) was added. The mixture was stirred at 20° C. for 1 hr. The reaction mixture was adjusted to pH 3 with 4 N HCl aqueous solution, the mixture was concentrated in vacuo and purified by reversed-phase HPLC.

### Intermediates

### Intermediate I-01: (3S)-3-pyrazin-2-ylisoxazolidine hydrochloride salt (responding to formula (III))

Step 1: I-01a: Tert-butyl-dimethyl-[(E)-3-pyrazin-2-ylallyloxy]silane 2-Bromopyrazine (2.5 g), (E)-3-(tert-butyldimethylsilyloxy)propene-1-yl-boronic acid pinacol ester (5.3 ml) and caesium carbonate (9.73 g) were dissolved in a mixture of dioxane (42 ml) and water (10.5 ml). Then Ar was bubbled through the solution for 5 min and chloro(2-dicyclohexylphosphino-2′,4′,6′-RTiisopropyl-1,1′-biphenyl)[2-(2′-amino-1,1′-biphenyl]palladium(II) (590 mg) was added. Ar was again bubbled through the solution for 5 min and the mixture was refluxed for 1 h with stirring under Ar. After cooling water and EA were added. The aqueous phase was extracted with EA (2×). The combined organic phases were dried over sodium sulphate, filtered and concentrated in vacuo to yield 6.36 g of the title compound that was directly used in the next step.

Step 2: I-01: (E)-3-Pyrazin-2-ylprop-2-en-1-ol Tert-butyl-dimethyl-[(E)-3-pyrazin-2-ylallyloxy]silane (6.36 g) was dissolved in THF (100 ml), the mixture was cooled to 0° C. and tetrabutylammonium fluoride (31.75 ml, 1 M in THF) was added. After 2 h solid NaHCO₃ was added with stirring. After 1.5 h the suspension was filtered and the filtrate concentrated in vacuo. The residue was purified by silica gel chromatography (200 g SiO₂, 100% DCM for 5 min; from 100% DCM to 10% ethanol in 45 min; then 10% EtOH for 15 min). The pure product containing fractions were combined and the solvent was removed in vacuo to yield 1.82 g of the title compound.
LC/MS: m/z=137.2 [M+H]+; RT: 0.64 min (LC/MS-method A)

Step 3: I-01c: (E)-3-Pyrazin-2-ylprop-2-enal(E)-3-Pyrazin-2-ylprop-2-en-1-ol (1.82 mg) was dissolved in DCM (90 ml) and MnO₂ (23.24 g) was added with stirring. After 30 min the mixture was filtered and the filtrate concentrated in vacuo to yield 1.12 g of the title compound that was directly used in the next step.
LC/MS: m/z=135.1 [M+H]+; RT: 0.73 min (LC/MS-method A).

### Step 4: I-01d: Tert-butyl (3S)-5-hydroxy-3-pyrazin-2-yl-isoxazolidine-2-carboxylate

[Diphenyl-[(2S)-pyrrolidin-2-yl]methoxy]-triimethyl-silane (700 mg) was dissolved in DCM (40 ml) and the mixture was cooled to 0° C. (E)-3-Pyrazin-2-ylprop-2-enal (1.12 g) dissolved in DCM (10 ml) and tert-butyl N-hydroxycarbamate (1.36 g) were added with stirring. After standing in the refrigerator (4° C.) overnight further silyl ether (0.1 eq.) and carbamate (0.5 eq.) were added and the mixture kept for 24 h in the refrigerator. Then saturated NH₄Cl solution was added. The aqueous phase was extracted with DCM (2×), the combined organic phases were dried over sodium sulphate, filtered and concentrated in vacuo. The residue was purified by RP prep-HPLC in 6 runs (flow 75 ml/min, 90% H₂O/10% ACN to 10% H₂O/90% ACN in 17.5 min; Agilent Prep C18—10 μm, 30×250 mm). The pure product containing fractions were combined, the ACN was removed in vacuo and the aqueous phase was lyophilised to yield 358 mg of the title compound.
LC/MS: m/z=268.3 [M+H]+; RT: 1.19 min (LC/MS-method A).

### Step 5: I-01e: Tert-butyl N-hydroxy-N-[(1S)-3-hydroxy-1-pyrazin-2-yl-propyl]carbamate

Tert-butyl (3S)-5-hydroxy-3-pyrazin-2-yl-isoxazolidine-2-carboxylate (360 mg) was dissolved in methanol (20 ml), cooled to 0° C. and NaBH₄ (50 mg) was added with stirring. After 1 h saturated NH₄Cl solution was added. The aqueous phase was extracted with DCM (5×), the combined organic phases were dried over sodium sulphate, filtered and concentrated in vacuo. The residue was purified by RP prep-HPLC (flow 75 ml/min, 90% H₂O/10% ACN to 10% H₂O/90% ACN in 17.5 min; Agilent Prep C18—10 μm, 30×250 mm). The pure product containing fractions were combined, the ACN was removed in vacuo and the aqueous phase was lyophilised to yield 209 mg of the title compound.
LC/MS: m/z=270.3 [M+H]+; RT: 1.06 min (LC/MS-method A)

### Step 6: I-01f: Tert-butyl (3S)-3-pyrazin-2-ylisoxazolidine-2-carboxylate

Tert-butyl N-hydroxy-N-[(1S)-3-hydroxy-1-pyrazin-2-yl-propyl]carbamate (205 mg) was dissolved in THF (5 ml) and with stirring Triphenylphosphine (290 mg) and DIAD (210 μl) were added. After stirring for 1 h the solvent was removed in vacuo and the residue was purified by RP prep-HPLC (flow 75 ml/min, 90% H₂O/10% ACN to 10% H₂O/90% ACN in 15 min; Agilent Prep C18—10 μm, 30×250 mm). The product containing fractions were combined, the ACN was removed in vacuo and the aqueous phase was lyophilised to yield 275 mg of the title compound, which was still containing about 50 mol % of reduced DIAD.
LC/MS: m/z=252.3 [M+H]+; RT: 1.38 min (LC/MS-method A).

### Step 7: I-01: (3S)-3-Pyrazin-2-ylisoxazolidine hydrochloride salt

Tert-butyl (3S)-3-pyrazin-2-ylisoxazolidine-2-carboxylate (205 mg) was dissolved in dioxane (6 ml) and HCl (9.42 ml, 4 M in dioxane) was added at rt with stirring. After standing overnight the solvent was removed in vacuo, the residue dissolved in ACN/water and lyophilised overnight to yield 256 mg of the title compound, which was still contaminated with ~50 mol % of reduced DIAD from step 6, but this did not interfere with the following step.
LC/MS: m/z=152.2 [M+H]+; RT: 0.46 min (LC/MS-method A).

### Intermediate I-02: (3S)-3-(2-Methylthiazol-4-yl)isoxazolidine Tifluoroacetic acid salt (Responding to Compound of Formula (III))

### Step 1: I-02a: Ethyl 2-methylthiazole-4-carboxylate

To a solution of thioacetamide (7.86 g, 104.61 mmol) in EtOH (60 ml), and ethyl 3-bromo-2-oxo-propanoate (20 g, 102.56 mmol) was added dropwise in 10 min and stirred for 12 h at 25° C. The mixture was added to 300 ml 1 N HCl, stirred 0.5 h, then the pH was adjusted to 8, and the solution extracted with EA (200 ml*3), dried with Na₂SO₄ and concentrated. The crude product was triturated with PE: EA=5:1 (30 ml) to give the title compound (10.6 g, 58% yield) as a brown solid.

¹H NMR (CDCl₃, 400 MHz): δ ppm 8.04 (1H), 4.43 (2H), 2.78 (3H), 1.41 (3H).

### Step 2: I-02b: 2-Methylthiazole-4-carbaldehyde

To a solution of ethyl 2-methylthiazole-4-carboxylate (9.6 g, 56.07 mmol, 1 eq) in DCM (96 ml) was added DIBAL-H (1 M, 84.10 ml). The mixture was stirred at −78° C. for 3 h. The reaction mixture was quenched by MeOH (5 ml), warmed to 25° C., filtered and concentrated under reduced pressure, purified by flash silica gel chromatography (PE:EA=1:0-5:1) to give the title compound (5.5 g, 76% yield) as yellow oil.

¹H NMR (CDCl₃, 400 MHz): δ ppm 9.99 (1H), 8.05 (1H), 2.79 (3H).

### Step 3: I-02c: (E)-3-(2-Methylthiazol-4-yl)prop-2-enal

A mixture of 2-methylthiazole-4-carbaldehyde (5.5 g, 34.60 mmol), (formylmethylene) Triphenylphosphorane (10.53 g, 34.60 mmol) in THF (55 ml) was degassed and purged with N₂ for 3 times, and then the mixture was stirred at 70° C. for 3 h under N₂ atmosphere. The reaction mixture was concentrated under reduced pressure to give a residue. The residue was purified by silica gel column chromatography (PE: EA=4:1) to give the title compound (4.5 g, 80% yield) as yellow oil.

¹H NMR (CDCl₃, 400 MHz): δ ppm 9.70 (1H), 7.45 (1H), 7.39 (1H), 6.93 (1H), 2.76 (3H).

### Step 4: I-02d: Tert-butyl (3S)-5-hydroxy-3-(2-methylthiazol-4-yl)isoxazolidine-2-carboxylate

To a solution of [diphenyl-[(2R)-pyrrolidin-2-yl]methoxy]-Trimethyl-silane (1.78 g, 5.48 mmol) in CHCl₃ (42 ml) was added (E)-3-(2-methylthiazol-4-yl)prop-2-enal (4.2 g, 27.41 mmol) and tert-butyl N-hydroxycarbamate (4.02 g, 30.16 mmol) at 0° C. The mixture was warmed to 25° C. smoothly, stirred for 12 h. The reaction mixture was concentrated under reduced pressure to give a residue. The crude product was purified by preparative RP-LC (column: Agela C18, 20 μm; 120 Å; mobile phase: water/0.1% sat. aqueous NH₃ solution—ACN; B %: 5%-30%, 10 min; flow rate: 25 ml/min) to give the title compound (3.9 g, 50% yield) as yellow oil.

### Step 5: I-02e: Tert-butyl N-hydroxy-N-[(1S)-3-hydroxy-1-(2-methylthiazol-4-yl)propyl]carbamate

To a solution of tert-butyl (3S)-5-hydroxy-3-(2-methylthiazol-4-yl)isoxazolidine-2-carboxylate (3.4 g, 11.87 mmol) in MeOH (34 ml) was added NaBH₄ (494.10 mg, 13.06 mmol) at 0° C. The mixture was stirred for 2 h. The reaction mixture was quenched with saturated NH₄Cl solution (2 ml) at 0° C., diluted with water (200 ml), extracted with EA (150 ml*3). The combined organic layers were washed with brine (100 ml), dried over Na₂SO₄, filtered, and concentrated under reduced pressure to give a residue. The residue was purified by silica gel column chromatography (PE: EA=2:1~1:1) to give the title compound (2.44 g, 70% yield) as a yellow oil.

¹H NMR (CDCl₃, 400 MHz): δ ppm 7.02 (1H), 5.46 (1H), 3.78 (2H), 2.71 (3H), 2.39-2.28 (1H), 2.17-2.09 (1H), 1.49 (9H).

### Step 6: I-02f: Tert-butyl (3S)-3-(2-methylthiazol-4-yl)isoxazolidine-2-carboxylate

To a solution of tert-butyl N-hydroxy-N-[(1S)-3-hydroxy-1-(2-methylthiazol-4-yl)propyl]carbamate (2.4 g, 8.32 mmol) in THF (24 ml) was added Tributylphosphane (2.69 g, 13.32 mmol) and DIAD (2.19 g, 10.82 mmol) at 0° C., stirred for 1 h. The reaction mixture was concentrated under reduced pressure to give a residue. The residue was purified by prep. RP LC (flow: 100 ml/min; gradient: from 95% H₂O (0.1% FA)/5% ACN to 40% H₂O (0.1% FA)/60% ACN in 15 min; 40% H₂O (0.1% FA)/60% ACN in 3 min; column: Welch Ultimate XB_C18, 20-40 μm, 120 Å, I.D.72 mm*H300 mm) and flash silica gel column chromatography (PE:EA=1:0~4:1) to give the title compound, which was further purified by SFC (column: Daicel Chiralpak AD (250 mm*30 mm, 10 μm); mobile phase A: CO₂, B: 0.1% aqueous NH₃ solution in methanol; B %: 15%-15%, 5.6; 60 min; flow rate: 50 ml/min; back pressure: 100 bar) to yield the title compound (483 mg, 99.6% e.e.) as an off white solid.
LC/MS: m/z 171.2 [M+H−100+]; RT 0.822 min (Method C)

¹H NMR (CDCl₃, 400 MHz): δ ppm 7.07 (1H), 5.37 (1H), 4.18-4.11 (1H), 3.92 (1H), 2.71-2.69 (4H), 2.56-2.47 (1H), 1.50 (9H).

Analytical SFC: RT 0.93 min (99.8%, Method I); RT of R-enantiomer: 0.54 min (Method I)

### Step 7: I-02: (3S)-3-(2-Methylthiazol-4-yl)isoxazolidine trifluoroacetic acid salt

Tert-butyl (3S)-3-(2-methylthiazol-4-yl)isoxazolidine-2-carboxylate (155 mg) was dissolved in DCM (8 ml) and TFA (442 μl) was added. After stirring for 3 h at rt, the solvent was removed, and the residue lyophilised from water/ACN to yield 179 mg of crude material.
LC/MS: m/z=171.1 [M+H]⁺; RT: 0.46 min (LC/MS-method B)

### Intermediate I-03: (3S)-3-(6-Methyl-3-pyridyl)isoxazolidine (TFA salt) (Responding to Compound of Formula (III))

### Step 1: I-03a. 5-[(E)-3,3-diethoxyprop-1-enyl]-2-methyl-pyridine

A mixture of 5-bromo-2-methylpyridine (20 g, 116.26 mmol, 1 eq), 3,3-diethoxyprpene (60.54 g, 465.06 mmol, 70.89 mL, 4 eq), Pd(OAc)₂ (1.31 g, 5.81 mmol, 0.05 eq), K₂CO₃ (32.14 g, 232.53 mmol, 2 eq), KCl (8.67 g, 116.26 mmol, 1 eq), tetrabutylammonium acetate (70.11 g, 232.53 mmol, 70.82 mL, 2 eq) in DMF (400 mL) was degassed and purged with N2 for 3 times, and then the mixture was stirred at 80° C. for 16 hr under N₂ atmosphere. The reaction mixture was concentrated, diluted with water (1000 mL), extracted with EA (300 mL*3). The combined organic layers were washed with brine (300 mL*3), dried over Na₂SO₄, filtered, and concentrated under reduced pressure to give 5-[(E)-3,3-diethoxyprop-1-enyl]-2-methyl-pyridine (27 g, crude) as red liquid, which was used for the next step directly.

### Step 2: I-03b. (E)-3-(6-methyl-3-pyridyl)prop-2-enal

A solution of 5-[(E)-3,3-diethoxyprop-1-enyl]-2-methyl-pyridine (27 g, 122.01 mmol, 1 eq) in HCl (1 M, 244 mL, 2 eq) was stirred at 25° C. for 0.5 hr. The mixture was adjusted with saturated NaHCO₃ solution (230 mL) to pH=6-7, extracted with EA (200 mL*3). The combined organic layers were washed with brine (500 mL*2), dried over anhydrous Na₂SO₄, filtered, and concentrated under reduced pressure to give a residue. The residue was purified by silica gel column chromatography (PE:EA=10:1~2:3) to give (E)-3-(6-methyl-3-pyridyl)prop-2-enal (14.2 g, 79.06% yield) as yellow solid.

LC/MS: m/z 148.1 [M+1]+: RT: 0.21 min (Method D).

¹H NMR (CHCl₃, 400 MHz) δ ppm 9.65 (d, J=7.6 Hz, 1H), 8.59 (d, J=2.2 Hz, 1H), 7.72 (dd, J=2.4, 8.1 Hz, 1H), 7.40 (d, J=16.0 Hz, 1H), 7.17 (d, J=8.1 Hz, 1H), 6.67 (dd, J=7.6, 16.1 Hz, 1H), 2.55 (s, 3H).

### Step 3: I-03c. tert-butyl (3S)-5-hydroxy-3-(6-methyl-3-pyridyl)isoxazolidine-2-carboxylate

To a solution of [diphenyl-[(2R)-pyrrolidin-2-yl]methoxy]-Trimethyl-silane (6.95 g, 21.34 mmol, 0.2 eq) in CHCl₃ (160 mL) was added (E)-3-(6-methyl-3-pyridyl)prop-2-enal (15.7 g, 106.68 mmol, 1 eq) in one portion at 0° C., the mixture was stirred at 0° C. for 30 min, then tert-butyl N-hydroxycarbamate (15.62 g, 117.34 mmol, 1.1 eq) was added. The mixture was warmed to 25° C. smoothly, stirred for 12 hr. The reaction mixture was concentrated under reduced pressure to give a residue. The residue was purified by Prep. RP LC (flow: 400 mL/min; gradient: from 95% H₂O (0.1% FA)/5% ACN to 85% H₂O (0.1% FA)/15% ACN in 20 min; 85% H₂O (0.1% FA)/15% ACN in 20 min; 85% H₂O (0.1% FA)/15% ACN to 79% H₂O (0.1% FA)/21% ACN in 10 min; 79% H₂O (0.1% FA)/21% ACN in 15 min; column: Phenomenex luna C18, 10 μm, 100 Å, I.D. 150 mm*H400 mm) to give compound tert-butyl (3S)-5-hydroxy-3-(6-methyl-3-pyridyl)isoxazolidine-2-carboxylate (15.2 g, 50.84% yield) as yellow oil.

LC/MS: m/z 281.2 [M+1]⁺; RT 0.677 min (Method D).

¹H NMR (DMSO-d6, 400 MHz) δ ppm 8.36 (d, J=2.2 Hz, 1H), 7.57 (dd, J=2.3, 8.1 Hz, 1H), 7.22 (d, J=7.9 Hz, 1H), 6.95 (br d, J=1.5 Hz, 1H), 5.63 (t, J=3.9 Hz, 1H), 5.19 (t, J=8.3 Hz, 1H), 2.58 (dd, J=8.4, 12.4 Hz, 1H), 2.44 (s, 3H), 2.13 (ddd, J=4.3, 8.2, 12.1 Hz, 1H), 1.37 (s, 9H).

### Step 4: I-03d. N-hydroxy-N-[(1S)-3-hydroxy-1-(6-methyl-3-pyridyl)propyl]carbamate

To a solution of tert-butyl (3S)-5-hydroxy-3-(6-methyl-3-pyridyl)isoxazolidine-2-carboxylate (15.2 g, 54.22 mmol, 1 eq) in MeOH (155 mL) was added NaBH₄ (2.26 g, 59.65 mmol, 1.1 eq) in portions at 0° C. The mixture was stirred at 0° C. for 2 hr under N₂ atmosphere. The mixture was quenched with saturated NH₄Cl solution (100 mL), diluted with water (100 mL), extracted with EA (200 mL*2), dried with Na₂SO₄, filtered and concentrated to give a residue. The residue was purified by Prep. RP LC (flow: 100 mL/min; gradient: from 100% H₂O (0.1% FA)/0% ACN to 78% H₂O (0.1% FA)/22% ACN in 10 min; 78% H₂O (0.1% FA)/22% ACN to 78% H₂O (0.1% FA)/22% ACN in 20 min; column: Welch Ultimate XB_C18, 20-401 μm, 120 Å, I.D. 32 mm*H210 mm) to give tert-butyl N-hydroxy-N-[(1S)-3-hydroxy-1-(6-methyl-3-pyridyl)propyl]carbamate (7.45 g, 46.23% yield, 83.492% e.e.) as yellow gum.

LC/MS: m/z 283.1 [M+1]+; RT=0.485 min (Method D).

¹H NMR (DMSO-d6 400 MHz) δ ppm 9.15 (s, 1H), 8.37 (d, J=1.8 Hz, 1H), 7.62 (dd, J=2.2, 7.9 Hz, 1H), 7.21 (d, J=8.1 Hz, 1H), 5.15 (dd, J=6.8, 8.3 Hz, 1H), 4.51 (t, J=4.8 Hz, 1H), 3.45 (qd, J=5.6, 11.0 Hz, 1H), 2.44 (s, 3H), 2.23-2.05 (m, 1H), 1.93-1.81 (m, 1H), 1.37 (s, 9H).

Chiral SFC RT for s enantiomer: 1.648 min (Method J).

### Step 5: I-03e. tert-butyl (3S)-3-(6-methyl-3-pyridyl)isoxazolidine-2-carboxylate

To a solution of N-hydroxy-N-[(1S)-3-hydroxy-1-(6-methyl-3-pyridyl)propyl]carbamate (7.45 g, 26.39 mmol, 1 eq) in THF (75 mL) was added tributylphosphane (8.54 g, 42.22 mmol, 10.42 mL, 1.6 eq) and DIAD (6.94 g, 34.30 mmol, 6.67 mL, 1.3 eq) at 0° C. The mixture was warmed to 25° C. smoothly, stirred for 2 hr. The reaction mixture was concentrated under reduced pressure to give a residue. The residue was purified by Prep. RP LC (flow: 200 mL/min; gradient: from 80% H₂O (0.1% FA)/20% ACN to 60% H₂O (0.1% FA)/40% ACN in 12 min; 60% H₂O (0.1% FA)/40% ACN in 15 min; column: Welch Ultimate XB_C18, 20 μm, 100 Å, I.D. 75 mm*H348 mm) to give I-03e (5.2 g, 74.61% yield) as yellow oil.

5.2 g of I-03e was further separated by SFC (Column: Chiralpak IC-3 50×4.6 mm I.D., 3 um; Mobile phase: Phase A for CO₂, and Phase B for MeOH (0.05% DEA); Gradient elution: MeOH (0.05% DEA) in CO₂ from 5% to 40%; Flow rate: 3 mL/min; Detector: PDA; Column Temp: 35C; Back Pressure: 100 Bar) to give tert-butyl (3S)-3-(6-methyl-3-pyridyl)isoxazolidine-2-carboxylate (peak1, 4.7 g, >99.9% e.e.) as yellow oil and (3R)-3-(6-methyl-3-pyridyl)isoxazolidine-2-carboxylate (peak2, 313 mg, 99.13% e.e.) as yellow oil.

LC/MS: m/z 265.1 [M+1]+; RT: 0.755 min (Method D).

¹HNMR (DMSO-d6 400 MHz,) 5=8.38 (d, J=2.3 Hz, 1H), 7.59 (dd, J=2.4, 8.0 Hz, 1H), 7.23 (d, J=8.0 Hz, 1H), 5.16 (dd, J=6.0, 8.5 Hz, 1H), 4.15 (dt, J=3.0, 7.8 Hz, 1H), 3.74 (ddd, J=7.0, 8.2, 9.1 Hz, 1H), 2.79 (dddd, J=3.1, 6.8, 8.8, 12.1 Hz, 1H), 2.44 (s, 3H), 2.16 (dddd, J=6.0, 7.6, 9.3, 12.2 Hz, 1H), 1.38 (s, 9H).
Chiral SFC: RT for s enantiomer: 1.463 min. (Method K, 100%)
Chiral SFC: RT for s enantiomer: 2.042 min. (Method K, 100%)

### I-03: (3S)-3-(6-Methyl-3-pyridyl)isoxazolidine (Responding to Compound of Formula (III))

Tert-butyl (S)-3-(6-methylpyridin-3-yl)isoxazolidine-2-carboxylate (500 mg, 1.89 mmol) was dissolved in DCM (15 ml) and TFA (1.5 ml, 19.47 mmol) was added at RT with stirring. After standing overnight the solvent was removed in vacuo. The residue was lyophilised to yield 693 mg of the title compound.

LC/MS: m/z=165.4 [M+H]+; RT: 0.22 min (LC/MS-method A).

### Intermediate I-04: 3-(5-Fluoro-3-pyridyl)isoxazolidine HCl/TFA salt (Responding to Compound of Formula (III))

### Step 1: I 04-1a: (E)-3-(5-Fluoro-3-pyridyl)prop-2-enal

A mixture of 3-bromo-5-fluoro-pyridine (48 g), prop-2-enal (45.87 g), Pd(OAc)₂ (3.06 g), benzyl(triethyl)ammonium chloride (62.12 g) and TEA (82.80 g) in DMF (400 ml) was stirred at 70° C. under N₂ atmosphere for 12 h. The mixture was concentrated, diluted with water (800 ml), extracted with ethyl acetate (500 ml×3), washed with brine (1 l), dried with Na₂SO₄, filtered and concentrated. The residue was purified by flash silica gel column chromatography (PE:EA=1:1) to yield 26.7 g of the title compound.

¹H NMR (CDCl₃ 400 MHz): δ ppm 9.77 (1H), 8.62 (1H), 8.55 (1H), 7.60 (1H), 7.50 (1H), 6.78 (1H).

### Step 2: I-04-1b: Tert-butyl (S)-3-(5-fluoro-3-pyridyl)-5-hydroxy-isoxazolidine-2-carboxylate

To a solution of Diphenyl-[(2S)-pyrrolidin-2-yl]methoxy]-Trimethyl-silane (21.54 g, 66.16 mmol, 0.2 eq) in CHCl₃ (500 mL) was added (E)-3-(5-Fluoropyridin-3-yl)acrylaldehyde (50 g, 330.82 mmol, 1 eq), the mixture was stirred at 0° C. for 30 min, then tert-butyl N-hydroxycarbamate (48.45 g, 363.91 mmol, 1.1 eq) was added at 0° C. The mixture was warmed to 25° C. smoothly, stirred for 12 hr. LCMS showed desired mass was detected. The reaction mixture was concentrated under reduced pressure to give a residue. The residue was purified by Prep. RP LC (flow: 400 mL/min; gradient: from 85% H₂O (0.1% FA)/15% ACN to 65% H₂O (0.1% FA)/35% ACN in 60 min; 65% H₂O (0.1% FA)/35% ACN to 65% H₂O (0.1% FA)/35% ACN in 20 min; column: Phenomenex luna C18, 15 μm, 100 Å, I.D. 150 mm*H400 mm) to give Tert-butyl (S)-3-(5-fluoro-3-pyridyl)-5-hydroxy-isoxazolidine-2-carboxylate (45 g, 158.29 mmol, 47.85% yield) as yellow solid.

LC/MS: m/z=285.2 [M+H]⁺; RT: 0.775 min (LC/MS-method C).

### Step 3: I-04-1c: Tert-butyl N-[(1S)-(5-fluoro-3-pyridyl)-3-hydroxy-propyl]-N-hydroxy-carbamate

To a solution of tert-butyl 3-(5-fluoro-3-pyridyl)-5-hydroxy-isoxazolidine-2-carboxylate (45 g, 158.29 mmol, 1 eq) in MeOH (450 mL) was added NaBH₄ (5.99 g, 158.29 mmol, 1 eq) at 0° C., then the mixture was stirred at 0° C. for 15 min. LCMS showed desired mass was detected. The reaction mixture was quenched with saturated NH₄Cl solution (40 mL), diluted with water (100 mL), extracted with EtOAc (600 mL*3). The combined organic layers were washed with brine (1200 mL), dried with Na₂SO₄, filtered and concentrated under reduced pressure to give a residue. The residue was purified by Prep. RP LC (flow: 400 mL/min; gradient: from 90% H₂O (0.1% FA)/10% ACN to 64% H₂O (0.1% FA)/36% ACN in 51 min; 64% H₂O (0.1% FA)/36% ACN to 64% H₂O (0.1% FA)/36% ACN in 11 min; column: Phenomenex luna C18, 15 μm, 100 Å, I.D. 150 mm*H400 mm) to give Tert-butyl 3-(5-fluoro-3-pyridyl)isoxazolidine-2-carboxylate (35 g, 77.23% yield) as yellow oil.

LC/MS: m/z=287.2 [M+H]⁺; RT: 0.685 min (LC/MS-method C).

Step 4: I-04-1d: Tert-butyl (3S)-3-(5-fluoro-3-pyridyl)isoxazolidine-2-carboxylate To a solution of tert-butyl 3-(5-fluoro-3-pyridyl)isoxazolidine-2-carboxylate (33 g, 115.26 mmol, 1 eq) in THF (400 mL) was added tributylphosphane (37.31 g, 184.42 mmol, 45.50 mL, 1.6 eq) and DIAD (30.30 g, 149.84 mmol, 29.13 mL, 1.3 eq) at 0° C., the mixture was warmed to 25° C. smoothly, stirred for 12 hr. LCMS showed desired mass was detected.

The reaction mixture was concentrated under reduced pressure to give a residue. The residue was combined with the former batch on 2 g scale and purified by Prep. RP LC (flow: 400 mL/min; gradient: from 75% H₂O (0.1% FA)/25% ACN to 52% H₂O (0.1% FA)/48% ACN in 50 min; 52% H₂O (0.1% FA)/48% ACN to 52% H₂O (0.1% FA)/48% ACN in 11 min; column: Phenomenex luna C18, 15 μm, 100 Å, I.D. 150 mm*H400 mm) and silica gel column chromatography (PE:EA=4:1~2:1) to give Intermediate I-04 as a mixture of two isomers (23.5 g, 75.99% yield, 71.3% e.e.) as yellow oil.

The product was further separated by SFC (column: DAICEL CHIRALPAK AD (250 mm*30 mm, 10 um); mobile phase: [0.1% NH₃H2O MEOH]; B %: 35%-35%, 5.4; 400 min) to give I-04-1d-Peak 1 (2.5 g, 10.48% yield, >99.9% e.e.) as yellow oil and I-04-Peak 2 (18 g, 75.91% yield, >99.9% e.e.) as yellow oil.

LC/MS: m/z=269.2 [M+H]⁺; RT: 0.741 min (LC/MS-method C).

¹H NMR (CDCl₃, 400 MHz): δ ppm 8.47-8.33 (m, 2H), 7.45 (td, J=2.0, 9.3 Hz, 1H), 5.28 (dd, J=5.6, 8.8 Hz, 1H), 4.20 (dt, J=3.5, 7.9 Hz, 1H), 3.89 (dt, J=7.1, 8.6 Hz, 1H), 2.84 (dddd, J=3.4, 7.1, 8.8, 12.3 Hz, 1H), 2.28 (dddd, J=5.6, 7.8, 9.1, 12.3 Hz, 1H), 1.47 (s, 9H).
Chiral SFC: RT for S isomer 1.297 min (100%, Method I)
Chiral SFC: RT for R isomer 0.691 min (100%, Method I)

### Step 5: I-04-1: 3-(5-Fluoro-3-pyridyl)isoxazolidine TFA salt

Tert-butyl 3-(5-fluoro-3-pyridyl)isoxazolidine-2-carboxylate (174.4 mg) was solved in dichloromethane (5 ml) and Trifluoroacetic acid (0.5 ml) and stirred overnight. The mixture was evaporated under reduced pressure and lyophilized twice to give 158 mg of the title compound.

LC/MS: m/z=169.2 [M+H]+; RT: 0.69 min (LC/MS-method A)

### I-04-2: (3S)-3-(5-Fluoro-3-pyridyl)isoxazolidine HCl salt (Responding to Compound of Formula (III))

### Step 1: I-04-2a: Tert-butyl 3-(5-fluoro-3-pyridyl)-5-hydroxy-isoxazolidine-2-carboxylate

To a solution of [diphenyl-[(2S)-pyrrolidin-2-yl]methoxy]-Trimethyl-silane (11.50 g) in chloroform (260 ml) was added (E)-3-(5-fluoro-3-pyridyl)prop-2-enal (26.7 g) and tert-butyl N-hydroxycarbamate (28.23 g) at 0° C. The mixture was warmed to 20° C. smoothly and stirred for 12 h. The reaction mixture was concentrated. The residue was purified by RP-LC (flow: 400 ml/min; gradient: from 90% H₂O (0.1% FA)/10% ACN to 60% H₂O (0.1% FA)/40% ACN in 50 min; 60% H₂O (0.1% FA)/40% ACN to 60% H₂O (0.1% FA)/40% ACN in 25 min; column: Phenomenex luna C18, 15 μm, 100 Å, I.D. 150 mm×H 400 mm) to yield 25 g of the title compound.

¹H NMR (CDCl₃ 400 MHz): δ ppm 8.45-8.37 (2), 7.44 (1H), 5.94-5.84 (1H), 5.39 (1H), 2.84 (1H), 2.33-2.20 (1H), 1.47 (9H).

### I-04-2b: Tert-butyl N-[(1S)-1-(5-fluoro-3-pyridyl)-3-hydroxy-propyl]-N-hydroxy-carbamate

To a solution of tert-butyl 3-(5-fluoro-3-pyridyl)-5-hydroxy-isoxazolidine-2-carboxylate (25 g) in methanol (250 ml) was added NaBH₄ (3.99 g) at 0° C. The mixture was stirred at 0° C. for 1 h. The reaction mixture was quenched with saturated NH₄Cl solution (50 ml), and then diluted with water (800 ml), extracted with ethyl acetate (11×3). The combined organic layers were washed with brine (1 l), dried with Na₂SO₄, filtered and concentrated. The crude product was purified by RP-LC (flow: 200 ml/min; gradient: from 90% H2O (0.1% FA)/10% ACN to 60% H2O (0.1% FA)/40% ACN in 15 min; 60% H2O (0.1% FA)/40% ACN to 60% H2O (0.1% FA)/40% ACN in 8 min; column: Welch Ultimate XB_C18, 20-40 μm, 120 Å, I.D. 95 mm×H 365 mm) to yield 23.5 g of the title compound (enantiomeric ratio: 94.7 (S): 5.3 (R)).

¹H NMR (CDCl₃ 400 MHz): δ ppm 8.49-8.30 (2H), 7.66-7.51 (1H), 5.33 (1H), 3.89-3.71 (2H), 2.41 (1H), 2.05 (1H), 1.47 (9H).
Chiral SFC: S Isomer: Tert-butyl N-[(1S)-1-(5-fluoro-3-pyridyl)-3-hydroxy-propyl]-N-hydroxy-carbamate: RT 0.90 min, 94.7% (Method I)
Chiral SFC: R Isomer: Tert-butyl N-[(1R)-1-(5-fluoro-3-pyridyl)-3-hydroxy-propyl]-N-hydroxy-carbamate: RT 0.97 min, 5.3% (Method I)

### Step 3. I-04-2c: Tert-butyl (3S)-3-(5-fluoro-3-pyridyl)isoxazolidine-2-carboxylate

To a solution of tert-butyl N-[1-(5-fluoro-3-pyridyl)-3-hydroxy-propyl]-N-hydroxy-carbamate (23.5 g) in THF (235 ml) was added tributylphosphate (26.57 g) and DIAD (21.58 g) at 0° C. The mixture was warmed to 25° C. smoothly, stirred for 12 hours under N₂ atmosphere. Then the reaction mixture was concentrated. The residue was purified by RP HPLC (flow: 400 ml/min; gradient: from 80% H₂O (0.1% FA)/20% ACN to 56% H₂O (0.1% FA)/44% ACN in 44 min; 56% H₂O (0.1% FA)/44% ACN to 56% H₂O (0.1% FA)/44% ACN in 19 min; column: Phenomenex luna C18, 15 μm, 100 Å, I.D. 150 mm×H 400 mm) and silica gel column chromatography (PE:EA=10:1 to 0:1) to yield 14.3 g of the title compound.

¹H NMR (CDCl₃ 400 MHz): δ ppm 8.42 (1H), 8.38 (1H), 7.46 (1H), 5.28 (1H), 4.21 (1H), 3.90 (1H), 2.85 (1H), 2.29 (1H), 1.48 (9H).
Chiral HPLC: (Chiralcel AD-H, 4.6 mm×250 mm, 5 μm; EtOH+0.1% IPA; flow 0.75 ml/min; T 30° C.).
Tert-butyl (3R)-3-(5-fluoro-3-pyridyl)isoxazolidine-2-carboxylate; RT 5.47 min (5.3%).
Tert-butyl (3S)-3-(5-fluoro-3-pyridyl)isoxazolidine-2-carboxylate: RT 10.18 min (94.7%).

### I-04-2: (3S)-3-(5-fluoro-3-pyridyl)isoxazolidine HCl salt (Responding to Compound of Formula (III))

Following the general procedure 2, 129 mg of the title compound was obtained.

LC/MS: m/z=169.2 [M+H]+; RT: 0.68 min (LC/MS-method A).

### Intermediate I-05: (3S)-3-(5-Fluoro-6-methyl-3-pyridyl)isoxazolidine (Responding to Compound of Formula (III))

### Step 1: I-05a: (E)-3-(6-chloro-5-fluoro-3-pyridyl)prop-2-enal

A mixture of 5-bromo-2-chloro-3-fluoro-pyridine (35 g, 166.32 mmol, 1 eq), acrolein (37.30 g, 665.30 mmol, 44.51 mL, 4 eq), Pd(OAc)₂ (3.73 g, 16.63 mmol, 0.1 eq), benzyl(triethyl)ammonium chloride (37.88 g, 166.32 mmol, 1 eq) and TEA (50.49 g, 498.97 mmol, 69.45 mL, 3 eq) in DMF (360 mL) was stirred at 80° C. for 8 hr under N2 atmosphere. The reaction mixture was concentrated, diluted with water (2000 mL), extracted with EA (1500 mL*3). The combined organic layers were washed with brine (2000 mL*3), dried over anhydrous Na₂SO₄, filtered and concentrated under reduced pressure to give a residue. The residue was purified by silica gel column chromatography (PE:EA=1:0~5:1) to give compound (E)-3-(6-chloro-5-fluoro-3-pyridyl)prop-2-enal (14 g, 45.36% yield) as yellow solid.

¹H NMR (CDCl₃, 400 MHz): δ ppm 9.76 (d, J=7.4 Hz, 1H), 8.40 (d, J=2.0 Hz, 1H), 7.67 (dd, J=2.0, 8.4 Hz, 1H), 7.46 (d, J=16.1 Hz, 1H), 6.75 (dd, J=7.3, 16.1 Hz, 1H).

### Step 2: I-05b: (E)-3-(5-fluoro-6-methyl-3-pyridyl)prop-2-enal

A mixture of compound (E)-3-(6-chloro-5-fluoro-3-pyridyl)prop-2-enal (8 g, 43.11 mmol, 1 eq), methylboronic acid (7.74 g, 129.32 mmol, 3 eq), Pd(PPh₃)₄ (9.96 g, 8.62 mmol, 0.2 eq) and K₂CO₃ (17.87 g, 129.32 mmol, 3 eq) in dioxane (80 mL) was stirred at 100° C. for 12 hr. The reaction mixture was diluted with water (200 mL), extracted with EA (150 mL*3). The combined organic layers were washed with brine (300 mL*3), dried over anhydrous Na₂SO₄, filtered and concentrated under reduced pressure to give a residue. The residue was purified by silica gel column chromatography (PE:EA=1:0~4:1) to give (E)-3-(5-fluoro-6-methyl-3-pyridyl)prop-2-enal (3.6 g, 50.56% yield) as yellow solid.

LC/MS; m/z 166.2 [M+1]+: RT: 0.586 min (Method C).

¹H NMR (CDCl3 400 MHz): δ ppm 9.74 (d, J=7.5 Hz, 1H), 8.48 (s, 1H), 7.57-7.43 (m, 2H), 6.73 (dd, J=7.5, 16.1 Hz, 1H), 2.59 (d, J=2.9 Hz, 3H).

### Step 3: I-05 c; tert-butyl (3S)-3-(5-fluoro-6-methyl-3-pyridyl)-5-hydroxy-isoxazolidine-2-carboxylate

To a solution of [diphenyl-[(2R)-pyrrolidin-2-yl]methoxy]-trimethyl-silane (2.56 g, 7.87 mmol, 0.2 eq) in CHCl₃ (60 mL) was added (E)-3-(5-fluoro-6-methyl-3-pyridyl)prop-2-enal (6.5 g, 39.35 mmol, 1 eq) at 0° C., the mixture was stirred at 0° C. for 0.5 hr. Then tert-butyl N-hydroxycarbamate (5.76 g, 43.29 mmol, 1.1 eq) was added at 0° C., the mixture was warmed to 20° C. smoothly, stirred for 12 hr. The reaction mixture was concentrated under reduced pressure to give a residue. The residue was purified by Prep. RP LC (flow: 200 mL/min; gradient: from 85% H₂O (0.1% FA)/15% ACN to 50% H₂O (0.1% FA)/50% ACN in 28 min; 50% H₂O (0.1% FA)/50% ACN to 50% H₂O (0.1% FA)/50% ACN in 10 min; column: Welch Ultimate XB_C18 20-40 μm, 120 Å, I.D. 75 mm*H348 mm) to give tert-butyl (3S)-3-(5-fluoro-6-methyl-3-pyridyl)-5-hydroxy-isoxazolidine-2-carboxylate (6.3 g, 53.66% yield) as brown oil.

LC/MS: m/z 299.2 [M+1]+; RT: 0.692 min (Method C).

¹H NMR (CDCl₃ 400 MHz): δ ppm 8.27 (s, 1H), 7.34 (dd, J=1.8, 10.0 Hz, 1H), 5.90 (d, J=4.2 Hz, 1H), 5.60 (br s, 1H), 5.33 (t, J=8.3 Hz, 1H), 2.80 (dd, J=8.4, 12.5 Hz, 1H), 2.52 (d, J=2.8 Hz, 3H), 2.30-2.19 (m, 1H), 1.45 (s, 9H).

### Step 4. I-05d: tert-butyl N-[(1S)-1-(5-fluoro-6-methyl-3-pyridyl)-3-hydroxy-propyl]-N-hydroxy-carbamate

To a solution of tert-butyl (3S)-3-(5-fluoro-6-methyl-3-pyridyl)-5-hydroxy-isoxazolidine-2-carboxylate (5 g, 16.76 mmol, 1 eq) in MeOH (50 mL) was added NaBH₄ (507.26 mg, 13.41 mmol, 0.8 eq) at 0° C., then the mixture was stirred at 0° C. for 15 min. The reaction mixture was quenched with saturated NH₄Cl solution (20 mL), diluted with water (100 mL), extracted with EA (100 mL*3). The combined organic layers were washed with brine (200 mL*3), dried over anhydrous Na₂SO₄, filtered and concentrated under reduced pressure to give a residue. The residue was combined with the former batch on a 2 g scale and purified by Prep. RP LC (flow: 400 mL/min; gradient: from 95% H₂O (0.1% FA)/5% ACN to 65% H₂O (0.1% FA)/35% ACN in 28 min; 65% H₂O (0.1% FA)/35% ACN to 65% H₂O (0.1% FA)/35% ACN in 10 min; Welch Ultimate XB_C18 20-40 μm, 120 Å I.D. 75 mm*H348 mm) to give tert-butyl N-[(1S)-1-(5-fluoro-6-methyl-3-pyridyl)-3-hydroxy-propyl]-N-hydroxy-carbamate (6.5 g, crude, 94.5% e.e) as light yellow oil.

LC/MS: m/z 301.2 [M+1]⁺; RT: 0.705 min (Method C).

¹H NMR (CDCl3, 400 MHz): δ ppm 8.20 (s, 1H), 7.99 (br d, J=4.6 Hz, 1H), 7.45 (dd, J=1.7, 10.1 Hz, 1H), 5.33 (dd, J=4.6, 10.9 Hz, 1H), 3.86-3.71 (m, 2H), 2.76-2.44 (m, 1H), 2.44-2.35 (m, 4H), 2.04-1.95 (m, 1H), 1.49 (s, 9H)
Chiral SFC: RT for s enantiomer: 1.41 min (97.3%, Method L)
Chiral SFC: RT for R enantiomer: 1.34 min (2.7%, Method L).

### Step 5: I-05e: 3.5. tert-butyl (3S)-3-(5-fluoro-6-methyl-3-pyridyl)isoxazolidine-2-carboxylate

To a solution of tert-butyl N-[(1S)-1-(5-fluoro-6-methyl-3-pyridyl)-3-hydroxy-propyl]-N-hydroxy-carbamate (6 g, 19.98 mmol, 1 eq) in THF (60 mL) was added Tributylphosphate (6.47 g, 31.97 mmol, 7.89 mL, 1.6 eq) and DIAD (5.25 g, 25.97 mmol, 5.05 mL, 1.3 eq) at 0° C., the mixture was warmed to 20° C. smoothly, stirred for 12 hr. The mixture was combined with the former batch on a 500 mg scale and concentrated under reduced pressure to give a residue. The residue was purified by Prep. RP LC (flow: 400 mL/min; gradient: from 90% H₂O (0.1% FA)/10% ACN to 60% H₂O (0.1% FA)/40% ACN in 20 min; 60% H₂O (0.1% FA)/40% ACN to 60% H₂O (0.1% FA)/40% ACN in 8 min; column: Welch Ultimate XB_C18 20-40 μm, 120 Å, I.D. 75 mm*H348 mm) and silica gel column chromatography (PE:EA=1:0~1:2) to give tert-butyl (3S)-3-(5-fluoro-6-methyl-3-pyridyl)isoxazolidine-2-carboxylate (4.6 g, 80.01% yield, 98.1% purity, 91.2% e.e.) as a colorless oil. The product was further separated by SFC (column: DAICEL CHIRALPAK IC (250 mm*30 mm, 5 um); mobile phase: [0.1% NH₃H₂O MEOH]; B %: 30%-30%, 4.0 min; 70 min) to give I-05e (3.5 g, 74.66% yield, >99.9% e.e.) as yellow oil.

LC/MS: m/z 283.2 [M+1]+; RT: 0.814 min (Method C).

¹H NMR (CDCl₃, 400 MHz): δ ppm 8.28 (s, 1H), 7.40 (dd, J=1.8, 10.0 Hz, 1H), 5.24 (dd, J=5.6, 8.7 Hz, 1H), 4.20 (dt, J=3.4, 8.0 Hz, 1H), 3.89 (dt, J=7.1, 8.6 Hz, 1H), 2.82 (dddd, J=3.5, 7.1, 8.8, 12.3 Hz, 1H), 2.52 (d, J=2.9 Hz, 3H), 2.27 (dddd, J=5.6, 7.8, 9.0, 12.3 Hz, 1H), 1.48 (s, 9H).
Chiral SFC: RT 1.13 min (100%, Method K)

### Intermediate I-05: (3S)-3-(5-Fluoro-6-methyl-3-pyridyl)isoxazolidine TFA Salt (Responding to Compound of Formula (III))

Following general procedure 3, 656 mg of the title compound were obtained. LC/MS: m/z=183.1 [M+H]⁺; RT: 0.79 min (Method A).

### Intermediate I-28: (3S)-3-(2-Methyoxazol-4-yl)isoxazolidine HCl salt

### Step 1: 1-28a. 2-methyloxazole-4-carbaldehyde

As in Step 5 for I-05 starting with methyl 2-methyloxazole-4-carboxylate (10.0 g, 0.0709 mol, 1.00 eq.) 2-methyloxazole-4-carbaldehyde (crude, containing lots of solvent toluene), which was used for next step without purification.

### Step 2: I-28 b. (E)-3-(2-methyloxazol-4-yl)prop-2-enal

As in Step 5 for I-05b starting with 2-methyloxazole-4-carbaldehyde (84.9 g, 0.279 mol) to give (E)-3-(2-methyloxazol-4-yl)prop-2-enal (12.1 g, 0.0829 mol, 29.7% yield over 2 steps).

¹H NMR (400 MHz, CDCl₃) δ=9.66 (d, J=8.0 Hz, 1H), 7.80 (s, 1H), 7.28 (d, J=15.6 Hz, 1H), 6.82 (dd, J=15.6, 8.0 Hz, 1H), 2.51 (s, 3H).

### Step 3. I-28 c. tert-butyl rac-(3S)-5-hydroxy-3-(2-methyloxazol-4-yl)isoxazolidine-2-carboxylate

As in Step 5 for I-05c starting with (E)-3-(2-methyloxazol-4-yl)prop-2-enal (6.40 g, 0.0467 mol to afford tert-butyl rac-(3S)-5-hydroxy-3-(2-methyloxazol-4-yl)isoxazolidine-2-carboxylate (8.50 g, 67.0% purity 45.1% yield) as a brown gum.

¹H NMR (400 MHz, DMSO-d₆) δ=7.80 (s, 1H), 6.88-6.80 (m, 1H), 5.58 (s, 1H), 5.13 (t, J=7.6 Hz, 1H), 2.38-2.34 (m, 5H), 1.40 (s, 9H).

### Step 4. I-28d. tert-butyl N-hydroxy-N-[(1S)-3-hydroxy-1-(2-methyloxazol-4-yl)propyl]carbamate

As in Step 5 for 1.05d but starting from tert-butyl rac-(3S)-5-hydroxy-3-(2-methyloxazol-4-yl)isoxazolidine-2-carboxylate (14.5 g, 67.0% purity, 0.0354 mol) to afford tert-butyl N-hydroxy-N-[(1S)-3-hydroxy-1-(2-methyloxazol-4-yl)propyl]carbamate (8.10 g, 80.0% purity, 0.0238 mol, 67.2% yield) as a yellow gum.

¹H NMR (400 MHz, DMSO-d₆) δ=8.87 (s, 1H), 7.72 (d, J=1.2 Hz, 1H), 5.08 (dd, J=8.4, 5.6 Hz, 1H), 4.44 (t, J=4.8 Hz, 1H), 3.47-3.38 (m, 2H), 2.35 (s, 3H), 1.97-1.85 (m, 2H), 1.41 (s, 9H).

### Step 5. I-28 e. tert-butyl (3S)-3-(2-methyloxazol-4-yl)isoxazolidine-2-carboxylate

As in Step 5 for I.05e starting from tert-butyl N-hydroxy-N-[(1S)-3-hydroxy-1-(2-methyloxazol-4-yl)propyl]carbamate (8.10 g, 80.0% purity, 0.0238 mol, 1.00 eq.) to give the crude product (3S)-3-(2-methyloxazol-4-yl)isoxazolidine-2-carboxylate (5.80 g, 48.4% e.e.) as a white solid. The above product was recrystallized in co-solvents PE/EtOAc (v/v 20/1) to afford pure tert-butyl (3S)-3-(2-methyloxazol-4-yl)isoxazolidine-2-carboxylate (2.30 g, 98.8% e.e., 39.7% yield).

¹H NMR (400 MHz, CDCl₃) δ=7.47 (d, J=0.8 Hz, 1H), 5.20 (dd, J=8.8, 4.4 Hz, 1H), 4.15 (td, J=7.6, 4.8 Hz, 1H), 3.89 (q, J=7.6 Hz, 1H), 2.66-2.56 (m, 1H), 2.52-2.45 (m, 1H), 2.43 (s, 3H), 1.50 (s, 9H).

LCMS (ESI) m/z=277.2 [M+Na]⁺ and 531.3 [2M+Na]⁺; RT 0.50 min (Method Q).
Chiral SFC: RT 0.773 min (100%, Method G)

### Step 6. I-28. (3S)-3-(2-Methyloxazol-4-yl)isoxazolidine HCl salt

Following the general procedure 2, 1.50 g, −66.7% purity was obtained as a white solid, and was used as such.
Intermediate I-29; (3S)-3-(5-methylpyrazin-2-yl)isoxazolidine

As described in WO2021245070 A1.

### Intermediate I-30: (3S)-3-(6-methylpyrazin-2-yl)isoxazolidine

As described in WO2021245070 A1.

### Intermediate I-31: (3S)-3-(5-methyl-3-pyridyl)isoxazolidine

As described in WO2021245070 A1.

### General Synthesis of Intermediates I-06 to I-13 and I-32-I-36

### Intermediate I-06: 4-Piperidyl-[(3S)-3-pyrazin-2-ylisoxazolidin-2-yl]methanone trifluoro acetic acid salt (responding to compound of formula (IIb)) (following scheme 2)

### Step 1: 1-06a: Tert-butyl 4-[(3S)-3-pyrazin-2-ylisoxazolidine-2-carbonyl]piperidine-1-carboxylate

Following the general procedure 1: 1-Tert-butoxycarbonyl-piperidine-4-carboxylic acid (1.3 g) was dissolved in dry DMF (25 ml) and DIPEA (2.99 ml) and HATU (3.8 g) were added with stirring. After 15 min (3S)-3-pyrazin-2-ylisoxazolidine TFA salt (1.3 g) dissolved in dry DMF (10 ml) was added with stirring. After 2 h sat. sodium bicarbonate solution was added, and the aqueous phase was extracted with EA (3×). The combined organic phases were washed with brine, dried over sodium sulphate, filtered, and concentrated in vacuo. The residue was purified by silica gel chromatography (80 g SiO₂, 100% n-heptane to 100% EA in 20 min). The product containing fractions were combined and the solvent was removed in vacuo. The residue was further purified by RP prep-HPLC (flow 50 ml/min; 90% H₂O/10% ACN to 10% H₂O/90% ACN in 15 min; Agilent Prep C18—10 μm, 30×250 mm). The product containing fractions were combined, the ACN was removed in vacuo and the aqueous phase was lyophilized over night to yield 1.07 g of the title compound.

LC/MS: m/z=363.3 [M+H]⁺; RT: 1.76 min (LC/MS-method A).

### Step 2: 1-06: 4-Piperidyl-[(3S)-3-pyrazin-2-ylisoxazolidin-2-yl]methanone trifluoro acetic acid salt

Following general procedure 3, 1.75 g of the title compound were obtained.

LC/MS: m/z=263.2 [M+H]⁺; RT: 0.39 min (Method A).

In analogy, the following intermediates were synthesized:

### Intermediate I-07: (3S)-3-(2-methylthiazol-4-yl)isoxazolidin-2-yl]-(4-piperidyl)methanone; 2,2,2-trifluoroacetic acid (Responding to Compound of Formula (IIb)) (Following Scheme 2)

### Step 1: Intermediate I-07

Following general procedure 1 and starting from 1-(tert-butoxycarbonyl)piperidine-4-carboxylic acid (162.1 g, 0.697 mmol) and (3S)-3-(2-methylthiazol-4-yl)isoxazolidine; 2,2,2-trifluoroacetic acid (179 mg, 0.63 mmol) tert-butyl tert-butyl 4-[(3S)-3-(2-methylthiazol-4-yl)isoxazolidine-2-carbonyl]piperidine-1-carboxylate was obtained (240 mg, 72%).

LC/MS: m/z=382.2 [M+H]⁺; RT: 0.736 min (Method B).

### Step 2: Intermediate I-09—(3S)-3-(2-methylthiazol-4-yl)isoxazolidin-2-yl]-(4-piperidyl)methanone; 2,2,2-trifluoroacetic acid

Following general procedure 3 and starting from tert-butyl (S)-4-(3-(5-fluoropyridin-3-yl)isoxazolidine-2-carbonyl)piperidine-1-carboxylate (600 mg, 1.58 mmol) the title compound was obtained in 99% yield.

LC/MS: m/z=282.1 [M+H]⁺; RT: 0.668 min (Method E).

### Intermediate I-08

### Step 1: benzyl 4-[(3S)-3-(6-methyl-3-pyridyl)isoxazolidine-2-carbonyl]piperidine-1-carboxylate (responding to compound of formula (IIb)) (following scheme 2)

Following the general procedure 1, from Intermediate I-03 (5 g, 18.28 mmol, 1 eq, 3HCl) and 1-benzyloxycarbonylpiperidine-4-carboxylic acid (5.05 g, 19.19 mmol, 1.05 eq) and stirred at 20° C. for 2 hours, the title compound was obtained (7.4 g, 93.94% yield) as a red oil.

LC/MS m/z 410.3 [M+1]⁺; RT 0.776 (Method C)

¹H NMR (CDCl₃, 400 MHz) δ ppm 8.46 (d, J=2.0 Hz, 1H), 7.56 (dd, J=2.3, 8.1 Hz, 1H), 7.40-7.29 (m, 5H), 7.17 (d, J=8.1 Hz, 1H), 5.40 (dd, J=6.1, 8.7 Hz, 1H), 5.13 (s, 2H), 4.30 (dt, J=3.4, 7.8 Hz, 1H), 4.26-4.08 (m, 2H), 3.98-3.87 (m, 1H), 3.03-2.81 (m, 4H), 2.57 (s, 3H), 2.43-2.31 (m, 2H), 2.01-1.81 (m, 1H), 1.79-1.62 (m, 3H).

### [(3S)-3-(6-methyl-3-pyridyl)isoxazolidin-2-yl]-(4-piperidyl)methanone

To a solution of benzyl 4-[(3S)-3-(6-methyl-3-pyridyl)isoxazolidine-2-carbonyl]piperidine-1-carboxylate (6.4 g, 15.63 mmol, 1 eq) in ACN (64 mL) was added Pd/C (640 mg, 10% purity) under N₂ atmosphere. The suspension was degassed and purged with H₂ atmosphere for 3 times. The mixture was stirred under H₂ (15 Psi) at 20° C. for 3 hr. LC/MS showed desired mass was detected. The mixture was filtered and concentrated. The residue was purified by prep-HPLC (column: Kromasil Eternity XT 250*80 mm*10 um; mobile phase: [water (10 mM NH₄HCO₃)-ACN]; B %: 10%-40%, 18 min) to give I-08 (2.2 g, 44.26% yield, e.e. 95.3%) as white solid.

LC/MS, m/z 276.0 [M+1]+; RT 1.377 min (Method D)

¹H NMR (CDCl₃, 400 MHz) δ ppm 8.46 (d, J=2.1 Hz, 1H), 7.51 (dd, J=2.3, 8.1 Hz, 1H), 7.12 (d, J=7.9 Hz, 1H), 5.40 (dd, J=6.2, 8.6 Hz, 1H), 4.28 (dt, J=3.4, 7.8 Hz, 1H), 3.95-3.86 (m, 1H), 3.14 (dt, J=4.1, 8.4 Hz, 2H), 2.96-2.80 (m, 2H), 2.76-2.63 (m, 2H), 2.54 (s, 3H), 2.40-2.29 (m, 1H), 1.88 (br dd, J=2.5, 13.0 Hz, 1H), 1.73 (br s, 1H), 1.68 (td, J=4.2, 8.3 Hz, 2H), 1.65-1.58 (m, 1H).

Chiral SFC RT for S enantiomer 1.361 min (97.6%, Method P).

### Intermediate I-09: (S)-(3-(5-fluoropyridin-3-yl)isoxazolidin-2-yl)(piperidin-4-yl)methanone, 2,2,2-trifluoroacetic acid (Responding to Compound of Formula (IIb)) (Following Scheme 2)

### Step 1: (S)-4-(3-(5-fluoropyridin-3-yl)isoxazolidine-2-carbonyl)piperidine-1-carboxylate

Following general procedure 1 and starting from 1-(tert-butoxycarbonyl)piperidine-4-carboxylic acid (1.43 g, 6.22 mmol) and (S)-3-(5-fluoropyridin-3-yl)isoxazolidine 2,2,2-trifluoroacetate (2.5 g, 5.66 mmol) corresponding to intermediate I-04-1, tert-butyl (S)-4-(3-(5-fluoropyridin-3-yl)isoxazolidine-2-carbonyl)piperidine-1-carboxylate was obtained (3.2 g, 55%).

LC/MS: m/z=280.1 [M+1-Boc]⁺; RT: 0.79 min (Method B).

### Step 2: (S)-(3-(5-fluoropyridin-3-yl)isoxazolidin-2-yl)(piperidin-4-yl)methanone, 2,2,2-trifluoroacetic acid

Following general procedure 3, the title compounds was obtained in 99% yield.

LC/MS: m/z=280.2 [M+H]⁺; RT: 0.39 min (Method B).

### Intermediate I-10 [(3S)-3-(5-fluoro-6-methyl-3-pyridyl)isoxazolidin-2-yl]-(4-piperidyl)methanone (Responding to Compound of Formula (IIb))

### Step 1

To a solution of 1-(tert-butoxycarbonyl)piperidine-4-carboxylic acid (1.71 g, 7.46 mmol, 1.2 eq) in DMF (15 mL) was added HBTU (4.72 g, 12.44 mmol, 2 eq) and DIEA (4.82 g, 37.32 mmol, 6.50 mL, 6 eq), then the mixture was stirred at 25° C. for 15 min. Intermediate I-05 (1.36 g, 6.22 mmol, 1 eq, HCl) was added and the mixture was stirred at 25° C. under N2 atmosphere for 12 hr. LCMS showed desired mass was detected. The mixture was filtered, purified by Prep. RP LC (flow: 200 ml/min; gradient: from 85% H2O (0.1% FA)/15% ACN to 50% H2O (0.1% FA)/50% ACN in 20 min; 50% H2O (0.1% FA)/50% ACN in 10 min; column: Welch Ultimate XB_C18 20-40 μm; 120 Å, I.D. 75 mm*H348 mm) and silica gel column chromatography (PE: EA=2:1~0:1) to give I-10 (1.5 g, 61.30% yield, >99.9% e.e.) as yellow solid.

LC/MS: m/z=394.3 [M+1-Boc]⁺; RT: 0.800 min (Method C).

¹H NMR (CDCl₃, 400 MHz): δ ppm 8.26 (s, 1H), 7.24 (dd, J=1.8, 9.9 Hz, 1H), 5.40 (dd, J=6.2, 8.7 Hz, 1H), 4.28 (dt, J=3.4, 7.7 Hz, 1H), 4.18-4.04 (m, 2H), 3.90 (ddd, J=6.7, 8.2, 9.2 Hz, 1H), 2.96-2.76 (m, 4H), 2.49 (d, J=2.9 Hz, 3H), 2.34 (dddd, J=6.1, 7.5, 9.3, 12.4 Hz, 1H), 1.93-1.83 (m, 1H), 1.72-1.59 (m, 3H), 1.45 (s, 9H).

Chiral SFC: RT 1.999 min (100%, Method M).

### Intermediate I-11: 5-azaspiro[2.5]octan-8-yl-[(3S)-3-(6-methyl-3-pyridyl)isoxazolidin-2-yl]methanone, 2,2,2 Trifluoroacetic acid (Responding to Compound of Formula (IIb))

### Step 1: tert-butyl 8-[(3S)-3-(6-methyl-3-pyridyl)isoxazolidine-2-carbonyl]-5-azaspiro[2.5]octane-5-carboxylate

A mixture of 5-tert-butoxycarbonyl-5-azaspiro[2.5]octane-8-carboxylic acid (800 mg, 3.13 mmol), (3S)-3-(6-methyl-3-pyridyl)isoxazolidine (943 mg, 3.45 mmol, 3HCl), HATU (2.38 g, 6.27 mmol), DIEA (2.73 mL, 15.67 mmol) in DMF (8 mL) was stirred at 25° C. for 12 hours. The reaction mixture was filtered and concentrated under reduced pressure to give a residue. The residue was purified by prep-HPLC (column: Phenomenexluna C18 150*40 mm*15 um; mobile phase: [water (0.225% FA)-ACN]; B %: 20%-50%, 10 min) to afford the title compound as a yellow solid as a mixture of two isomers (936 mg, 74%).

¹H NMR (400 MHz, CDCl₃) δ=3.92-3.66 (m, 1H), 3.40 (d, J=13.6 Hz, 1H), 3.31 (t, J=10.8 Hz, 1H), 3.13 (d, J=13.6 Hz, 1H), 2.15 (t, J=4.8 Hz, 1H), 2.06-1.96 (m, 1H), 1.96-1.84 (m, 1H), 0.68 (d, J=9.2 Hz, 1H), 0.64-0.53 (m, 2H), 0.47-0.35 (m, 1H).

LC/MS: m/z 402.1 [M+1]⁺; RT: 0.683 min (Method C).

Chital SFC: RT 1.216 (Isomer 1, 44.76%, Method O) 1.697 (Isomer 2, 55.2%, Method O) Step 2: 5-azaspiro[2.5]octan-8-yl-[(3S)-3-(6-methyl-3-pyridyl)isoxazolidin-2-yl]methanone, TFA salt.

To a solution of tert-butyl 8-[(3S)-3-(6-methyl-3-pyridyl)isoxazolidine-2-carbonyl]-5-azaspiro[2.5]octane-5-carboxylate (300 mg, 747 μmol) in DCM (1.5 mL) was added TFA (332 μL, 4.48 mmol). The mixture was stirred at 25° C. for 2 hours. The reaction mixture was concentrated under reduced pressure to give the title compound as a yellow solid (240 mg).

LC/MS: m/z 301.9 [M+1]⁺; RT: 0.128 min (broad peak) (Method D).

### Intermediate 11-(R)-(8R)-5-azaspiro[2.5]octan-8-yl]-[(3S)-3-(6-methyl-3-pyridyl)isoxazolidin-2-yl]methanone- and

### Intermediate 11-(S)—(8S)-5-azaspiro[2.5]octan-8-yl]-[(3S)-3-(6-methyl-3-pyridyl)isoxazolidin-2-yl]methanone

were obtained in a similar manner but using coupling the two isomers (8R) and (8S) of 5-tert-butoxycarbonyl-5-azaspiro[2.5]octane-8-carboxylic acid that were obtained by chiral separation of racemic 5-tert-butoxycarbonyl-5-azaspiro[2.5]octane-8-carboxylic acid (12.0 g, 0.0470 mol) were separated by SFC (column: DAICEL CHIRALPAK IG 250 mm*30 mm*5 um; Condition: C02-MeOH, B %: 15%, isocratic elution mode) to afford tert-butyl ((8R)-5-tert-butoxycarbonyl-5-azaspiro[2.5]octane-8-carboxylic acid (5.90 g, 0.0231 mol, 49.2% yield) as a yellow oil and (8S)-tert-butoxycarbonyl-5-azaspiro[2.5]octane-8-carboxylic acid (4.20 g, 0.0165 mol, 35.0% yield) as a yellow oil. RT 0.808 Isomer 1 (R,S); RT 0.855 (Isomer 2 (S,S) (Method K).
Chiral SFC: 1.53 min (100% ee, (S,S) isomer); 1.788 (99.3% ee, (R,S)-isomer (Method H)

Stereochemistry of (S,S)-isomers was unambiguously assigned via X-Ray structure.

### Intermediate I-32. (8R)-5-azaspiro[2.5]octan-8-yl-[(3S)-3-(5-fluoro-6-methyl-3-pyridyl)isoxazolidin-2-yl]methanone (HCl salt)

was synthesized using general procedure 1 and general procedure 2, but starting from (8R)-5-tert-butoxycarbonyl-5-azaspiro[2.5]octane-8-carboxylic acid (250 mg, 0.979 mmol, 1.00 eq.) and I-05 (800 mg, crude, −24% purity, 0.00106 mol, 1.10 eq.) to afford (8R)-5-azaspiro[2.5]octan-8-yl-[(3S)-3-(5-fluoro-6-methyl-3-pyridyl)isoxazolidin-2-yl]methanone

(HCl salt) I-32 (390 mg, crude, −77.9% purity) as a white solid.

LCMS: m/z=320.2 [M+H]⁺. RT 0.455 (Method C)

### Intermediate I-33. (8R)-5-azaspiro[2.5]octan-8-yl-[(3S)-3-(2-methylthiazol-4-yl)isoxazolidin-2-yl]methanone (HCL salt)

was synthesized using general procedure 1 and general procedure 2 but starting with (8R)-5-tert-butoxycarbonyl-5-azaspiro[2.5]octane-8-carboxylic acid (600 mg, 0.00235 mol) and I-02 762 mg, crude, −63.0% purity) to afford 5-azaspiro[2.5]octan-8-yl-[(3S)-3-(2-methylthiazol-4-yl)isoxazolidin-2-yl]methanone

LCMS: m/z=308.2 [M+H]⁺. RT 0.463 (Method Q)

(1H NMR (400 MHz, CDCl3) δ=8.53 (d, J=1.2 Hz, 1H), 8.40 (s, 1H), 7.90 (d, J=6.0 Hz, 1H), 5.56 (d, J=6.0 Hz, 1H), 5.51 (dd, J=8.8, 6.0 Hz, 1H), 4.75 (t, J=13.2 Hz, 2H), 4.38-4.28 (m, 2H), 4.22-4.13 (m, 2H), 4.02-3.94 (m, 1H), 3.89 (dd, J=9.2, 3.6 Hz, 2H), 3.33 (s, 3H), 3.07-2.98 (m, 1H), 2.97-2.86 (m, 2H), 2.85-2.77 (m, 1H), 2.75-2.65 (m, 1H), 2.56 (s, 3H), 1.99-1.87 (m, 1H), 1.82-1.67 (m, 3H).

### Intermediate I-34. [(3S)-3-(5-methyl-3-pyridyl)isoxazolidin-2-yl]-(4-piperidyl)methanone (HCl salt)

was synthesized using general procedure 1 and general procedure 2, using 1-tert-butoxycarbonylpiperidine-4-carboxylic acid (2.16 g, 0.00945 mol, 1.00 eq.) and I-31 (7.50 g, crude, −20.6% purity, 0.00945 mol, 1.00 eq.) to give I-34:HCl Salt [(3S)-3-(5-methyl-3-pyridyl)isoxazolidin-2-yl]-(4-piperidyl)methanone (4.26 g, crude, −56.6% purity) which was used as such in following reactions.

### Intermediate I-35. (8R)-5-azaspiro[2.5]octan-8-yl-[(3S)-3-pyrazin-2-ylisoxazolidin-2-yl]methanone (HCl salt)

was synthesized using general procedure 1 and general procedure 2 but starting with (8R)-5-tert-butoxycarbonyl-5-azaspiro[2.5]octane-8-carboxylic acid (2.00 g, 0.00783 mol) and I-01 (2.15 g, crude −66.0% purity, 0.00947 mol, 1.20 eq.) to afford (8R)-5-azaspiro[2.5]octan-8-yl-[(3S)-3-pyrazin-2-ylisoxazolidin-2-yl]methanone (HCl salt) I-35 (1.47 g, 0.00510 mol, 66.0% yield) as a white solid.

¹H NMR (400 MHz, CD₃OD) δ=8.66 (d, J=1.2 Hz, 1H), 8.61-8.57 (m, 1H), 8.53 (d, J=2.8 Hz, 1H), 5.49 (dd, J=8.8, 5.6 Hz, 1H), 4.41-4.33 (m, 1H), 4.11-4.04 (m, 1H), 3.71 (d, J=12.4 Hz, 1H), 3.30-3.25 (m, 1H), 3.22-3.06 (m, 1H), 2.98-2.84 (m, 1H), 2.74 (t, J=4.2 Hz, 1H), 2.67-2.51 (m, 2H), 2.28-2.11 (m, 2H), 0.79-0.56 (m, 4H).

LCMS: m/z=289.2 [M+H]⁺. RT 0.348 min (Method C)
Chiral SFC: RT 1.234, 100% ee (Method T)

### Intermediate I-36. [(3S)-3-(5-methylpyrazin-2-yl)isoxazolidin-2-yl]-(4-piperidyl)methanone (HCl salt)

was synthesized using general procedure 1 and general procedure 2 but starting with 1-tert-butoxycarbonylpiperidine-4-carboxylic acid (3.14 g, 0.0137 mol) and I-29 (3.14 g, 0.0137 mol, 1.20 eq.) to afford [(3S)-3-(5-methylpyrazin-2-yl)isoxazolidin-2-yl]-(4-piperidyl)methanone (HCl salt) I-36 (2.5 g, 65% purity) as a white solid. This compound was used in the next steps without further purification.

### Intermediate AA-1: Methyl 1-(6-chloropyrimidin-4-yl)piperidine-4-carboxylate (Responding to Compound of Formula (IX))

A mixture of 4,6-dichloropyrimidine (80 g, 536.99 mmol, 1 eq.), methyl piperidine-4-carboxylate (96.47 g, 536.99 mmol, 1 eq., HCl), DIEA (208.21 g, 1.61 mol, 280.60 ml, 3 eq.) in n-BuOH (700 ml) was degassed and purged with N₂, and then the mixture was stirred at 80° C. for 2 hrs under N₂ atmosphere. The reaction mixture was concentrated under reduced pressure to remove n-BuOH. Then diluted with H₂O (100 ml) and EA (200 ml), adjusted to pH 6 with 1 N HCl solution and extracted with ethyl acetate (200 ml×3). The combined organic layer was washed with NaHCO₃ aqueous solution (500 ml), brine (500 ml), dried over Na₂SO₄, filtered and concentrated to give the title compound (135.4 g crude, 529.53 mmol, 97% yield) as a yellow solid.

LC/MS: m/z=256.1 [M+H]⁺; RT: 0.737 min (LC/MS method C).

¹H NMR (400 MHz, DMSO-d₆): δ ppm 8.32 (s, 1H), 6.97 (s, 1H), 4.38-4.19 (m, 2H), 3.62 (s, 3H), 3.16-3.02 (m, 2H), 2.71 (tt, J=4.0, 10.8 Hz, 1H), 1.96-1.84 (m, 2H), 1.60-1.44 (m, 2H).

### Intermediate AA-2: Methyl 1-(4-bromo-5-fluoro-pyrimidin-2-yl)piperidine-4-carboxylate (Responding to Compound of Formula (IX))

### Step 1: 2-Chloro-5-fluoro-pyrimidin-4-amine

A mixture of 2,4-dichloro-5-fluoro-pyrimidine (100 g, 600 mmol) in NH₃·H2O (200 ml) was stirred at 60° C. for 1 h. The reaction mixture was filtered to yield 2-Chloro-5-fluoro-pyrimidin-4-amine (78 g, 68%). ¹H NMR (400 MHz, DMSO-d₆): δ ppm 8.09 (1H), 7.81 (2H).

### Step 2: Methyl 1-(4-amino-5-fluoro-pyrimidin-2-yl)piperidine-4-carboxylate

A mixture of 2-chloro-5-fluoro-pyrimidin-4-amine (67 g, 456 mmol), methyl piperidine-4-carboxylate hydrochloride salt (122.37 g, 720 mmol) and DIPEA (205.42 g) in n-BuOH (670 ml) was stirred at 100° C. for 12 h under N₂ atmosphere. Then the reaction mixture was concentrated under reduced pressure to remove the solvent. The residue was diluted with water (500 ml) and extracted with EA (300 ml×3). The combined organic layers were washed with brine (500 ml), dried over Na₂SO₄, filtered and concentrated. The residue was purified by silica gel column chromatography (PE:EA=10:1 to 4:1) to yield ethyl 1-(4-amino-5-fluoro-pyrimidin-2-yl)piperidine-4-carboxylate (73 g, 63%).

¹H NMR (400 MHz, CDCl₃): δ ppm 7.81 (1H), 4.82 (2H), 4.49 (2H), 3.69 (3H), 3.00-2.87 (2H), 2.52 (1H), 1.92 (2H), 1.76-1.57 (2H).

Step 3: Methyl 1-(4-bromo-5-fluoro-pyrimidin-2-yl)piperidine-4-carboxylate To a mixture of methyl 1-(4-amino-5-fluoro-pyrimidin-2-yl)piperidine-4-carboxylate (68.8 g, 270 mmol) and CuBr₂ (120.87 g) in DCM (688 ml) was added isopentyl nitrite (63.40 g) at 0° C. The mixture was warmed to 25° C. smoothly and stirred for 12 h. Then the reaction mixture was filtered, the filtrate was diluted with water (500 ml) and extracted with DCM (500 ml×3). The combined organic layers were washed with brine (1000 ml), dried over Na₂SO₄, filtered and concentrated under reduced pressure to give a residue. The residue was purified by silica gel column chromatography (PE:EA=20:1 to 10:1) to yield Intermediate AA-2 (26 g, 32%).

¹H NMR (400 MHz, CDCl₃): δ ppm 8.06 (1H), 4.53 (2H), 3.71 (3H), 3.14-2.98 (2H), 2.58 (1H), 2.03-1.93 (2H), 1.80-1.62 (2H).

### Intermediate AA-3: methyl 1-(2-chloro-5-fluoro-pyrimidin-4-yl)piperidine-4-carboxylate (Responding to Compound of Formula (IX))

To a solution of compound 2,4-dichloro-5-fluoro-pyrimidine (10 g, 59.89 mmol, 1 eq) and methyl piperidine-4-carboxylate (11.30 g, 62.89 mmol, 1.05 eq, HCl) in DCM (100 mL) was added TEA (9.09 g, 89.84 mmol, 12.50 mL, 1.5 eq). The mixture was stirred at 25° C. for 12 hr. TLC indicated the starting material was consumed completely and many new spots formed. The reaction was messy according to TLC. The reaction mixture was diluted with water 100 mL and extracted with DCM (100 mL*3). The combined organic layers were washed with brine 100 mL, dried over Na₂SO₄, filtered and concentrated under reduced pressure to give a residue. The residue was purified by column chromatography (SiO₂, PE/EA=1/0 to 10/1). Intermediate AA3 (13.55 g, 83% yield) was obtained as a yellow oil.

¹H NMR (CDCl₃, 400 MHz,) δ=7.85 (d, J=6.3 Hz, 1H), 4.35 (td, J=3.7, 13.7 Hz, 2H), 3.64 (s, 3H), 3.14 (ddd, J=2.9, 11.2, 13.7 Hz, 2H), 2.57 (tt, J=4.2, 10.6 Hz, 1H), 1.98-1.89 (m, 2H), 1.81-1.65 (m, 2H).

### Intermediate AA-4: methyl 1-(4-chloro-1,3,5-triazin-2-yl)piperidine-4-carboxylate (Responding to Compound of Formula (IX))

To a solution of 2,4-dichloro-1,3,5-triazine (25 g, 166.70 mmol, 1 eq) in dioxane (300 mL) was added DIEA (60.98 mL, 350.08 mmol, 2.1 eq) and methyl piperidine-4-carboxylate (29.95 g, 166.70 mmol, 1 eq, HCl). The mixture was stirred at 25° C. for 1 hr. TLC indicated that the starting material was consumed completely and two new spots were formed. The reaction mixture was concentrated, diluted with water (300 mL), extracted with EA (200 mL*2), washed with brine (300 mL), dried with Na₂SO₄, filtrated and concentrated. Intermediate AA-4 (44.5 g, crude) was obtained as a white solid and used in the next step without further purification.

¹H NMR (400 MHz, CDCl₃) δ=8.34 (s, 1H), 4.68-4.47 (m, 2H), 3.71 (s, 3H), 3.19 (dddd, J=3.1, 7.8, 10.9, 13.7 Hz, 2H), 2.64 (tt, J=4.1, 10.6 Hz, 1H), 2.04-1.97 (m, 2H), 1.80-1.67 (m, 2H).

### Intermediate AA-5: methyl 1-(2-chloropyrimidin-4-yl)piperidine-4-carboxylate (Responding to Compound of Formula (IX))

A mixture of 2,4-dichloropyrimidine (2 g, 13.42 mmol, 1 eq.), methyl piperidine-4-carboxylate (1.92 g, 13.42 mmol, 1 eq.), DIEA (5.21 g, 40.27 mmol, 7.02 mL, 3 eq.) in n-BuOH (20 mL) was degassed and purged with N₂, and then the mixture was stirred at 80° C. for 2 hrs under N₂ atmosphere. LC/MS showed no starting material remained. Several new peaks were shown on LC/MS and −74% of desired mass was detected. The mixture was diluted with H₂O (30 mL) and extracted with EtOAc (30 mL*2). The combined organic layers were dried over anhydrous Na₂SO₄, filtered and concentrated under reduced pressure. The residue was purified by flash silica gel chromatography (ISCO®; 25 g SepaFlash® Silica Flash Column, Eluent of 0~50% Ethyl acetate in Petroleum ether gradient @50 mL/min) to give Intermediate AA-5 (985 mg, 7% yield, as a white solid.

LC/MS: m/z 256.1[M+1]⁺, RT 0.759 min (Method C).

¹H NMR: (CDCl₃, 400 MHz) δ ppm 8.03 (d, J=6.2 Hz, 1H), 6.41 (d, J=6.1 Hz, 1H), 4.25 (br s, 2H), 3.71 (s, 3H), 3.12 (ddd, J=3.0, 11.0, 13.7 Hz, 2H), 2.63 (tt, J=4.1, 10.6 Hz, 1H), 2.06-1.96 (m, 2H), 1.81-1.68 (m, 2H).

### Intermediate AA-6. tert-butyl 1-(4-oxazol-2-ylpyrimidin-2-yl)piperidine-4-carboxylate (Responding to Compound of Formula (IX))

### Step 1a

A mixture of Methyl 2-chloropyrimidine-4-carboxylate (860 mg, 4.9 mmol), tert-butyl piperidine-4-carboxylate; hydrochloride (1 g, 4.5 mmol) and DIPEA (2.75 mL, 3.5 eq.) in ACN (15 mL) was stirred in the MW at 100° C. for 1 hr. LC/MS showed conversion to desired product. The solvent was evaporated and the residue purified by column chromatography on Silica gel (n-Hep/EA gradient) to give methyl 2-(4-tert-butoxycarbonyl-1-piperidyl)pyrimidine-4-carboxylate (1.1 g, 76%).

LC/MS: m/z 322.2 [M+1]⁺, RT 2.39 min (Method A).

### Step 1b

Following the general procedure for ester hydrolysis, the desired product 2-(4-tert-butoxycarbonyl-1-piperidyl)pyrimidine-4-carboxylic acid; hydrochloride (950 mg, 80%) was obtained starting from methyl 2-(4-tert-butoxycarbonyl-1-piperidyl)pyrimidine-4-carboxylate (1.1 g, 3.43 mmol).

LC/MS: m/z 308.2 [M+1]⁺, RT 1.99 min (Method A).

### Step 2a

Following the general procedure for amide coupling and using 2-(4-tert-butoxycarbonyl-1-piperidyl)pyrimidine-4-carboxylate (195 mg, 0.64 mmol) and AMINOACETALDEHYDE DIMETHYL ACETAL (0.274 mL, 2.5 mmol) and DIPEA 3 eq, the desired product tert-butyl 1-[4-(2,2-dimethoxyethylcarbamoyl)pyrimidin-2-yl]piperidine-4-carboxylate (61.7 mg, 28%).

LC/MS: m/z 395.2 [M+1]⁺, RT 2.3 min (Method A).

### Step 2b

To a solution of tert-butyl 1-[4-(2,2-dimethoxyethylcarbamoyl)pyrimidin-2-yl]piperidine-4-carboxylate (40 mg, 0.10 mmol) in Dioxane (3 mL), HCl (4N in Dioxane, 1 mL) was added and the mixture stirred at rt overnight. The mixture was neutralized with saturated aqueous NaHCO₃ and extracted with DCM (3×). The combined organic layers were dried over MgSO₄, filtered and the residue was evaporated. The crude product tert-butyl 1-[4-(2-oxoethylcarbamoyl)pyrimidin-2-yl]piperidine-4-carboxylate (30 mg; 85%) was used in the following step without further purification.

LC/MS: m/z 395.2 [M+1]+, RT 2.3 min (Method A).

### Step 3

To a solution of 1-[4-(2-oxoethylcarbamoyl)pyrimidin-2-yl]piperidine-4-carboxylate (150 mg; 0.43 mmol) in THF (12 mL), Burgess reagent (307 mg, 1,292 mmol) was added. The mixture was stirred in the MW at 80° C. for 20 min, and LC/MS showed full conversion to product. T The mixture was neutralized with saturated aqueous NaHCO₃ and extracted with DCM (3×). The combined organic layers were dried over MgSO4, filtered and the residue was evaporated and the residue purified by chromatography on silica gel (nHept/EE gradient−(nHep_100%-80% EE=30 min.). to give Intermediate AA-6 (71 mg, 50%).

LC/MS: m/z 331.2 [M+1]+, RT 2.35 min (Method A).

¹H NMR (400 MHz, DMSO-d6) δ ppm 8.53 (d, J=5.01 Hz, 1H), 8.35 (s, 1H), 7.50 (s, 1H), 7.20 (d, J=4.89 Hz, 1H), 4.58 (m, 2H), 3.11 (m, 2H), 2.57 (m, 1H), 2.50 (u), 1.88 (br dd, J=13.45, 3.18 Hz, 2H), 1.48 (m, 2H), 1.41 (s, 9H), 1.24 (br d, J=3.79 Hz, 1H).

### Intermediate I-12:1-[6-(2-Methylimidazol-1-yl)pyrimidin-4-yl]piperidine-4-carboxylic acid (Responding to Compound of Formula (IIa)) (Following SM3 of Scheme 3 and Step 2 of SM1 of Scheme 1)

### I-12-a: Methyl 1-[6-(2-methylimidazol-1-yl)pyrimidin-4-yl]piperidine-4-carboxylate

A mixture of Intermediate-AA-1 (3.97 g, 15.53 mmol, 1 eq.), 2-methyl-1H-imidazole (2.55 g, 31.05 mmol, 2 eq.), K₂CO₃ (8.58 g, 62.10 mmol, 4 eq.) and CuI (1.48 g, 7.76 mmol, 0.5 eq.) in DMSO (40 ml) was degassed and purged with N₂ for 3 times. The mixture was stirred at 120° C. for 12 hrs under N₂ atmosphere. The reaction mixture was filtered, diluted with water (400 ml) and extracted with ethyl acetate (200 ml×3). The combined organic layer was washed with brine (300 ml), dried over Na₂SO₄, filtered, concentrated and purified by flash silica gel chromatography (eluent: Petroleum ether and ethyl acetate, gradient: 35% to 70% ethyl acetate and eluent: MeOH and DCM, gradient, 5% to 15% MeOH) to give the title compound I-12a (2.3 g, 7.63 mmol, 49% yield) as a yellow oil.

LC/MS: m/z=302.1 [M+H]⁺; RT: 0.615 min (LC/MS method C).

¹H NMR (400 MHz, CDCl₃): δ ppm 8.54 (s, 1H), 7.30 (s, 1H), 7.04 (s, 1H), 6.41 (s, 1H), 4.32 (br d, J=13.1 Hz, 2H), 3.73 (s, 3H), 3.18 (ddd, J=3.0, 11.0, 13.7 Hz, 2H), 2.67 (s, 3H), 2.08-2.03 (m, 2H), 1.84-1.74 (m, 3H).

### Intermediate I-12: 1-[6-(2-Methylimidazol-1-yl)pyrimidin-4-yl]piperidine-4-carboxylic acid

To a solution of methyl 1-[6-(2-methylimidazol-1-yl)pyrimidin-4-yl]piperidine-4-carboxylate (4.46 g, 14.80 mmol, 1 eq.) in THF (50 ml) LiOH·H₂O (1 M, 29.60 ml, 2 eq.) was added. The mixture was stirred at 20° C. for 1 hr. The reaction mixture was adjusted to pH 3 with 4 N HCl aqueous solution, the mixture was concentrated in vacuo and purified by reversed-phase HPLC (column: Welch Ultimate XB_C18, 20-40 μm, 120 Å; eluent: water (0.1% formic acid) and acetonitrile, gradient: 100% to 10% acetonitrile in 20 min, 10% acetonitrile for 20 min, flow: 100 ml/min) to give the title compound (3.5 g, 10.81 mmol, 73% yield, HCl) as a yellow solid.

LC/MS: m/z=288.2 [M+H]⁺; RT: 0.700 min (LC/MS method D).

¹H NMR (400 MHz, D2O): 6 ppm 8.50 (s, 1H), 7.70 (d, J=2.3 Hz, 1H), 7.47 (d, J=2.2 Hz, 1H), 7.08 (s, 1H), 4.34-4.17 (m, 2H), 3.34-3.26 (m, 2H), 2.83-2.76 (m, 1H), 2.73 (s, 3H), 2.06 (br dd, J=3.4, 13.5 Hz, 2H), 1.75-1.65 (m, 2H).

### Intermediate I-13: 1-[6-(2-Methylpyrazol-3-yl)pyrimidin-4-yl]piperidine-4-carboxylic acid (Responding to Compound of Formula (IIa)) (Following SM3 of Scheme 3 and Step 2 of SM1 of Scheme 1)

### I-13a: Methyl 1-[6-(2-methylpyrazol-3-yl)pyrimidin-4-yl]piperidine-4-carboxylate

To a solution of Intermediate-AA-1 (25 g, 97.77 mmol, 1 eq.) in dioxane (200 ml) and H₂O (40 ml) K₂CO₃ (27.03 g, 195.54 mmol, 2 eq.), 1-methyl-5-(4,4,5,5-teRTamethyl-1,3,2-dioxaborolan-2-yl)pyrazole (20.34 g, 97.77 mmol, 1 eq.) and Pd(dppf)Cl₂ (7.15 g, 9.78 mmol, 0.1 eq.) were added. The mixture was stirred at 120° C. for 2 hrs under N₂ atmosphere. The residue was diluted with H₂O (200 ml) and extracted with ethyl acetate (300 ml×2). The combined organic layer was dried over Na₂SO₄, filtered, concentrated under reduced pressure and purified by flash silica gel chromatography (column: 220 g SepaFlash® Silica Flash, eluent: Petroleum ether and ethyl acetate, gradient: 0% to 20% ethyl acetate, flow: 100 ml/min) to give the title compound (29.3 g, 88% yield) as a yellow oil.

LC/MS: m/z=302.1 [M+H]⁺; RT: 0.644 min (LC/MS method C).

### Intermediate I-13: 1-[6-(2-Methylpyrazol-3-yl)pyrimidin-4-yl]piperidine-4-carboxylic acid

To a solution of methyl 1-[6-(2-methylpyrazol-3-yl)pyrimidin-4-yl]piperidine-4-carboxylate (29.3 g, 97.23 mmol, 1 eq.) in THF (250 ml) and H₂O (50 ml) LiOH—H₂O (12.24 g, 291.69 mmol, 3 eq.) was added. The mixture was stirred at 25° C. for 12 hrs, adjusted to pH 5 with 1 N HCl solution, concentrated under reduced pressure and filtered. The filter cake was washed with H₂O (50 ml) and triturated with ethyl acetate at 20° C. for 1 hr to give Intermediate I-13 (19.4 g, 64.89 mmol, 67% yield) as a gray solid.

LC/MS: m/z=288.4 [M+H]⁺; RT: 0.295 min (LC/MS method C).

¹H NMR (400 MHz, DMSO-d₆): δ ppm 12.30 (s, 1H), 8.57 (d, J=0.9 Hz, 1H), 7.48 (d, J=2.0 Hz, 1H), 7.13 (d, J=0.9 Hz, 1H), 6.95 (d, J=2.0 Hz, 1H), 4.37 (d, J=12.6 Hz, 2H), 4.14 (s, 3H), 3.17-3.02 (m, 2H), 2.64-2.54 (m, 1H), 1.97-1.83 (m, 2H), 1.58-1.41 (m, 2H).

### Intermediate I-14: 1-(5-fluoro-4-pyrazol-1-yl-pyrimidin-2-yl)piperidine-4-carboxylic acid (Responding to Compound of Formula (IIa)) (Following SM3 of Scheme 3 and Step 2 of SM1 of Scheme 1)

### Step 1; I-18a

To a solution of Intermediate AA-2 (5 g, 15.72 mmol, 1.2 eq), Imidazole (891.60 mg, 13.10 mmol, 1 eq) in DMSO (50 mL) was added CuI (498.86 mg, 2.62 mmol, 0.2 eq) and proline (603.14 mg, 5.24 mmol, 0.4 eq), K₂CO₃ (5.43 g, 39.29 mmol, 3 eq). The mixture was stirred at 90° C. for 72 hr under N₂. LC/MS showed desired mass was given. The reaction mixture was diluted with H₂O 200 mL and extracted with EA 20 mL (100 mL*3). The combined organic layers were washed with brine 200 mL, dried over Na₂SO₄, filtered and concentrated under reduced pressure to give a residue. The residue was purified by column chromatography (SiO₂, Petroleum ether/Ethyl acetate=5/1 to 4/1) to give Intermediate I-14—Methyl ester (2.2 g, 7.21 mmol, 55.02% yield) as a white solid.

LC/MS: m/z 306.8 [M+1]⁺; RT: 0.872 min (Method C).

¹H NMR (CDCl₃, 400 MHz) δ=8.46 (d, J=2.7 Hz, 1H), 8.35 (d, J=4.0 Hz, 1H), 7.89 (d, J=1.2 Hz, 1H), 6.56-6.45 (m, 1H), 4.61 (dt, J=13.5, 3.6 Hz, 2H), 3.72 (s, 3H), 3.20-3.02 (m, 2H), 2.61 (tt, J=11.0, 3.9 Hz, 1H), 2.02 (br dd, J=13.5, 3.4 Hz, 2H), 1.85-1.58 (m, 3H).

### Step 2—Intermediate I-14

A mixture of Intermediate 14—Methyl ester (2.2 g, 7.21 mmol, 1 eq), LiOH·H₂O (1 M, 14.41 mL, 2 eq) in THF (4 mL) and H₂O (12 mL), and then the mixture was stirred at 25° C. for 2 hr. LC/MS showed desired mass was given. The reaction mixture was filtered and concentrated under reduced pressure to give a residue. The crude product was triturated with EA for 20 min to give Intermediate I-14 (2 g, 6.87 mmol, 95.29% yield) as a white solid.

LC/MS: m/z 292.1 [M+1]⁺; RT: 0.541 min (Method C).

¹H NMR (CDCl3, 400 MHz) b 8.36 (d, J=2.8 Hz, 1H), 8.27 (d, J=4.0 Hz, 1H), 7.82 (d, J=1.1 Hz, 1H), 6.44 (dd, J=2.6, 1.8 Hz, 1H), 4.53 (dt, J=13.6, 3.7 Hz, 2H), 3.13 ?3.00 (m, 2H), 2.57 (tt, J=10.9, 3.9 Hz, 1H), 1.97 (br dd, J=13.6, 3.4 Hz, 2H), 1.77 ?1.62 (m, 2H).

### Intermediate I-15: 1 1-[5-fluoro-4-(2-methylpyrazol-3-yl)pyrimidin-2-yl]piperidine-4-carboxylic acid (Responding to Compound of Formula (IIa)) (Following SM3 of Scheme 3 and Step 2 of SM1 of Scheme 1)

### Step 1

To a solution of Intermediate AA-2 (3 g, 9.43 mmol, 1 eq.), 1-methyl-5-(4,4,5,5-tetramethyl-1,3,2-dioxaborolan-2-yl)pyrazole (2.94 g, 14.14 mmol, 1.5 eq.), K₂CO₃ (1.95 g, 14.14 mmol, 1.5 eq.), Pd(PPh₃)₄ (344.99 mg, 471.49 μmol, 0.05 eq.) in dioxane (36 mL) and H₂O (6 mL) was degassed and purged with N₂ for 3 times, and then the mixture was stirred at 80° C. for 15 hrs under N₂ atmosphere. LC/MS showed desired mass was detected and TLC showed new spots were formed. The residue was diluted with H₂O (100 mL) and extracted with CH₂Cl₂ (100 mL*2). The combined organic layers was dried over anhydrous Na₂SO₄, filtered and concentrated under reduced pressure. The residue was purified by flash silica gel chromatography (ISCO@; 45 g SepaFlash@Silica Flash Column, Eluent of 0-50% Ethyl acetate in Petroleum ether gradient @60 mL/min) to give Intermediate I-15-Methyl ester (2.9 g, 9.08 mmol, 96.31% yield) as a yellow solid.

LC/MS: m/z 320.4 [M+1]⁺; RT: 0.922 min (Method C).

¹H NMR: (CDCl₃, 400 MHz) δ ppm 8.29 (d, J=2.9 Hz, 1H), 7.58 (d, J=2.1 Hz, 1H), 6.91 (dd, J=2.1, 4.2 Hz, 1H), 4.59 (td, J=3.6, 13.4 Hz, 2H), 4.30 (s, 3H), 3.73 (s, 3H), 3.22-3.05 (m, 2H), 2.63 (tt, J=4.0, 11.0 Hz, 1H), 2.02 (br dd, J=3.4, 13.5 Hz, 2H), 1.83-1.67 (m, 2H).

### Step 2: 1 1-[5-fluoro-4-(2-methylpyrazol-3-yl)pyrimidin-2-yl]piperidine-4-carboxylic acid

To a solution of Intermediate I-15-Methyl ester (2.8 g, 8.77 mmol, 1 eq.), LiOH—H₂O (735.90 mg, 17.54 mmol, 2 eq.) in THF (14 mL) and H₂O (14 mL) was stirred at 25° C. for 3 hrs. LC/MS showed desired mass was detected. The reaction mixture was concentrated under reduced pressure to remove solvent and adjusted pH to 4-5 with 1M HCl, then diluted with H₂O (30 mL) and stirred for 5 mins, filtered and washed with H₂O to give a crude solid. The residue was triturated with Petroleum ether: Ethyl acetate (1:1) at 25° C. for 30 mins to give Intermediate I-15 (2.2 g, 7.21 mmol, 82.18% yield) as a yellow solid.

LC/MS: m/z 305.9 [M+1]+; RT: 0.647 min (Method C)

¹H NMR: (DMSO-d6, 400 MHz) δ ppm 12.26 (br s, 1H), 8.55 (d, J=2.9 Hz, 1H), 7.60 (d, J=2.0 Hz, 1H), 6.85 (dd, J=2.1, 4.1 Hz, 1H), 4.45 (td, J=3.5, 13.3 Hz, 2H), 3.20-3.01 (m, 2H), 2.77-2.61 (m, 1H), 1.90 (br dd, J=3.2, 13.3 Hz, 2H), 1.64-1.40 (m, 2H)

### Intermediate I-16: 1-(5-fluoro-4-oxazol-2-yl-pyrimidin-2-yl)piperidine-4-carboxylic acid (Responding to Compound of Formula (IIa)) (Following SM3 of Scheme 3 and Step 2 of SM1 of Scheme 1)

### Step 1. 1-(5-fluoro-4-oxazol-2-yl-pyrimidin-2-yl)piperidine-4-carboxylic acid methyl ester

A degassed mixture of Intermediate AA-2 (4 g, 12.57 mmol, 1 eq) and tributyl(oxazol-2-yl)stannane (9.00 g, 25.15 mmol, 2 eq) were stirred in dioxane (30 mL) at ambient temperature (25° C.). This yellow solution was degassed with an N₂ flux for 10 min and Pd(PPh₃)₄ (1.45 g, 1.26 mmol, 0.1 eq) was added. The solution was stirred at 100° C. for 12 h. LC/MS showed the reaction worked well. the solvent was removed, dissolved into H₂O (50 mL), extracted with EA (50 mL×2), washed with brine (40 mL), dried over anhydrous Na₂SO₄, filtered and concentrated to give the crude product. The residue was purified by flash silica gel chromatography (Eluent of 10-30% EA/PE gradient) to give Intermediate I-16—methyl ester (2.88 g, 72%) as a yellow solid.

LC/MS: m/z 306.9 [M+H]⁺; RT: 0.682 min (Method C).

¹H NMR (400 MHz, DMSO-d6) δ=8.64 (d, J=2.9 Hz, 1H), 8.42 (s, 1H), 7.57 (s, 1H), 4.53-4.47 (m, 2H), 3.62 (s, 3H), 3.13-3.05 (m, 2H), 2.66 (tt, J=3.8, 11.1 Hz, 1H), 1.92 (dd, J=3.0, 13.1 Hz, 2H), 1.58-1.47 (m, 2H).

### Step 2-1-(5-fluoro-4-oxazol-2-yl-pyrimidin-2-yl)piperidine-4-carboxylic acid

To a solution of Intermediate AA2-Methyl ester (50 mg, 0.16 mmol), LiOH (5.8 mg, 1.5 eq.) in THF (3 mL) and H₂O (0.75 mL) was stirred at 25° C. for 4.5 hrs. LC/MS showed desired mass was detected. The reaction mixture was concentrated under reduced pressure to remove solvent and adjusted pH to 4-5 with 1M HCl, then extracted with EA (2×), dried, filtered and evaporated to give Intermediate I-16 (55 mg, 100%).

LC/MS: m/z 293.1 [M+H]⁺; RT: 1.47 min (Method A).

¹H NMR (DMSO-d6, 400 MHz) δ ppm 12.24 (br s, 1H), 8.64 (d, J=2.93 Hz, 1H), 8.42 (s, 1H), 7.57 (s, 1H), 4.50 (m, 2H), 3.10 (m, 2H), 1.92 (br dd, J=9.84, 3.48 Hz, 2H), 1.89 (br s, 1H), 1.52 (m, 2H), 1.18 (t, J=7.09, 7.09 Hz, 1H).

### Intermediate I-17: 1-(5-fluoro-4-oxazol-2-yl-pyrimidin-2-yl)piperidine-4-carboxylic-acid (Responding to Compound of Formula (IIa)) (Following SM3 of Scheme 3 and Step 2 of SM1 of Scheme 1)

### Step 1

A degassed mixture of tributyl(oxazol-2-yl)stannane (3 g, 10.96 mmol, 1 eq) and Intermediate AA-3 (7.85 g, 21.92 mmol, 2 eq) were stirred in dioxane (30 mL) at ambient temperature (25° C.). This yellow solution was degassed with an N₂ flux for 10 min and Pd(PPh₃)₄ (1.27 g, 1.10 mmol, 0.1 eq) was added. The solution was stirred at 100° C. for 12 h. LC/MS showed the reaction worked well, the solvent was removed, dissolved into H2O (50 mL), extracted with EA (50 mL*2), washed with brine (40 mL), dried over anhydrous Na₂SO₄, filtered and concentrated to give the crude product. The residue was purified by flash silica gel chromatography (Eluent of 20-60% Ethyl acetate/Petroleum ether gradient). Then purified by reversed-phase HPLC (0.1% FA condition). Intermediate I-17-methyl ester (2.38 g, 79% yield) was obtained as a yellow solid.

¹H NMR (DMSO-δ6; 400 MHz): δ=8.37 (br d, J=6.6 Hz, 1H), 8.27 (s, 1H), 7.43 (s, 1H), 4.40 (br d, J=13.2 Hz, 2H), 3.62 (s, 3H), 3.24 (br t, J=12.5 Hz, 2H), 2.78-2.69 (m, 1H), 1.97 (br d, J=13.3 Hz, 2H), 1.64 (q, J=11.5 Hz, 2H).

### Step 2: 1-(5-fluoro-4-oxazol-2-yl-pyrimidin-2-yl)piperidine-4-carboxylic-acid

To a solution of Intermediate I-17—methyl ester (2.28 g, 7.44 mmol, 1 eq) in THF (23 mL) was added LiOH·H₂O (1 M, 14.89 mL, 2 eq). The mixture was stirred at 25° C. for 3 hr. LC/MS showed Reactant 1 was consumed completely and one main peak with desired mass was detected. The reaction mixture was adjusted to pH=3 with 1 N HCl aqueous solution, the precipitate was collected through filtration and dried in vacuo. Intermediate I-17 (2 g, 92%) was obtained as a white solid.

LC/MS: m/z 292.1 [M+H]⁺; 0.710 min (Method C).

¹H NMR (DMSO 400 MHz): δ=12.31 (s, 1H), 8.37 (d, J=6.8 Hz, 1H), 8.27 (s, 1H), 7.43 (s, 1H), 4.43-4.35 (m, 2H), 3.29-3.19 (m, 2H), 2.61 (tt, J=4.0, 10.9 Hz, 1H), 1.95 (br dd, J=3.2, 13.5 Hz, 2H), 1.67-1.56 (m, 2H).

### Intermediate I-18: 1-(4-thiazol-2-yl-1,3,5-triazin-2-yl)piperidine-4-carboxylic acid (Responding to Compound of Formula (IIa)) (Following SM3 of Scheme 3 and Step 2 of SM1 of Scheme 1)

### Step 1: 1-(4-thiazol-2-yl-1,3,5-triazin-2-yl)piperidine-4-carboxylic acid methyl ester

To a solution of thiazole (6.5 g, 76.36 mmol, 549.45 μL, 1 eq) in THF (200 mL) at −78° C. under N₂ was added dropwise n-BuLi (2.5 M, 31.15 mL, 1.02 eq) and the mixture was stirred at −78° C. for 0.5 hr. Then tributyl(chloro)stannane (24.85 g, 76.36 mmol, 20.54 mL, 1 eq) was added and the mixture was warmed to 20° C. for 1 hr. The mixture was concentrated, and residue was taken up in hexanes (120 mL), the resulting precipitate was removed by filtration and the filtrate was concentrated to give tributyl(thiazol-2-yl)stannane (29 g) which was used in the next step without further purification.

A mixture of tributyl(thiazol-2-yl)stannane (5.83 g, 15.58 mmol, 2 eq), Intermediate AA-4 (2 g, 7.79 mmol, 1 eq), Pd(PPh₃)₄ (900.36 mg, 779.16 μmol, 0.1 eq) in dioxane (20 mL) was degassed and purged with N₂ for 3 times, and then the mixture was stirred at 100° C. for 4 hr under N₂ atmosphere. LC/MS showed no sm left and desired m/z was detected. TLC indicated some spots was formed. The mixture was concentrated and purified by flash silica gel chromatography (ISCO@; 40 g SepaFlash@Silica Flash Column, Eluent of 30-55% Ethyl acetate/Petroleum ethergradient @80 mL/min), to give Intermediate I-18-methyl ester (520 mg, 22% yield) was obtained as a yellow solid.

LC/MS: m/z 306.2 [M+H]+; 0.798 min (Method C).

### Step 2: 1-(4-thiazol-2-yl-1,3,5-triazin-2-yl)piperidine-4-carboxylic acid

To a solution of Intermediate I-18-methyl ester (2.1 g, 6.88 mmol, 1 eq) in THF (5 mL) was added LiOH (1 M, 13.75 mL, 2 eq). The mixture was stirred at 20° C. for 2 hr. LC/MS showed one main peak was desired m/z. The reaction mixture was concentrated and washed by EA(8 mL*2), the pH was adjust to 3 by 1N HCl, extracted by EA(10 mL*3), dried over Na₂SO₄, filtrated and concentrated. Intermediate I-18 (1.2 g, 63%) was obtained as a yellow solid.

LC/MS: m/z 292.1 [M+H]⁺; 0.731 min (Method C).

¹H NMR (DMSO, 400 MHz) δ=12.27 (br s, 1H), 8.67 (s, 1H), 8.10 (br s, 1H), 8.04 (br s, 1H), 4.68-4.48 (m, 2H), 3.21 (q, J=11.0 Hz, 2H), 2.61 (br t, J=10.6 Hz, 1H), 2.04-1.92 (m, 2H), 1.54 (q, J=11.5 Hz, 2H).

### Intermediate I-19: 1-[4-(2-methylimidazol-1-yl)-1,3,5-triazin-2-yl]piperidine-4-carboxylic acid (Responding to Compound of Formula (IIa)) (Following SM3 of Scheme 3 and Step 2 of SM1 of Scheme 1)

### Step 1

To a solution of 2-methyl imidazole (1.44 g, 17.53 mmol, 48.56 μL, 1.5 eq) in ACN (40 mL) was added K₂CO₃ (3.23 g, 23.37 mmol, 2 eq) and Intermediate AA-4 (3 g, 11.69 mmol, 1 eq). The mixture was stirred at 60° C. for 12 hr. LC/MS showed no Intermediate AA-3 remained. Several new peaks were shown on LC/MS and −56% of desired compound was detected. The residue was diluted with H₂O 50 mL and extracted with ethyl acetate 50 mL (50 mL*2). The combined organic layers were dried over Na₂SO₄, filtered and concentrated under reduced pressure to give a residue. The residue was purified by flash silica gel chromatography (ISCO®; 4 g SepaFlash® Silica Flash Column, Eluent of 0-100% Ethyl acetate/Petroleum ethergradient @80 mL/min) to give Intermediate I-19—methyl ester (2.8 g, 8.98 mmol, 76.87% yield, 97% purity) as a yellow oil.

LC/MS m/z 303.2 [M+1]+; RT. 0.634 min (Method C).

¹H NMR (CDCl3d, 400 MHz) 8.55-8.49 (m, 1H), 7.84 (d, J=1.6 Hz, 1H), 6.97-6.92 (m, 1H), 4.68 (br d, J=13.8 Hz, 1H), 4.62-4.49 (m, 1H), 3.73 (s, 3H), 3.28-3.19 (m, 2H), 2.84-2.81 (m, 3H), 2.72-2.62 (m, 1H), 2.03 (br s, 1H), 1.83-1.72 (m, 3H).

### Step 2: 1-[4-(2-methylimidazol-1-yl)-1,3,5-triazin-2-yl]piperidine-4-carboxylic acid

A mixture of Intermediate I-19—methyl ester (2.8 g, 9.26 mmol, 1 eq), THF (24 mL), LiOH—H₂O (1.55 g, 37.05 mmol, 4 eq) in H₂O (6 mL) was degassed, and then the mixture was stirred at 25° C. for 2 hr. LC/MS showed none of Intermediate I-19—methyl ester remained. Several new peaks were shown on LC/MS and −99% of desired compound was detected. pH was adjusted to 3-4 with 1 N HCl to come out the Intermediate I-19 (2.24 g, 7.61 mmol, 82.21% yield, 98% purity) as a white solid.

LC/MS: m/z 289.1 [M+1]+; RT. 0.675 min (Method D).

¹H NMR (CDCl₃, 400 MHz) 13.37 (s, 1H), 12.61 (d, J=1.1 Hz, 1H), 11.70-11.62 (m, 1H), 9.34-9.19 (m, 3H), 7.95 (br d, J=2.4 Hz, 3H), 7.46 (s, 2H), 7.37 (br t, J=3.8 Hz, 1H), 6.75-6.68 (m, 2H), 6.36-6.26 (m, 2H).

### Intermediate I-20: 1-[4-(2-methylpyrazol-3-yl)pyrimidin-2-yl]piperidine-4-carboxylic acid (Responding to Compound of Formula (IIa)) (Following SM4 of Scheme 3 and Step 2 of SM1 of Scheme 1)

### Step 1

A mixture of 2,4-dichloropyrimidine (5 g, 33.56 mmol, 1 eq.) in dioxane (40 mL) was added 1-methyl-5-(4,4,5,5-tetramethyl-1,3,2-dioxaborolan-2-yl)pyrazole (6.98 g, 33.56 mmol, 1 eq.), K₂CO₃ (9.28 g, 67.12 mmol, 2 eq.), Pd(dppf)Cl₂ (2.74 g, 3.36 mmol, 0.1 eq.) and H₂O (10 mL) was degassed and purged with N₂, and then the mixture was stirred at 80° C. for 12 hrs under N₂ atmosphere. LC/MS showed no starting material remained, several new peaks were shown on LC/MS and −40% of desired mass was detected. The residue was diluted with H₂O (60 mL) and extracted with EA (60 mL*2). The combined organic layers were dried over anhydrous Na₂SO₄, filtered, and concentrated under reduced pressure. The residue was purified by reversed-phase HPLC (0.1% FA condition) to give 2-chloro-4-(2-methylpyrazol-3-yl)pyrimidine (4.43 g, 20.50 mmol, 61.08% yield, 90% purity) as a yellow solid.

LC/MS: m/z 195.1[M+1]+; RT: 0.723 min (Method D).

¹H NMR: (CDCl₃, 400 MHz) δ ppm 8.64 (d, J=5.3 Hz, 1H), 7.56 (d, J=2.1 Hz, 1H), 7.49 (d, J=5.3 Hz, 1H), 6.82 (d, J=2.1 Hz, 1H), 4.47-4.30 (m, 3H).

### Step 2

A mixture of 2-chloro-4-(2-methylpyrazol-3-yl)pyrimidine (5.43 g, 27.92 mmol, 1 eq.), methyl piperidine-4-carboxylate (4.00 g, 27.92 mmol, 1 eq.), DIEA (10.82 g, 83.75 mmol, 14.59 mL, 3 eq.) in n-BuOH (60 mL) was degassed, and then the mixture was stirred at 80° C. for 2 hrs under N₂ atmosphere. LC/MS showed −8% of starting material remained.

Several new peaks were shown on LC/MS and −84% of desired compound was detected.

The residue was diluted with H₂O (60 mL) and extracted with EA (60 mL*2). The combined organic layers were dried over anhydrous Na₂SO₄, filtered and concentrated under reduced pressure. The residue was purified by flash silica gel chromatography (ISCO®; 80 g SepaFlash® Silica Flash Column, Eluent of 0-70% Ethyl acetate in Petroleum ether gradient @50 mL/min) to give Intermediate I-20-methyl ester (6.4 g, 20.81 mmol, 74.56% yield, 98% purity) as a yellow oil.

LC/MS: m/z 302.2 [M+1]⁺; RT: 0.823 min (Method C).

¹H NMR: (CDCl₃, 400 MHz) δ ppm 8.35 (d, J=5.3 Hz, 1H), 7.50 (d, J=2.0 Hz, 1H), 6.76 (d, J=5.1 Hz, 1H), 6.69 (d, J=1.9 Hz, 1H), 4.67 (td, J=3.7, 13.4 Hz, 2H), 4.28 (s, 3H), 3.71 (s, 3H), 3.20-3.09 (m, 2H), 2.64 (tt, J=4.0, 10.9 Hz, 1H), 2.06-1.98 (m, 2H), 1.82-1.69 (m, 2H)

### Step 3: 1-[4-(2-methylpyrazol-3-yl)pyrimidin-2-yl]piperidine-4-carboxylic acid

A mixture of Intermediate I-20-methyl ester (2 g, 6.64 mmol, 1 eq.), LiOH—H₂O (557.02 mg, 13.27 mmol, 2 eq.) and H₂O (0.8 mL) in THF (3.2 mL) was stirred at 25° C. for 2 hrs under N₂ atmosphere. LC/MS showed no starting material remained. Several new peaks were shown on LC/MS and −99% of desired compound was detected. The residue was diluted with H₂O (30 mL) and extracted with EA (30 mL*2). The combined organic layers were dried over anhydrous Na₂SO₄, filtered and concentrated under reduced pressure. The residue was adjusted pH to 3-4 with 1 N HCl and filtered to give Intermediate I-20 (1.8 g, 5.95 mmol, 89.67% yield, 95% purity) as a white solid.

LC/MS: m/z 288.4 [M+1]+; RT: 0.890 min (Method D).

¹H NMR: (D₂O, 400 MHz) δ ppm 8.13 (d, J=5.0 Hz, 1H), 7.48-7.44 (m, 1H), 6.72 (br d, J=5.1 Hz, 1H), 6.69 (d, J=0.9 Hz, 1H), 4.35 (br d, J=13.2 Hz, 2H), 4.00 (s, 3H), 2.91 (br t, J=12.0 Hz, 2H), 2.44-2.34 (m, 1H), 1.86 (br t, J=5.4 Hz, 2H), 1.48 (dq, J=3.9, 12.2 Hz, 2H).

### Intermediate I-21: 1-(4-oxazol-2-ylpyrimidin-2-yl)piperidine-4-carboxylic acid TFA salt (Responding to Compound of Formula (IIa)) (Following Step 2 of SM1 of Scheme 1)

To a mixture of tert-butyl 1-(4-oxazol-2-ylpyrimidin-2-yl)piperidine-4-carboxylate (150 mg) in 50 mL DCM, 0.2 mL of TFA were added and the mixture stirred at rt overnight. Then additional 0.2 mL of TFA were added and the mixture stirred at 40° C. bis no further conversion was observed by LC/MS. The mixture was then evaporated, the residue taken up in toluol and evaporated (2 times) and then dissolved in ACN/H2O and freezed dried to give Intermediate I-21 as a white solid (73.8 mg/71%).

LC/MS: m/z 275.1 [M+1]⁺; RT: 1.38 min (Method A).

¹H NMR (DMSO-d6, 400 MHz,) 6 ppm 8.53 (d, J=4.89 Hz, 1H), 8.35 (s, 1H), 7.50 (s, 1H), 7.20 (d, J=4.89 Hz, 1H), 4.59 (m, 2H), 3.12 (m, 2H), 2.57 (m, 1H), 1.92 (br dd, J=13.39, 3.12 Hz, 2H), 1.51 (m, 2H).

### Intermediate I-22: 1-[4-(2-methylimidazol-1-yl)pyrimidin-2-yl]piperidine-4-carboxylic acid (Responding to Compound of Formula (IIa)) (Following SM4 of Scheme 3 and Step 2 of SM1 of Scheme 1)

### Step 1: ethyl 1-[4-(2-methylimidazol-1-yl)pyrimidin-2-yl]piperidine-4-carboxylate)

To a mixture of 2-chloro-4-(2-methylimidazol-1-yl)pyrimidine (340.44 mg, 1.75 mmol) in ACN (5 mL), ethyl piperidine-4-carboxylate (0,245 mL, 1.6 mmol) was added, followed by DIPEA (0,692 mL, 3.97 mmol). The mixture stirred in the MW for 1 h at 120° C. LC/MS showed complete conversion to product and the reaction mixture was evaporated, and the residue purified by chromatography on silica gel (A: DCM; B: DCM/MeOH 9/1; Gradient 0% B->50% B) to give the desired product Intermediate I-22-ethyl ester with rest of DIPEA, which was used as such in the following step.

### Step 2: 1-[4-(2-methylimidazol-1-yl)pyrimidin-2-yl]piperidine-4-carboxylic acid

A solution of Intermediate I-22-ethyl ester (ethyl 1-[4-(2-methylimidazol-1-yl)pyrimidin-2-yl]piperidine-4-carboxylate) (501.5 mg, 1.59 mmol) in NaOH (2N in THF/MeOH 1:1:1) (10 mL), was stirred at rt overnight. The solvent was evaporated and the residue acidified with 1M H₂SO₄. The resulting solution was concentrated to 6 mL and the residue purified by HPLC to give Intermediate I-22 (284 mg, 62%).

LC/MS: m/z 288.2 [M+1]⁺; RT: 0.88 min (Method A).

¹H NMR: ¹H NMR (DMSO-d6, 400 MHz) δ ppm 8.49 (d, J=5.38 Hz, 1H), 7.78 (d, J=1.59 Hz, 1H), 7.08 (m, 1H), 6.87 (d, J=5.38 Hz, 1H), 4.50 (br d, J=13.08 Hz, 2H), 3.13 (m, 2H), 2.67 (s, 3H), 2.59 (m, 1H), 2.50 (u), 1.91 (m, 2H), 1.52 (m, 2H).

### Intermediate I-23: 1-[4-(4-methylpyrazol-1-yl)pyrimidin-2-yl]piperidine-4-carboxylic acid (Responding to Compound of Formula (IIa)) (Following SM4 of Scheme 3 and Step 2 of SM1 of Scheme 1)

### Step 1: ethyl 1-[4-(4-methylpyrazol-1-yl)pyrimidin-2-yl]piperidine-4-carboxylate

To a mixture of 2-chloro-4-(4-methyl-1 h-pyrazol-1-yl)pyrimidine (687.98 mg, 3.39 mmol) in ACN (18 mL), ethyl piperidine-4-carboxylate (500 mg, 2.74 mmol) was added, followed by DIPEA (2.15 mL, 12.3 mmol). The mixture stirred in the MW for 30 min at 120° C. LC/MS showed complete conversion to product and the reaction mixture was evaporated, and the residue purified by chromatography on silica gel (Heptane:EA gradient) to give the desired product Intermediate I-23-ethyl ester (865 mg, 89%).

LC/MS: m/z 316.3 [M+1]⁺; RT: 0.844 min (Method B).

### Step 2: 1-[4-(4-methylpyrazol-1-yl)pyrimidin-2-yl]piperidine-4-carboxylic acid

A solution of Intermediate I-23-ethyl ester (ethyl 1-[4-(2-methylimidazol-1-yl)pyrimidin-2-yl]piperidine-4-carboxylate) (865 mg, 2.74 mmol) in NaOH (2N in THF/MeOH 1:1:1) (42 mL) was stirred at rt overnight. The solvent was evaporated and the residue acidified with 1M H2SO4. The resulting solution was concentrated to 6 mL and the residue purified by HPLC to give Intermediate I-23 (753 mg, 96%).

LC/MS: m/z 288.3 [M+1]⁺; RT: 0.644 min (Method B).

¹H NMR (DMSO-d6, 400 MHz) δ ppm 12.23 (br s, 1H), 8.46 (s, 1H), 8.39 (d, J=5.38 Hz, 1H), 7.68 (s, 1H), 6.98 (d, J=5.26 Hz, 1H), 4.58 (br d, J=13.20 Hz, 2H), 3.10 (br t, J=11.06, 11.06 Hz, 2H), 2.57 (m, 1H), 2.11 (s, 3H), 1.92 (m, 1H), 1.89 (br s, 1H), 1.51 (m, 2H).

### Intermediate I-24: 1-[2-(2-methylpyrazol-3-yl)pyrimidin-4-yl]piperidine-4-carboxylic acid (Responding to Compound of Formula (IIa)) (Following SM3 of Scheme 3 and Step 2 of SM1 of Scheme 1)

### Step 1: 1-[2-(2-methylpyrazol-3-yl)pyrimidin-4-yl]piperidine-4-carboxylic acid methyl ester

To a solution of Intermediate AA-5 (2.85 g, 13.69 mmol, 1 eq.) in dioxane (28 mL) was added K₂CO₃ (3.78 g, 27.38 mmol, 2 eq.), 1-methyl-5-(4,4,5,5-tetramethyl-1,3,2-dioxaborolan-2-yl)pyrazole (3.5 g, 13.69 mmol, 1 eq.) H₂O (7 mL) and Pd(dppf)Cl₂ (1.12 g, 1.37 mmol, 0.1 eq.) and the mixture was stirred at 120° C. for 12 hrs under N₂ atmosphere. LC/MS showed no starting material remained. Several new peaks were shown on LC/MS and −27% of desired compound was detected. The residue was diluted with H₂O (100 mL) and extracted with EA (30 mL*2). The combined organic layers were dried over anhydrous Na₂SO₄, filtered and concentrated under reduced pressure. The residue was purified by flash silica gel chromatography (ISCO®; 40 g SepaFlash® Silica Flash Column, Eluent of 30~70% EA/PE gradient @30 mL/min) to give Intermediate I-24 methyl ester (3.72 g, 70.% yield, 80% purity) as a yellow oil.

LC/MS: m/z 302.1 [M+1]⁺; RT 0.399 min (Method C).

### Step 2: 1-[2-(2-methylpyrazol-3-yl)pyrimidin-4-yl]piperidine-4-carboxylic acid

A mixture of Intermediate I-24 methyl ester (3.52 g, 11.68 mmol, 1 eq.), LiOH—H₂O (980.27 mg, 23.36 mmol, 2 eq.) and H₂O (8 mL) in THF (32 mL), and then the mixture was stirred at 25° C. for 12 hrs under N₂ atmosphere. LC/MS showed no starting material remained. Several new peaks were shown on LC/MS and −65% of desired compound was detected. The residue was diluted with H₂O (40 mL) and extracted with EtOAc (40 mL*2). The combined organic layers were dried over anhydrous Na₂SO₄, filtered and concentrated under reduced pressure. The residue was purified by reversed-phase HPLC (0.1% FA condition) to give Intermediate I-24 (1.3 g, 38%) as a white solid.

LC/MS: m/z 288.1 [M+1]⁺; RT 0.348 min (Method C).

¹H NMR: (DMSO-d6, 400 MHz) δ ppm 8.27 (d, J=6.2 Hz, 1H), 7.45 (d, J=1.8 Hz, 1H), 6.88 (d, J=1.8 Hz, 1H), 6.79 (d, J=6.4 Hz, 1H), 4.38-4.26 (m, 2H), 4.21 (s, 3H), 3.18-3.06 (m, 2H), 2.68 (br s, 1H), 2.64-2.55 (m, 1H), 1.92 (br dd, J=2.9, 13.2 Hz, 2H), 1.62-1.45 (m, 2H).

### Intermediate I-25. 1-[2-(4-methylpyrazol-1-yl)pyrimidin-4-yl]piperidine-4-carboxylic acid (Responding to Compound of Formula (IIa)) (Following SM4 of Scheme 3 and Step 2 of SM1 of Scheme 1)

### Step 1

To a mixture of 4-chloro-2-(4-methylpyrazol-1-yl)pyrimidine (680 mg, 3.39 mmol) and ethyl piperidine carboxylate (500 mg, 3.08 mmol) in ACN (18 mL), DIPEA (2.5 mL; 2.8 mmol) was added. The mixture was stirred for 30 min at 120° C. in the MW. LCMS showed full conversion to desired product. The mixture was diluted with EtOAc and extracted with saturated aq. NH₄Cl. The aqueous phase was extracted with EE (2×) and the combined organic layers dried over MgSO₄, concentrated and purified by column chromatography on Silica gel (Heptane:EE gradient), to give Intermediate I-25 ethyl ester as a white solid (517 mg, 52%).

LC/MS: m/z 316.4 [M+1]⁺; RT 0.836 min (Method B).

### Step 2: 1-[2-(4-methylpyrazol-1-yl)pyrimidin-4-yl]piperidine-4-carboxylic acid

A solution of Intermediate I-25 ethyl ester (517 mg, 1.64 mmol) in 2N NaOH/THF/MeOH (1:1:1) was stirred at RT overnight. Then the organic solvents were evaporated and the resulting aqueous phase acidified with 1N H₂SO₄. The precipitate was filtered and dried in oven to give Intermediate I-25 as a white solid (381 mg, 81%).

LC/MS: m/z 288.2 [M+1]⁺; RT 1.66 min (Method A).

¹H NMR (DMSO-d6, 600 MHz) δ ppm 12.25 (br s, 1H), 8.46 (s, 1H), 8.39 (d, J=5.32 Hz, 1H), 7.68 (s, 1H), 6.98 (d, J=5.32 Hz, 1H), 4.58 (dt, J=13.34, 3.51, 3.51 Hz, 2H), 3.10 (m, 2H), 2.57 (m, 1H), 2.11 (s, 3H), 1.90 (br dd, J=13.48, 3.39 Hz, 2H), 1.51 (m, 2H).

### Intermediate I-26. 1-[6-(4-methylpyrazol-1-yl)pyrimidin-4-yl]piperidine-4-carboxylic acid (responding to compound of formula (IIa)) (following SM4 of scheme 3 and step 2 of SM1 of scheme 1)

### Step 1

To a mixture of 4-chloro-6-(4-methylpyrazol-1-yl)pyrimidine (695 mg, 3.39 mmol) and ethyl piperidine carboxylate (500 mg, 3.08 mmol) in CAN (18 mL), DIPEA (2.5 mL; 2.8 mmol) was added. The mixture was stirred for 30 min at 120° C. in the MW. LCMS showed full conversion to desired product. The mixture was diluted with EA and extracted with saturated aq. NH4Cl. The aqueous phase was extracted with EA (2×) and the combined organic layers dried over MgSO₄, concentrated and purified by column chromatography on Silica gel (Heptane:EtOAc gradient), to give Intermediate I-26 ethyl ester as a white solid (883 mg, 90%).

LC/MS: m/z 316.3 [M+1]⁺; RT 0.869 min (Method B).

### Step 2: 1-[6-(4-methylpyrazol-1-yl)pyrimidin-4-yl]piperidine-4-carboxylic acid

A solution of Intermediate I-26 ethyl ester (883 mg, 2.8 mmol) in 2N NaOH/THF/MeOH (1:1:1) was stirred at RT overnight. Then the organic solvents were evaporated and the resulting aqueous phase acidified with 1N H₂SO₄. The precipitate was filtered and dried in oven to give Intermediate I-26 as a white solid (679.9 mg, 84%).

LC/MS: m/z 288.1 [M+1]⁺; RT 1.63 min (Method A).

¹H NMR (DMSO-d6, 400 MHz) δ ppm 12.27 (br s, 1H), 8.41 (s, 1H), 8.34 (s, 1H), 7.67 (s, 1H), 7.06 (s, 1H), 4.29 (br d, J=10.76 Hz, 2H), 3.13 (m, 2H), 2.58 (m, 1H), 2.10 (s, 3H), 1.91 (br dd, J=13.45, 3.30 Hz, 2H), 1.53 (m, 2H).

### Intermediate I-27. 1-[4-(2-methylpyrazol-3-yl)-1,3,5-triazin-2-yl]piperidine-4-carboxylic acid (Responding to Compound of Formula (IIa)) (Following SM3 of Scheme 3 and Step 2 of SM1 of Scheme 1)

### Step 1

To a solution of I-AA 4 (10 g, 38.96 mmol, 1 eq) in dioxane (80 mL) was added K₂CO₃ (10.77 g, 77.92 mmol, 2 eq), Pd(dppf)Cl₂ (2.85 g, 3.90 mmol, 0.1 eq), H2O (20 mL) and 1-methyl-5-(4,4,5,5-tetramethyl-1,3,2-dioxaborolan-2-yl)pyrazole (8.11 g, 38.96 mmol, 1 eq). The mixture was stirred at 80° C. for 12 hr. LCMS showed no starting material remained. Several new peaks were shown on LCMS and −41% of desired compound was detected. The residue was diluted with H₂O 100 mL and extracted with ethyl acetate 100 mL (100 mL*2). The combined organic layers were dried over Na₂SO₄, filtered and concentrated under reduced pressure to give a residue. The residue was purified by flash silica gel chromatography (ISCO®; 20 g SepaFlash® Silica Flash Column, Eluent of 0-80% Ethyl acetate/Petroleum ether gradient @80 mL/min) to give Intermediate I-27—Methyl ester (11.3 g, 33.26 mmol, 85.39% yield, 89% purity) as a white solid.

LCMS: m/z 303.2 [M+1]⁺; RT 0.836 min (Method C).

¹H NMR (CDCl₃, 400 MHz) 8.59 (s, 1H), 7.50 (d, J=1.7 Hz, 1H), 7.09 (d, J=1.7 Hz, 1H), 4.66 (br d, J=10.6 Hz, 2H), 4.34-4.30 (s, 3H), 3.72 (s, 3H), 3.20 (br t, J=12.3 Hz, 2H), 2.66 (tt, J=4.0, 10.6 Hz, 1H), 2.04-1.98 (m, 2H), 1.83-1.71 (m, 2H).

### Step 2: 1-[4-(2-methylpyrazol-3-yl)-1,3,5-triazin-2-yl]piperidine-4-carboxylic acid

A mixture of Intermediate I-27—Methyl ester (11.3 g, 37.38 mmol, 1 eq), H₂O (20 mL), LiOH—H₂O (6.27 g, 149.51 mmol, 4 eq) in THF (90 mL) was degassed, and then the mixture was stirred at 25° C. for 2 hr. LCMS showed no of starting material remained. Several new peaks were shown on LCMS and ~14% of desired compound (low ionization of COOH) was detected. The reaction mixture was filtered and concentrated under reduced pressure to give a residue. The crude product was purified by reversed-phase HPLC(flow: 200 mL/min; gradient: from 5% ACN/95% H₂O (0.1% FA) to 40% ACN/60% H₂O (0.1% FA) in 25 min; 40% ACN/60% H₂O (0.1% FA) in 20 min; column: Welch Ultimate XB_C18, 20-40 μm, 120 Å) to give Intermediate I-27 (8.5 g, 78% yield) as a white solid.

LCMS: m/z 289.4 [M+1]⁺; RT 0.842 (Method D)

¹H NMR (DMSO-d₆, 400 MHz) 12.80-12.01 (m, 1H), 8.65 (s, 1H), 7.53 (d, J=2.0 Hz, 1H), 7.05 (d, J=1.9 Hz, 1H), 4.59-4.50 (m, 2H), 4.24 (s, 3H), 3.26-3.17 (m, 2H), 2.65-2.58 (m, 1H), 2.00-1.89 (m, 2H), 1.63-1.46 (m, 2H).

### Intermediate I-37: 1-[4-(2-oxo-1-piperidyl)pyrimidin-2-yl]piperidine-4-carboxylic acid

To a mixture of Ar—Cl 19 (5.19 g, 0.0245 mol, 1.00 eq.) and Piperidine-4-carboxylic acid (3.48 g, 0.0270 mol, 1.10 eq.) in co-solvent dioxane/H₂O (v/v=2/1, 110 mL) was added TEA (6.82 mL, 0.0490 mol, 2.00 eq.). The resulting mixture was heated to 90° C. for 16 h. The mixture was directly concentrated to remove most of the solvent dioxane, and then washed with EtOAc (5 mL) once to remove the impurity soluble in organic solvents. The left aqueous phase was adjusted to pH ~5 with citric acid (sat. aq.), and the precipitate formed was collected by filtration and washed with a little water to afford Intermediate I-37 (6.12 g, 0.0201 mol, 82.0% yield) as a white solid.

¹H NMR (400 MHz, DMSO-d₆) δ=8.20 (d, J=5.6 Hz, 1H), 7.23 (d, J=5.6 Hz, 1H), 4.48 (d, J=13.2 Hz, 2H), 3.88 (t, J=6.0 Hz, 2H), 3.09-2.95 (m, 2H), 2.58-2.50 (m, 3H), 1.92-1.73 (m, 6H), 1.56-1.39 (m, 2H).

LCMS m/z=305.2 [M+H]⁺; RT 0.478 (Method Q)

### Intermediate I-38: 1-[4-(4-oxo-5-azaspiro[2.4]heptan-5-yl)pyrimidin-2-yl]piperidine-4-carboxylic acid

I-38 was synthesized in a similar manner to Intermediate I-37, but using Ar—Cl 3 (300 mg, 0.001 mol, 1.00 eq.) and Piperidine-4-carboxylic acid (191 mg, 0.001 mol, 1.10 eq.) to afford crude Intermediate I-38 (610 mg, 60.0% purity, 0.00116 mol, 86.3% yield, contaminated with TEA) as a yellow solid.

¹H NMR (400 MHz, DMSO-d₆) δ=8.21 (d, J=5.6 Hz, 1H), 7.42 (d, J=5.6 Hz, 1H), 4.49 (d, J=13.2 Hz, 2H), 4.04-3.99 (m, 2H), 3.05-2.98 (m, 2H), 2.53-2.51 (m, 1H), 2.17-2.12 (m, 2H), 1.86 (dd, J=13.2, 2.8 Hz, 2H), 1.55-1.39 (m, 2H), 1.04-1.01 (m, 2H), 0.96-0.91 (m, 2H).

MS (ESI) m/z=317.2 [M+H]⁺.(Cpd 13, Method Q)

### Intermediate I-39: 1-[4-(4-oxo-5-azaspiro[2.5]octan-5-yl)pyrimidin-2-yl]piperidine-4-carboxylic acid

I-39 was synthesized in a similar manner to Intermediate I-37, but using Ar—Cl 19 (190 mg, 0.000799 mol, 1.00 eq.) and Piperidine-4-carboxylic acid (124 mg, 0.000959 mol, 1.20 eq.) to afford I-39 (210 mg, 0.000636 mol, 79.5% yield) as a white solid.

¹H NMR (400 MHz, DMSO-d₆) δ=8.17 (d, J=5.6 Hz, 1H), 7.19 (d, J=5.6 Hz, 1H), 4.47 (d, J=13.2 Hz, 2H), 3.98 (t, J=6.0 Hz, 2H), 3.07-2.95 (m, 2H), 2.56-2.53 (m, 1H), 2.02-1.92 (m, 2H), 1.86 (dd, J=13.2, 2.8 Hz, 2H), 1.77-1.71 (m, 2H), 1.53-1.39 (m, 2H), 1.19 (q, J=3.2 Hz, 2H), 0.73 (q, J=3.2 Hz, 2H).

LCMS: m/z=331.2 [M+H]⁺. RT 0.497 (Method Q)

### Intermediate I-40: 1-[4-(2-oxooxazolidin-3-yl)pyrimidin-2-yl]piperidine-4-carboxylic acid

I-40 was synthesized in a similar manner to Intermediate I-37, but using Ar—Cl 2 (350 mg, 0.00167 mol, 1.00 eq.) and Piperidine-4-carboxylic acid (258 mg, 0.00200 mol, 1.20 to afford crude I-40, that was used as such for the synthesis of Example 65.

### Intermediate I-41: 1-[5-fluoro-4-(2-methylimidazol-1-yl)pyrimidin-2-yl]piperidine-4-carboxylic acid

I-40 was synthesized in a similar manner to Intermediate I-37, but using Ar—Cl 30 (2.00 g, 0.00941 mol, 1.00 eq.) and Piperidine-4-carboxylic acid (1.34 g, 0.0103 mol, 1.10 eq.) to afford 1-[5-fluoro-4-(2-methylimidazol-1-yl)pyrimidin-2-yl]piperidine-4-carboxylic acid (2.20 g, 0.00721 mol, 76.6% yield) as a white solid.

¹H NMR (400 MHz, CD₃OD) δ=8.58 (d, J=2.8 Hz, 1H), 7.55 (dd, J=3.2, 1.6 Hz, 1H), 7.11 (d, J=1.6 Hz, 1H), 4.65 (dt, J=13.6, 3.6 Hz, 2H), 3.29-3.20 (m, 2H), 2.75-2.70 (m, 1H), 2.67 (s, 3H), 2.12-2.03 (m, 2H), 1.80-1.68 (m, 2H).

LCMS (ESI) m/z=306.2 [M+H]⁺. RT 0.454 (Method Q)

### Intermediate I-42. 1-[4-(2-oxopyrrolidin-1-yl)pyrimidin-2-yl]piperidine-4-carboxylic acid

I-42 was synthesized in a similar manner to Intermediate I-37, but using Ar—Cl3 (5.47 g, 0.0277 mol, 1.00 eq.) and Piperidine-4-carboxylic acid (3.93 g, 0.0304 mol, 1.10 eq.) to afford 1-[4-(2-oxopyrrolidin-1-yl)pyrimidin-2-yl]piperidine-4-carboxylic acid (3.85 g, 0.0133 mol, 47.9% yield) as a white sold.

1H NMR (400 MHz, DMSO-d6) δ=12.63-11.90 (br s, 1H), 8.21 (d, J=6.0 Hz, 1H), 7.50 (d, J=6.0 Hz, 1H), 4.50-4.42 (m, 2H), 3.93 (t, J=7.2 Hz, 2H), 3.13-3.01 (m, 2H), 2.61-2.51 (m, 3H), 2.07-1.97 (m, 2H), 1.92-1.83 (m, 2H), 1.55-1.43 (m, 2H).

LC/Ms m/z=291.0 [M+H]+; RT 0.664 (Method Q)

### Intermediate I-43. 1-[6-(2-oxopyrrolidin-1-yl)pyrimidin-4-yl]piperidine-4-carboxylic acid

I-43 was synthesized in a similar manner to Intermediate I-37, but using Ar—Cl 31 (2.80 g, 0.0142 mol, 1.00 eq.) and Piperidine-4-carboxylic acid (3.66 g, 0.0283 mol, 2.00 eq.) to afford I-43 (3.40 g, 0.0117 mol, 82.7% yield) as a white sold.

¹H NMR (400 MHz, CD₃OD) δ=8.30 (s, 1H), 7.64 (s, 1H), 4.29 (d, J=13.2 Hz, 2H), 4.06-3.97 (m, 2H), 3.10 (ddd, J=13.6, 11.2, 2.8 Hz, 2H), 2.68-2.58 (m, 3H), 2.11 (quin, J=7.6 Hz, 2H), 2.03-1.93 (m, 2H), 1.71-1.56 (m, 2H).

### Intermediate I-44: 1-[4-(3-methoxy-2-oxo-1-piperidyl)pyrimidin-2-yl]piperidine-4-carboxylic acid

I-44 was synthesized as Intermediate 42 starting from Ar—Cl 28 (2.0 g, 78% purity, 0.82 mmol) and Piperidine-4-carboxylic acid (1.25 g, 1.5 eq) and the compounds was used in the next step as crude material.

### Intermediate I-45: 1-[4-(2-methyl-5-nitro-pyrazol-3-yl)pyrimidin-2-yl]piperidine-4-carboxylic acid

I-45 was synthesized as Intermediate 42 starting from Ar—Cl 32 (416 mg, 1.74 mmol) and Piperidine-4-carboxylic acid (235 mg, 1.05 eq) to afford Intermediate I-45 which was used in the next step as crude material (61% purity).

### Intermediate I-46. 1-[5-fluoro-4-(2-oxooxazolidin-3-yl)pyrimidin-2-yl]piperidine-4-carboxylic acid

This was synthesized as Intermediate 42 starting from Ar—Cl 5 (1.0 g, 84% purity, 3.86 mmol) and Piperidine-4-carboxylic acid (584 mg, 1.10 eq) to afford Intermediate I-46 as a white solid (1.10 g, 46%).

¹H NMR (400 MHz, DMSO-d₆) δ=12.61-11.92 (br s, 1H), 8.39 (d, J=3.2 Hz, 1H), 4.50 (t, J=7.6 Hz, 2H), 4.39 (d, J=13.2 Hz, 2H), 4.12 (t, J=7.6 Hz, 2H), 3.10-2.95 (m, 2H), 2.49-2.46 (m, 1H), 1.93-1.79 (m, 2H), 1.56-1.39 (m, 2H).

LC/Ms m/z=311.2 [M+H]+; RT 0.494 (Method Q).

### Intermediate I-47. 1-[5-fluoro-4-(4-oxo-5-azaspiro[2.4]heptan-5-yl)pyrimidin-2-yl]piperidine-4-carboxylic acid

This was synthesized as Intermediate 42 starting from Ar—Cl 34 (400 mg, 1.66 mmol) and Piperidine-4-carboxylic acid (235 mg, 1.05 eq) to afford Intermediate I-47 as a white solid (386 mg, 69%).

¹H NMR (400 MHz, DMSO-d₆) δ=12.24 (br s, 1H), 8.37 (d, J=3.2 Hz, 1H), 4.50-4.30 (m, 2H), 3.95 (t, J=7.2 Hz, 2H), 3.10-2.96 (m, 2H), 2.53-2.51 (m, 1H), 2.26-2.17 (m, 2H), 1.86 (dd, J=13.2, 2.8 Hz, 2H), 1.56-1.40 (m, 2H), 1.02-0.97 (m, 2H), 0.97-0.92 (m, 2H).

LC/Ms m/z=335.2 [M+H]+; RT 0.534 (Method Q)

### General scheme for the synthesis of examples 1, 2, 3, 4, 5, 6, 22, 23, 29, 30, 31, 32, 33, 34 (responding to compound of formula (I)) (following SM2 of scheme 1)

### Preparation of Aryl Chlorides (I-37)

Their structures are gathered in table 2, herein after.

TABLE 2
Aryl chloride derivatives

| | |
|---|---|
| 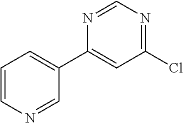 [SMILES: Clc1cc(-c2cccnc2)ncn1] | Ar-Cl 1 |
| 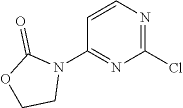 [SMILES: O=C1OCCN1c1ccnc(Cl)n1] | Ar-Cl 2 |
| 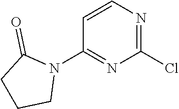 [SMILES: O=C1CCCN1c1ccnc(Cl)n1] | Ar-Cl 3 |
| 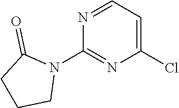 [SMILES: O=C1CCCN1c1nccc(Cl)n1] | Ar-Cl 4 |
| 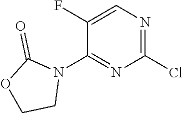 [SMILES: O=C1OCCN1c1nc(Cl)ncc1F] | Ar-Cl 5 |
| 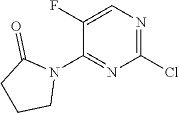 [SMILES: O=C1CCCN1c1nc(Cl)ncc1F] | Ar-Cl 6 |
| 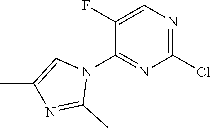 | Ar-Cl 7 |
| 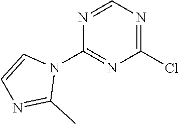 | Ar-Cl 8 |
| 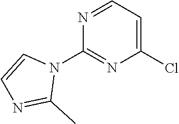 | Ar-Cl 9 |
| 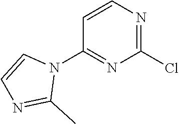 | Ar-Cl 10 |
| 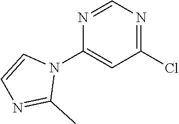 | Ar-Cl 11 |
| 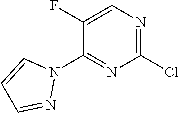 [SMILES: Fc1cnc(Cl)nc1-n1cccn1] | Ar-Cl 12 |
| 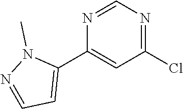 | Ar-Cl 13 |
| 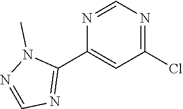 | Ar-Cl 14 |
| 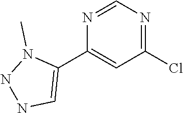 | Ar-Cl 15 |
| 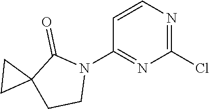 [SMILES: O=C1N(c2ccnc(Cl)n2)CCC12CC2] | Ar-Cl 16 |
| 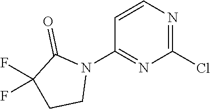 [SMILES: O=C1N(c2ccnc(Cl)n2)CCC1(F)F] | Ar-CI 17 |
| 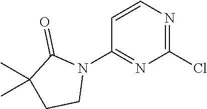 | Ar-Cl 18 |
| 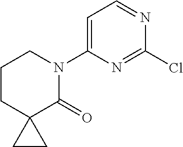 [SMILES: O=C1N(c2ccnc(Cl)n2)CCCC12CC2] | Ar-Cl 19 |
| 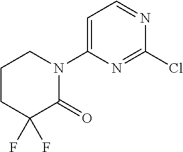 [SMILES: O=C1N(c2ccnc(Cl)n2)CCCC1(F)F] | Ar-Cl 20 |
| 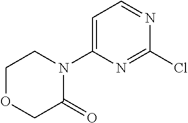 [SMILES: O=C1COCCN1c1ccnc(Cl)n1] | Ar-Cl 21 |
| 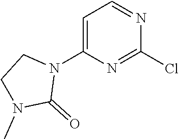 | Ar-Cl 22 |
| 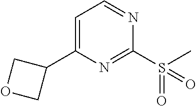 | Ar-Cl 23 |
| 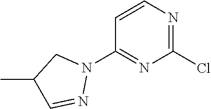 | Ar-Cl 24 |
| 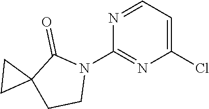 [SMILES: O=C1N(c2nccc(Cl)n2)CCC12CC2] | Ar-Cl 25 |
| 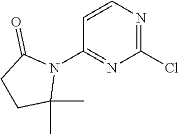 | Ar-Cl 26 |
| 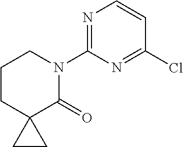 [SMILES: O=C1N(c2nccc(Cl)n2)CCCC12CC2] | Ar-Cl 27 |
| 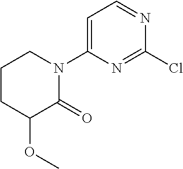 [SMILES: COC1CCCN(c2ccnc(Cl)n2)C1=O] | Ar-Cl 28 |
| 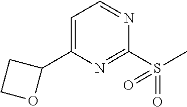 | Ar-Cl 29 |
| 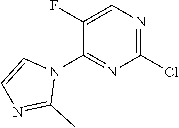 | Ar-Cl 30 |
| 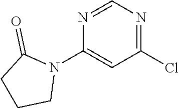 [SMILES: O=C1CCCN1c1cc(Cl)ncn1] | Ar-Cl 31 |
| 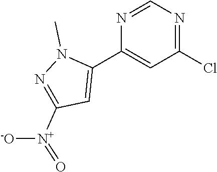 | Ar-Cl 32 |
| 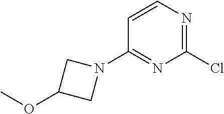 | Ar-Cl 33 |
| 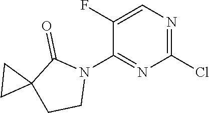 [SMILES: O=C1N(c2nc(Cl)ncc2F)CCC12CC2] | Ar-Cl 34 |
| 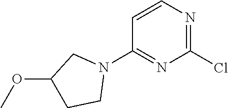 | Ar-Cl 35 |
| 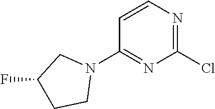 [SMILES: F[C@H]1CCN(c2ccnc(Cl)n2)C1] | Ar-Cl 36 |
| 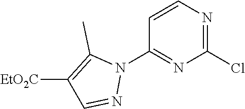 [SMILES: CCOC(=O)c1cnn(-c2ccnc(Cl)n2)c1C] | Ar-Cl 37 |

Aryl chloride 1: (4-chloro-6(pyridin-3-yl)pyrimidine—commercially available

### Aryl Chloride 2:

To a solution of 2-oxazolidinone (292.3 mg, 3.29 mmol) in DMF (10 mL), NaH (60% in Mineraloil, 3.62 mmol) was added. After stirring for 15 min., 2,4-dichloropyrimidine (0.5 g, 3.29 mmol) was added in two portions. The mixture was stirred for 1 h at rt, and then LC/MS showed full conversion to desired product. The reaction mixture was quenched with water and then extracted with EA (3×). The organic layer was then washed with water and with saturated NaCl aqueous solution, dried over Na₂SO₄, filtered and the solvent was evaporated. The product was purified by Silica-gel chromatography to give the desired product Ar—Cl 2 (394 mg, 60%).

LCMS: m/z 200.4 [M+1]⁺; RT 0.435 (Method B)

Aryl Chloride 3 and aryl chloride 4:

To a solution of 2-oxazolidinone (285.6 mg, 3.29 mmol) in DMF (10 mL), NaH (60% in Mineralöl, 3.62 mmol) was added. After stirring for 15 min., 2,4-dichloropyrimidine (0.5 g, 3.29 mmol) was added in two portions. The mixture was stirred for 1 h at rt, and then LC/MS showed full conversion to desired product. The reaction mixture was quenched with water and then extracted with EA (3×). The organic layer was then washed with water and with saturated NaCl aqueous solution, dried over Na2SO4, filtered and the solvent was evaporated. The product was purified by Silica-gel chromatography to give the desired products Ar—Cl 3 (105 mg, 16%) and Ar—Cl 4 (82 mg, 13%).
LCMS Ar—Cl-3: m/z 200.1 [M+1]⁺; RT 0.428 (Method B)
LCMS Ar—Cl 4: m/z 200.1 [M+1]⁺; RT 0.527 (Method B)

### Aryl Chloride 5: 3-(2-chloro-5-fluoro-pyrimidin-4-yl)oxazolidin-2-one

To a solution of 2-oxazolidinone (52 mg, 0.59 mmol) in DMF (10 mL), NaH (60% in Mineraloil, 26 mg, 0.65 mmol) was added. After stirring for 15 min., 2,4-dichloro-5-fluoro-pyrimidine (100 mg, 0.59 mmol) was added in two portions. The mixture was stirred for 1 h at rt, and then LC/MS showed full conversion to desired product. The reaction mixture was quenched with water and then extracted with EA (3×). The organic layer was then washed with water and with saturated NaCl aqueous solution, dried over Na₂SO₄, filtered and the solvent was evaporated. The product was purified by Silica-gel chromatography to give the desired products Ar—Cl 5 (105 mg, 81%).

LCMS: m/z 217.0 [M+1]⁺; RT 0.94 min (Method A).

### Aryl Chloride 6: 1-(2-chloro-5-fluoro-pyrimidin-4-yl)pyrrolidin-2-one

To a solution of 2-pyrrolidone (50 mg, 0.59 mmol) in DMF (10 mL), NaH (60% in Mineraloil, 26 mg, 0.59 mmol) was added. After stirring for 15 min., 2,4-dichloro-5-fluoro-pyrimidine (100 mg, 0.59 mmol) was added in two portions. The mixture was stirred for 1 h at rt, and then LC/MS showed full conversion to desired product. The reaction mixture was quenched with water and then extracted with EA (3×). The organic layer was then washed with water and with saturated NaCl aqueous solution, dried over Na₂SO₄, filtered and the solvent was evaporated. The product was purified by Silica-gel chromatography to give the desired products Ar—Cl 6 (38 mg, 19%).

LCMS: m/z 216.1 [M+1]⁺; RT 0.477 min (Method B).

### Aryl Chloride 7: 2-chloro-4-(2,4-dimethylimidazol-1-yl)-5-fluoro-pyrimidine

To a solution of 2,4-dimethyl-1H-imidazole (25 mg, 0.26 mmol) and 2,4-dichloro-5-fluoro-pyrimidine (47.7 mg, 0.28 mmol) in dry ACN (10 mL), Cs₂CO₃ (127 mg) was added. The mixture was stirred for 15 min. at rt, and then LC/MS showed full conversion to desired product. The reaction mixture was quenched with water and then extracted with EA (3×). The organic layer was then washed with water and with saturated NaCl aqueous solution, dried over Na₂SO₄, filtered and the solvent was evaporated. The product was purified by Silica-gel chromatography to give the desired products Ar—Cl 7 (22 mg, 37%).

LCMS: m/z 227.1 [M+1]⁺; RT 0.584 min (Method E).

### Aryl Chloride 8-1-(2-chloro-5-fluoro-pyrimidin-4-yl)pyrrolidin-2-one

To a solution of 2-pyrrolidone (50.9 mg, 0.59 mmol) in DMF (3 mL), NaH (60% in Mineraloil, 26 mg, 0.65 mmol) was added. After stirring for 15 min., 2,4-dichloro-5-fluoro-pyrimidine (100 mg, 0.59 mmol) was added in two portions. The mixture was stirred for 1 h at rt, and then LC/MS showed full conversion to desired product. The reaction mixture was quenched with water and then extracted with EA (3×). The organic layer was then washed with water and with saturated NaCl aqueous solution, dried over Na₂SO₄, filtered and the solvent was evaporated. The product was purified by Silica-gel chromatography to give the desired products Ar—Cl 8 (38 mg, 29%).

LCMS: m/z 216.1 [M+1]⁺; RT 0.477 min (Method B).

### Aryl Chloride 9 and Aryl Chloride 10

To a solution of 2-methylimidazole (200 mg, 2.41 mmol) and 2,4-dichloropyrimidine (0.4 g, 2.65 mmol) in ACN (8 mL), Cs₂CO₃ (1.18 g, 3.62 mmol) was added. After stirring overnight at rt, LC/MS showed full conversion to desired products. The reaction mixture was quenched with water and then extracted with EA (3×). The organic layer was then washed with water and with saturated NaCl aqueous solution, dried over Na₂SO₄, filtered and the solvent was evaporated. The product was purified by Silica-gel chromatography, followed by prep. HPLC to give the desired products Ar—Cl 9 (101 mg, 22%) and Ar—Cl 10 (151 mg, 32%)
LCMS Ar—Cl 9: m/z 200.1 [M+1]⁺; RT 0.470 (Method E)
LCMS Ar—Cl 10: m/z 200.1 [M+1]⁺; RT 0.581 (Method E)

### Aryl Chloride 11-4-chloro-6-(2-methyl-1H-imidazol-yl)pyrimidine, commercially available

### Aryl Chloride 12

To a solution of pyrazole (43.48 mg, 0.63 mmol) and 2,4-dichloro-5-fluoro-pyrimidine (100 mg, 0.57 mmol) in dry ACN (10 mL), Cs₂CO₃ (0.41 g, 1.25 mmol) was added. The mixture was stirred for 30 min. at rt, and then LC/MS showed full conversion to desired product. The reaction mixture was quenched with water and then extracted with EA (3×). The organic layer was then washed with water and with saturated NaCl aqueous solution, dried over Na2SO4, filtered and the solvent was evaporated. The product was purified by prep. HPLC chromatography to give the desired products Ar—Cl 12 (92 mg, 81%).

LCMS: m/z 199.0 [M+1]+; RT 0.601 (Method B)

### Aryl Chloride 13: 4-chloro-6-(2-methylpyrazol-3-yl)pyrimidine

A mixture of 4,6-dichloropyrimidine (5 g, 33.56 mmol, 1 eq.) in dioxane (40 mL) was added 1-methyl-5-(4,4,5,5-tetramethyl-1,3,2-dioxaborolan-2-yl)pyrazole (6.98 g, 33.56 mmol, 1 eq.), K₂CO₃ (9.28 g, 67.12 mmol, 2 eq.), Pd(dppf)Cl₂ (2.74 g, 3.36 mmol, 0.1 eq.) and H₂O (10 mL) was degassed and purged with N₂, and then the mixture was stirred at 80° C. for 12 hrs under N₂ atmosphere. LC/MS showed no starting material remained, several new peaks were shown on LC/MS and ~40% of desired mass was detected. The residue was diluted with H₂O (60 mL) and extracted with EA (60 mL*2). The combined organic layers were dried over anhydrous Na₂SO₄, filtered, and concentrated under reduced pressure. The residue was purified by reversed-phase HPLC (0.1% FA condition) to give Ar—Cl 13 (4.43 g, 20.50 mmol, 61.08% yield, 90% purity) as a yellow solid.

### Aryl chloride 14: 4-chloro-6-(2-methyl-1,2,4-triazol-3-yl)pyrimidine

To a solution of 5-Bromo-1-methyl-1,2,4-triazole (6.00 g, 0.0370 mol, 1.00 eq.) in THF (180 mL) was added n-BuLi (2.5 M in THF, 17.8 mL, 0.0444 mol, 1.20 eq.) dropwise at −78° C. After stirring for 30 min at that temperature, ZnCl₂ (1 M in THF, 111 mL, 0.111 mol, 3.00 eq.) was slowly added. After being stirred at −78° C. for 30 min, the resulting mixture was taken up to 25° C. and stirred for 1 h. Afterwards, the mixture of 4-6 dichloropyrimidine (11.0 g, 0.0741 mol, 2.00 eq.) and Pd(PPh₃)₄ (2.14 g, 0.00185 mol, 5%) in THF (60 mL) was added at 25° C. The resulting mixture was stirred at 70° C. for 16 h. The reaction was quenched by NH₄Cl (sat. aq., 100 mL), diluted with water (100 mL) and then extracted with EtOAc (3×150 mL). The combined organic layers were washed with brine (300 mL), dried over anhydrous Na₂SO₄ solid, filtered and concentrated under reduced pressure to give a residue. The residue was purified by column chromatography (SiO₂, PE/EtOAc=10/1 to 2/1) to afford Ar—Cl 14 (448 mg, 94.0% purity, 0.00215 mol, 5.81% yield) as a white solid.

¹H NMR (400 MHz, CDCl₃) δ=9.09 (s, 1H), 8.27 (s, 1H), 8.00 (s, 1H), 4.43 (s, 3H).

LCMS: m/z=196.1 [M+H]⁺. RT 0.518 min (Method C)

### Aryl chloride 15: 4-chloro-6-(3-methyltriazol-4-yl)pyrimidine

The mixture of Tributyl-(3-methyltriazol-4-yl) stanne (4.00 g, 50.0% purity, 0.00537 mol, 1.00 eq.), 4,6-dichloropyrimidine (1.04 g, 0.00699 mol, 1.30 eq.), LiCl (0.0456 g, 0.00107 mol, 20%) and Pd(PPh₃)₂Cl₂ (0.377 g, 0.000537 mol, 10%) in DMF (75 mL) was degassed with N₂ flux for 10 min. The solution was stirred at 70° C. for 16 h. The reaction mixture was filtered and concentrated under reduced pressure to remove the most of solvent. The residue was diluted with water (100 mL) and extracted with EtOAc (3×100 mL). The combined organic layers were washed with brine (3×100 mL), dried over anhydrous Na₂SO₄ solid, filtered and concentrated under reduced pressure to give a residue. The residue was purified by column chromatography (SiO₂, PE/EtOAc=5/1 to 2/1) to afford Ar—Cl 15 (456 mg, 77.0% purity, 0.00180 mol, 33.4% yield) as a yellow solid.

¹H NMR (400 MHz, CDCl₃) δ=9.09 (d, J=0.8 Hz, 1H), 8.19 (s, 1H), 7.67 (d, J=0.8 Hz, 1H), 4.49 (s, 3H).

LCMS (ESI) m/z=196.1 [M+H]⁺. RT 0.434 (Method C)

### Aryl chloride 16: 5-(2-chloropyrimidin-4-yl)-5-azaspiro[2.4]heptan-4-one

A mixture of 2,4-Dichloropyrimidine (2.01 g, 0.013 mol, 1.50 eq.), Pd(OAc)₂ (0.203 g, 0.001 mol, 10%), XantPhos (1.04 g, 0.002 mol, 20%), Cs₂CO₃ (4.40 g, 0.0135 mol, 1.50 eq.) and 5-azaspiro[2.4]heptan-4-one (1.00 g, 0.009 mol, 1.00 eq.) in THF (30 mL) was degassed and purged with N₂ for 3 times at 0° C., and the resulting mixture was stirred at 70° C. for 2 h. After filtration through celite and washing with DCM, the filtrate was concentrated to give a residue. The residue was purified by column chromatography (SiO₂, PE/EtOAc=1/0 to 10/1) to afford Ar—Cl 16 (1.70 g, 84.5% yield) as a yellow oil.

¹H NMR (400 MHz, CDCl₃) 5=8.43 (d, J=5.6 Hz, 1H), 8.32 (d, J=5.6 Hz, 1H), 4.18 (t, J=7.2 Hz, 2H), 2.27 (t, J=7.2 Hz, 2H), 1.34-1.28 (m, 2H), 1.01-0.95 (m, 2H).

LCMS (ESI) m/z=224.1 [M+H]⁺. RT 0.575 (Method C)

### Aryl chloride 17: 1-(2-chloropyrimidin-4-yl)-3,3-difluoro-pyrrolidin-2-one

Was synthesized in a similar manner to Ar—Cl 16 using 3,3-difluoro-pyrrolidin-2-one (1.47 g, 0.00991 mol, 2.00 eq.) and 2,4-dichloropyrimidine (739 mg, 0.00496 mol, 1.00 eq.), to give Ar—Cl 17 (640 mg, 55%) as a white solid.

¹H NMR (400 MHz, CDCl₃) δ=8.60 (d, J=5.6 Hz, 1H), 8.36 (d, J=5.6 Hz, 1H), 4.15 (t, J=6.8 Hz, 2H), 2.76-2.60 (m, 2H).

### Aryl chloride 18: 1-(2-chloropyrimidin-4-yl)-3,3-dimethyl-pyrrolidin-2-one

Was synthesized in a similar manner to Ar—Cl 16 using 3,3-dimethyl-pyrrolidin-2-one (3 g, 0.0265 mol, 1.00 eq.), 2,4-dichloropyrimidine (6.3 g, 1.5 eq.) to give Ar—Cl 18 (5.10 g, 85%) as a white solid.

### Aryl chloride 19: 1 5-(2-chloropyrimidin-4-yl)-5-azaspiro[2.5]octan-4-one

Was synthesized in a similar manner to Ar—Cl 16 using 5-azaspiro[2.5]octan-4-one (300 mg, 2.4 mmol, 1.00 eq.), 2,4-dichloropyrimidine (536 mg, 1.5 eq.) to give Ar—Cl 19 (456 mg, 79%) as a white solid.

¹H NMR (400 MHz, CDCl₃) δ=8.40 (d, J=6.0 Hz, 1H), 8.17 (d, J=6.0 Hz, 1H), 4.17-4.08 (m, 2H), 2.13-2.03 (m, 2H), 1.85-1.76 (m, 2H), 1.46 (q, J=3.6 Hz, 2H), 0.79 (q, J=3.6 Hz, 2H).

### Aryl chloride 20: 1-(2-chloropyrimidin-4-yl)-3,3-difluoro-piperidin-2-one

Was synthesized in a similar manner to Ar—Cl 16 using 3,3-difluoro-piperidin-2-one (2.5 g, 0.0185 mol, 1.00 eq.), 2,4-dichloropyrimidine (3.3 g, 1.2 eq.) to give Ar—Cl 20 (220 mg, 5%) as a white solid.

¹H NMR (400 MHz, DMSO-d₆) δ=8.72 (d, J=5.6 Hz, 1H), 8.07 (d, J=5.6 Hz, 1H), 3.99 (t, J=6.0 Hz, 2H), 2.48-2.38 (m, 2H), 2.12-2.00 (m, 2H).

### Aryl chloride 21: 4-(2-chloropyrimidin-4-yl)morpholin-3-one

Was synthesized in a similar manner to Ar—Cl 16 using morpholin-3-one (5 g, 0.0495 mol, 1.00 eq.), 2,4-dichloropyrimidine (8.84 g, 1.2 eq.) to give Ar—Cl 21 (7.70 g, 71%) as a white solid.

¹H NMR (400 MHz, DMSO-d₆) δ=8.65 (d, J=5.6 Hz, 1H), 8.30 (d, J=5.6 Hz, 1H), 4.33 (s, 2H), 4.02-3.93 (m, 4H).

### Aryl chloride 22: 1-(2-chloropyrimidin-4-yl)-3-methyl-imidazolidin-2-one

Was synthesized in a similar manner to Ar—Cl 16 using 3-methyl-imidazolin-2-one (2 g, 0.0470 mol, 1.00 eq.), 2,4-dichloropyrimidine (4.46 g, 1.5 eq.) to give Ar—Cl 22 (3.90 g, 92%) as a yellowish solid.

¹H NMR (400 MHz, CDCl₃) 5=8.45 (d, J=6.0 Hz, 1H), 8.34 (d, J=6.0 Hz, 1H), 4.13-4.05 (m, 2H), 3.42-3.51 (m, 2H), 2.85-2.76 (s, 3H).

### Aryl chloride 23: 2 methylsulfonyl-4-(oxetan-3-yl)pyrimidine

Step 1: A mixture of 4-chloro-2-methylsulfanyl-pyrimidine (4.00 g, 0.0249 mol, 1.00 eq.), 3-bromooxetane (4.43 g, 0.0324 mol, 1.30 eq.), Ir[dF(CF₃)ppy]₂(dtbpy)(PF₆) (0.279 g, 0.000249 mol, 1 mol %, CAS: 870987-63-6), NiCl₂·dtbbpy (0.0496 g, 0.000125 mol, 5 mol ‰, CAS: 1034901-50-2), TTMSS (6.19 g, 0.0249 mol, 1.00 eq., CAS: 1873-77-4) and Na₂CO₃ (5.28 g, 0.0498 mol, 2.00 eq.) in DME (150 mL) was degassed and purged with N₂, and then the mixture was stirred at 25° C. for 16 h irradiated with a 455 nm blue LED. The reaction mixture was filtered, concentrated under reduced pressure to give a residue. The residue was purified by column chromatography (SiO₂, PE/EtOAc=2/1 to 0/1) to afford -methylsulfanyl-4-(oxetan-3-yl)pyrimidine (3.58 g, 0.0197 mol, 78.9% yield) as a brown solid.

¹H NMR (400 MHz, CDCl₃) δ=8.48 (d, J=5.2 Hz, 1H), 6.93 (d, J=5.2 Hz, 1H), 5.05-4.98 (m, 2H), 4.97-4.89 (m, 2H), 4.32-4.22 (m, 1H), 2.61 (s, 3H).

Step 2: To a mixture of methylsulfanyl-4-(oxetan-3-yl)pyrimidine (1.76 g, 0.00966 mol, 1.00 eq.) in THF (50 mL) was added the solution of oxone (4.88 g, 0.0290 mol, 3.00 eq.) in water (10 mL) at 0° C. After stirring at 25° C. for 18 h, another batch of oxone (12.2 g, 0.0725 mol, 7.50 eq.) was additionally added at 0° C. The resulting mixture was further stirred at 25° C. for 18 h. The mixture was diluted with water (100 mL), and then extracted with DCM (4×100 mL). The combined organic layers were washed with brine (200 mL), dried over anhydrous Na₂SO₄ solid, filtered and concentrated under reduced pressure to give a residue. The residue was purified by column chromatography (SiO₂, PE/EtOAc=3/1 to 0/1) to afford Ar—Cl 23 (573 mg, 0.00267 mol, 27.7% yield) as a white solid.

¹H NMR (400 MHz, CDCl₃) δ=8.91 (d, J=5.2 Hz, 1H), 7.60 (d, J=5.2 Hz, 1H), 5.11 (dd, J=8.4, 6.0 Hz, 2H), 4.91 (t, J=6.0 Hz, 2H), 4.55-4.42 (m, 1H), 3.41 (s, 3H).

### Aryl chloride 24: 2-chloro-4-(4-methylpyrazol-1-yl)pyrimidine

To a solution of 4-methyl pyrazole (1.00 g, 0.00671 mol, 1.33 eq.) in ACN (10 mL) were added 2,4-dichloropyrimidine (0.413 g, 0.00503 mol, 1.00 eq.) and Cs2CO3 (1.75 g, 0.00537 mol, 1.07 eq.) at 0° C. The mixture was stirred at 25° C. for 2 h. The mixture was filtered, washed with EtOAc (50 mL) and then concentrated under reduced pressure to give a residue. The residue was purified by column chromatography (SiO2, PE/EtOAc=10/1 to 3/1) to afford Ar—Cl 24 (0.790 g, 60% purity, 44.0%) as a yellow solid.

1H NMR (400 MHz, CDCl3) δ=8.56 (d, J=5.6 Hz, 1H), 8.31 (s, 1H), 7.78 (d, J=5.6 Hz, 1H), 7.62 (s, 1H), 2.15 (s, 3H).

LCMS (ESI) m/z=238.2 [M+H]⁺. RT 0.539 min (Method C)

### Aryl chloride 25: 1-(4-chloropyrimidin-2-yl)-3,3-dimethyl-pyrrolidin-2-one

Step 1: Was synthesized in a similar manner to Ar—Cl 16 using 3,3-dimethylpyrrolidin-2-one (946 mg, 0.00836 mol, 1.50 eq.), and 2-chloro-4-methylsulfanyl-pyrimidine (895 mg, 0.00557 mol, 1.00 eq.) to give 3,3-dimethyl-1-(4-methylsulfanylpyrimidin-2-yl)pyrrolidin-2-one (1.40 g, 84.0% purity, 88.9%) as a yellow oil.

¹H NMR (400 MHz, CDCl₃) δ=8.28 (d, J=5.2 Hz, 1H), 6.89 (d, J=5.2 Hz, 1H), 3.99 (t, J=7.2 Hz, 2H), 2.61 (s, 3H), 1.97 (t, J=7.2 Hz, 2H), 1.28 (s, 6H).

LCMS (ESI) m/z=238.2 [M+H]⁺. RT 0.522 min (Method C)

Step 2: To a solution of 3,3-dimethyl-1-(4-methylsulfanylpyrimidin-2-yl)pyrrolidin-2-one (1.40 g, 84.0% purity, 0.00496 mol, 1.00 eq.) in ACN (20 mL) was added conc. HCl (0.661 mL, 0.00793 mol, 1.60 eq.) and SO₂Cl₂ (1.64 mL, 0.0198 mol, 4.00 eq.) in sequence at 0° C. After being stirred at 25° C. for 30 min, the mixture was poured into ice-cooled NaHCO₃ solution (sat. aq., 30 mL) and then extracted with EtOAc (3×30 mL). The combined organic layers were washed with brine (50 mL), dried over anhydrous Na₂SO₄ solid, filtered and concentrated under reduced pressure to give a residue. The residue was purified by column chromatography (SiO₂, PE/EtOAc=15/1 to 2/1) to afford Ar—Cl 25 (282 mg, 78.0% purity, 0.000975 mol, 19.7% yield) as a yellow oil.

¹H NMR (400 MHz, CDCl₃) δ=8.57 (d, J=5.2 Hz, 1H), 7.06 (d, J=5.2 Hz, 1H), 3.98 (t, J=7.2 Hz, 2H), 2.00 (d, J=7.2 Hz, 2H), 1.28 (s, 6H).

LCMS (ESI) m/z=226.2 [M+H]⁺. RT 0.546 min (Method C).

### Aryl chloride 26: 1-(2-chloropyrimidin-2-yl)-5,5-dimethyl-pyrrolidin-2-one

Was synthesized in a similar manner to Ar—Cl 16 using 5,5-dimethylpyrrolidlin-2-one (2 g, 0.0470 mol, 1.00 eq.), 2,4-dichloropyrimidine (4.94 g, 1.5 eq.) to give Ar—Cl 26 (2.70 g, 96% purity, 52%) as a green solid.

¹H NMR (400 MHz, CDCl₃) δ=8.45 (d, J=5.6 Hz, 1H), 8.09 (d, J=5.6 Hz, 1H), 2.63 (t, J=8.4 Hz, 2H), 2.05-1.98 (m, 2H), 1.68 (s, 6H).

MS (ESI) m/z=226.1 [M+H]⁺. RT 0.582 (Method C).

### Aryl chloride 27: 5-(4-chloropyrimidin-2-yl)-5-azaspiro[2.5]octan-4-one

To a solution of 5-azaspiro[2.5]octan-4-one (770 mg, 0.00615 mol, 1.00 eq.) in DMF (15 mL) was added NaH (60%, 0.271 g, 0.00677 mol, 1.10 eq.) at 0° C. under N₂ atmosphere. After stirring at 25° C. for 30 min. 4-chloro-2-methylsulfonyl-pyrimidine (1.78 g, 0.00923 mol, 1.50 eq.) was added at 0° C., and the mixture was stirred at 25° C. for 12 h. The mixture was quenched with pre-cooled NH₄Cl (sat. aq., 20 mL) and extracted with EtOAc (3×25 mL). The combined organic layers were washed with brine (3×30 mL), dried over anhydrous Na₂SO₄ solid, filtered and concentrated under reduced pressure to give a residue. The residue was purified by column chromatography (SiO₂, PE/EtOAc=10/1 to 1/1) to afford Ar—Cl 27 (341 mg, 79.0% purity, 18.4%) as a colorless oil.

¹H NMR (400 MHz, CDCl₃) δ=8.59 (d, J=5.2 Hz, 1H), 7.13 (d, J=5.2 Hz, 1H), 4.08-4.02 (m, 2H), 2.14-2.07 (m, 2H), 1.88-1.81 (m, 2H), 1.49 (q, J=3.6 Hz, 2H), 0.72 (q, J=3.6 Hz, 2H).

LCMS (ESI) m/z=238.2 [M+H]⁺. RT 0.540 (Method C).

### Aryl chloride 28: 3-(2-chloropyrimidin-4-yl)oxazolidin-2-one

Was synthesized in a similar manner to Ar—Cl 16 using oxazolidin-2-one (5.0 g, 57.4 mmol, 1.00 eq.), 2,3-dichloropyrimidine (1.28 g, 86.1 mmol, 1.00 eq.), to give Ar—Cl 28 (4.40 g, 36%).

### Aryl chloride 29: 2-methylsulfonyl-4-(oxetan-2-yl)pyrimidine

Step 1: A mixture of 4-chloro-2-methylsufamyl-pyrimidine (1.50 g, 0.00934 mol, 1.00 eq.), oxetane-2-carboxylic acid (1.43 g, 0.0140 mol, 1.50 eq.), Ir[dF(CF₃)ppy]₂(dtbpy)(PF₆) (0.210 g, 0.000187 mol, 2%, CAS: 870987-63-6), NiCl₂·dtbbpy (0.186 g, 0.000467 mol, 5%, CAS: 1034901-50-2), phthalimide (1.37 g, 0.00934 mol, 1.00 eq., CAS: 85-41-6) and BTMG (2.40 g, 0.0140 mol, 1.50 eq., CAS: 29166-72-1) in DMSO (100 mL) was degassed and purged with N₂, and then the mixture was stirred at 25° C. for 16 h irradiated with a 455 nm blue LED. The reaction mixture was diluted with brine (100 mL) and then extracted with EtOAc (3×100 mL). The combined organic layers were dried over anhydrous Na₂SO₄ solid, filtered and concentrated to give a residue (4.23 g). The residue was purified by column chromatography (SiO₂, PE/EtOAc=8/1 to 2/1) to afford 2-methylsulfamyl-4-(oxetan-2-yl)pyrimidine (1.42 g, 78.0% purity, 0.00609 mol, 65.2% yield, contaminated by phthalimide) as a yellow solid.

¹H NMR (400 MHz, CDCl₃) δ=8.58 (d, J=4.8 Hz, 1H), 7.30 (d, J=4.8 Hz, 1H), 5.71-5.61 (m, 1H), 4.92-4.79 (m, 1H), 4.75-4.66 (m, 1H), 3.24-3.09 (m, 1H), 2.74-2.61 (m, 1H), 2.60-2.59 (m, 1H), 2.57 (s, 3H).

LCMS: m/z=183.2 [M+H]+. RT 0.496 min (Method C)

Step 2: To a solution of 2-methylsulfamyl-4-(oxetan-2-yl)pyrimidine (2.07 g, 0.0114 mol, 1.00 eq.) in MeOH (40 mL) was added the solution of oxone (5.73 g, 0.0341 mol, 3.00 eq.) in water (10 mL) in one portion at 0° C. After stirring at 25° C. for 22 h, another batch of oxone (2.87 g, 0.0170 mol, 1.50 eq.) was additionally added at 0° C. The resulting mixture was further stirred at 25° C. for 5 h. The mixture was quenched with Na₂SO₃ (sat. aq., 30 mL) dropwise at 0° C. After stirring at 25° C. for 1 h, it was directly concentrated under reduced pressure to remove most of MeOH, and then extracted with EtOAc (3×50 mL). The combined organic layers were washed with brine (100 mL), dried over anhydrous Na₂SO₄ solid, filtered and concentrated to give a residue. The residue was purified by column chromatography (SiO₂, PE/EtOAc=3/1 to 1/1) to afford Ar—Cl 29 (0.374 g, 0.00175 mol, 15.4% yield) as a yellow solid.

¹H NMR (400 MHz, CDCl₃) δ=8.98 (d, J=4.8 Hz, 1H), 7.94 (d, J=4.8 Hz, 1H), 5.87 (dd, J=8.8, 6.4 Hz, 1H), 5.00-4.87 (m, 1H), 4.76-4.66 (m, 1H), 3.37 (s, 3H), 3.34-3.22 (m, 1H), 2.75-2.64 (m, 1H).

### Aryl Chloride 30: 2-chloro-5-fluoro-4-(2-methylimidazol-1-yl)pyrimidine

To a solution of 2-Methyl-1H-Imidazole (2.00 g, 0.0244 mol, 1.00 eq.) and 2,4-Dichloro-5-fluoropyrimidine (8.13 g, 0.0487 mol, 2.00 eq.) in ACN (25 mL) was added Cs₂CO₃ (9.13 g, 0.0280 mol, 1.15 eq.) in one portion at 0° C. After stirring for 2 h at 25° C., the resulting mixture was stirred at 80° C. for 3 h. The reaction mixture was diluted with ice-cooled water (100 mL), concentrated under reduced pressure to remove most of ACN, and then extracted with EtOAc (2×80 mL) and n-BuOH (80 mL). The combined organic layers were dried over anhydrous Na₂SO₄ solid, filtered and concentrated under reduced pressure to give a residue. The residue was purified by column chromatography (SiO₂, PE/EtOAc=3/1 to 0/1) to give Ar—Cl 30 (4.00 g, 0.0188 mol, 77.2% yield) as a yellow solid.

¹H NMR (400 MHz, CDCl₃) δ=8.63 (d, J=2.8 Hz, 1H), 7.40 (dd, J=3.6, 1.6 Hz, 1H), 7.08 (d, J=1.6 Hz, 1H), 2.70 (s, 3H).

### Aryl Chloride 31: 1-(6-chloropyrimidin-4-yl)pyrrolidin-2-one

Was synthesized in a similar manner to Ar—Cl 16 using pyrrolidine-2-one (2.14 mL, 1.05 eq.), 4,6-dichloropyrimidine (4 g, 1.5 eq.) to give Ar—Cl 31 (4.7 g, 88%) as yellowish solid.

¹H NMR (400 MHz, CDCl₃) δ=8.71 (s, 1H), 8.47 (s, 1H), 4.08 (t, J=7.6 Hz, 2H), 2.69 (t, J=7.6 Hz, 2H), 2.18 (quin, J=7.6 Hz, 2H).

LCMS: m/z=198.1 [M+H]+. RT, 0.462 min (Method Q)

### Aryl Chloride 32: 4-chloro-6-(2-methyl-5-nitro-pyrazol-3-yl)pyrimidine

The solution of 4-chloro-6-(2-methylpyrazol-3-yl)pyrimidine (530 mg, 0.00272 mol, 1.00 eq.) in TFAA (6 mL) in Pump 1 and HNO3 (98% purity, 0.290 mL, 0.00408 mol, 1.50 eq.) in Pump 2 were simultaneously pumped to the flow reactor (25° C.). The reaction mixture was collected with a bottle (contained 20 mL ice-cooled water) after running 25 mins. The resulting reaction mixture was filtered and the filter cake was dried in vacuo to afford Ar—Cl 32 (416 mg, 0.00174 mol, 63.8% yield). 1H NMR (400 MHz, CD3OD) δ=8.81 (d, J=5.2 Hz, 1H), 7.92 (d, J=5.2 Hz, 1H), 7.74 (s, 1H), 4.37 (s, 3H).

### Aryl Chloride 33: 2-chloro-4-(3-methoxyazetidin-1-yl)pyrimidine

To a solution of 3-Methoxyacetidine (300 mg, 0.00243 mol, 1.00 eq.) in ACN (15 mL) was added TEA (0.841 mL, 0.00607 mol, 2.50 eq.) and 2,4-dichloropyrimidine (434 mg, 0.00291 mol, 1.20 eq.) at 0° C. The mixture was stirred at 25° C. for 16 h. The mixture was diluted with precooled NH₄Cl solution (sat. aq., 100 mL) and then extracted with EtOAc (3×100 mL). The combined organic layers were washed with brine (100 mL), dried over anhydrous Na₂SO₄ solid, filtered and concentrated under reduced pressure to give a residue. The residue was purified by column chromatography (SiO₂, PE/EtOAc=4/1 to 1/1) to afford Ar—Cl 33 (387 mg, 0.00194 mol, 79.9% yield) as a white solid.

¹H NMR (400 MHz, CDCl₃) δ=8.01 (d, J=6.0 Hz, 1H), 6.08 (d, J=6.0 Hz, 1H), 4.40-4.34 (m, 1H), 4.33-4.21 (m, 2H), 4.00 (d, J=6.8 Hz, 2H), 3.35 (s, 3H).

LCMS: m/z=200.2 [M+H]+. RT, 0.435 min (Method Q)

### Aryl Chloride 34: 5-(2-chloro-5-fluoro-pyrimidin-4-yl)-5-azaspiro[2.4]heptan-4-one

was synthesized in a similar manner to Ar—Cl 16 using 5-azaspiro[2.4]heptan-4-one (1 g, 9.0 mmol, 1.00 eq.), 2,4-dichloro-5-fluoropyrimidine (2.25 g, 1.5 eq.) to give Ar—Cl 34 (1.8 g, 83%) as a white solid.

¹H NMR (400 MHz, CDCl₃) δ=8.42 (d, J=2.4 Hz, 1H), 4.12-4.06 (m, 2H), 2.30 (t, J=7.2 Hz, 2H), 1.34-1.29 (m, 2H), 1.01-0.95 (m, 2H).

LCMS: m/z=242.2 [M+H]+. RT, 0.544 min (Method Q)

### Aryl Chloride 35: 2-chloro-4-[(3R or 3S)-3-methoxypyrrolidin-1-yl]pyrimidine

Isomer R was synthesized in a similar manner to Ar—Cl 33 using (3R)-3-methoxypyrrolidine hydrochloride (300 mg, 2.2 mmol) and 2,4-dichloropyrimidine (487 mg, 3.2 mmol) to give Ar—Cl 35 (R-isomer) (398 mg, 85%) as a white solid)

¹H NMR (400 MHz, CDCl₃) δ=8.00 (d, J=6.0 Hz, 1H), 6.20 (s, 1H), 4.12-3.98 (m, 1H), 3.96-3.39 (m, 4H), 3.36 (s, 3H), 2.39-2.04 (m, 2H).

Isomer S was synthesized in a similar manner but using (3S)-3-methoxypyrrolidine (120 mg, 0.56 mmol).

### Aryl Chloride 36: 2-chloro-4-[(3S)-3-fluoropyrrolidin-1-yl]pyrimidine

Was synthesized in a similar manner to Ar—Cl 33 using (3S)-3-fluoropyrrolidine 300 mg, 2.39 mmol), 2,4-dichloropyrimidine 534 mg, 3.58 mmol) to give Ar—Cl 36 (447 mg, 93%) as a white solid 1H NMR (400 MHz, CDCl3) δ=8.05 (d, J=6.0 Hz, 1H), 6.24 (s, 1H), 5.49-5.26 (m, 1H), 4.15-3.89 (m, 1H), 3.83-3.42 (m, 3H), 2.55-2.35 (m, 1H), 2.32-2.05 (m, 1H).

### Aryl Chloride 37: ethyl 1-(2-chloropyrimidin-4-yl)-5-methyl-pyrazole-4-carboxylate

To a solution of (2-chloropyrimidine-4yl)hydrazine (1.60 g, crude above) in EtOH (16 mL) was added HCl (1 M in water, 1.6 mL) and ethyl (2E)-2-(dimethylaminomethylene)-3-oxo-butanoate (3.07 g, 0.0166 mol) in sequence at 0° C. The resulting mixture was further stirred at 0° C. for 1 h. The mixture was added NaHCO₃ (sat., aq., 2 mL) and water (30 mL), and then extracted with EtOAc (2×50 mL). The combined organic layers were washed with brine (20 mL), dried over anhydrous Na2SO4 solid, filtered and concentrated under reduced pressure to give a residue. The residue was purified by column chromatography to afford Ar—Cl 37 (570 mg, 0.00214 mol, 11.5%) as a white solid.

1H NMR (400 MHz, CDCl3) δ=8.67 (d, J=5.6 Hz, 1H), 8.07 (s, 1H), 7.93 (d, J=5.6 Hz, 1H), 4.35 (q, J=7.2 Hz, 2H), 3.09 (s, 3H), 1.39 (t, J=7.2 Hz, 3H).

### General procedure for the coupling of Intermediate (XX) with Aryl-Chloride

A solution of intermediate I-06 to I-11 or I-32 to I-35 (1.05 eq) and corresponding Ar—Cl (1q), base (3-10 eq) in the corresponding solvent was made. The mixture was heated to the corresponding temperature for 2-16 h until LC/MS shows full conversion. The mixture was filtered through a filter and directly purified by prep. HPLC or previously was quenched with precooled NH₄Cl solution (sat. aq.) and then extracted with EtOAc. The combined organic layers were washed with brine, dried over anhydrous Na₂SO₄ solid, filtered and concentrated under reduced pressure to give a residue. The residue was purified by column chromatography, NPLC or prep-HPLC to afford the desired products.

The following methods were used for the Examples described below:

| Coupling Methods | Base | Solvent | Temperature |
|---|---|---|---|
| Method A | TEA | DMF | 25° C. |
| Method B | DIPEA | MeCN | 60° C. |
| Method C | DIPEA | DMSO | 60° C. |
| Method D | TEA | DMF | 60° C. |
| Method E | TEA | MeCN | 60° C. |
| Method F | TEA | MeCN | 25° C. |
| Method G | TEA | EtOH | 25° C. |
| Method H | DIPEA | DMF | 25° C. |
| Method I | K2CO3 | MeCN | 120° C. |
| Method J | DIPEA | MeCN | 100° C., MW |
| Method K | TEA | DMSO | 60° C. |

### Example 1: (S)-(3-(5-fluoropyridin-3-yl)isoxazolidin-2-yl)(1-(6-(pyridin-3-yl)pyrimidin-4-yl)piperidin-4-yl)methanone (Method I)

Intermediate I-09 (50 mg, 80.47 μmol) and Ar—Cl1 (24.35 mg, 120.70 μmol) and potassium carbonate (24.47 mg, 177.02 μmol) were introduced in a reaction vessel. Then acetonitrile (1.5 ml) was added and the mixture was heated for 2 h at 120° C. in der MW. After this time, LC/MS shows full conversion and the reaction was filtered through a filter and purified by prep. HPLC. The corresponding fractions were lyophilized to give Example 1 as a white solid (35 mg, 60%).

Following examples were synthesized in a similar manner using the starting materials, intermediates and coupling methods as indicated above and in table 3: 1, 2, 3, 4, 5, 6, 22, 23, 29, 30, 31, 32, 33, 34, 36, 39, 40, 41, 42, 43, 44, 49, 51, 52, 53, 58, 59, 60, 61, 62, 63, 66, 70, 71, 72, 73, 74, 75, 76, 78, 79, 80, 83, 85, 87, 88, 89, 90, 91, 92, 98, 99, 100 and 101.

TABLE 3

| Example | Intermediate | Aryl-chloride | Coupling Method |
|---|---|---|---|
| 1 | I-09 | Ar-Cl1 | I |
| 2 | I-06 | Ar-Cl2 | J |
| 3 | I-06 | Ar-Cl3 | J |
| 4 | I-06 | Ar-Cl4 | J |
| 5 | I-06 | Ar-Cl5 | J |
| 6 | I-06 | Ar-C16 | J |
| 22 | I-06 | Ar-Cl7 | J |
| 23 | I-07 | Ar-Cl8 | J |
| 29 | I-06 | Ar-Cl9 | J |
| 30 | I-06 | Ar-Cl10 | J |
| 31 | I-06 | Ar-Cl11 | J |
| 32 | I-06 | Ar-Cl12 | J |
| 33 | I-11 | Ar-Cl11 | J |
| 34 | I-11 | Ar-Cl13 | J |
| 36 | I-34 | Ar-Cl2 | E |
| 39 | I-35 | Ar-Cl13 | C |
| 40 | I-07 | Ar-Cl14 | D |
| 41 | I-33 | Ar-Cl14 | K |
| 42 | I-33 | Ar-Cl15 | A |
| 43 | I-37 | Ar-Cl14 | A |
| 44 | I-11 (R) | Ar-Cl3 | C |
| 49 | I-10 | Ar-Cl14 | A |
| 51 | I-34 | Ar-Cl16 | B |
| 52 | I-07 | Ar-Cl16 | B |
| 53 | I-09 | Ar-Cl16 | B |
| 58 | I-06 | Ar-Cl17 | B |
| 59 | I-07 | Ar-Cl17 | B |
| 60 | I-10 | Ar-Cl18 | B |
| 61 | I-10 | Ar-Cl16 | B |
| 62 | I-09 | Ar-Cl22 | B |
| 63 | I-11 (R) | Ar-Cl21 | B |
| 66 | I-08 | Ar-Cl20 | B |
| 70 | I-08 | Ar-Cl16 | B |
| 71 | I-09 | Ar-Cl4 | D |
| 72 | I-09 | Ar-Cl19 | D |
| 73 | I-08 | Ar-Cl17 | B |
| 74 | I-34 | Ar-Cl17 | B |
| 75 | I-09 | Ar-Cl17 | B |
| 76 | I-34 | Ar-Cl20 | B |
| 78 | I-11 (R) | Ar-Cl11 | C |
| 79 | I-11 (S) | Ar-Cl11 | C |
| 80 | I-11 (S) | Ar-Cl13 | C |
| 83 | I-35 (R) | Ar-Cl24 | C |
| 84 | I-08 | Ar-Cl25 | G |
| 85 | I-11 (R) | Ar-Cl4 | G |
| 87 | I-08 | Ar-Cl4 | A |
| 88 | I-08 | Ar-Cl26 | B |
| 89 | I-10 | Ar-Cl26 | B |
| 90 | I-08 | Ar-Cl27 | K, 25° C. |
| 91 | I-09 | Ar-Cl2 | C |
| 92 | I-07 | Ar-Cl3 | B |
| 98 | I-10 | Ar-Cl29 | K, 25° C. |
| 99 | I-10 | Ar-Cl29 | K, 25° C. |
| 100 | I-35 (R) | Ar-Cl3 | B |
| 101 | I-10 | Ar-Cl23 | K |

Compound (98) & (99) are separated isomers but were not structurally exact identified.

### Example 2: (S)-3-(2-(4-(3-(pyrazin-2-yl)isoxazolidine-2-carbonyl)piperidin-1-yl)pyrimidin-4-yl)oxazolidin-2-one

Starting from intermediate I-06 TFA Salt (40 mg, 60.0 μmol) and Ar—Cl2 (12.4 mg, 60.0 μmol) and DIPEA (90 μL, 0.51 mmol) in acetonitrile (0.5 ml): MW, 100° C., 1 h, to give example 2 as a white solid (7.9 mg, 33%) after prep. HPLC purification.

### Example 3: (S)-1-(4-(4-(3-(pyrazin-2-yl)isoxazolidine-2-carbonyl)piperidin-1-yl)pyrimidin-2-yl)pyrrolidin-2-one

Starting from intermediate I-06 TFA salt (33 mg, 50.0 μmol) and Ar—Cl 3 (9.9 mg, 50.0 μmol) and DIPEA (60 μL, 0.37 mmol) in acetonitrile (0.5 ml): MW, 100° C., 1 h (two times). To give example 3 as a white solid (8.5 mg, 44%) chromatographic purification on silica gel (4 g Silica gel, Gradient: 5 Min 100% DCM/30 Min 100% DCM to 5% EtOH, then 10 Min 5% EtOH.

### Example 4: (S)-1-(2-(4-(3-(pyrazin-2-yl)isoxazolidine-2-carbonyl)piperidin-1-yl)pyrimidin-4-yl)pyrrolidin-2-one

Starting from intermediate I-06TFA salt (37 mg, 56.0 μmol) and Ar—Cl 4 (56.0 μmol) and DIPEA (60 μL, 0.37 mmol) in acetonitrile (0.5 ml): MW, 100° C., 1 h, to give example 4 as a white solid (8.0 mg, 30%) after prep. HPLC purification without TFA.

### Example 5: 3-[5-fluoro-2-[4-[(3S)-3-pyrazin-2-ylisoxazolidine-2-carbonyl]-1-piperidyl]pyrimidin-4-yl]oxazolidin-2-one

Starting from intermediate I-06 TFA salt (46 mg, 69.0 μmol) and Ar—Cl 5 (69.0 μmol) and DIPEA (90 μL, 0.51 mmol) in acetonitrile (0.5 ml): MW, 100° C., 1 h, to give example 5 as a white solid (8.0 mg, 30%) after prep. HPLC purification without TFA.

### Example 6: 1-[5-fluoro-2-[4-[(3S)-3-pyrazin-2-ylisoxazolidine-2-carbonyl]-1-piperidyl]pyrimidin-4-yl]pyrrolidin-2-one

Starting from intermediate I-06 TFA salt (40 mg, 60.0 μmol) and Ar—Cl 6 (60.0 μmol) and DIPEA (90 μL, 0.51 mmol) in acetonitrile (0.5 ml): MW, 100° C., 1 h, to give example 6 as a white solid (10.3 mg, 34%) after prep. HPLC purification without TFA.

### Example 22: [1-[4-(2,4-dimethylimidazol-1-yl)-5-fluoro-pyrimidin-2-yl]-4-piperidyl]-[(3S)-3-pyrazin-2-ylisoxazolidin-2-yl]methanone

Starting from intermediate I-06 TFA salt (36 mg, 54.0 μmol) and Ar—Cl 7 (54.0 μmol) and DIPEA (90 μL, 0.51 mmol) in acetonitrile (0.5 ml): MW, 100° C., 1 h, to give example 22 as a white solid (14.8 mg, 61%) after silica gel purification with a combiflash 12 g silica gel; Gradient: 5 Min 100% DCM/30 Min 100% DCM to 5% EtOH; then 10 Min 5% EtOH.

### Example 23: [1-[4-(2-methylimidazol-1-yl)-1,3,5-triazin-2-yl]-4-piperidyl]-[(3S)-3-(2-methylthiazol-4-yl)isoxazolidin-2-yl]methanone

Starting from intermediate I-07 TFA salt (30 mg, 80.0 μmol) and Ar—Cl 8 (29.7 mg, 150.0 μmol) and DIPEA (70 μL, 0.38 mmol) in acetonitrile (1.5 ml): MW, 100° C., 1 h, to give example 23 as a white solid (6.5 mg, 19%) after silica gel purification followed by prep. HPLC without TFA.

### Example 29: (S)-(1-(2-(2-methyl-1H-imidazol-1-yl)pyrimidin-4-yl)piperidin-4-yl)(3-(pyrazin-2-yl)isoxazolidin-2-yl)methanone

Starting from intermediate I-06 TFA salt (40 mg, 60.0 μmol) and Ar—Cl 9 (13.2 mg, 70.0 μmol) and DIPEA (50 μL, 0.28 mmol) in acetonitrile (0.5 ml): MW, 100° C., 1 h, to give example 29 as a white solid (21.6 mg, 91%) after silica gel purification followed by prep. HPLC without TFA.

### Example 30: (S)-(1-(4-(2-methyl-1H-imidazol-1-yl)pyrimidin-2-yl)piperidin-4-yl)(3-(pyrazin-2-yl)isoxazolidin-2-yl)methanone

Starting from intermediate I-06 TFA salt (40 mg, 60.0 μmol) and Ar—Cl 10 (13.2 mg, 70.0 μmol) and DIPEA (50 μL, 0.28 mmol) in acetonitrile (0.5 ml): MW, 100° C., 1 h, to give example 30 as a white solid (19.9 mg, 84%) after silica gel purification followed by prep. HPLC without TFA.

### Example 31: (S)-(1-(6-(2-methyl-1H-imidazol-1-yl)pyrimidin-4-yl)piperidin-4-yl)(3-(pyrazin-2-yl)isoxazolidin-2-yl)methanone

Starting from intermediate I-06TFA salt (30 mg, 50.0 μmol) and Ar—Cl 11 (11 mg, 50.0 μmol) and DIPEA (30 μL, 0.18 mmol) in acetonitrile (1.5 ml): MW, 100° C., 1.5 h, to give example 31 as a white solid (12 mg, 63%) after prep. HPLC (once without TFA and once with TFA).

### Example 32: (S)-(1-(5-fluoro-4-(1H-pyrazol-1-yl)pyrimidin-2-yl)piperidin-4-yl)(3-(pyrazin-2-yl)isoxazolidin-2-yl)methanone

Starting from intermediate I-06 TFA salt (80 mg, 110.0 μmol) and Ar—Cl 12 (12.1 mg, 60.0 μmol) and DIPEA (80 μL, 0.45 mmol) in acetonitrile (3.5 ml): MW, 100° C., 1.5 h, to give example 32 as a white solid (17.9 mg, 38%) after silica gel purification followed by prep. HPLC (without TFA).

### Example 33-[5-[6-(2-methylimidazol-1-yl)pyrimidin-4-yl]-5-azaspiro[2.5]octan-8-yl]-[(3S)-3-(6-methylpyridin-3-yl)-1,2-oxazolidin-2-yl]methanone

The title compound was prepared in a similar manner to Example 1, using 5-azaspiro[2.5]octan-8-yl-[3-(6-methyl-3-pyridyl)isoxazolidin-2-yl]methanone I-11 (80 mg, 265 μmol), Ar—Cl 11 4-chloro-6-(2-methylimidazol-1-yl)pyrimidine (56.8 mg, 292 μmol), DIEA (370 μL, 2.12 mmol) in DMSO (1 mL). The residue was purified by prep-HPLC (column: Phenomenex Synergi C18 150*25 mm*10 um; mobile phase: [water (0.225% FA)-ACN]; B %: 0%-25%, 10 min) to afford the title compound as a white solid (40 mg, 32%) and as a 1:1 mixture of two diastereomers.

### Example 34-[5-[6-(2-methylpyrazol-3-yl)pyrimidin-4-yl]-5-azaspiro[2.5]octan-8-yl]-[(3S)-3-(6-methylpyridin-3-yl)-1,2-oxazolidin-2-yl]methanone

The title compound was prepared in a similar manner to Example 1, using 5-azaspiro[2.5]octan-8-yl-[3-(6-methyl-3-pyridyl)isoxazolidin-2-yl]methanone I-11 (80 mg, 265 μmol), Ar—Cl 13 4-chloro-6-(2-methylpyrazol-3-yl)pyrimidine (56.8 mg, 292 μmol), DIEA (370 μL, 2.12 mmol) in DMSO (1 mL). The residue was purified by prep-HPLC (column: Phenomenex Synergi C18 150*25 mm*10 um; mobile phase: [water (0.225% FA)-ACN]; B %: 0%-30%, 10 min) to afford Example 34 as a white solid (40 mg, 32%) and as a 1:1 mixture of two diastereomers.

### Example 36: 3-[2-[4-[(3S)-3-(5-methyl-3-pyridyl)isoxazolidine-2-carbonyl]-1-piperidyl]pyrimidin-4-yl]oxazolidin-2-one

The title compound was prepared in a similar manner to Example 1, using I-34 (512 mg, 56% purity, 0.105 mmol), Ar—Cl 2 (150 mg, 0.752 mmol), TEA (10 eq) in MeCN (5 mL) at 60° C. The residue was purified by prep-HPLC to afford Example 36 as a white solid (195 mg, 59%).

Chiral SFC: RT 0.875 min (100% ee) (Method AH)

### Example 39: [(8R)-5-[6-(2-methylpyrazol-3-yl)pyrimidin-4-yl]-5-azaspiro[2.5]octan-8-yl]-[(3S)-3-pyrazin-2-yi-1,2-oxazolidin-2-yl]methanone

The title compound was prepared in a similar manner to Example 1, using I-35 (200 mg, 0.69 mmol), Ar—Cl 13 4-chloro-6-(2-methylpyrazol-3-yl)pyrimidine (148 mg, 0.76 mmol), DIPEA (0.361 mL, 3 eq) in DMSO (1 mL) at 60° C. The residue was purified by prep-HPLC to afford Example 39 as a white solid (206 mg, 66%).

Chiral SFC: RT 1.973 min (100% ee) (Method AP)

### Example 40: [(3S)-3-(2-methyl-1,3-thiazol-4-yl)-1,2-oxazolidin-2-yl]-[1-[6-(2-methyl-1,2,4-triazol-3-yl)pyrimidin-4-yl]piperidin-4-yl]methanone

The title compound was prepared in a similar manner to Example 1, using I-07 (390 mg, 62% purity, 102 μmol), Ar—Cl 14 (200 mg, 102 μmol), TEA (1.42 mL, 10 eq) in DMF (4 mL) at rt. The residue was purified by prep-HPLC to afford Example 40 as a white solid (234 mg, 52%).

Chiral SFC: RT 1.751 min (100% ee) (Method Z)

### Example 41: [(3S)-3-(2-methyl-1,3-thiazol-4-yl)-1,2-oxazolidin-2-yl]-[(8R)-5-[6-(2-methyl-1,2,4-triazol-3-yl)pyrimidin-4-yl]-5-azaspiro[2.5]octan-8-yl]methanone

The title compound was prepared in a similar manner to Example 1, using I-33 (264 mg, 767 μmol), Ar—Cl 14 (150 mg, 767 μmol), TEA (1.07 mL, 10 eq) in DMSO (1 mL). The residue was purified by prep-HPLC to afford Example 41 as a white solid (271 mg, 76%).

Chiral SFC: RT 1.162 min (100% ee) (Method AI)

### Example 42: [(3S)-3-(2-methyl-1,3-thiazol-4-yl)-1,2-oxazolidin-2-yl]-[(8R)-5-[6-(3-methyltriazol-4-yl)pyrimidin-4-yl]-5-azaspiro[2.5]octan-8-yl]methanone

The title compound was prepared in a similar manner to Example 1, using I-33 (311 mg, 89% purity, 901 μmol), Ar—Cl 15 (218 mg, 77% purity, 858 μmol), TEA (1.19 mL, 10 eq) in DMF (2 mL) at 25° C. The residue was purified by prep-HPLC to afford Example 42 as a white solid (107 mg, 27%).

Chiral SFC: RT 1.703 min (100% ee) (Method AC)

### Example 43: [(3S)-3-(5-fluoro-6-methylpyridin-3-yl)-1,2-oxazolidin-2-yl]-[(8R)-5-[6-(2-methyl-1,2,4-triazol-3-yl)pyrimidin-4-yl]-5-azaspiro[2.5]octan-8-yl]methanone

The title compound was prepared in a similar manner to Example 1, using I-32 (203 mg, 636 μmol), Ar—Cl 14 (124 mg, 636 μmol), TEA (883 μL, 10 eq) in DMF (2 mL) at rt. The residue was purified by prep-HPLC to afford Example 43 as a white solid (162 mg, 53%).

Chiral SFC: RT 1.595 min (100% ee) (Method AH)

### Example 44: 1-[2-[(8R)-8-[(3S)-3-(6-methylpyridin-3-yl)-1,2-oxazolidine-2-carbonyl]-5-azaspiro[2.5]octan-5-yl]pyrimidin-4-yl]pyrrolidin-2-one

The title compound was prepared in a similar manner to Example 1, using I-11 (R) (480 mg, 50% purity, 860 μmol), Ar—Cl 3 (187 mg, 940 μmol), DIPEA (1.5 mL, 10 eq) in DMSO (3 mL). The residue was purified by prep-HPLC to afford Example 44 as a white solid.

Chiral SFC: RT 1.256 min (100% ee) (Method X)

### Example 49: [(3S)-3-(5-fluoro-6-methylpyridin-3-yl)-1,2-oxazolidin-2-yl]-[1-[6-(2-methyl-1,2,4-triazol-3-yl)pyrimidin-4-yl]piperidin-4-yl]methanone

The title compound was prepared in a similar manner to Example 1, using I-10 (246 mg, 839 μmol), Ar—Cl 14 (164 mg, 839 μmol), TEA (1.17 mL, 10 eq) in DMF (6 mL) at 25° C. The residue was purified by prep-HPLC to afford Example 49 as a white solid (159 mg, 42%).

Chiral SFC: RT 1.239 min (100% ee) (Method X)

### Example 51: 5-[2-[4-[(3S)-3-(5-methylpyridin-3-yl)-1,2-oxazolidine-2-carbonyl]piperidin-1-yl]pyrimidin-4-yl]-5-azaspiro[2.4]heptan-4-one

The title compound was prepared in a similar manner to Example 1, using I-34 (686 mg, 54% purity, 134 μmol), Ar—Cl 16 (250 mg, 112 μmol), DIPEA (10 eq) in MeCN at 60° C. The residue was purified by prep-HPLC to afford Example 51 as a white solid (313 mg, 60%).

Chiral SFC: RT 1.515 min (100% ee) (Method AG)

### Example 52: 5-[2-[4-[(3S)-3-(2-methyl-1,3-thiazol-4-yl)-1,2-oxazolidine-2-carbonyl]piperidin-1-yl]pyrimidin-4-yl]-5-azaspiro[2.4]heptan-4-one

The title compound was prepared in a similar manner to Example 1, using I-07 (629 mg, 60% purity, 134 μmol), Ar—Cl 16 (250 mg, 112 μmol), DIPEA (10 eq) in MeCN at 60° C. The residue was purified by prep-HPLC to afford Example 52 as a white solid (129 mg, 25%).

Chiral SFC: RT 1.831 min (100% ee) (Method AJ)

### Example 53: 5-[2-[4-[(3S)-3-(5-fluoropyridin-3-yl)-1,2-oxazolidine-2-carbonyl]piperidin-1-yl]pyrimidin-4-yl]-5-azaspiro[2.4]heptan-4-one

The title compound was prepared in a similar manner to Example 1, using I-09 (681 mg, 55% purity, 134 μmol), Ar—Cl 16 (250 mg, 112 μmol) DIPEA (10 eq) in MeCN at 60° C. The residue was purified by prep-NPLC to afford Example 53 as a white solid (167 mg, 32%).

Chiral SFC: RT 2.054 min (100% ee) (Method AJ)

### Example 58: 3,3-difluoro-1-[2-[4-[(3S)-3-pyrazin-2-yi-1,2-oxazolidine-2-carbonyl]piperidin-1-yl]pyrimidin-4-yl]pyrrolidin-2-one

The title compound was prepared in a similar manner to Example 1, using I-06 (210 mg, 62% purity, 496 μmol), Ar—Cl 17 (120 mg, 514 μmol), DIPEA (10 eq) in MeCN at 60° C. The residue was purified by prep-HPLC to afford Example 58 as a white solid (126 mg, 55%).

Chiral SFC: RT 1.850 min (100% ee) (Method X)

### Example 59: 3,3-difluoro-1-[2-[4-[(3S)-3-(2-methyl-1,3-thiazol-4-yl)-1,2-oxazolidine-2-carbonyl]piperidin-1-yl]pyrimidin-4-yl]pyrrolidin-2-one

The title compound was prepared in a similar manner to Example 1, using I-07 (471 mg, 38%, 642 μmol), Ar—Cl 17 (150 mg, 642 μmol), DIPEA (10 eq) in MeCN at 60° C. The residue was purified by prep-HPLC to afford Example 59 as a white solid (225 mg, 72%).

Chiral SFC: RT 0.910 min (100% ee) (Method AH)

### Example 60: 1-[2-[4-[(3S)-3-(5-fluoro-6-methylpyridin-3-yl)-1,2-oxazolidine-2-carbonyl]piperidin-1-yl]pyrimidin-4-yl]-3,3-dimethylpyrrolidin-2-one

The title compound was prepared in a similar manner to Example 1, using I-10 (858 mg, 77% purity, 143 μmol), Ar—Cl 18 (540 mg, 227 μmol), DIPEA (10 eq) in MeCN at 60° C. The residue was purified by prep-HPLC to afford Example 60 as a white solid (300 mg, 27%).

Chiral SFC: RT min (100% ee) (Method X)

### Example 61: 5-[2-[4-[(3S)-3-(5-fluoro-6-methylpyridin-3-yl)-1,2-oxazolidine-2-carbonyl]piperidin-1-yl]pyrimidin-4-yl]-5-azaspiro[2.4]heptan-4-one

The title compound was prepared in a similar manner to Example 1, using I-10 (404 mg, 65% purity, 8.94 mmol), Ar—Cl 16 (200 mg, 8.94 mmol), DIPEA (10 eq) in MeCN at 60° C. The residue was purified by prep-HPLC to afford Example 61 as a white solid 245 mg, 56%).

Chiral SFC: RT min (100% ee) (Method X)

### Example 62: 1-[2-[4-[(3S)-3-(5-fluoropyridin-3-yl)-1,2-oxazolidine-2-carbonyl]piperidin-1-yl]pyrimidin-4-yl]-3-methylimidazolidin-2-one

The title compound was prepared in a similar manner to Example 1, using I-09 (350 mg, 877 μmol), Ar—Cl 22 (205 mg, 965 μmol), DIPEA (10 eq) in MeCN at 60° C. The residue was purified by prep-HPLC to afford Example 62 as a white solid (257 mg, 64%).

Chiral SFC: RT min (100% ee) (Method X)

### Example 63: 4-[2-[(8R)-8-[(3S)-3-(6-methylpyridin-3-yl)-1,2-oxazolidine-2-carbonyl]-5-azaspiro[2.5]octan-5-yl]pyrimidin-4-yl]morpholin-3-one

The title compound was prepared in a similar manner to Example 1, using I-11 (R) (665 mg, 24% purity, 530 μmol), Ar—Cl 21 (113 mg, 530 μmol), DIPEA (10 eq) in MeCN at 60° C. The residue was purified by prep-HPLC to afford Example 63 as a white solid (98 mg, 39%).

Chiral SFC: RT min (100% ee) (Method X)

### Example 66: 3,3-difluoro-1-[2-[4-[(3S)-3-(6-methylpyridin-3-yl)-1,2-oxazolidine-2-carbonyl]piperidin-1-yl]pyrimidin-4-yl]piperidin-2-one

The title compound was prepared in a similar manner to Example 1, using I-08 (145 mg, 54% purity, 283 μmol), Ar—Cl 20 (70 mg, 283 μmol), DIPEA (10 eq) in MeCN at 60° C. The residue was purified by prep-HPLC to afford Example 66 as a white solid (16 mg, 11%).

Chiral SFC: RT min (100% ee) (Method X)

### Example 70: 5-[2-[4-[(3S)-3-(6-methylpyridin-3-yl)-1,2-oxazolidine-2-carbonyl]piperidin-1-yl]pyrimidin-4-yl]-5-azaspiro[2.4]heptan-4-one

The title compound was prepared in a similar manner to Example 1, using I-09 (629 mg, 54% purity, 123 μmol), Ar—Cl 17 (250 mg, 112 μmol), DIPEA (10 eq) in MeCN at 60° C. The residue was purified by prep-HPLC to afford Example 70 as a white solid (159 mg, 30%).

Chiral SFC: RT min (100% ee) (Method X)

### Example 71: 1-[4-[4-[(3S)-3-(5-fluoropyridin-3-yl)-1,2-oxazolidine-2-carbonyl]piperidin-1-yl]pyrimidin-2-yl]pyrrolidin-2-one

The title compound was prepared in a similar manner to Example 1, using I-09 (450 mg, 58% purity, 935 μmol), Ar—Cl 4 (210 mg, 1.06 mmol), TEA (10 eq) in DMF (6 mL) at 25° C. The residue was purified by prep-HPLC to afford Example 71 as a white solid (80 mg, 17%).

Chiral SFC: RT min (100% ee) (Method AK)

### Example 72: 5-[2-[4-[(3S)-3-(5-fluoropyridin-3-yl)-1,2-oxazolidine-2-carbonyl]piperidin-1-yl]pyrimidin-4-yl]-5-azaspiro[2.5]octan-4-one

The title compound was prepared in a similar manner to Example 1, using I-09 (427 mg, 66% purity, 1.01 mmol), Ar—Cl 19 (200 mg, 841 μmol), TEA (10 eq) in DMF (5 mL) at 60° C. The residue was purified by prep-HPLC to afford Example 72 as a white solid (183 mg, 45%).

Chiral SFC: RT min (100% ee) (Method X)

### Example 73: 3,3-difluoro-1-[2-[4-[(3S)-3-(6-methylpyridin-3-yl)-1,2-oxazolidine-2-carbonyl]piperidin-1-yl]pyrimidin-4-yl]pyrrolidin-2-one

The title compound was prepared in a similar manner to Example 1, using I-08 (263 mg, 54% purity, 514 μmol), Ar—Cl 17 (120 mg, 514 μmol),), DIPEA (10 eq) in MeCN at 60° C. The residue was purified by prep-HPLC to afford Example 73 as a white solid (101 mg, 41%).

Chiral SFC: RT min (100% ee) (Method X)

### Example 74: 3,3-difluoro-1-[2-[4-[(3S)-3-(5-methylpyridin-3-yl)-1,2-oxazolidine-2-carbonyl]piperidin-1-yl]pyrimidin-4-yl]pyrrolidin-2-one

The title compound was prepared in a similar manner to Example 1, using I-34 (312 mg, 57% purity, 642 μmol), Ar—Cl 17 (150 mg, 642 μmol), DIPEA (10 eq) in MeCN at 60° C. The residue was purified by prep-HPLC to afford Example 74 as a white solid (151 mg, 50%).

Chiral SFC: RT min (100% ee) (Method Z)

### Example 75: 3,3-difluoro-1-[2-[4-[(3S)-3-(5-fluoropyridin-3-yl)-1,2-oxazolidine-2-carbonyl]piperidin-1-yl]pyrimidin-4-yl]pyrrolidin-2-one

The title compound was prepared in a similar manner to Example 1, using I-09 (460 mg, 39% purity, 642 μmol), Ar—Cl 17 (150 mg, 642 μmol), DIPEA (10 eq) in MeCN at 60° C. The residue was purified by prep-HPLC to afford Example 75 as a white solid (103 mg, 34%).

Chiral SFC: RT min (100% ee) (Method X)

### Example 76: 3,3-difluoro-1-[2-[4-[(3S)-3-(5-methylpyridin-3-yl)-1,2-oxazolidine-2-carbonyl]piperidin-1-yl]pyrimidin-4-yl]piperidin-2-one

The title compound was prepared in a similar manner to Example 1, using I-34 (138 mg, 57% purity, 283 μmol), Ar—Cl 20 (100 mg, 283 pmol DIPEA (10 eq) in MeCN at 60° C. The residue was purified by prep-HPLC to afford Example 76 as a white solid (25 mg, 18%).

Chiral SFC: RT min (100% ee) (Method X)

### Example 78: [5-[6-(2-methylimidazol-1-yl)pyrimidin-4-yl]-5-azaspiro[2.5]octan-8-yl]-[(3S)-3-(6-methylpyridin-3-yl)-1,2-oxazolidin-2-yl]methanone

The title compound was prepared in a similar manner to Example 1, using I-11 (R) (225 mg, 747 μmol), Ar—Cl 10 (160 mg, 822 μmol), DIPEA (10 eq) in DMSO (3 mL). The residue was purified by prep-HPLC to afford Example 78 as a white solid (307 mg, 81%).

Chiral SFC: RT min (100% ee) (Method F)

### Example 79: [5-[6-(2-methylimidazol-1-yl)pyrimidin-4-yl]-5-azaspiro[2.5]octan-8-yl]-[(3S)-3-(6-methylpyridin-3-yl)-1,2-oxazolidin-2-yl]methanone

The title compound was prepared in a similar manner to Example 1, using I-11 (S) (225 mg, 747 μmol), Ar—Cl 10 (160 mg, 822 μmol), DIPEA (10 eq) in DMSO (3 mL). The residue was purified by prep-HPLC to afford Example 79 as a white solid (294 mg, 78%).

Chiral SFC: RT min (100% ee) (Method F)

### Example 80: [5-[6-(2-methylpyrazol-3-yl)pyrimidin-4-yl]-5-azaspiro[2.5]octan-8-yl]-[(3S)-3-(6-methylpyridin-3-yl)-1,2-oxazolidin-2-yl]methanone

The title compound was prepared in a similar manner to Example 1, using I-11 (S) (800 mg, 28% purity, 747 μmol), Ar—Cl 13 (160 mg, 822 μmol), DIPEA (10 eq) in DMSO (3 mL) at 60° C. The residue was purified by prep-HPLC to afford Example 80 as a white solid (304 mg, 79%).

Chiral SFC: RT min (100% ee) (Method F)

### Example 83: [(8R)-5-[4-(4-methylpyrazol-1-yl)pyrimidin-2-yl]-5-azaspiro[2.5]octan-8-yl]-[(3S)-3-pyrazin-2-yl-1,2-oxazolidin-2-yl]methanone

The title compound was prepared in a similar manner to Example 1, using I-35 (R) (178 mg, 617 μmol), Ar—Cl 24 (167 mg, 60% purity, 360 μmol), DIPEA (268 μL, 3 eq) in DMSO (1 mL). The residue was purified by prep-HPLC to afford Example 83 as a white solid (154 mg, 67%).

Chiral SFC: RT min (100% ee) (Method AB)

### Example 84: 3,3-dimethyl-1-[4-[4-[(3S)-3-(6-methylpyridin-3-yl)-1,2-oxazolidine-2-carbonyl]piperidin-1-yl]pyrimidin-2-yl]pyrrolidin-2-one

The title compound was prepared in a similar manner to Example 1, using I-08 (588 mg, 78% purity 166 μmol), Ar—Cl 25 (268 mg, 78% purity, 923 μmol), TEA (2.15 mL, 154 mmol) in EtOH (5 mL) at RT. The residue was purified by prep-HPLC to afford Example 84 as a white solid (111 mg, 26%).

Chiral SFC: RT 0.834 min (100% ee) (Method N)

### Example 85: 1-[4-[(8R)-8-[(3S)-3-(6-methylpyridin-3-yl)-1,2-oxazolidine-2-carbonyl]-5-azaspiro[2.5]octan-5-yl]pyrimidin-2-yl]pyrrolidin-2-one

The title compound was prepared in a similar manner to Example 1, using I-11 (R) (575 mg, 73% purity, 139 μmol), Ar—Cl 4 (250 mg, 127 μmol), TEA (1.41 μL, 8 eq) in EtOH (2 mL). The residue was purified by prep-HPLC to afford Example 85 as a white solid (87 mg, 15%).

Chiral SFC: RT 1.390 min (99.99% ee) (Method Z)

### Example 87: 1-[4-[4-[(3S)-3-(6-methylpyridin-3-yl)-1,2-oxazolidine-2-carbonyl]piperidin-1-yl]pyrimidin-2-yl]pyrrolidin-2-one

The title compound was prepared in a similar manner to Example 1, using I-08 (492 mg, 78%, 139 μmol), Ar—Cl 4 (275 mg, 139 μmol), TEA DMF (10 mL). The residue was purified by prep-HPLC to afford Example 87 as a white solid (54 mg, 9%).

Chiral SFC: RT 1.894 min (99.99% ee) (Method Y)

### Example 88: 5,5-dimethyl-1-[2-[4-[(3S)-3-(6-methylpyridin-3-yl)-1,2-oxazolidine-2-carbonyl]piperidin-1-yl]pyrimidin-4-yl]pyrrolidin-2-one

The title compound was prepared in a similar manner to Example 1, using I-08 (680 mg, 54% purity, 133 μmol), Ar—Cl 26 (250 mg, 111 μmol), DIPEA (10 eq) in MeCN (5 mL). The residue was purified by prep-HPLC to afford Example 88 as a white solid (143 mg, 28%).

Chiral SFC: RT 1.678 min (100% ee) (Method P)

### Example 89: 1-[2-[4-[(3S)-3-(5-fluoro-6-methylpyridin-3-yl)-1,2-oxazolidine-2-carbonyl]piperidin-1-yl]pyrimidin-4-yl]-5,5-dimethylpyrrolidin-2-one

The title compound was prepared in a similar manner to Example 1, using I-10 (450 mg, 63% purity, 966 μmol), Ar—Cl 26 (240 mg, 106 μmol), DIPEA (10 eq) in MeCN (4 mL) at 60° C. The residue was purified by prep-HPLC to afford chiral

Example 89 as a white solid (161 mg, 35%).

Chiral SFC: RT 1.619 min (100% ee) (Method P)

### Example 90: 5-[4-[4-[(3S)-3-(6-methylpyridin-3-yl)-1,2-oxazolidine-2-carbonyl]piperidin-1-yl]pyrimidin-2-yl]-5-azaspiro[2.5]octan-4-one

The title compound was prepared in a similar manner to Example 1, using I-08 (881 mg, 54%, 172 μmol), Ar—Cl 27 (341 mg, 143 μmol), TEA (10 eq) in DMF (10 mL) at 25° C. The residue was purified by prep-HPLC to afford Example 90 as a white solid (187 mg, 27%).

Chiral SFC: RT 1.401 min (100% ee) (Method X)

### Example 91: 3-[2-[4-[(3S)-3-(5-fluoropyridin-3-yl)-1,2-oxazolidine-2-carbonyl]piperidin-1-yl]pyrimidin-4-yl]-1,3-oxazolidin-2-one

The title compound was prepared in a similar manner to Example 1, using I-09 (254 mg, 1 mmol), Ar—Cl 2 (200 mg, 1 mmol), DIPEA (5 eq) in MeCN (10 mL) at 60° C. The residue was purified by prep-HPLC to afford Example 91 as a white solid (171 mg, 39%).

Chiral SFC: RT 1.910 min (100% ee) (Method X)

### Example 92: 1-[2-[4-[(3S)-3-(2-methyl-1,3-thiazol-4-yl)-1,2-oxazolidine-2-carbonyl]piperidin-1-yl]pyrimidin-4-yl]pyrrolidin-2-one

The title compound was prepared in a similar manner to Example 1, using I-07 (360 mg, 78% purity, 998 μmol), Ar—Cl 3 (187 mg, 948 μmol), DIPEA (10 eq) in MeCN (4 mL) at 60° C. The residue was purified by prep-HPLC to afford Example 92 as a white solid (189 mg, 43%).

Chiral SFC: RT 1.474 min (100% ee) (Method H)

### Example 98 and Example 99: [(3S)-3-(5-fluoro-6-methylpyridin-3-yl)-1,2-oxazolidin-2-yl]-[1-[4-(oxetan-2-yl)pyrimidin-2-yl]piperidin-4-yl]methanone (isomers 1 and 2)

To a solution of Intermediate I-10 (842 mg, crude, ~44.9% purity, 0.00129 mol, 0.932 eq.) in DMSO (6 mL) was added TEA (2.88 mL, 0.0208 mol, 15.0 eq.) and Ar—Cl 29 (333 mg, 89.0% purity, 0.00138 mol, 1.00 eq.) at 25° C. The mixture was stirred at 90° C. for 23 h. The mixture was quenched with precooled NH4Cl (sat. aq., 30 mL), and then extracted with EtOAc (3×30 mL). The combined organic layers were washed with brine (4×50 mL), dried over anhydrous Na2SO4 solid, filtered and concentrated under reduced pressure to give a residue. The residue was purified by column chromatography (SiO2, PE/EtOAc=1/1 to 0/1 to DCM/MeOH=I/O to 6/1) and NPLC (column: Welch Ultimate XB—SiOH 250*50*10 um; mobile phase: [Hexane-EtOH]; gradient: 1%-30% B over 15 min) and then lyophilized to afford Example 98 (0.457 g, 0.00107 mol, 77.3% yield) as a yellow oil. The two isomers were separated by SFC: (condition: DAICEL CHIRALPAK IC (250 mm*30 mm, 10 um); mobile phase: (CO₂-iPrOH (1‰ NH₃—H₂O); B %: 65%, isocratic elution mode).

Chiral SFC: RT 0.80 min (184.27 mg, 99.9% e.e. Example 98) and RT: 1.11 min (192.32 mg, 98.9% e.e, Example 99) (Method U)

### Example 100: 1-[2-[(8R)-8-[(3S)-3-pyrazin-2-yi-1,2-oxazolidine-2-carbonyl]-5-azaspiro[2.5]octan-5-yl]pyrimidin-4-yl]pyrrolidin-2-one

The title compound was prepared in a similar manner to Example 1, using I-35—(R) (150 mg, 520 μmol), Ar—Cl 3 (103 mg, 520 μmol), DIPEA (5 eq) in MeCN (3 mL) at 60° C. The residue was purified by prep-HPLC to afford Example 100 as a white solid (109 mg, 47%).

Chiral SFC: RT 1.590 min (100% ee) (Method W)

### Example 101: [(3S)-3-(5-fluoro-6-methylpyridin-3-yl)-1,2-oxazolidin-2-yl]-[1-[4-(oxetan-3-yl)pyrimidin-2-yl]piperidin-4-yl]methanone

The title compound was prepared in a similar manner to Example 1, using I-09 (354 mg, 90% purity, 1.09 mmol), Ar—Cl 23 (250 mg, 1.17 mmol), TEA (15 eq) in DMSO (5 mL) at 90° C. The residue was purified by prep-HPLC to afford Example 101 as a yellow gum (101 mg, 20%).

Chiral SFC: RT 1.804 min (100% ee) (Method P)

### Example 103: 5-[5-fluoro-2-[4-[(3S)-3-(5-methylpyridin-3-yl)-1,2-oxazolidine-2-carbonyl]piperidin-1-yl]pyrimidin-4-yl]-5-azaspiro[2.4]heptan-4-one

The title compound was prepared in a similar manner to Example 1, using I-34 (355 mg, 73% purity, 0.93 mmol), Ar—Cl 34 (150 mg, 0.62 mmol), TEA (10 eq) in ACN (4 mL) at 60° C. The residue was purified by prep-HPLC to afford Example 103 as a white solid (141 mg, 47%).

Chiral SFC: RT 1.585 min (100% ee) (Method X)

### Example 104: 5-[5-fluoro-2-[4-[(3S)-3-(5-fluoropyridin-3-yl)-1,2-oxazolidine-2-carbonyl]piperidin-1-yl]pyrimidin-4-yl]-5-azaspiro[2.4]heptan-4-one

The title compound was prepared in a similar manner to Example 1, using I-10 (477 mg, 67% purity, 0.9 mmol), Ar—Cl 34 (150 mg, 0.62 mmol), TEA (10 eq) in ACN (4 mL) at 60° C. The residue was purified by prep-HPLC to afford Example 103 as a white solid (121 mg, 41%).

Chiral SFC: RT 1.542 min (100% ee) (Method X)

### Example 105: 5-[5-fluoro-2-[4-[(3S)-3-(5-methylpyrazin-2-yl)-1,2-oxazolidine-2-carbonyl]piperidin-1-yl]pyrimidin-4-yl]-5-azaspiro[2.4]heptan-4-one

The title compound was prepared in a similar manner to Example 1, using I-36 (319 mg, 64% purity, 0.7 mmol), Ar—Cl 34 (150 mg, 0.62 mmol), TEA (10 eq) in ACN (4 mL) at 60° C. The residue was purified by prep-HPLC to afford Example 103 as an off-white solid (127 mg, 42%).

Chiral SFC: RT 0.778 min (100% ee) (Method AC)

### Example 106: [1-[4-[(3S)-3-fluoropyrrolidin-1-yl]pyrimidin-2-yl]piperidin-4-yl]-[(3S3-(5-methylpyrazin-2-yl)-1,2-oxazolidin-2-yl]methanone

The title compound was prepared in a similar manner to Example 1, using I-36 (212 mg, 64% purity, 0.49 mmol), Ar—Cl 36 (120 mg, 0.62 mmol), TEA (10 eq) in ACN (4 mL) at 80° C. The residue was purified by prep-HPLC to afford Example 103 as an off-white solid (70.8 mg, 32%).

Chiral SFC: RT 2.089 min (100% ee) (Method X)

### Example 107 [1-[4-[(3R)-3-methoxypyrrolidin-1-yl]pyrimidin-2-yl]piperidin-4-yl]-[(3S)-3-(5-methylpyrazin-2-yl)-1,2-oxazolidin-2-yl]methanone

The title compound was prepared in a similar manner to Example 1, using I-36 (200 mg, 64% purity, 0.47 mmol), Ar—Cl 35 (R-isomer) (120 mg, 0.56 mmol), DEA (10 eq) in ACN (4 mL) at 80° C. The residue was purified by prep-HPLC to afford Example 107 as an off-white solid (53 mg, 25%).

Chiral SFC: RT 0.934 min (100% ee) (Method AH)

### Example 108 [1-[4-[(3S)-3-methoxypyrrolidin-1-yl]pyrimidin-2-yl]piperidin-4-yl]-[(3S)-3-(5-methylpyrazin-2-yl)-1,2-oxazolidin-2-yl]methanone

The title compound was prepared in a similar manner to Example 1, using I-36 (200 mg, 64% purity, 0.47 mmol), Ar—Cl 35 (S-isomer) (120 mg, 0.56 mmol), DEA (10 eq) in ACN (4 mL) at 80° C. The residue was purified by prep-HPLC to afford Example 108 as an off-white solid (64 mg).

Chiral SFC: RT 0.934 min (100% ee) (Method AC)

### Example 109 5-[5-fluoro-2-[4-[(3S3-(6-methylpyridin-3-yl)-1,2-oxazolidine-2-carbonyl]piperidin-1-yl]pyrimidin-4-yl]-5-azaspiro[2.4]heptan-4-one

The title compound was prepared in a similar manner to Example 1, using I-08 (2432 mg, 77% purity, 0.68 mmol), Ar—Cl 34 (150 mg, 0.62 mmol), TEA (10 eq) in ACN (4 mL) at 60° C. The residue was purified by prep-HPLC to afford Example 109 as a white solid (83 mg, 28%).

Chiral SFC: RT 1.504 min (100% ee) (Method M)

### Example 110 1-[5-fluoro-2-[4-[(3S3-(5-methylpyrazin-2-yl)-1,2-oxazolidine-2-carbonyl]piperidin-1-yl]pyrimidin-4-yl]pyrrolidin-2-one

The title compound was prepared in a similar manner to Example 1, using I-36 (298 mg, 65% purity, 0.69 mmol), Ar—Cl 6 (150 mg, 0.69 mmol), TEA (10 eq) in ACN (4 mL) at 60° C. The residue was purified by prep-HPLC to afford Example 110 as a white solid (120 mg, 38%).

Chiral SFC: RT 1.734 min (100% ee) (Method X)

### Example 113 [1-[4-(3-methoxyazetidin-1-yl)pyrimidin-2-yl]piperidin-4-yl]-[(3S3-(5-methylpyrazin-2-yl)-1,2-oxazolidin-2-yl]methanone and

The title compound was prepared in a similar manner to Example 1, using I-36 (200 mg, 65% purity, 0.62 mmol), Ar—Cl 33 (93 mg, 0.62 mmol), TEA (10 eq) in ACN (4 mL) at 60° C. The residue was purified by prep-HPLC to afford Example 113 as a white solid (47 mg, 23%).

Chiral SFC: RT 1.485 min (100% ee) (Method X)

### Example 114: 3-[5-fluoro-2-[4-[(3S3-(5-fluoropyridin-3-yl)-1,2-oxazolidine-2-carbonyl]piperidin-1-yl]pyrimidin-4-yl]-1,3-oxazolidin-2-one

The title compound was prepared in a similar manner to Example 1, using I-09 (355 mg, 61% purity), Ar—Cl 5 (200 mg, 84%), TEA (10 eq) in Dioxane:H2O (4 mL) at 60° C. The residue was purified by prep-HPLC to afford Example 114 as a white solid (57 mg, 13%).

Chiral SFC: RT 1.783 min (100% ee) (Method X)

### Example 25. [1-(5-fluoro-2-pyrimidin-5-yl-pyrimidin-4-yl)-4-piperidyl]-[(3S)-3-pyrazin-2-ylisoxazolidin-2-yl]methanone

### Step 1

Starting from Intermediate I-06 (150 mg, 260 μmol) and 2,4-dichloro-5-fluoropyrimidine (29 mg, 280 mmol) and DIPEA (230 μL, 1.29 mmol) in MeCN (3 mL): MW, 100° C., 2 h, to give [1-(2-chloro-5-fluoro-pyrimidin-4-yl)-4-piperidyl]-[(3S)-3-pyrazin-2-ylisoxazolidin-2-yl]methanone (84 mg, 83%) after column chromatography on silica gel.

LCMS: m/z 393.2 [M+1]⁺; RT 1.68 (Method A)

### Step 2

To a solution of [1-(2-chloro-5-fluoro-pyrimidin-4-yl)-4-piperidyl]-[(3S)-3-pyrazin-2-ylisoxazolidin-2-yl]methanone (22 mg, 0.056 mmol, 1 eq) in dioxane (2 mL) was added Cs₂CO₃ (36 mg, 0.112 mmol, 2 eq), H2O (0.5 mL) and Pyrimidine-5-boronic acid pinacol ester (12 mg, 0.058 mmol, 1 eq). The mixture was rinsed for 5 min with Argon and then Pd(dppf)Cl₂ (4 mg, 0.005 mmol) was added. The mixture was stirred at 100° C. for 15 min. LCMS showed no starting material remained. The residue was quenched with saturated aqueous NaCl and extracted with EE. The combined organic layers were dried over Na2SO4, filtered and concentrated under reduced pressure to give a residue. The residue was purified by flash silica gel chromatography to give Example 25 (4.3 mg, 17%).

### Example 27: (S)-(1-(5-fluoro-4-(thiazol-5-yl)pyrimidin-2-yl)piperidin-4-yl)(3-(pyrazin-2-yl)isoxazolidin-2-yl)methanone

### Step 1

Starting from Intermediate I-06 (250 mg, 0.350 mμmol) and 2-chloro-5-fluoropyrimidine-4-ol (262 mg, 1.77 mmol) and DIPEA (360 μL, 2.12 mmol) in MeCN (4 mL): MW, 120° C., 1 h, to give (S)-(1-(5-fluoro-4-hydroxypyrimidin-2-yl)piperidin-4-yl)(3-(pyrazin-2-yl)isoxazolidin-2-yl)methanone (83 mg, 68%) after column chromatography on silica gel.

LCMS: m/z 375.2 [M+1]⁺; RT 1.00 (Method A)

### Step 2

To a solution of (S)-(1-(5-fluoro-4-hydroxypyrimidin-2-yl)piperidin-4-yl)(3-(pyrazin-2-yl)isoxazolidin-2-yl)methanone (85 mg, 230 μmo, 1) in dichloromethane (5 mL), POCl₃ (1.47 g, 11.35 mmol) was added and the mixture stirred at 70° C. for 1 h. The reaction was cooled with an ice bath and quenched slowly with saturated NaHCO₃. The mixture was extracted with dichloromethane (3×) and the combined organic layers dried over MgSO₄ and purified by silica gel chromatography to give (S)-(1-(4-chloro-5-fluoropyrimidin-2-yl)piperidin-4-yl)(3-(pyrazin-2-yl)isoxazolidin-2-yl)methanone (30 mg, 34%).

LCMS: m/z 393.1 [M+1]⁺; RT2.03 (Method A)

### Step 3

To a solution of (S)-(1-(4-chloro-5-fluoropyrimidin-2-yl)piperidin-4-yl)(3-(pyrazin-2-yl)isoxazolidin-2-yl)methanone (25 mg, 60 μmol) in dioxane (2 mL) was added Cs₂CO₃ (42.3 mg, 130 μmol), H2O (0.5 mL) and 5-(4,4,5,5-Tetramethyl-1,3,2-dioxaborolan-2-yl)thiazole (14 mg, 70 μmol). The mixture was rinsed for 5 min with Argon and then Pd(dppf)Cl₂(CH₂Cl₂) (5.3 g, 10 μmol) was added. The mixture was stirred at 100° C. for 15 min. LCMS showed no starting material remained. The residue was quenched with saturated aqueous NaCl and extracted with EE. The combined organic layers were dried over Na2SO4, filtered and concentrated under reduced pressure to give a residue. The residue was purified by silica gel chromatography to give Example 27 (25 mg, 88%).

### Example 28: (S)-(1-(6-(1-methyl-1H-pyrazol-5-yl)pyrimidin-4-yl)piperidin-4-yl)(3-(pyrazin-2-yl)isoxazolidin-2-yl)methanone

### Step 1

Starting from I-06 TFA (330 mg, 0.47 mmol) and 4,6-dichloropyrimidine (78 mg, 0.51 mmol) with DIEA (4eq), (S)-(1-(6-chloropyrimidin-4-yl)piperidin-4-yl)(3-(pyrazin-2-yl)isoxazolidin-2-yl)methanone (175 mg, 100%)) was obtained.

LC/MS: m/z 375.2 [M+1]⁺: RT 1.48 min (Method A)

¹H NMR (400 MHz, DMSO-d6) d ppm 8.62 (s, 1H), 8.61 (m, 1H), 8.57 (d, J=2.45 Hz, 1H), 8.31 (s, 1H), 6.96 (s, 1H), 5.43 (dd, J=8.68, 6.36 Hz, 1H), 4.34 (td, J=7.61, 7.61, 3.48 Hz, 3H), 4.00 (m, 1H), 3.08 (m, 3H), 2.85 (m, 1H), 2.50 (u), 1.89 (br d, J=12.84 Hz, 1H), 1.77 (br d, J=13.08 Hz, 1H), 1.49 (m, 2H)

### Step 2: (S)-(1-(6-(1-methyl-1H-pyrazol-5-yl)pyrimidin-4-yl)piperidin-4-yl)(3-(pyrazin-2-yl)isoxazolidin-2-yl)methanone

A mixture of (S)-(1-(6-chloropyrimidin-4-yl)piperidin-4-yl)(3-(pyrazin-2-yl)isoxazolidin-2-yl)methanone (40 mg, 110 μmol) in DME (2.25 mL) was added 1-methyl-5-(4,4,5,5-tetramethyl-1,3,2-dioxaborolan-2-yl)pyrazole (25 mg, 120 μmol, 1 eq.), K₂CO₃ (46 mg, 0.43 mmol), Pd(dppf)Cl₂ (17 mg, 20 μmol) and H₂O (0.75 mL) was degassed and purged with N₂. The mixture was heated in the MW for 20 min at 100° C. The reaction mixture was diluted with EA and extracted with water. The organic layer was concentrated and the residue purified by HPLC to give Example 28 (15.2 mg, 35%) as a white solid.

### Example 48: [(3S)-3-(5-fluoro-6-methylpyridin-3-yl)-1,2-oxazolidin-2-yl]-[(8R)-5-[4-(2-methylpyrazol-3-yl)-1,3,5-triazin-2-yl]-5-azaspiro[2.5]octan-8-yl]methanone (following SM2 flowed by a Suzuki reaction)

Step 1: In a similar manner to Example 1, by using I-32 (R) HCl salt (370 mg, 78% purity, 892 μmol) and 2,4-dichloro-1,3,5-triazine (3134 mg, 892 μmol, 1.0 eq) with DIEA (1.55 mL. 10 eq) in DMF to give [(8R)-5-(4-chloro-1,3,5-triazin-2-yl)-5-azaspiro[2.5]octan-8-yl]-[(3S)-3-(5-fluoro-6-methyl-3-pyridyl)isoxazolidin-2-yl]methanone as off-white solid (242 mg, 63%).

LCMS: m/z=433.2 [M+H]⁺. RT 0.609 min (Method Q).

Step 2: To a solution of [(8R)-5-(4-chloro-1,3,5-triazin-2-yl)-5-azaspiro[2.5]octan-8-yl]-[(3S)-3-(5-fluoro-6-methyl-3-pyridyl)isoxazolidin-2-yl]methanone (242 mg, 0.559 mmol, 1.00 eq.) in dioxane (6 mL) and water (0.5 mL) was added 1-methyl-5-(4,4,5,5-tetramethyl-1,3,2-dioxaborolan-2-yl)pyrazole (128 mg, 0.615 mmol, 1.00 eq.), Pd(dppf)Cl₂ (0.0409 g, 55.9 umol, 10%) and K₂CO₃ (0.155 g, 1.12 mmol, 2.00 eq.). The mixture was stirred at 80° C. for 12 h under N₂ atmosphere. The reaction mixture was filtered and concentrated under reduced pressure to give a residue. The residue was purified by column chromatography (SiO₂, PE/EtOAc=10/1 to EtOAc/MeOH=10/1) to afford the crude product (120 mg, 93% purity). The above crude product was further purified by prep-HPLC (column: Phenomenex luna C18 150*25 mm*10 um; mobile phase: [water (HCOOH)-ACN]; gradient: 41%-71% B over 10 min) to afford Example 48 (89.40 mg, 33.4% yield) as a white solid.

### Example 68: 5-[5-fluoro-2-[4-[(3S)-3-(5-fluoro-6-methylpyridin-3-yl)-1,2-oxazolidine-2-carbonyl]piperidin-1-yl]pyrimidin-4-yl]-5-azaspiro[2.4]heptan-4-one

Starting from I-05 TFA salt (1 eq) and I-47 (200 mg, 0.59 mmol) with pyridine (0.38 mL, 8 eq) the desired product Example 68 was obtained as white solid (161.4 mg, 54%).

Chiral SFC: RT 1.588 min (99.99% ee) (Method X)

### Example 69: [(8R)-5-[4-(2-methylpyrazol-3-yl)-1,3,5-triazin-2-yl]-5-azaspiro[2.5]octan-8-yl]-[(3S)-3-(6-methylpyridin-3-yl)-1,2-oxazolidin-2-yl]methanone (Following SM2 Followed by a Suzuki Reaction)

Step 1: In a similar manner to Example 1, by using I-11 (R) HCl salt (2.47 g, 73% purity, 5.82 mmol) and 2,4-dichloro-1,3,5-triazine (1.34 g, 8.97 mmol, 1.0 eq) with DIEA (8.33 mL. 8 eq) in DMF at 25° C., to give [(8R)-5-(4-chloro-1,3,5-triazin-2-yl)-5-azaspiro[2.5]octan-8-yl]-[(3S)-3-(6-methyl-3-pyridyl)isoxazolidin-2-yl]methanone (1.05 g, 58% purity, 58%) as a yellow oil.

Step 2: To a solution of [(8R)-5-(4-chloro-1,3,5-triazin-2-yl)-5-azaspiro[2.5]octan-8-yl]-[(3S)-3-(6-methyl-3-pyridyl)isoxazolidin-2-yl]methanone (1.05 g, 58% purity, 147 mmol)- and 1-methyl-5-(4,4,5,5-tetramethyl-1,3,2-dioxaborolan-2-yl)pyrazole (450 mg, 2.20 mmol, 1.00 eq.), Pd(dppf)Cl₂ (0.107 g, 0.000147 mol, 10%) and CuCl (0.174 g, 0.00176 mol, 1.20 eq.) in DMF (8 mL) was added K₃PO₄ (0.935 g, 0.00440 mol, 3.00 eq.) under N₂ atmosphere, and the resulting mixture was heated to 100° C. for 18 h. The mixture was quenched with precooled NH₄Cl (sat. aq., 50 mL) and then extracted with EtOAc (3×50 mL). The combined organic layers were washed with brine (3×50 mL), dried over anhydrous Na₂SO₄ solid, filtered and concentrated under reduced pressure to give a residue. The residue was purified by prep-HPLC (column: Phenomenex Luna C18 150*25 mm*10 um; mobile phase: [water (0.5‰ NH₃—H₂O)-ACN]; gradient: 31%-61% B over 10 min), and further purified by prep-HPLC (column: Phenomenex Luna C18 150*25 mm*10 um; mobile phase: [water (2.25‰ HCOOH)-ACN]; gradient: 21%-45% B over 10 min), and finally lyophilized to afford Example 69 (297.58 mg, 29.5% yield) as a white solid.

Chiral SFC: RT 1.414 min (98.66% ee) (Method X)

### General scheme for the synthesis of examples 7, 8, 9, 10, 11, 12, 13, 14, 15, 16, 17, 18, 19, 20, 21, 24, 26, 28, 35, 37, 38, 45, 46, 47, 50, 54, 55, 56, 57, 64, 65, 67, 77, 81, 82, 86, 93, 94, 95, 96 and 97 from intermediates I-12 to I-27 and I-37 to I-46

Examples 7, 8, 9, 10, 11, 12, 13, 14, 15, 16, 17, 18, 19, 20, 21, 24, 26, 28 and 35 were synthesized by coupling intermediates I-01 to I-05 with the corresponding intermediates I-12 to I-27 using the general procedure (Hatu coupling).

### Example 7: [1-[6-(2-methylimidazol-1-yl)pyrimidin-4-yl]-4-piperidyl]-[(3S)-3-(2-methylthiazol-4-yl)isoxazolidin-2-yl]methanone

Starting from I-12 (146 mg, 0.51 mmol) and I-02 HCl Salt (100 mg, 0.4838 mmol) with DIPEA (0,379 mL, 2.18 mmol, 4.5eq) the desired product Example 7 (76 mg, 36% yield) was obtained as a white solid.

### Example 8: 1-[4-(2-methylpyrazol-3-yl)-1,3,5-triazin-2-yl]-4-piperidyl]-[(3S)-3-(6-methyl-3-pyridyl)isoxazolidin-2-yl]methanone

Starting from I-03 HCl salt (146.16 mg, 728.40 μmol, 1.05 eq, HCl) and Intermediate I-27 (200 mg, 693.71 μmol, 1 eq), Example 8 was obtained (115 mg, 263.35 μmol, 37.96% yield, 99.5% purity) as a white solid.

Chiral SFC: 1.218 min (100%, Method F).

### Example 9: [1-[4-(4-methylpyrazol-1-yl)pyrimidin-2-yl]-4-piperidyl]-[(3S)-3-(6-methyl-3-pyridyl)isoxazolidin-2-yl]methanone

Starting from I-03 TFA Salt (32 mg, 100 μmmol) and I-23 (31 mg, 110 μmol, 1.1 eq) with DIPEA (4eq), the desired product Example 9 was obtained as a white solid (30 mg, 69%).

### Example 10: [1-(5-fluoro-4-pyrazol-1-yl-pyrimidin-2-yl)-4-piperidyl]-[(3S)-3-(6-methyl-3-pyridyl)isoxazolidin-2-yl]methanone

Starting from I-03 (160 mg, 500 μmol) and I-14 (153 mg, 525 μmol, 1.1 eq) with DIPEA (4eq), the desired product Example 10 was obtained as a white solid (152 mg, 70%).

### Example 11: [1-(4-oxazol-2-ylpyrimidin-2-yl)-4-piperidyl]-[(3S)-3-pyrazin-2-ylisoxazolidin-2-yl]methanone

Starting from I-01 (7.5 mg, 50 μmol) and I-21 (153 mg, 53 μmol, 1.1 eq) with DIPEA (4eq), the desired product Example 11 was obtained as a white solid (18 mg, 89%).

Chiral SFC: RT 1.80 min (100%, Method AD).

### Example 12: [1-(5-fluoro-4-oxazol-2-yl-pyrimidin-2-yl)-4-piperidyl]-[(3S)-3-pyrazin-2-ylisoxazolidin-2-yl]methanone

Starting from I-01 (7.5 mg, 50 μmol) and I-16 (153 mg, 53 μmol, 1.1 eq) with DIPEA (4eq), the desired product Example 12 was obtained as a white solid (19.5 mg/61%).

### Example 13: [1-(5-fluoro-2-oxazol-2-yl-pyrimidin-4-yl)-4-piperidyl]-[(3S)-3-pyrazin-2-ylisoxazolidin-2-yl]methanone

Starting from I-01 (13 mg, 86 μmol) and I-17 (25 mg, 85 μmol), with DIPEA (4eq), the desired product Example 13 was obtained as a white solid (63%).

Chiral SFC: RT 1.37 min (100%, Method X).

### Example 14: [(3S)-3-pyrazin-2-ylisoxazolidin-2-yl]-[1-(4-thiazol-2-yi-1,3,5-triazin-2-yl)-4-piperidyl]methanone

Starting from I-01 (13 mg, 86 μmol) and I-18 (25 mg, 85 μmol) with DIPEA (4eq), the desired product Example 14 was obtained as a white solid (25 mg/69%).

Chiral SFC: RT 1.762 min (100%, Method AF).

### Example 15: [1-[4-(2-methylpyrazol-3-yl)pyrimidin-2-yl]-4-piperidyl]-[(3S)-3-(6-methyl-3-pyridyl)isoxazolidin-2-yl]methanone

Starting from I-03 TFA (32.27 mg, 100 μmol) and I-20.17 mg, 110 μmol, 1.1 eq) with DIPEA (4eq), the desired product Example 15 was obtained as a white solid (27 mg/62%).

### Example 16: [1-[2-(4-methylpyrazol-1-yl)pyrimidin-4-yl]-4-piperidyl]-[(3S)-3-(6-methyl-3-pyridyl)isoxazolidin-2-yl]methanone

Starting from I-03 TFA (32.27 mg, 100 μmol) and I-25 (32 mg, 110 μmol, 1.1 eq) with DIPEA (4eq), the desired product Example 16 was obtained as a white solid (29 mg/67%).

### Example 17: [1-[6-(4-methylpyrazol-1-yl)pyrimidin-4-yl]-4-piperidyl]-[(3S)-3-(6-methyl-3-pyridyl)isoxazolidin-2-yl]methanone

Starting from I-03 TFA (32.27 mg, 100 μmol) and I-26 (31.5 mg, 110 μmol, 1.1 eq) with DIPEA (4eq), the desired product Example 17 was obtained as a white solid (40 mg/92%).

### Example 18: [1-[6-(2-methylpyrazol-3-yl)pyrimidin-4-yl]-4-piperidyl]-[(3S)-3-(6-methyl-3-pyridyl)isoxazolidin-2-yl]methanone

Starting from I-03 TFA (32.27 mg, 100 μmol) and I-13 (30.1 mg, 110 μmol, 1.1 eq) with DIPEA (4eq), the desired product Example 18 was obtained as a white solid.

### Example 19: [1-[5-fluoro-4-(2-methylpyrazol-3-yl)pyrimidin-2-yl]-4-piperidyl]-[(3S)-3-(6-methyl-3-pyridyl)isoxazolidin-2-yl]methanone

Starting from I-03 TFA (32.3 mg, 100 μmol) and I-15 (32.1 mg, 110 μmol, 1.1 eq) with DIPEA (4eq), the desired product Example 19 was obtained as a white solid.

### Example 20: [(3S)-3-(5-fluoro-6-methyl-3-pyridyl)isoxazolidin-2-yl]-[1-[4-(2-methylimidazol-1-yl)pyrimidin-2-yl]-4-piperidyl]methanone

Starting from I-05 TFA (29.6 mg, 100 μmol) and I-22 (30.1 mg, 110 μmol, 1.1 eq) with DIPEA (4eq), the desired product Example 20 was obtained as a white solid (27.6 mg, 69%).

Chiral SFC: RT 1.024 min (100%, Method AN).

### Example 21: [(3S)-3-(5-fluoro-6-methyl-3-pyridyl)isoxazolidin-2-yl]-[1-[6-(2-methylimidazol-1-yl)pyrimidin-4-yl]-4-piperidyl]methanone

Starting from I-05 TFA (30.5 mg, 100 μmol) and I-12 (27.1 mg, 110 μmol, 1.1 eq) with DIPEA (4eq), the desired product Example 21 was obtained as a white solid (31.5 mg, 70%).

### Example 24: [1-[2-(2-methylpyrazol-3-yl)pyrimidin-4-yl]-4-piperidyl]-[(3S)-3-pyrazin-2-ylisoxazolidin-2-yl]methanone

Starting from I-01 TFA (30.5 mg, 100 μmol) and I-24 (27.1 mg, 110 μmol, 1.1 eq) with DIPEA (4eq), the desired product Example 24 was obtained as a white solid (31.5 mg, 70%).

### Example 26: (S)-(1-(4-(2-methyl-1H-imidazol-1-yl)-1,3,5-triazin-2-yl)piperidin-4-yl)(3-(pyrazin-2-yl)isoxazolidin-2-yl)methanone

Starting from I-01 HCl (146.16 mg, 728.40 μmol) and I-19 (200 mg, 693.71 μmol) with DIEA (4eq), the desired product Example 26 was obtained as a white solid (155 mg, 51%).

### Example 35: [1-[4-(2-methylimidazol-1-yl)-1,3,5-triazin-2-yl]piperidin-4-yl]-[(3S)-3-(6-methylpyridin-3-yl)-1,2-oxazolidin-2-yl]methanone

Starting from I-03 HCl Salt (146.16 mg, 1.05 eq) and I-19 (200 mg, 693.71 μmol, 1 eq) with DIEA (537.94 mg, 4.16 mmol, 724.99 μL, 6 eq), the desired product Example 35 was obtained as a white solid (155 mg, 51. %).

### Example 37: [(3S)-3-(6-methylpyridin-3-yl)-1,2-oxazolidin-2-yl]-[1-[4-(1,3-oxazol-2-yl)pyrimidin-2-yl]piperidin-4-yl]methanone

Starting from I-03 HCl Salt (1.1 eq) and I-12 (200 mg, 7687 μmol, 1 eq) with DIEA (6.87 mmol, 1.20 mL, 10 eq), the desired product Example 37 was obtained as a white gum (99 mg, 40%).

Chiral SFC: RT 0.979 min (98.66% ee) (Method X)

### Example 38: [(3S)-3-(6-methylpyridin-3-yl)-1,2-oxazolidin-2-yl]-[1-[4-(1,3-thiazol-2-yl)-1,3,5-triazin-2-yl]piperidin-4-yl]methanone

Starting from I-03 HCl Salt (1.1 eq) and I-18 (200 mg, 687 μmol, 1 eq) with DIEA (6.87 mmol, 1.20 mL, 10 eq), the desired product Example 38 was obtained as an off-white gum (99 mg, 40%).

Chiral SFC: RT 1.246 min (98.66% ee) (Method X)

### Example 45: [(3S)-3-(5-fluoro-6-methylpyridin-3-yl)-1,2-oxazolidin-2-yl]-[1-[4-(2-methylpyrazol-3-yl)-1,3,5-triazin-2-yl]piperidin-4-yl]methanone

Starting from I-05 HCl Salt (1.0 eq) and I-27 (243 mg, 842 μmol, 1 eq) with DIEA (7.01 mmol, 1.16 mL, 10 eq), the desired product Example 45 was obtained as a white solid (199 mg, 51%).

Chiral SFC: RT 1.478 min 99.99% ee) (Method AF)

### Example 47: 1-[2-[4-[(3S)-3-pyrazin-2-yi-1,2-oxazolidine-2-carbonyl]piperidin-1-yl]pyrimidin-4-yl]piperidin-2-one

Starting from I-01 TFA salt (1 eq) and I-37 (300 mg, 90% purity, 0.93 mmol, 1.0 eq) with DIEA (1.61 mL. 10 eq) the desired product Example 47 was obtained as off-white solid (173 mg, 43%).

Chiral SFC: RT 1.421 min (99.99% ee) (Method AL)

### Example 50: 1-[2-[4-[(3S)-3-(5-methylpyridin-3-yl)-1,2-oxazolidine-2-carbonyl]piperidin-1-yl]pyrimidin-4-yl]pyrrolidin-2-one

Starting from I-31 TFA salt (1 eq) and I-42 (475 mg, 1.5 mmol, 1.2 eq) with DIEA (3.63 mL. 16 eq) the desired product Example 50 was obtained as white solid (260.5 mg, 45%).

Chiral SFC: RT 0.715 min (99.99% ee) (Method Z)

### Example 54: 5-[2-[4-[(3S)-3-(2-methyl-1,3-oxazol-4-yl)-1,2-oxazolidine-2-carbonyl]piperidin-1-yl]pyrimidin-4-yl]-5-azaspiro[2.4]heptan-4-one

Starting from I-28 TFA salt (1 eq) and I-38 (564 mg, 1.1 mmol, 1.2 eq) with DIEA (1.88 mL. 9 eq) the desired product Example 54 was obtained as white solid (125.9 mg, 28%).

Chiral SFC: RT 1.324 min (99.99% ee) (Method AG)

### Example 55: 1-[2-[4-[(3S)-3-(5-methylpyrazin-2-yl)-1,2-oxazolidine-2-carbonyl]piperidin-1-yl]pyrimidin-4-yl]pyrrolidin-2-one

Starting from I-29 TFA salt (1 eq) and I-42 (351 mg, 1.21 mmol, 1.2 eq) with DIEA (1.88 mL. 109 eq) the desired product Example 55 was obtained as white solid (324.4 mg, 65%).

Chiral SFC: RT 1.441 min (99.99% ee) (Method AJ)

### Example 56: 1-[2-[4-[(3S)-3-(5-fluoro-6-methylpyridin-3-yl)-1,2-oxazolidine-2-carbonyl]piperidin-1-yl]pyrimidin-4-yl]pyrrolidin-2-one

Starting from I-05 HCl salt (1 eq) and I-42 (270 mg, 1.0 mmol, 1.2 eq) with DIEA (1.62 mL. 10 eq) the desired product Example 56 was obtained as white solid (328 mg, 77%).

Chiral SFC: RT 1.998 min (99.99% ee) (Method Z)

### Example 57: 5-[2-[4-[(3S)-3-pyrazin-2-yi-1,2-oxazolidine-2-carbonyl]piperidin-1-yl]pyrimidin-4-yl]-5-azaspiro[2.5]octan-4-one

Starting from I-01 TFA salt (1 eq) and I-39 (190 mg, 0.05 mmol, 1.2 eq) with DIEA (1.53 mL, 16 eq) the desired product Example 57 was obtained as white solid (140.2 mg, 56%).

Chiral SFC: RT 2.014 min (99.99% ee) (Method Z)

### Example 64: [1-[5-fluoro-4-(2-methylimidazol-1-yl)pyrimidin-2-yl]piperidin-4-yl]-[(3S)-3-(5-fluoropyridin-3-yl)-1,2-oxazolidin-2-yl]methanone

Starting from I-04 TFA salt (1 eq) and I-41 (341 mg, 1.1 mmol, 1.2 eq) with DIEA (3.12 mL, 16 eq) the desired product Example 64 was obtained as white solid (258.8 mg, 51%).

Chiral SFC: RT 1.325 min (99.99% ee) (Method X)

### Example 65: 3-[2-[4-[(3S)-3-(5-fluoro-6-methylpyridin-3-yl)-1,2-oxazolidine-2-carbonyl]piperidin-1-yl]pyrimidin-4-yl]-1,3-oxazolidin-2-one

Starting from I-05 TFA salt (1 eq) and I-40 (584 mg, 2.0 mmol, 1.2 eq, crude material) with DIEA (1.45 mL, 5 eq) the desired product Example 65 was obtained as white solid (194 mg, 25%).

Chiral SFC: RT 1.881 min (99.99% ee) (Method X)

### Example 67: [1-[5-fluoro-2-(1,3-oxazol-2-yl)pyrimidin-4-yl]piperidin-4-yl]-[(3S)-3-(6-methylpyridin-3-yl)-1,2-oxazolidin-2-yl]methanone

Starting from I-03 HCl salt (1 eq) and I-17 (119 mg, 0.41 mmol, 1.2 eq) with DIEA (1.06 mL, 15 eq) the desired product Example 67 was obtained as white solid (76.9 mg, 43%).

Chiral SFC: RT 2.402 min (99.99% ee) (Method Z)

### Example 77: [1-[5-fluoro-4-(2-methylimidazol-1-yl)pyrimidin-2-yl]piperidin-4-yl]-[(3S)-3-(6-methylpyridin-3-yl)-1,2-oxazolidin-2-yl]methanone

Starting from I-03 TFA salt (1 eq) and I-41 (440 mg, 1.4 mmol, 1.2 eq, crude material) with DIEA (1.45 mL, 5 eq) the desired product Example 77 was obtained as white solid (272 mg, 42%).

Chiral SFC: RT 1.248 min (99.99% ee) (Method X)

### Example 81: 1-[2-[4-[(3S)-3-(6-methylpyridin-3-yl)-1,2-oxazolidine-2-carbonyl]piperidin-1-yl]pyrimidin-4-yl]pyrrolidin-2-one

Starting from I-03 TFA salt (1 eq) and I-42 (250 mg, 0.88 mmol, 1.2 eq, crude material) with DIEA (1.5 mL, 10 eq) the desired product Example 81 was obtained as white solid (294.3 mg, 78%).

Chiral SFC: RT 2.096 min (99.99% ee) (Method AL)

### Example 82: 1-[6-[4-[(3S)-3-(6-methylpyridin-3-yl)-1,2-oxazolidine-2-carbonyl]piperidin-1-yl]pyrimidin-4-yl]pyrrolidin-2-one

Starting from I-03 TFA salt (1 eq) and I-43 (250 mg, 0.86 mmol, 1.2 eq, crude material) with DIEA (1.50 mL, 10 eq) the desired product Example 82 was obtained as white solid (237.7 mg, 61%).

Chiral SFC: RT 1.355 min (99.99% ee) (Method AC)

### Example 86: 1-[6-[4-[(3S)-3-(5-fluoropyridin-3-yl)-1,2-oxazolidine-2-carbonyl]piperidin-1-yl]pyrimidin-4-yl]pyrrolidin-2-one

Starting from I-04 TFA salt (1 eq, crude material, 29% purity) and I-43 (260 mg, 897 mmol, 1.2 eq, crude material) with DIEA (2.40 mL, 20 eq) the desired product Example 86 was obtained as white solid (141 mg, 46%)

Chiral SFC: RT 1.390 min (99.99% ee) (Method X)

### Example 93: (3R)-3-methoxy-1-[2-[4-[(3S)-3-(6-methylpyridin-3-yl)-1,2-oxazolidine-2-carbonyl]piperidin-1-yl]pyrimidin-4-yl]piperidin-2-one and

### Example 94: (3S)-3-methoxy-1-[2-[4-[(3S)-3-(6-methylpyridin-3-yl)-1,2-oxazolidine-2-carbonyl]piperidin-1-yl]pyrimidin-4-yl]piperidin-2-one

Starting from I-03 TFA salt (1 eq) and I-44 (1.12 mL of crude solution) with DIEA (2.56 mL, 5 eq) the desired product was obtained as a mixture of Example 93+Example 94 as white solid. The two diastereomers were separated by SFC (241 mg, 42%). Column: DAICEL CHIRALPAK IC (250 mm*30 mm, 10 um); mobile phase: [CO₂—MeOH (1‰ NH₃—H₂O)]; B %: 60%, isocratic elution mode).
Example 93: RT 2.02 (49.29 mg, 99.9% e.e (Method AL)
Example 94: RT: 2.48 (50.64 mg, 98.5% e.e.) (Method AL)

### Example 95: 1-[2-[4-[(3S)-3-(2-methyl-1,3-oxazol-4-yl)-1,2-oxazolidine-2-carbonyl]piperidin-1-yl]pyrimidin-4-yl]pyrrolidin-2-one

Starting from I-28 TFA salt (1 eq) and I-42 (564 mg, 65% purity, 1.3 mmol, 1.2 eq) with DIEA (1.55 mL, 12 eq) the desired product Example 95 was obtained as white solid (107 mg, 32%).

Chiral SFC: RT 1.416 min (99.99% ee) (Method AH)

### Example 96: (3R)-1-[2-[4-[(3S)-3-(5-fluoropyridin-3-yl)-1,2-oxazolidine-2-carbonyl]piperidin-1-yl]pyrimidin-4-yl]-3-methoxypiperidin-2-one and

### Example 97: (3S)-1-[2-[4-[(3S)-3-(5-fluoropyridin-3-yl)-1,2-oxazolidine-2-carbonyl]piperidin-1-yl]pyrimidin-4-yl]-3-methoxypiperidin-2-one

Starting from I-05 TFA salt (1 eq) and I-44 (1.77 mL of crude solution) with DIEA (1.45 mL, 5 eq) the desired product was obtained as a mixture of Example 96+Example 97 as white solid (243 mg, 40%). The two diastereomers were separated by SFC and NPLC: condition: column: DAICEL CHIRALPAK IC (250 mm*30 mm, 10 um); mobile phase: [CO₂—MeOH (1‰ NH₃H2O)]; B %: 65%, isocratic elution mode). And the first fraction was further purified by NPLC (column: Welch Ultimate XB—SiOH 250*50*10 um; mobile phase: [Hexane-EtOH]; gradient: 1%-30% B over 15 min).

Example 96: RT 1.82 (121.00 mg, 99.9% e.e., (Method AM), 49.7% yield) as an off-white solid.

Example 97: RT 1.33 (79.66 mg, 99.9% e.e., (Method AM) 32.6% yield) as an off-white solid.

### Example 102: [1-[4-(5-amino-2-methylpyrazol-3-yl)pyrimidin-2-yl]piperidin-4-yl]-[(3S)-3-(5-fluoro-6-methylpyridin-3-yl)-1,2-oxazolidin-2-yl]methanone

STEP 1: Starting from I-05 TFA salt (1 eq) and I-45 (490 mg, 61% purity, 0.88 mmol, 1.2 eq, crude material) with pyridine (3 eq) in dichloromethane (5 mL) to afford the desired intermediate as white solid (350 mg, 69% purity, 55%).

LCMS: m/z=497.3; RT 0.553 (method Q)

### Step 2

To a solution of the crude intermediate from step 1 (300 mg, 69.0% purity, 0.000417 mol, 1.00 eq.) and NH₄Cl (0.376 g, 0.0125 mol, 30.0 eq.) in MeOH (10 mL) was added Zn dust (0.267 g, 0.00417 mol, 10.0 eq.) in portions. The mixture was stirred at 25° C. for 1 h. The mixture was filtered and washed with MeOH (10 mL). The filtrate was concentrated under reduced pressure to give a residue which was purified by HPLC to afford Example 102 (65.91 mg, 34% yield) as a white solid.

Chiral SFC: RT 1.734 min (100% ee) (Method X)

Following examples were synthesized in a specific route comprising a coupling step different from the coupling methods A to K above

### Example 111: [1-[4-(4-amino-5-methylpyrazol-1-yl)pyrimidin-2-yl]piperidin-4-yl]-[(3S3-(5-fluoro-6-methylpyridin-3-yl)-1,2-oxazolidin-2-yl]methanone

### Step 1. Synthesis of ethyl [2-[4-[(3S)-3-(5-fluoro-6-methyl-3-pyridyl)isoxazolidine-2-carbonyl]-1-piperidyl]pyrimidin-4-yl]-5-methyl-pyrazole-4-carboxylate

The title compound was prepared in a similar manner to Example 1, using I-10 (503 mg, 44% purity, 0.69 mmol), Ar—Cl 37 (200 mg, 1 eq), DIEA (10 eq) in ACN (4 mL) at 25° C. The residue was purified by prep-HPLC to afford the title compound as a white solid (340 mg, 86%).

¹H NMR (400 MHz, CD₃OD) δ=8.41 (d, J=5.6 Hz, 1H), 8.24 (s, 1H), 8.01 (s, 1H), 7.49 (dd, J=10.4, 1.6 Hz, 1H), 7.08 (d, J=5.6 Hz, 1H), 5.42 (t, J=7.6 Hz, 1H), 4.79-4.68 (m, 2H), 4.37 (td, J=8.0, 3.2 Hz, 1H), 4.32 (q, J=7.2 Hz, 2H), 4.07-3.99 (m, 1H), 3.23-3.07 (m, 3H), 3.01-2.93 (m, 4H), 2.48 (d, J=2.8 Hz, 3H), 2.41-2.31 (m, 1H), 2.01-1.97 (m, 1H), 1.90-1.79 (m, 1H), 1.75-1.59 (m, 2H), 1.37 (t, J=7.2 Hz, 3H).

### Step 2. Synthesis of [2-[4-[(3S)-3-(5-fluoro-6-methyl-3-pyridyl)isoxazolidine-2-carbonyl]-1-piperidyl]pyrimidin-4-yl]-5-methyl-pyrazole-4-carboxylic acid

The title compound was synthesized following the general procedure for ester hydrolysis to afford the above intermediate as a white solid (90 mg, 28%).

LCMS: m/z=496.3 [M+H]+, RT 0.459 (Method Q)

¹H NMR (400 MHz, CD₃OD) δ=8.40 (d, J=5.6 Hz, 1H), 8.24 (s, 1H), 8.00 (s, 1H), 7.49 (dd, J=10.0, 1.6 Hz, 1H), 7.08 (d, J=5.6 Hz, 1H), 5.42 (t, J=7.6 Hz, 1H), 4.77-4.70 (m, 2H), 4.37 (td, J=7.6, 3.2 Hz, 1H), 4.07-3.99 (m, 1H), 3.24-3.07 (m, 3H), 3.02-2.93 (m, 4H), 2.48 (d, J=2.8 Hz, 3H), 2.41-2.31 (m, 1H), 2.01 (d, J=13.2 Hz, 1H), 1.90-1.79 (m, 1H), 1.75-1.60 (m, 2H).

Chiral SFC: RT 0.960 min (100% ee) (Method N)

### Step 3. Synthesis of tert-butyl N-[1-[2-[4-[(3S)-3-(5-fluoro-6-methyl-3-pyridyl)isoxazolidine-2-carbonyl]-1-piperidyl]pyrimidin-4-yl]-5-methyl-pyrazol-4-yl]carbamate

To a solution [2-[4-[(3S)-3-(5-fluoro-6-methyl-3-pyridyl)isoxazolidine-2-carbonyl]-1-piperidyl]pyrimidin-4-yl]-5-methyl-pyrazole-4-carboxylic acid (70.0 mg, 0.000141 mol, 1.00 eq.) in t-BuOH (2 mL) was added TEA (0.0589 mL, 0.000424 mol, 3.00 eq.) and DPPA (0.0971 g, 0.000353 mol, 2.50 eq.) in sequence under N2 atmosphere. The resulting mixture was stirred at 85° C. for 16 h. The mixture was concentrated under reduced pressure to give a residue. The residue was purified by prep-TLC (EtOAc/MeOH=10/1) to afford the tittle (35.0 mg, 6.18 umol, 43.7% yield) as a gray solid.

LCMS RT 0.501 min, (ESI) m/z=567.4 [M+H]+(Method Q).

### Step 4. Synthesis of [1-[4-(4-amino-5-methylpyrazol-1-yl)pyrimidin-2-yl]piperidin-4-yl]-[(3S)-3-(5-fluoro-6-methylpyridin-3-yl)-1,2-oxazolidin-2-yl]methanone (Example 111)

A mixture of tert-butyl N-[1-[2-[4-[(3S)-3-(5-fluoro-6-methyl-3-pyridyl)isoxazolidine-2-carbonyl]-1-piperidyl]pyrimidin-4-yl]-5-methyl-pyrazol-4-yl]carbamate (35.0 mg, 61.8 umol, 1.00 eq.) in HCl (4 M in dioxane, 0.5 mL) was stirred at 25° C. for 1 h. The mixture was directly concentrated and then dissolved into NaHCO₃ solution (sat. aq., 0.5 mL). The mixture obtained was purified by prep-HPLC (column: Waters Xbridge 150*25 mm*5 um; mobile phase: water-ACN; B %: 18%-48% over 5 min; flowrate: 30 mL/min) to afford Example 111 (3.97 mg, 13.8% yield) as a yellow solid.

Chiral SFC: RT 0.737 min (100% ee) (Method N)

### Example 112: [1-[4-(4-amino-5-methylpyrazol-1-yl)pyrimidin-2-yl]piperidin-4-yl]-[(3S)-3-(6-methylpyridin-3-yl)-1,2-oxazolidin-2-yl]methanone

The title compound was synthesized in a similar manner to Example 111, but using I-08 (499 mg, 1.8 mmol) to afford Example 112 as a white solid (110 mg, 41%) Chiral SFC: RT 1.814 min (100% ee) (Method X)

### Example 115 3-[5-fluoro-2-[4-[(3S)-3-(5-fluoro-6-methylpyridin-3-yl)-1,2-oxazolidine-2-carbonyl]piperidin-1-yl]pyrimidin-4-yl]-1,3-oxazolidin-2-one

Starting from I-05 TFA salt (1 eq) and I-46 (200 mg, 0.65 mmol) with DIEA (0.9 mL, 8 eq) the desired product Example 114 was obtained as white solid (175.7 mg, 57%).

Chiral SFC: RT 1.757 min (99.99% ee) (Method X)

### Example 116: 3-[5-fluoro-2-[4-[(3S)-3-(5-methylpyridin-3-yl)-1,2-oxazolidine-2-carbonyl]piperidin-1-yl]pyrimidin-4-yl]-1,3-oxazolidin-2-one

The title compound was prepared in a similar manner to Example 1, using I-34 (376 mg, 61% purity), Ar—Cl 5 (200 mg, 84%), TEA (10 eq) in DMF (4 mL) at 65° C. The residue was purified by prep-HPLC to afford Example 116 as a white solid (106 mg, 30%).

Chiral SFC: RT 1.712 min (100% ee) (Method X).

### Solubility Assay

Prior to the solubility measurement of a compound batch, its purity was determined through UHPLC/MS.

Solubility buffer system was 50 mM phosphate buffer pH 7.4

Solubility was determined from DMSO Stock solutions (10 mM), using a high throughput HPLC method as described below.
Instrument: Waters Acquity UPLC with Binary Pump
Software Waters Empower 3 (Build 3471)
Gradient: Eluent A: Water+25 mM Ammonium Acetate, Eluent B: Acetonitrile.

98% A/2% B for 0.05 min.; then from 98% A to 98% B in 2.3 min, then 98% B for 0.65 min, then to 98% A in 0.05 min, flow rate: 0.9 ml/min, column: 2.1×50 mm Waters ACQUITY UPLC BEH C18, 1.7 μm, 55° C.

UV data: retention time ad λ=220 nm given in min

### Determination of Actual Sample Concentration by UPLC-ELSD:

Thawing of microtiter plates with samples, followed by centrifugation (10 min, 4000 rcf)
Dilution of DMSO stock solution 1:40 (5+195 μL) with DMSO
Determination of concentration by UPLC-ELSD (Evaporative Light Scattering Detector), injection volume: 4 μL

### Calibration

UPLC-UV_{220 nm} analysis of 1:40 diluted DMSO stock solutions from UPLC-ELSD run
Determination of peak area-to-real concentration (by UPLC-ELSD) relationship for calibration

### Solubility Determination:

Replating of DMSO stock solutions in a U-shape multi-titer plate (96 wells) with the target amount of 100 μg of compound in each well (Condition: concentration in DMSO stock solution measured via ELSD >3 mM and Purity >75% by LC-UV_{220 nm})
Evaporation of DMSO solvent in vacuo (SpeedVac)
Addition of 160 μL buffer and a magnetic bead to each well, plate sealed (peeling foil) with heat sealer
Overnight shaking (for 16 hours) at 25° C., protected from light on an Eppendorf thermomixer with 600 rpm
Centrifugation (5 min, 4000 rcf) followed by manual replating into a filter plate (Millipore MultiScreen Filter Plate 0.45 μm low-binding hydrophilic PTFE)
Filtering by centrifugation (15 min at 4000 rcf) into a V-shape multi-titer plate, plate sealed (piercing foil) with heat sealer
UPLC-UV_{220 nm} analysis of filtrated supernatant, injection of 2-8 μL (dependent on aqueous solubility prediction by ACD software) to reach an amplitude of 1 absorbance unit (A.U.) in the photo diode array detector
Calculation of filtrated supernatant's concentration based on peak area-to-real concentration ratio from previous calibration run

Results of Solubility determination are shown in Table 4 hereinafter.

TABLE 4

| | Solubility |
|---|---|
| | pH 7.4 |
| Example | (μM) |
| 1 | >1150.8 |
| 2 | >1175.2 |
| 3 | 708 |
| 4 | >1180.7 |
| 5 | >1127.6 |
| 6 | >1132.6 |
| 7 | >1137.6 |
| 8 | 327.5 |
| 9 | 172.8 |
| 10 | 215.8 |
| 11 | >2779.0 |
| 12 | >1175.3 |
| 13 | >1175.3 |
| 14 | >1177.9 |
| 15 | 14.3 |
| 16 | 168.9 |
| 17 | 12.8 |
| 18 | >1153.4 |
| 19 | 7.1 |
| 20 | 21.4 |
| 21 | >1107.4 |
| 22 | >1105.0 |
| 23 | >1135.0 |
| 24 | >1189.1 |
| 25 | 69.2 |
| 26 | >1186.4 |
| 27 | >1132.5 |
| 28 | >1189.1 |
| 29 | >1189.1 |
| 30 | >1189.1 |
| 31 | >1189.1 |
| 32 | >1178.0 |
| 33 | >1088.0 |
| 34 | >1088.0 |
| 34 I | >1088.0 |
| 35 | 202.3 |
| 37 | 34.7 |
| 38 | 274.7 |
| 39 | >1119.8 |
| 40 | 136.2 |
| 41 | >1071.7 |
| 42 | 338.4 |
| 43 | >1044.9 |
| 44 | 380.1 |
| 45 | 904.3 |
| 46 | >1135.1 |
| 47 | >1142.9 |
| 48 | 231.1 |
| 49 | >1105.0 |
| 50 | >1145.5 |
| 51 | 142.5 |
| 52 | 321.4 |
| 53 | 144 |
| 54 | >1105.0 |
| 55 | >1142.9 |
| 56 | >1100.1 |
| 57 | >1078.7 |
| 58 | 162.2 |
| 59 | 216.9 |
| 60 | <2.1 |
| 61 | 103.6 |
| 62 | >1097.7 |
| 63 | >1044.8 |
| 64 | >1097.8 |
| 65 | 209.2 |
| 66 | 4.3 |
| 67 | >1140.4 |
| 69 | 601 |
| 70 | 120.6 |
| 71 | >1135.1 |
| 72 | 217.7 |
| 73 | 88.5 |
| 74 | 38.7 |
| 75 | 98.4 |
| 76 | 39.3 |
| 77 | >1107.4 |
| 78 | >1088.0 |
| 79 | >1088.0 |
| 80 | >1088.0 |
| 81 | >1145.5 |
| 82 | >1145.5 |
| 83 | 233.6 |
| 84 | >1076.3 |
| 85 | >1081.0 |
| 86 | >1135.1 |
| 87 | >1145.5 |
| 88 | >1076.3 |
| 89 | 98 |
| 90 | >1049.2 |
| 91 | >1130.1 |
| 92 | 157.3 |
| 93 | >1040.5 |
| 94 | >1040.5 |
| 95 | >1172.4 |
| 96 | >1031.9 |
| 97 | >1031.9 |
| 98 | 288.9 |
| 99 | >1169.7 |
| 100 | 269.4 |
| 102 | >1071 |
| 103 | >1040 |
| 104 | >1032 |

The compounds according to the disclosure show solubility values at pH7.4 which are mostly higher than 200 μM. Advantageously, most of the compounds exhibit solubility higher than 1000 μM.

### Biological Activity

### Evaluation of Receptor-Interacting Protein Kinase 1 Inhibition.

The catalytic activity of RIPK1 was measured by monitoring the conversion of Adenosine triphosphate (ATP) to Adenosine diphosphate (ADP) due to autophosphorylation using an ADP-Glo kinase kit (Promega, catalog no. V9104).

In detail, 2 μl recombinantly produced hRIPK1 (aa 1-375) fusion protein (end concentration 3.6 μg/ml) and 2 μl compound (end concentration 33300-1.69 nM; DMSO end concentration 1%) were incubated for 30 minutes at room temperature and then 2 μl ATP (ADP Glo kit, end concentration 50 μM) were added. After another 240 minutes incubation at room temperature, 5 μl Promega ADP-Glo reagent I was added to quench the reaction and deplete unconsumed ATP. After an incubation period of 30 minutes, 10 μl Promega ADP-Glo detection reagent II was added resulting in conversion of ADP to ATP, which generates a light-reaction between luciferase and luciferin. Luminescence was quantified after 30 minutes with a Pherastar FS (BMG LABTECH, Ortenberg).

For the dose response experiments an IC₅₀ value with 95% confidence interval was calculated using the 4-parameter logistic model according to Ratkowsky and Reedy with constraints for lower and upper asymptotes at 0% and 100%. The adjustment was obtained by nonlinear regression using the Levenberg Marquardt algorithm.

### Cellular Assay in U937 Cells to Measure the Activity of RIPK1-Inhibitors on Cell Death (Necroptosis).

Upon TNF-Receptor I ligation, Ser/Thr kinase RIPK1 is recruited to a transient receptor complex I. Upon modification of RIPK1 which promotes activation of RIPK1, complex IIb can form, that involves recruitment of RIPK3 and MLKL (mixed lineage-kinase domain-like protein) which then translocates from the cytosol to the plasma membrane to execute cell death (Cai, Z. et al, Nat. Cell Biol. (2014)16:55-65).

Cell death was quantified in 96 well plates by determination of the amount of live cell using a CellTiter 96 AQueous reagent (Promega), a calorimetric method to measure the number of live cells by reducing tetrazolium compound [3-(4,5-dimethylthiazol-2-yl)-5-(3-carboxymethoxyphenyl)-2-(4-sulfophenyl)-2H-tetrazolium, inner salt; MTS] into formazan. Absorbance of formazan was read at 490 nm. The inhibitory activity of the test compound was quantified in a concentration response curve (CRC) experiment.

Compounds were obtained as 10 mM stock solutions and were diluted 1 to 10 volumes with DMSO to yield a 1 mM solution. From this solution 2 μl were diluted with 998 μl growth medium. 100 μl of 2 μM compound solution was further diluted sequentially with a dilution factor of 2.5 by adding 150 μl growth medium. A total of 10 concentrations were tested ranging from 10 μM to 0.26 nM or from 1 μM to 0.07 nM.

U937 cells were cultured in RPMI1640 Glutamax and 10% heat inactivated FBS. 50 μl cell suspension containing 1×106 cells/ml supplemented with 50 μM zVAD.fmk (Benzyloxycarbonyl-Val-Ala-Asp (OMe) fluoromethylketone) and 100 ng/ml recombinant human TNFα were dispensed in each well of a 96-well plate. 50 μl of compound dilutions, as described in Cellular assay in U937 cells, were added and the cell suspension incubated overnight (18 to 24 hrs) at 37° C., 5% CO₂ in a humidified atmosphere (95% rH). High (no compound) and low control (no TNFα, zVAD.fmk) were tested with 7 replicates; all compound concentrations were tested in duplicates on each experimental plate.

CellTiter 96 Aqueous reagent was mixed (100 μl PMS (phenazine methosulfate) solution/2 ml MTS (3-(4,5-dimethyldiazol-2-yl)-5-(3-carboxymethoxyphenyl)-2-(4-sulfophenyl)-2H-tetrazolium, inner salt) solution) and 20 μl were added per well. After 4 hrs incubation at 37° C. (5% CO₂ 95% rH) optical density was measured at 490 nm on a microplate reader (Tecan Infinite M1000).

The % inhibition is expressed as percentage of the maximal inhibition value obtained in the absence of TNFα/zVAD.fmc. For each dose response experiment an IC₅₀ value with 95% confidence interval was calculated using the 4-parameter logistic model according to Ratkowsky and Reedy without constraints using an internal application (Biost@t-Speed LTS V2.3).

Results of biological activity are shown in Table 5 (ADP-Glo IC₅₀ (μM) and U937 IC₅₀ (μM)).

TABLE 5
activity data of all examples

| | ADP Glo | U937 cell |
|---|---|---|
| Cpd | assay IC₅₀ | assay IC₅₀ |
| No | [nM] | [nM] |
| 1 | 0.028 | 0.037 |
| 2 | 0.039 | 0.05 |
| 3 | 0.015 | 0.028 |
| 4 | 0.575 | 6.66 |
| 5 | 0.015 | 0.005 |
| 6 | 0.019 | 0.008 |
| 7 | 0.017 | 0.009 |
| 8 | 0.016 | 0.003 |
| 9 | 0.015 | 0.029 |
| 10 | 0.013 | 0.004 |
| 11 | 0.014 | 0.01 |
| 12 | 0.013 | 0.001 |
| 13 | 0.017 | 0.027 |
| 14 | 0.021 | 0.009 |
| 15 | 0.009 | 0.001 |
| 16 | 0.029 | 0.053 |
| 17 | 0.026 | 0.039 |
| 18 | 0.024 | 0.07 |
| 19 | 0.01 | 0 |
| 20 | 0.015 | 0.001 |
| 21 | 0.021 | 0.073 |
| 22 | 0.014 | 0.002 |
| 23 | 0.01 | 0.001 |
| 24 | 0.029 | 0.009 |
| 25 | 0.003 | 0.015 |
| 26 | 0.016 | 0.005 |
| 27 | 0.011 | 0.001 |
| 28 | 0.022 | 0.043 |
| 29 | 0.02 | 0.006 |
| 30 | 0.008 | 0.003 |
| 31 | 0.027 | 0.032 |
| 32 | 0.014 | 0.001 |
| 33 | 0.0384 | 0.0164 |
| 34 | 0.054 | 0.0529 |
| 34 I | 0.01 | 0.08 |
| 35 | 0.013 | 0.002 |
| 36 | 0.023 | 0.025 |
| 37 | 0.045 | 0.041 |
| 38 | 0.011 | 0.051 |
| 39 | 0.029 | 0.02 |
| 40 | 0.022 | 0.04 |
| 41 | 0.038 | 0.044 |
| 42 | 0.022 | 0.012 |
| 43 | 0.038 | 0.057 |
| 44 | 0.022 | 0.025 |
| 45 | 0.019 | 0.001 |
| 46 | 0.022 | 0.018 |
| 47 | 0.029 | 0.071 |
| 48 | 0.011 | 0.003 |
| 49 | 0.024 | 0.015 |
| 50 | 0.023 | 0.033 |
| 51 | 0.021 | 0.026 |
| 52 | 0.012 | 0.024 |
| 53 | 0.01 | 0.009 |
| 54 | 0.022 | 0.051 |
| 55 | 0.022 | 0.078 |
| 56 | 0.02 | 0.021 |
| 57 | 0.025 | 0.059 |
| 58 | 0.016 | 0.065 |
| 59 | 0.017 | 0.08 |
| 60 | 0.063 | 0.506 |
| 61 | 0.015 | 0.032 |
| 62 | 0.03 | 0.041 |
| 63 | 0.036 | 0.044 |
| 64 | 0.014 | 0.001 |
| 65 | 0.022 | 0.006 |
| 66 | 0.037 | 3.444 |
| 67 | 0.057 | 0.135 |
| 68 | 9.801 | 10 |
| 69 | 0.023 | 0.009 |
| 70 | 0.028 | 0.107 |
| 71 | 0.214 | 2.337 |
| 72 | 0.025 | 0.058 |
| 73 | 0.018 | 0.551 |
| 74 | 0.023 | 0.175 |
| 75 | 0.019 | 0.058 |
| 76 | 0.032 | 0.485 |
| 77 | 0.012 | 0.001 |
| 78 | 0.028 | 0.032 |
| 79 | 0.996 | n.a. |
| 80 | 1.549 | n.a. |
| 81 | 0.024 | 0.052 |
| 82 | 0.731 | 10 |
| 83 | 0.021 | 0.012 |
| 84 | 10.283 | 8.128 |
| 85 | 0.143 | 0.517 |
| 86 | 0.167 | 0.78 |
| 87 | 5.2 | >10 |
| 88 | 0.19 | 1.364 |
| 89 | 0.04 | 0.32 |
| 90 | 19.068 | 4.426 |
| 91 | 0.014 | 0.007 |
| 92 | 0.032 | 0.018 |
| 93 | 0.268 | 0.436 |
| 94 | 0.108 | 0.216 |
| 95 | 0.024 | 0.026 |
| 96 | 0.096 | 0.149 |
| 97 | 0.059 | 0.086 |
| 98 | 0.067 | 0.269 |
| 99 | 0.078 | 0.265 |
| 100 | 0.022 | 0.012 |
| 101 | 0.108 | 2.6 |
| 102 | 0.014 | 0.001 |
| 103 | 0.021 | 0.006 |
| 104 | 0.015 | 0.002 |
| 105 | 0.025 | — |
| 109 | 0.061 | — |
| 110 | 0.05 | — |
| 112 | 0.025 | — |
| 114 | 0.019 | — |

All the compounds according to the disclosure are potent RIPK1 inhibitors as they exhibit a reducing catalytic activity of RIPK1, highlighted by ADP Glo assay, with IC₅₀ lower than 500 nM. Most of the compounds even exhibit IC₅₀ values below 50 nM. Advantageously, most of the compounds exhibit IC₅₀ values below 30 nM, and even lower than 20 nM.

All the compounds according to the disclosure are potent RIPK1 inhibitors as they exhibit a cell death (necroptosis) in U937 cells, with IC₅₀ lower than 7000 nM. Most of the compounds even exhibit IC₅₀ values below 75 nM, and even lower than 50 nM. Advantageously, most of the compounds exhibit IC₅₀ values below 30 nM.

1. A compound of formula (I): 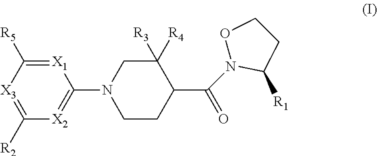
wherein
X₁, X₂, and X₃ are independently selected from CR₆ or N;
R₁ is a 5 or 6-membered heteroaryl group, wherein said heteroaryl is optionally substituted by 1 or 2 groups independently selected from: a halogen, a (C₁-C₆)alkyl group, and a —CN group;
R₂ is:
a 4-, 5- or 6-membered heteroaryl in which 1 to 3 ring atoms are independently selected from nitrogen, oxygen or sulfur, and wherein said heteroaryl is optionally substituted by 1 or 2 R₇;
a 5 or 6-membered heterocycle in which 1 to 3 ring atoms are independently selected from nitrogen and oxygen, and wherein the heterocycle is optionally substituted by 1, 2, or 3 R₈; or
a spiro(C₇-C₁₀)heterobicyclic in which 1 or 2 ring atoms are selected from nitrogen or oxygen, and wherein the heterocycle is optionally substituted by 1 or 2 R₉;
R₃ is H or a (C₁-C₄)alkyl group;
R₄ is H or a (C₁-C₄)alkyl group;
or alternatively R₃ and R₄ together form with the carbon atom to which they are attached a cyclopropyl or a cyclobutyl ring;
R₅ and R₆ are independently H, a (C₁-C₆)alkyl group, or a halogen;
each R₇ independently is a (C₁-C₆)alkyl group, a halogen, —NH₂, or —OH;
each R₉ independently is a (C₁-C₆)alkyl group, a halogen, or an oxo; and
each R₈ is independently OH, an oxo, a halogen, a (C₁-C₆)alkyl, O—(C₁-C₆)alkyl, —NH₂, —NH(C₁-C₆)alkyl, —N[(C₁-C₆)alkyl]₂, or a (C₁-C₆)fluoroalkyl;
or a pharmaceutically acceptable salt, solvate, or stereoisomer thereof.

2. The compound of claim 1, or a pharmaceutically acceptable salt thereof, wherein R₁ is a pyridinyl, a pyrazinyl, a pyrimidinyl, an oxazolyl, or a thiazolyl group, each of which is optionally substituted by 1 or 2 groups independently selected from: a halogen, a (C₁-C₂)alkyl group, and a —CN group.

3. The compound of claim 1, or a pharmaceutically acceptable salt thereof, wherein R₂ is an imidazolyl, a pyrazolyl, a triazolyl, an oxazolyl, a thiazolyl, a pyridinyl, or a pyrimidinyl group, each of which is optionally substituted by 1 or 2 R₇, wherein R₇ is independently selected from: a CH₃ group and a halogen.

4. The compound of claim 1, or a pharmaceutically acceptable salt thereof wherein R₂ is a piperidinyl, a morpholinyl, a pyrrolidinyl, a oxazolidinyl, an azetidinyl, or an oxetanyl group, each of which is optionally substituted by 1 or 2 R₈, wherein R₈ is independently selected from OH, oxo, a halogen, a (C₁-C₂)alkyl group, a O—(C₁-C₂)alkyl group, and CF₃.

5. The compound of claim 1, or a pharmaceutically acceptable salt thereof, wherein R₂ is a 7-oxa-2-azaspiro[3.5]nonan-2-yl group, a 5-azaspiro[2.5]octan-4-on-yl group, a 5-azaspiro[2.4]heptan-4-on-yl group, or a 2-oxa-7-azaspiro[3.5]nonan-7-yl group , each of which is optionally substituted by 1 or 2 groups selected from methyl and fluorine.

6. The compound of claim 1, or a pharmaceutically acceptable salt thereof, wherein:
R₃ is H or a (C₁-C₂)alkyl group;
R₄ is H, or a (C₁-C₂)alkyl group; or
alternatively, R₃ and R₄ are taken together with the carbon atom to which they are attached to form a cyclopropyl ring.

7. The compound of claim 1, wherein the compound is selected from:
(1) (S)-(3-(5-fluoropyridin-3-yl)isoxazolidin-2-yl)(1-(6-(pyridin-3-yl)pyrimidin-4-yl)piperidin-4-yl)methanone,
(2) (S)-3-(2-(4-(3-(pyrazin-2-yl)isoxazolidine-2-carbonyl)piperidin-1-yl)pyrimidin-4-yl)oxazolidin-2-one,
(3) (S)-1-(4-(4-(3-(pyrazin-2-yl)isoxazolidine-2-carbonyl)piperidin-1-yl)pyrimidin-2-yl)pyrrolidin-2-one,
(4) (S)-1-(2-(4-(3-(pyrazin-2-yl)isoxazolidine-2-carbonyl)piperidin-1-yl)pyrimidin-4-yl)pyrrolidin-2-one,
(5) 3-[5-fluoro-2-[4-[(3S)-3-pyrazin-2-ylisoxazolidine-2-carbonyl]-1-piperidyl]pyrimidin-4-yl]oxazolidin-2-one,
(6) 1-[5-fluoro-2-[4-[(3S)-3-pyrazin-2-ylisoxazolidine-2-carbonyl]-1-piperidyl]pyrimidin-4-yl]pyrrolidin-2-one,
(7) 1-[6-(2-methylimidazol-1-yl)pyrimidin-4-yl]-4-piperidyl]-[(3S)-3-(2-methylthiazol-4-yl)isoxazolidin-2-yl]methanone,
(8) 1-[4-(2-methylpyrazol-3-yl)-1,3,5-triazin-2-yl]-4-piperidyl]-[(3S)-3-(6-methyl-3-pyridyl)isoxazolidin-2-yl]methanone,
(9) [1-[4-(4-methylpyrazol-1-yl)pyrimidin-2-yl]-4-piperidyl]-[(3S)-3-(6-methyl-3-pyridyl)isoxazolidin-2-yl]methanone,
(10) [1-(5-fluoro-4-pyrazol-1-yl-pyrimidin-2-yl)-4-piperidyl]-[(3S)-3-(6-methyl-3-pyridyl)isoxazolidin-2-yl]methanone,
(11) [1-(4-oxazol-2-ylpyrimidin-2-yl)-4-piperidyl]-[(3S)-3-pyrazin-2-ylisoxazolidin-2-yl]methanone,
(12) [1-(5-fluoro-4-oxazol-2-yl-pyrimidin-2-yl)-4-piperidyl]-[(3S)-3-pyrazin-2-ylisoxazolidin-2-yl]methanone,
(13) [1-(5-fluoro-2-oxazol-2-yl-pyrimidin-4-yl)-4-piperidyl]-[(3S)-3-pyrazin-2-ylisoxazolidin-2-yl]methanone,
(14) [(3S)-3-pyrazin-2-ylisoxazolidin-2-yl]-[1-(4-thiazol-2-yl-1,3,5-triazin-2-yl)-4-piperidyl]methanone,
(15) [1-[4-(2-methylpyrazol-3-yl)pyrimidin-2-yl]-4-piperidyl]-[(3S)-3-(6-methyl-3-pyridyl)isoxazolidin-2-yl]methanone,
(16) [1-[2-(4-methylpyrazol-1-yl)pyrimidin-4-yl]-4-piperidyl]-[(3S)-3-(6-methyl-3-pyridyl)isoxazolidin-2-yl]methanone,
(17) [1-[6-(4-methylpyrazol-1-yl)pyrimidin-4-yl]-4-piperidyl]-[(3S)-3-(6-methyl-3-pyridyl)isoxazolidin-2-yl]methanone,
(18) [1-[6-(2-methylpyrazol-3-yl)pyrimidin-4-yl]-4-piperidyl]-[(3S)-3-(6-methyl-3-pyridyl)isoxazolidin-2-yl]methanone,
(19) [1-[5-fluoro-4-(2-methylpyrazol-3-yl)pyrimidin-2-yl]-4-piperidyl]-[(3S)-3-(6-methyl-3-pyridyl)isoxazolidin-2-yl]methanone,
(20) [(3S)-3-(5-fluoro-6-methyl-3-pyridyl)isoxazolidin-2-yl]-[1-[4-(2-methylimidazol-1-yl)pyrimidin-2-yl]-4-piperidyl]methanone,
(21) [(3S)-3-(5-fluoro-6-methyl-3-pyridyl)isoxazolidin-2-yl]-[1-[4-(2-methylimidazol-1-yl)pyrimidin-2-yl]-4-piperidyl]methanone,
(22) [1-[4-(2,4-dimethylimidazol-1-yl)-5-fluoro-pyrimidin-2-yl]-4-piperidyl]-[(3S)-3-pyrazin-2-ylisoxazolidin-2-yl]methanone,
(23) [1-[4-(2-methylimidazol-1-yl)-1,3,5-triazin-2-yl]-4-piperidyl]-[(3S)-3-(2-methylthiazol-4-yl)isoxazolidin-2-yl]methanone,
(24) [1-[2-(2-methylpyrazol-3-yl)pyrimidin-4-yl]-4-piperidyl]-[(3S)-3-pyrazin-2-ylisoxazolidin-2-yl]methanone,
(25) [1-(5-fluoro-2-pyrimidin-5-yl-pyrimidin-4-yl)-4-piperidyl]-[(3S)-3-pyrazin-2-ylisoxazolidin-2-yl]methanone,
(26) (S)-(1-(4-(2-methyl-1H-imidazol-1-yl)-1,3,5-triazin-2-yl)piperidin-4-yl)(3-(pyrazin-2-yl)isoxazolidin-2-yl)methanone,
(27) (S)-(1-(5-fluoro-4-(thiazol-5-yl)pyrimidin-2-yl)piperidin-4-yl)(3-(pyrazin-2-yl)isoxazolidin-2-yl)methanone,
(28) (S)-(1-(6-(1-methyl-1H-pyrazol-5-yl)pyrimidin-4-yl)piperidin-4-yl)(3-(pyrazin-2-yl)isoxazolidin-2-yl)methanone,
(29) (S)-(1-(2-(2-methyl-1H-imidazol-1-yl)pyrimidin-4-yl)piperidin-4-yl)(3-(pyrazin-2-yl)isoxazolidin-2-yl)methanone,
(30) (S)-(1-(4-(2-methyl-1H-imidazol-1-yl)pyrimidin-2-yl)piperidin-4-yl)(3-(pyrazin-2-yl)isoxazolidin-2-yl)methanone,
(31) (S)-(1-(6-(2-methyl-1H-imidazol-1-yl)pyrimidin-4-yl)piperidin-4-yl)(3-(pyrazin-2-yl)isoxazolidin-2-yl)methanone,
(32) (S)-(1-(5-fluoro-4-(1H-pyrazol-1-yl)pyrimidin-2-yl)piperidin-4-yl)(3-(pyrazin-2-yl)isoxazolidin-2-yl)methanone,
(33) [5-[6-(2-methylimidazol-1-yl)pyrimidin-4-yl]-5-azaspiro[2.5]octan-8-yl]-[(3S)-3-(6-methylpyridin-3-yl)-1,2-oxazolidin-2-yl]methanone,
(34) [5-[6-(2-methylpyrazol-3-yl)pyrimidin-4-yl]-5-azaspiro[2.5]octan-8-yl]-[(3S)-3-(6-methylpyridin-3-yl)-1,2-oxazolidin-2-yl]methanone,
(35) [1-[4-(2-methylimidazol-1-yl)-1,3,5-triazin-2-yl]piperidin-4-yl]-[(3S)-3-(6-methylpyridin-3-yl)-1,2-oxazolidin-2-yl]methanone,
(36) 3-[2-[4-[(3S)-3-(5-methyl-3-pyridyl)isoxazolidine-2-carbonyl]-1-piperidyl]pyrimidin-4-yl]oxazolidin-2-one,
(37) [(3S)-3-(6-methylpyridin-3-yl)-1,2-oxazolidin-2-yl]-[1-[4-(1,3-oxazol-2-yl)pyrimidin-2-yl]piperidin-4-yl]methanone,
(38) [(3S)-3-(6-methylpyridin-3-yl)-1,2-oxazolidin-2-yl]-[1-[4-(1,3-thiazol-2-yl)-1,3,5-triazin-2-yl]piperidin-4-yl]methanone,
(39) [(8R)-5-[6-(2-methylpyrazol-3-yl)pyrimidin-4-yl]-5-azaspiro[2.5]octan-8-yl]-[(3S)-3-pyrazin-2-yl-1,2-oxazolidin-2-yl]methanone,
(40) [(3S)-3-(2-methyl-1,3-thiazol-4-yl)-1,2-oxazolidin-2-yl]-[1-[6-(2-methyl-1,2,4-triazol-3-yl)pyrimidin-4-yl]piperidin-4-yl]methanone,
(41) [(3S)-3-(2-methyl-1,3-thiazol-4-yl)-1,2-oxazolidin-2-yl]-[(8R)-5-[6-(2-methyl-1,2,4-triazol-3-yl)pyrimidin-4-yl]-5-azaspiro[2.5]octan-8-yl]methanone,
(42) [(3S)-3-(2-methyl-1,3-thiazol-4-yl)-1,2-oxazolidin-2-yl]-[(8R)-5-[6-(3-methyltriazol-4-yl)pyrimidin-4-yl]-5-azaspiro[2.5]octan-8-yl]methanone,
(43) [(3S)-3-(5-fluoro-6-methylpyridin-3-yl)-1,2-oxazolidin-2-yl]-[(8R)-5-[6-(2-methyl-1,2,4-triazol-3-yl)pyrimidin-4-yl]-5-azaspiro[2.5]octan-8-yl]methanone,
(44) 1-[2-[(8R)-8-[(3S)-3-(6-methylpyridin-3-yl)-1,2-oxazolidine-2-carbonyl]-5-azaspiro[2.5]octan-5-yl]pyrimidin-4-yl]pyrrolidin-2-one,
(45) [(3S)-3-(5-fluoro-6-methylpyridin-3-yl)-1,2-oxazolidin-2-yl]-[1-[4-(2-methylpyrazol-(3-yl)-1,3,5-triazin-2-yl]piperidin-4-yl]methanone,
(46) 1-[2-[4-[(3S)-3-(5-fluoropyridin-3-yl)-1,2-oxazolidine-2-carbonyl]piperidin-1-yl]pyrimidin-4-yl]pyrrolidin-2-one,
(47) 1-[2-[4-[(3S)-3-pyrazin-2-yl-1,2-oxazolidine-2-carbonyl]piperidin-1-yl]pyrimidin-4-yl]piperidin-2-one,
(48) [(3S)-3-(5-fluoro-6-methylpyridin-3-yl)-1,2-oxazolidin-2-yl]-[(8 R)-5-[4-(2-methylpyrazol-3-yl)-1,3,5-triazin-2-yl]-5-azaspiro[2.5]octan-8-yl]methanone,
(49) [(3S)-3-(5-fluoro-6-methylpyridin-3-yl)-1,2-oxazolidin-2-yl]-[1-[6-(2-methyl-1,2,4-triazol-3-yl)pyrimidin-4-yl]piperidin-4-yl]methanone,
(50) 1-[2-[4-[(3S)-3-(5-methylpyridin-3-yl)-1,2-oxazolidine-2-carbonyl]piperidin-1-yl]pyrimidin-4-yl]pyrrolidin-2-one,
(51) 5-[2-[4-[(3S)-3-(5-methylpyridin-3-yl)-1,2-oxazolidine-2-carbonyl]piperidin-1-yl]pyrimidin-4-yl]-5-azaspiro[2.4]heptan-4-one,
(52) 5-[2-[4-[(3S)-3-(2-methyl-1,3-thiazol-4-yl)-1,2-oxazolidine-2-carbonyl]piperidin-1-yl]pyrimidin-4-yl]-5-azaspiro[2.4]heptan-4-one,
(53) 5-[2-[4-[(3S)-3-(5-fluoropyridin-3-yl)-1,2-oxazolidine-2-carbonyl]piperidin-1-yl]pyrimidin-4-yl]-5-azaspiro[2.4]heptan-4-one,
(54) 5-[2-[4-[(3S)-3-(2-methyl-1,3-oxazol-4-yl)-1,2-oxazolidine-2-carbonyl]piperidin-1-yl]pyrimidin-4-yl]-5-azaspiro[2.4]heptan-4-one,
(55) 1-[2-[4-[(3S)-3-(5-methylpyrazin-2-yl)-1,2-oxazolidine-2-carbonyl]piperidin-1-yl]pyrimidin-4-yl]pyrrolidin-2-one,
(56) 1-[2-[4-[(3S)-3-(5-fluoro-6-methylpyridin-3-yl)-1,2-oxazolidine-2-carbonyl]piperidin-1-yl]pyrimidin-4-yl]pyrrolidin-2-one,
(57) 5-[2-[4-[(3S)-3-pyrazin-2-yl-1,2-oxazolidine-2-carbonyl]piperidin-1-yl]pyrimidin-4-yl]-5-azaspiro[2.5]octan-4-one,
(58) 3,3-difluoro-1-[2-[4-[(3S)-3-pyrazin-2-yl-1,2-oxazolidine-2-carbonyl]piperidin-1-yl]pyrimidin-4-yl]pyrrolidin-2-one,
(59) 3,3-difluoro-1-[2-[4-[(3S)-3-(2-methyl-1,3-thiazol-4-yl)-1,2-oxazolidine-2-carbonyl]piperidin-1-yl]pyrimidin-4-yl]pyrrolidin-2-one,
(60) 1-[2-[4-[(3S)-3-(5-fluoro-6-methylpyridin-3-yl)-1,2-oxazolidine-2-carbonyl]piperidin-1-yl]pyrimidin-4-yl]-3,3-dimethylpyrrolidin-2-one,
(61) 5-[2-[4-[(3S)-3-(5-fluoro-6-methylpyridin-3-yl)-1,2-oxazolidine-2-carbonyl]piperidin-1-yl]pyrimidin-4-yl]-5-azaspiro[2.4]heptan-4-one,
(62) 1-[2-[4-[(3S)-3-(5-fluoropyridin-3-yl)-1,2-oxazolidine-2-carbonyl]piperidin-1-yl]pyrimidin-4-yl]-3-methylimidazolidin-2-one,
(63) 4-[2-[(8R)-8-[(3S)-3-(6-methylpyridin-3-yl)-1,2-oxazolidine-2-carbonyl]-5-azaspiro[2.5]octan-5-yl]pyrimidin-4-yl]morpholin-3-one,
(64) [1-[5-fluoro-4-(2-methylimidazol-1-yl)pyrimidin-2-yl]piperidin-4-yl]-[(3S)-3-(5-fluoropyridin-3-yl)-1,2-oxazolidin-2-yl]methanone,
(65) 3-[2-[4-[(3S)-3-(5-fluoro-6-methylpyridin-3-yl)-1,2-oxazolidine-2-carbonyl]piperidin-1-yl]pyrimidin-4-yl]-1,3-oxazolidin-2-one,
(66) 3,3-difluoro-1-[2-[4-[(3S)-3-(6-methylpyridin-3-yl)-1,2-oxazolidine-2-carbonyl]piperidin-1-yl]pyrimidin-4-yl]piperidin-2-one,
(67) [1-[5-fluoro-2-(1,3-oxazol-2-yl)pyrimidin-4-yl]piperidin-4-yl]-[(3S)-3-(6-methylpyridin-3-yl)-1,2-oxazolidin-2-yl]methanone,
(68) 5-[5-fluoro-2-[4-[(3S)-3-(5-fluoro-6-methylpyridin-3-yl)-1,2-oxazolidine-2-carbonyl]piperidin-1-yl]pyrimidin-4-yl]-5-azaspiro[2.4]heptan-4-one,
(69) [(8R)-5-[4-(2-methylpyrazol-3-yl)-1,3,5-triazin-2-yl]-5-azaspiro[2.5]octan-8-yl]-[(3S)-3-(6-methylpyridin-3-yl)-1,2-oxazolidin-2-yl]methanone,
(70) 5-[2-[4-[(3S)-3-(6-methylpyridin-3-yl)-1,2-oxazolidine-2-carbonyl]piperidin-1-yl]pyrimidin-4-yl]-5-azaspiro[2.4]heptan-4-one,
(71) 1-[4-[4-[(3S)-3-(5-fluoropyridin-3-yl)-1,2-oxazolidine-2-carbonyl]piperidin-1-yl]pyrimidin-2-yl]pyrrolidin-2-one,
(72) 5-[2-[4-[(3S)-3-(5-fluoropyridin-3-yl)-1,2-oxazolidine-2-carbonyl]piperidin-1-yl]pyrimidin-4-yl]-5-azaspiro[2.5]octan-4-one,
(73) 3,3-difluoro-1-[2-[4-[(3S)-3-(6-methylpyridin-3-yl)-1,2-oxazolidine-2-carbonyl]piperidin-1-yl]pyrimidin-4-yl]pyrrolidin-2-one,
(74) 3,3-difluoro-1-[2-[4-[(3S)-3-(5-methylpyridin-3-yl)-1,2-oxazolidine-2-carbonyl]piperidin-1-yl]pyrimidin-4-yl]pyrrolidin-2-one,
(75) 3,3-difluoro-1-[2-[4-[(3S)-3-(5-fluoropyridin-3-yl)-1,2-oxazolidine-2-carbonyl]piperidin-1-yl]pyrimidin-4-yl]pyrrolidin-2-one,
(76) 3,3-difluoro-1-[2-[4-[(3S)-3-(5-methylpyridin-3-yl)-1,2-oxazolidine-2-carbonyl]piperidin-1-yl]pyrimidin-4-yl]piperidin-2-one,
(77) [1-[5-fluoro-4-(2-methylimidazol-1-yl)pyrimidin-2-yl]piperidin-4-yl]-[(3S)-3-(6-methylpyridin-3-yl)-1,2-oxazolidin-2-yl]methanone,
(78) [5-[6-(2-methylimidazol-1-yl)pyrimidin-4-yl]-5-azaspiro[2.5]octan-8-yl]-[(3S)-3-(6-methylpyridin-3-yl)-1,2-oxazolidin-2-yl]methanone,
(79) [5-[6-(2-methylimidazol-1-yl)pyrimidin-4-yl]-5-azaspiro[2.5]octan-8-yl]-[(3S)-3-(6-methylpyridin-3-yl)-1,2-oxazolidin-2-yl]methanone,
(80) [5-[6-(2-methylpyrazol-3-yl)pyrimidin-4-yl]-5-azaspiro[2.5]octan-8-yl]-[(3S)-3-(6-methylpyridin-3-yl)-1,2-oxazolidin-2-yl]methanone,
(81) 1-[2-[4-[(3S)-3-(6-methylpyridin-3-yl)-1,2-oxazolidine-2-carbonyl]piperidin-1-yl]pyrimidin-4-yl]pyrrolidin-2-one,
(82) 1-[6-[4-[(3S)-3-(6-methylpyridin-3-yl)-1,2-oxazolidine-2-carbonyl]piperidin-1-yl]pyrimidin-4-yl]pyrrolidin-2-one,
(83) [(8R)-5-[4-(4-methylpyrazol-1-yl)pyrimidin-2-yl]-5-azaspiro[2.5]octan-8-yl]-[(3 S)-3-pyrazin-2-yl-1,2-oxazolidin-2-yl]methanone,
(84) 3,3-dimethyl-1-[4-[4-[(3S)-3-(6-methylpyridin-3-yl)-1,2-oxazolidine-2-carbonyl]piperidin-1-yl]pyrimidin-2-yl]pyrrolidin-2-one,
(85) 1-[4-[(8R)-8-[(3S)-3-(6-methylpyridin-3-yl)-1,2-oxazolidine-2-carbonyl]-5-azaspiro[2.5]octan-5-yl]pyrimidin-2-yl]pyrrolidin-2-one,
(86) 1-[6-[4-[(3S)-3-(5-fluoropyridin-3-yl)-1,2-oxazolidine-2-carbonyl]piperidin-1-yl]pyrimidin-4-yl]pyrrolidin-2-one,
(87) 1-[4-[4-[(3S)-3-(6-methylpyridin-3-yl)-1,2-oxazolidine-2-carbonyl]piperidin-1-yl]pyrimidin-2-yl]pyrrolidin-2-one,
(88) 5,5-dimethyl-1-[2-[4-[(3S)-3-(6-methylpyridin-3-yl)-1,2-oxazolidine-2-carbonyl]piperidin-1-yl]pyrimidin-4-yl]pyrrolidin-2-one,
(89) 1-[2-[4-[(3S)-3-(5-fluoro-6-methylpyridin-3-yl)-1,2-oxazolidine-2-carbonyl]piperidin-1-yl]pyrimidin-4-yl]-5,5-dimethylpyrrolidin-2-one,
(90) 5-[4-[4-[(3S)-3-(6-methylpyridin-3-yl)-1,2-oxazolidine-2-carbonyl]piperidin-1-yl]pyrimidin-2-yl]-5-azaspiro[2.5]octan-4-one,
(91) 3-[2-[4-[(3 S)-3-(5-fluoropyridin-3-yl)-1,2-oxazolidine-2-carbonyl]piperidin-1-yl]pyrimidin-4-yl]-1,3-oxazolidin-2-one,
(92) 1-[2-[4-[(3S)-3-(2-methyl-1,3-thiazol-4-yl)-1,2-oxazolidine-2-carbonyl]piperidin-1-yl]pyrimidin-4-yl]pyrrolidin-2-one,
(93) (3R)-3-methoxy-1-[2-[4-[(3S)-3-(6-methylpyridin-3-yl)-1,2-oxazolidine-2-carbonyl]piperidin-1-yl]pyrimidin-4-yl]piperidin-2-one,
(94) (3S)-3-methoxy-1-[2-[4-[(3S)-3-(6-methylpyridin-3-yl)-1,2-oxazolidine-2-carbonyl]piperidin-1-yl]pyrimidin-4-yl]piperidin-2-one,
(95) 1-[2-[4-[(3S)-3-(2-methyl-1,3-oxazol-4-yl)-1,2-oxazolidine-2-carbonyl]piperidin-1-yl]pyrimidin-4-yl]pyrrolidin-2-one,
(96) (3R)-1-[2-[4-[(3S)-3-(5-fluoropyridin-3-yl)-1,2-oxazolidine-2-carbonyl]piperidin-1-yl]pyrimidin-4-yl]-3-methoxypiperidin-2-one,
(97) (3S)-1-[2-[4-[(3S)-3-(5-fluoropyridin-3-yl)-1,2-oxazolidine-2-carbonyl]piperidin-1-yl]pyrimidin-4-yl]-3-methoxypiperidin-2-one,
(98) [(3S)-3-(5-fluoro-6-methylpyridin-3-yl)-1,2-oxazolidin-2-yl]-[1-[4-(oxetan-2-yl)pyrimidin-2-yl]piperidin-4-yl]methanone,
(99) [(3S)-3-(5-fluoro-6-methylpyridin-3-yl)-1,2-oxazolidin-2-yl]-[1-[4-(oxetan-2-yl)pyrimidin-2-yl]piperidin-4-yl]methanone,
(100) 1-[2-[(8R)-8-[(3S)-3-pyrazin-2-yl-1,2-oxazolidine-2-carbonyl]-5-azaspiro[2.5]octan-5-yl]pyrimidin-4-yl]pyrrolidin-2-one,
(101) [(3S)-3-(5-fluoro-6-methylpyridin-3-yl)-1,2-oxazolidin-2-yl]-[1-[4-(oxetan-3-yl)pyrimidin-2-yl]piperidin-4-yl]methanone,
(102) [1-[4-(5-amino-2-methylpyrazol-3-yl)pyrimidin-2-yl]piperidin-4-yl]-[(3S)-3-(5-fluoro-6-methylpyridin-3-yl)-1,2-oxazolidin-2-yl]methanone,
(103) 5-[5-fluoro-2-[4-[(3S)-3-(5-methylpyridin-3-yl)-1,2-oxazolidine-2-carbonyl]piperidin-1-yl]pyrimidin-4-yl]-5-azaspiro[2.4]heptan-4-one,
(104) 5-[5-fluoro-2-[4-[(3S)-3-(5-fluoropyridin-3-yl)-1,2-oxazolidine-2-carbonyl]piperidin-1-yl]pyrimidin-4-yl]-5-azaspiro[2.4]heptan-4-one,
(105) 5-[5-fluoro-2-[4-[(3S)-3-(5-methylpyrazin-2-yl)-1,2-oxazolidine-2-carbonyl]piperidin-1-yl]pyrimidin-4-yl]-5-azaspiro[2.4]heptan-4-one,
(106) [1-[4-[(3S)-3-fluoropyrrolidin-1-yl]pyrimidin-2-yl]piperidin-4-yl]-[(3S3-(5-methylpyrazin-2-yl)-1,2-oxazolidin-2-yl]methanone,
(107) [1-[4-[(3R)-3-methoxypyrrolidin-1-yl]pyrimidin-2-yl]piperidin-4-yl]-[(3S)-3-(5-methylpyrazin-2-yl)-1,2-oxazolidin-2-yl]methanone
(108) [1-[4-[(3S)-3-methoxypyrrolidin-1-yl]pyrimidin-2-yl]piperidin-4-yl]-[(3 S)-3-(5-methylpyrazin-2-yl)-1,2-oxazolidin-2-yl]methanone,
(109) 5-[5-fluoro-2-[4-[(3S)-3-(6-methylpyridin-3-yl)-1,2-oxazolidine-2-carbonyl]piperidin-1-yl]pyrimidin-4-yl]-5-azaspiro[2.4]heptan-4-one,
(110) 1-[5-fluoro-2-[4-[(3S)-3-(5-methylpyrazin-2-yl)-1,2-oxazolidine-2-carbonyl]piperidin-1-yl]pyrimidin-4-yl]pyrrolidin-2-one,
(111) [1-[4-(4-amino-5-methylpyrazol-1-yl)pyrimidin-2-yl]piperidin-4-yl]-[(3S)-3-(5-fluoro-6-methylpyridin-3-yl)-1,2-oxazolidin-2-yl]methanone,
(112) [1-[4-(4-amino-5-methylpyrazol-1-yl)pyrimidin-2-yl]piperidin-4-yl]-[(3S)-3-(6-methylpyridin-3-yl)-1,2-oxazolidin-2-yl]methanone,
(113) [1-[4-(3-methoxyazetidin-1-yl)pyrimidin-2-yl]piperidin-4-yl]-[(3S)-3-(5-methylpyrazin-2-yl)-1,2-oxazolidin-2-yl]methanone,
(114) 3-[5-fluoro-2-[4-[(3S)-3-(5-fluoropyridin-3-yl)-1,2-oxazolidine-2-carbonyl]piperidin-1-yl]pyrimidin-4-yl]-1,3-oxazolidin-2-one,
(115) 3-[5-fluoro-2-[4-[(3S)-3-(5-fluoro-6-methylpyridin-3-yl)-1,2-oxazolidine-2-carbonyl]piperidin-1-yl]pyrimidin-4-yl]-1,3-oxazolidin-2-one, and
(116) 3-[5-fluoro-2-[4-[(3S)-3-(5-methylpyridin-3-yl)-1,2-oxazolidine-2-carbonyl]piperidin-1-yl]pyrimidin-4-yl]-1,3-oxazolidin-2-one,
or a pharmaceutically acceptable salt, solvate, or stereoisomer thereof.

8. The compound of of claim 7, selected from the group consisting of compounds (3), (11), (14), (15), (38), (39), (40), (42), (43), (45), (48), (49), (52), (53), (54), (55), (57), (62), (65), (69) and (91), or a pharmaceutically acceptable salt, solvate, or stereoisomer thereof.

9. The compound of claim 8, wherein the compound is selected from the group consisting of compounds (3), (11), (14), (15), (38), (39), (40), (42), (43), (45) and (48), or a pharmaceutically acceptable salt, solvate, or stereoisomer thereof.

10. A pharmaceutical composition comprising the compound of claim 1, or a pharmaceutically acceptable salt, solvate, or stereoisomer thereof and at least one pharmaceutically acceptable excipient.

11. (canceled)

12. A method of treating or preventing a disease, disorder, or condition that is at least partly mediated by receptor-interacting protein kinase in a subject in need thereof, said method comprising administering to said subject a therapeutically effective amount of the compound of claim 1, or a pharmaceutically acceptable salt, solvate, or stereoisomer thereof.

13. A method of treating or preventing Parkinson's disease, Alzheimer's disease, amyotrophic lateral sclerosis (ALS)_{z} or, multiple sclerosis (MS) in a subject in need thereof, said method comprising administering to said subject a therapeutically effective amount of the compound of claim 1, or a pharmaceutically acceptable salt, solvate, or stereoisomer thereof.

14. The compound of claim 2, or a pharmaceutically acceptable salt thereof, wherein R₂ represents is an imidazolyl, a pyrazolyl, a triazolyl, an oxazolyl, a thiazolyl, a pyridinyl, or a pyrimidinyl group, each of which is optionally substituted by 1 or 2 R₇, wherein each R₇ is independently selected from a CH₃ group and a halogen.

15. The compound of claim 2, or a pharmaceutically acceptable salt thereof, wherein R₂ is a piperidinyl, a morpholinyl, a pyrrolidinyl, a oxazolidinyl, an azetidinyl or an oxetanyl group, each of which is optionally substituted by 1 or 2 R₈, wherein each R₈ is independently selected from OH, oxo, a halogen, a (C₁-C₂)alkyl group, a O—(C₁-C₂)alkyl group, and CF₃.

16. The compound of claim 3, or a pharmaceutically acceptable salt thereof, wherein R₇ is a CH₃ group.

17. The compound of claim 4, or a pharmaceutically acceptable salt thereof, wherein R₈ is oxo.

18. The compound of claim 14, or a pharmaceutically acceptable salt thereof, wherein R₇ is a CH₃ group.

19. The compound of claim 15, or a pharmaceutically acceptable salt thereof, wherein R₈ is oxo.

20. A pharmaceutical composition comprising the compound of claim 7, or a pharmaceutically acceptable salt, solvate, or stereoisomer thereof and at least one pharmaceutically acceptable excipient.
